SEQUENCE LISTING (84 sequences)

SEQ ID NO: 1 - protein, 73 aa, Homo sapiens
  MISC_FEATURE: Human Gro-beta
APLATELRCQCLQTLQGIHLKNIQSVKVKSPGPHCAQTEVIATLKNGQKACLNPASPMVKKIIEKMLKNGKSN

SEQ ID NO: 2 - protein, 107 aa, Homo sapiens
  MISC_FEATURE: UniProt ID No. P19875- human GRO-beta
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 3 - protein, 69 aa, Homo sapiens
  MISC_FEATURE: GRO-beta-delta-4
TELRCQCLQTLQGIHLKNIQSVKVKSPGPHCAQTEVIATLKNGQKACLNPASPMVKKIIEKMLKNGKSN

SEQ ID NO: 4 - protein, 380 aa, Homo sapiens
  MISC_FEATURE: FOS
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 5 - protein, 652 aa, Homo sapiens
  MISC_FEATURE: CD93
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 6 - protein, 338 aa, Homo sapiens
  MISC_FEATURE: FOSB
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETLTPEEEEKRRVRRERNKLAAAKCRNRRRELTDRLQAETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 7 - protein, 367 aa, Homo sapiens
  MISC_FEATURE: Dusp1
MVMEVGTLDAGGLRALLGERAAQCLLLDCRSFFAFNAGHIAGSVNVRFSTIVRRRAKGAMGLEHIVPNAELRGRLLAGAYHAVVLLDERSAALDGAKRDGTLALAAGALCREARAAQVFFLKGGYEAFSASCPELCSKQSTPMGLSLPLSTSVPDSAESGCSSCSTPLYDQGGPVEILPFLYLGSAYHASRKDMLDALGITALINVSANCPNHFEGHYQYKSIPVEDNHKADISSWFNEAIDFIDSIKNAGGRVFVHCQAGISRSATICLAYLMRTNRVKLDEAFEFVKQRRSIISPNFSFMGQLLQFESQVLAPHCSAEAGSPAMAVLDRGTSTTTVFNFPVSIPVHSTNSALSYLQSPITTSPSC

SEQ ID NO: 8 - protein, 331 aa, Homo sapiens
  MISC_FEATURE: Jun
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 9 - protein, 381 aa, Homo sapiens
  MISC_FEATURE: DUSP6
MIDTLRPVPFASEMAISKTVAWLNEQLELGNERLLLMDCRPQELYESSHIESAINVAIPGIMLRRLQKGNLPVRALFTRGEDRDRFTRRCGTDTVVLYDESSSDWNENTGGESVLGLLLKKLKDEGCRAFYLEGGFSKFQAEFSLHCETNLDGSCSSSSPPLPVLGLGGLRISSDSSSDIESDLDRDPNSATDSDGSPLSNSQPSFPVEILPFLYLGCAKDSTNLDVLEEFGIKYILNVTPNLPNLFENAGEFKYKQIPISDHWSQNLSQFFPEAISFIDEARGKNCGVLVHCLAGISRSVTVTVAYLMQKLNLSMNDAYDIVKMKKSNISPNFNFMGQLLDFERTLGLSSPCDNRVPAQQLYFTTPSNQNVYQVDSLQST

SEQ ID NO: 10 - protein, 297 aa, Homo sapiens
  MISC_FEATURE: CDK1
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 11 - protein, 674 aa, Homo sapiens
  MISC_FEATURE: Fignl1
MQTSSSRSVHLSEWQKNYFAITSGICTGPKADAYRAQILRIQYAWANSEISQVCATKLFKKYAEKYSAIIDSDNVESGLNNYAENILTLAGSQQTDSDKWQSGLSINNVFKMSSVQKMMQAGKKFKDSLLEPALASVVIHKEATVFDLPKFSVCGSSQESDSLPNSAHDRDRTQDFPESNRLKLLQNAQPPMVTNTARTCPTFSAPVGESATAKFHVTPLFGNVKKENHSSAKENIGLNVFLSNQSCFPAACENPQRKSFYGSGTIDALSNPILNKACSKTEDNGPKEDSSLPTFKTAKEQLWVDQQKKYHQPQRASGSSYGGVKKSLGASRSRGILGKFVPPIPKQDGGEQNGGMQCKPYGAGPTEPAHPVDERLKNLEPKMIELIMNEIMDHGPPVNWEDIAGVEFAKATIKEIVVWPMLRPDIFTGLRGPPKGILLFGPPGTGKTLIGKCIASQSGATFFSISASSLTSKWVGEGEKMVRALFAVARCQQPAVIFIDEIDSLLSQRGDGEHESSRRIKTEFLVQLDGATTSSEDRILVVGATNRPQEIDEAARRRLVKRLYIPLPEASARKQIVINLMSKEQCCLSEEEIEQIVQQSDAFSGADMTQLCREASLGPIRSLQTADIATITPDQVRPIAYIDFENAFRTVRPSVSPKDLELYENWNKTFGCGK

SEQ ID NO: 12 - protein, 685 aa, Homo sapiens
  MISC_FEATURE: Plk2
MELLRTITYQPAASTKMCEQALGKGCGADSKKKRPPQPPEESQPPQSQAQVPPAAPHHHHHHSHSGPEISRIIVDPTTGKRYCRGKVLGKGGFAKCYEMTDLTNNKVYAAKIIPHSRVAKPHQREKIDKEIELHRILHHKHVVQFYHYFEDKENIYILLEYCSRRSMAHILKARKVLTEPEVRYYLRQIVSGLKYLHEQEILHRDLKLGNFFINEAMELKVGDFGLAARLEPLEHRRRTICGTPNYLSPEVLNKQGHGCESDIWALGCVMYTMLLGRPPFETTNLKETYRCIREARYTMPSSLLAPAKHLIASMLSKNPEDRPSLDDIIRHDFFLQGFTPDRLSSSCCHTVPDFHLSSPAKNFFKKAAAALFGGKKDKARYIDTHNRVSKEDEDIYKLRHDLKKTSITQQPSKHRTDEELQPPTTTVARSGTPAVENKQQIGDAIRMIVRGTLGSCSSSSECLEDSTMGSVADTVARVLRGCLENMPEADCIPKEQLSTSFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICSQNEEYLLTYINEDRISTTFRLTTLLMSGCSSELKNRMEYALNMLLQRCN

SEQ ID NO: 13 - protein, 361 aa, Homo sapiens
  MISC_FEATURE: RSAD2
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 14 - protein, 431 aa, Homo sapiens
  MISC_FEATURE: SGK1
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 15 - protein, 310 aa, Homo sapiens
  MISC_FEATURE: Sdc1
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 16 - protein, 398 aa, Homo sapiens
  MISC_FEATURE: Serpine2
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITTGSENLHVSHILQKAKIEVSEDGTKASAATTAILIARSSPPWFIVDRPFLFFIRHNPTGAVLFMGQINKP

SEQ ID NO: 17 - protein, 314 aa, Homo sapiens
  MISC_FEATURE: Spp1
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 18 - protein, 280 aa, Homo sapiens
  MISC_FEATURE: Cdca8
MAPRKGSSRVAKTNSLRRRKLASFLKDFDREVEIRIKQIESDRQNLLKEVDNLYNIEILRLPKALREMNWLDYFALGGNKQALEEAATADLDITEINKLTAEAIQTPLKSAKTRKVIQVDEMIVEEEEEEENERKNLQTARVKRCPPSKKRTQSIQGKGKGKRSSRANTVTPAVGRLEVSMVKPTPGLTPRFDSRVFKTPGLRTPAAGERIYNISGNGSPLADSKEIFLTVPVGGGESLRLLASDLQRHSIAQLDPEALGNIKKLSNRLAQICSSIRTHK

SEQ ID NO: 19 - protein, 923 aa, Homo sapiens
  MISC_FEATURE: Nrp1
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA

SEQ ID NO: 20 - protein, 646 aa, Homo sapiens
  MISC_FEATURE: Mcam
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 21 - protein, 322 aa, Homo sapiens
  MISC_FEATURE: Pbk
MEGISNFKTPSKLSEKKKSVLCSTPTINIPASPFMQKLGFGTGVNVYLMKRSPRGLSHSPWAVKKINPICNDHYRSVYQKRLMDEAKILKSLHHPNIVGYRAFTEANDGSLCLAMEYGGEKSLNDLIEERYKASQDPFPAAIILKVALNMARGLKYLHQEKKLLHGDIKSSNVVIKGDFETIKICDVGVSLPLDENMTVTDPEACYIGTEPWKPKEAVEENGVITDKADIFAFGLTLWEMMTLSIPHINLSNDDDDEDKTFDESDFDDEAYYAALGTRPPINMEELDESYQKVIELFSVCTNEDPKDRPSAAHIVEALETDV

SEQ ID NO: 22 - protein, 262 aa, Mus musculus
  MISC_FEATURE: Akr1cl
GLAIRSKVADGTVRREDIFYTSKLPCTCHRPELVQPCLEQSLRKLQLDYVDLYLIHCPVSMKPGNDLIPTDENGKLLFDTVDLCDTWEAMEKCKDSGLAKSIGVSNFNRRQLEMILNKPGLRYKPVCNQVECHPYLNQSKLLDYCKSKDIVLVAYGALGSQRCKNWIEENAPYLLEDPTLCAMAEKHKQTPALISLRYLLQRGIVIVTKSFNEKRIKENLKVFEFHLPAEDMAVIDRLNRNYRYATARIISAHPNYPFLDEY

SEQ ID NO: 23 - protein, 521 aa, Homo sapiens
  MISC_FEATURE: Cyp11a1
MLAKGLPPRSVLVKGCQTFLSAPREGLGRLRVPTGEGAGISTRSPRPFNEIPSPGDNGWLNLYHFWRETGTHKVHLHHVQNFQKYGPIYREKLGNVESVYVIDPEDVALLFKSEGPNPERFLIPPWVAYHQYYQRPIGVLLKKSAAWKKDRVALNQEVMAPEATKNFLPLLDAVSRDFVSVLHRRIKKAGSGNYSGDISDDLFRFAFESITNVIFGERQGMLEEVVNPEAQRFIDAIYQMFHTSVPMLNLPPDLFRLFRTKTWKDHVAAWDVIFSKADIYTQNFYWELRQKGSVHHDYRGILYRLLGDSKMSFEDIKANVTEMLAGGVDTTSMTLQWHLYEMARNLKVQDMLRAEVLAARHQAQGDMATMLQLVPLLKASIKETLRLHPISVTLQRYLVNDLVLRDYMIPAKTLVQVAIYALGREPTFFFDPENFDPTRWLSKDKNITYFRNLGFGWGVRQCLGRRIAELEMTIFLINMLENFRVEIQHLSDVGTTFNLILMPEKPISFTFWPFNQEATQQ

SEQ ID NO: 24 - DNA, 1918 bp, Homo sapiens
  misc_feature: human FOS
aaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcaccctttcggagtccccgccccctccgctggggcttactccagggctggcgttgtgaagaccatgacaggaggccgagcgcagagcattggcaggaggggcaaggtggaacagttatctcctgaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcg

SEQ ID NO: 25 - protein, 380 aa, Mus musculus
  MISC_FEATURE: mouse FOS
MMFSGFNADYEASSSRCSSASPAGDSLSYYHSPADSFSSMGSPVNTQDFCADLSVSSANFIPTVTAISTSPDLQWLVQPTLVSSVAPSQTRAPHPYGLPTQSAGAYARAGMVKTVSGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEASTPESEEAFTLPLLNDPEPKPSLEPVKSISNVELKAEPFDDFLFPASSRPSGSETSRSVPDVDLSGSFYAADWEPLHSNSLGMGPMVTELEPLCTPVVTCTPGCTTYTSSFVFTYPEADSFPSCAAAHRKGSSSNEPSSDSLSSPTLLAL

SEQ ID NO: 26 - DNA, 2107 bp, Mus musculus
  misc_feature: FOS
cagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccacccagtctgctggggcttacgccagagcgggaatggtgaagaccgtgtcaggaggcagagcgcagagcatcggcagaaggggcaaagtagagcagctatctcctgaagaggaagagaaacggagaatccgaagggaacggaataagatggctgcagccaagtgccggaatcggaggagggagctgacagatacactccaagcggagacagatcaacttgaagatgagaagtctgcgttgcagactgagattgccaatctgctgaaagagaaggaaaaactggagtttattttggcagcccaccgacctgcctgcaagatccccgatgaccttggcttcccagaggagatgtctgtggcctccctggatttgactggaggtctgcctgaggcttccaccccagagtctgaggaggccttcaccctgccccttctcaacgaccctgagcccaagccatccttggagccagtcaagagcatcagcaacgtggagctgaaggcagaaccctttgatgacttcttgtttccggcatcatctaggcccagtggctcagagacctcccgctctgtgccagatgtggacctgtccggttccttctatgcagcagactgggagcctctgcacagcaattccttggggatggggcccatggtcacagagctggagcccctgtgtactcccgtggtcacctgtactccgggctgcactacttacacgtcttcctttgtcttcacctaccctgaagctgactccttcccaagctgtgccgctgcccaccgaaagggcagcagcagcaacgagccctcctccgactccctgagctcacccacgctgctggccctgtgagcagtcagagaaggcaaggcagccggcatccagacgtgccactgcccgagctggtgcattacagagaggagaaacacgtcttccctcgaaggttcccgtcgacctagggaggaccttacctgttcgtgaaacacaccaggctgtgggcctcaaggacttgcaagcatccacatctggcctccagtcctcacctcttccagagatgtagcaaaaacaaaacaaaacaaaacaaaaaaccgcatggagtgtgttgttcctagtgacacctgagagctggtagttagtagagcatgtgagtcaaggcctggtctgtgtctcttttctctttctccttagttttctcatagcactaactaatctgttgggttcattattggaattaacctggtgctggattgtatctagtgcagctgattttaacaatacctactgtgttcctggcaatagcgtgttccaattagaaacgaccaatattaaactaagaaaagataggactttattttccagtagatagaaatcaatagctatatccatgtactgtagtccttcagcgtcaatgttcattgtcatgttactgatcatgcattgtcgaggtggtctgaatgttctgacattaacagttttccatgaaaacgtttttattgtgttttcaatttatttattaagatggattctcagatatttatatttttattttatttttttctaccctgaggtctttcgacatgtggaaagtgaatttgaatgaaaaattttaagcattgtttgcttattgttccaagacattgtcaataaaagcatttaagttgaaaaaaaaaaaaaaaa

SEQ ID NO: 27 - DNA, 2577 bp, Homo sapiens
  misc_feature: CD93
cttctctgcgccggagtggctgcagctcacccctcagctccccttggggcccagctgggagccgagatagaagctcctgtcgccgctgggcttctcgcctcccgcagagggccacacagagaccgggatggccacctccatgggcctgctgctgctgctgctgctgctcctgacccagcccggggcggggacgggagctgacacggaggcggtggtctgcgtggggaccgcctgctacacggcccactcgggcaagctgagcgctgccgaggcccagaaccactgcaaccagaacgggggcaacctggccactgtgaagagcaaggaggaggcccagcacgtccagcgagtactggcccagctcctgaggcgggaggcagccctgacggcgaggatgagcaagttctggattgggctccagcgagagaagggcaagtgcctggaccctagtctgccgctgaagggcttcagctgggtgggcgggggggaggacacgccttactctaactggcacaaggagctccggaactcgtgcatctccaagcgctgtgtgtctctgctgctggacctgtcccagccgctccttcccagccgcctccccaagtggtctgagggcccctgtgggagcccaggctcccccggaagtaacattgagggcttcgtgtgcaagttcagcttcaaaggcatgtgccggcctctggccctggggggcccaggtcaggtgacctacaccacccccttccagaccaccagttcctccttggaggctgtgccctttgcctctgcggccaatgtagcctgtggggaaggtgacaaggacgagactcagagtcattatttcctgtgcaaggagaaggcccccgatgtgttcgactggggcagctcgggccccctctgtgtcagccccaagtatggctgcaacttcaacaatgggggctgccaccaggactgctttgaagggggggatggctccttcctctgcggctgccgaccaggattccggctgctggatgacctggtgacctgtgcctctcgaaacccttgcagctccagcccatgtcgtgggggggccacgtgcgtcctgggaccccatgggaaaaactacacgtgccgctgcccccaagggtaccagctggactcgagtcagctggactgtgtggacgtggatgaatgccaggactccccctgtgcccaggagtgtgtcaacacccctgggggcttccgctgcgaatgctgggttggctatgagccgggcggtcctggagagggggcctgtcaggatgtggatgagtgtgctctgggtcgctcgccttgcgcccagggctgcaccaacacagatggctcatttcactgctcctgtgaggagggctacgtcctggccggggaggacgggactcagtgccaggacgtggatgagtgtgtgggcccggggggccccctctgcgacagcttgtgcttcaacacacaagggtccttccactgtggctgcctgccaggctgggtgctggccccaaatggggtctcttgcaccatggggcctgtgtctctgggaccaccatctgggccccccgatgaggaggacaaaggagagaaagaagggagcaccgtgccccgtgctgcaacagccagtcccacaaggggccccgagggcacccccaaggctacacccaccacaagtagaccttcgctgtcatctgacgcccccatcacatctgccccactcaagatgctggcccccagtgggtccccaggcgtctggagggagcccagcatccatcacgccacagctgcctctggcccccaggagcctgcaggtggggactcctccgtggccacacaaaacaacgatggcactgacgggcaaaagctgcttttattctacatcctaggcaccgtggtggccatcctactcctgctggccctggctctggggctactggtctatcgcaagcggagagcgaagagggaggagaagaaggagaagaagccccagaatgcggcagacagttactcctgggttccagagcgagctgagagcagggccatggagaaccagtacagtccgacacctgggacagactgctgaaagtgaggtggccctagagacactagagtcaccagccaccatcctcagagctttgaactccccattccaaaggggcacccacatttttttgaaagactggactggaatcttagcaaacaattgtaagtctcctccttaaaggccccttggaacatgcaggtattttctacgggtgtttgatgttcctgaagtggaagctgtgtgttggcgtgccacggtggggatttcgtgactctataatgattgttactccccctcccttttcaaattccaatgtgaccaattccggatcagggtgtgaggaggccggggctaaggggctcccctgaatatcttctctgctcacttccaccatctaagaggaaaaggtgagttgctcatgctgattaggattgaaatgatttgtttctcttcctaggatgaaaactaaatcaattaattattcaaaaaaaaaaaaaaaaaaaaaaaaaaaaaaaaaaaaaaaaaaaaaa

SEQ ID NO: 28 - protein, 644 aa, Mus musculus
  MISC_FEATURE: CD93
MAISTGLFLLLGLLGQPWAGAAADSQAVVCEGTACYTAHWGKLSAAEAQHRCNENGGNLATVKSEEEARHVQQALTQLLKTKAPLEAKMGKFWIGLQREKGNCTYHDLPMRGFSWVGGGEDTAYSNWYKASKSSCIFKRCVSLILDLSLTPHPSHLPKWHESPCGTPEAPGNSIEGFLCKFNFKGMCRPLALGGPGRVTYTTPFQATTSSLEAVPFASVANVACGDEAKSETHYFLCNEKTPGIFHWGSSGPLCVSPKFGCSFNNGGCQQDCFEGGDGSFRCGCRPGFRLLDDLVTCASRNPCSSNPCTGGGMCHSVPLSENYTCRCPSGYQLDSSQVHCVDIDECQDSPCAQDCVNTLGSFHCECWVGYQPSGPKEEACEDVDECAAANSPCAQGCINTDGSFYCSCKEGYIVSGEDSTQCEDIDECSDARGNPCDSLCFNTDGSFRCGCPPGWELAPNGVFCSRGTVFSELPARPPQKEDNDDRKESTMPPTEMPSSPSGSKDVSNRAQTTGLFVQSDIPTASVPLEIEIPSEVSDVWFELGTYLPTTSGHSKPTHEDSVSAHSDTDGQNLLLFYILGTVVAISLLLVLALGILIYHKRRAKKEEIKEKKPQNAADSYSWVPERAESQAPENQYSPTPGTDC

SEQ ID NO: 29 - DNA, 3070 bp, Mus musculus
  misc_feature: CD93
gaaagcagcagtgcgcctctgctcccttcagagcacagcctggtgtcaaggtccaggttccaccggctgctgctgtcaccgcaggggagtctagcccctcccagaaggagacacagaagaatggccatctcaactggtttgttcctgctgctggggctccttggccagccctgggcaggggctgctgctgattcacaggctgtggtgtgcgaggggactgcctgctatacagcccattggggcaagctgagtgccgctgaagcccagcatcgctgcaatgagaatggaggcaatcttgccaccgtgaagagtgaggaggaggcccggcatgttcagcaagccctgactcagctcctgaagaccaaggcacccttggaagcaaagatgggcaaattctggatcgggctccagcgagagaagggcaactgtacgtaccatgatttgccaatgaggggcttcagctgggtgggtggtggagaggacacagcttattcaaactggtacaaagccagcaagagctcctgtatctttaaacgctgtgtgtccctcatactggacctgtccttgacacctcaccccagccatctgcccaagtggcatgagagtccctgtgggacccccgaagctccaggtaacagcattgaaggtttcctgtgcaagttcaacttcaaaggcatgtgtaggccactggcgctgggtggtccagggcgggtgacctataccacccctttccaggccactacctcctctctggaggctgtgccttttgcctctgtagccaatgtagcttgtggggatgaagctaagagtgaaacccactatttcctatgcaatgaaaagactccaggaatatttcactggggcagctcaggcccactctgtgtcagccccaagtttggttgcagtttcaacaacgggggctgccagcaggattgcttcgaaggtggcgatggctccttccgctgcggctgccggcctggatttcgactgctggatgatctagtaacttgtgcctccaggaacccctgcagctcaaacccatgcacaggaggtggcatgtgccattctgtaccactcagtgaaaactacacttgccgttgtcccagcggctaccagctggactctagccaagtgcactgtgtggatatagatgagtgccaggactccccctgtgcccaggattgtgtcaacactctagggagcttccactgtgaatgttgggttggttaccaacccagtggccccaaggaagaggcctgtgaagatgtggatgagtgtgcagctgccaactcgccctgtgcccaaggctgcatcaacactgatggctctttctactgctcctgtaaagagggctatattgtgtctggggaagacagtacccagtgtgaggatatagatgagtgttcggacgcaaggggcaatccatgtgattccctgtgcttcaacacagatggttccttcaggtgtggctgcccgccaggctgggagctggctcccaatggggtcttttgtagcaggggcactgtgttttctgaactaccagccaggcctccccaaaaggaagacaacgatgacagaaaggagagtactatgcctcctactgaaatgcccagttctcctagtggctctaaggatgtctccaacagagcacagacaacaggtctcttcgtccaatcagatattcccactgcctctgttccactagaaatagaaatccctagtgaagtatctgatgtctggttcgagttgggcacatacctccccacgacctccggccacagcaagccgacacatgaagattctgtgtctgcacacagtgacaccgatgggcagaacctgcttctgttttacatcctggggacggtggtggccatctcactcttgctggtgctggccctagggattctcatttatcataaacggagagccaagaaggaggagataaaagagaagaagcctcagaatgcagccgacagctattcctgggttccagagcgagcagagagccaagccccggagaatcagtacagcccaacaccagggacagactgctgaagactatgtggccttagagacagctgccactaccttcagagctaccttcttagatgagggggaagccacatcattctgaatgacttgactggactctcagcaaaaaaattgtgcaccttccacttaagaacctggtggcttgggataggcaggtattttcttggtgcctttgatatgtctgggggtgaaagctgtgtgttggtttgtcattgtggggagttttgtggatattgacagacctcactcaaacacccttttcaaatccaatagcaactggttcctctggttcctaattagggggaaaggagtcagaggggtgggacagggtggggggatggggcttcaaagttttttcttatcacttgatttatcatcgaaggagttactggtgctaattacaatggaaacagttcctttccatcacaggacagacacacctcaatcctccatggggtcaacaactatatacccccagtgaccccttaggcaaggacttgttgagaactgcatcacattttgacctgttctcaacagtacccatctatttcaggtgggatctctggacctttcctccttcccatcttgtctgcaatgtggcaaatggcttctttttgcatttttactccgcccccaccccaagctgaagttcatttgcagatcagcgattaagtctgaattgtgtggtggtcagtcttgtttccttttgtcaggggttattgtaaatgttagtaatttcgcctcaagccctcagtaagaacataaatattttaaaatatgtgcgtttgaaatctgtttcatgcatcctggaactgtgggatgctcaggcaagagtgactttagtctttcagtgaatgttgcccagaatgtgggtagggaaggctcacaggttactctcctccttagagctacaacataacattctgaggggagtcacagggttgcctttaaaaagtgggagctatgtcatgctttgagctttctgttaagcacctctcctaataaactctgaaaaaat

SEQ ID NO: 30 - DNA, 3775 bp, Homo sapiens
  misc_feature: FOSB
cattcataagactcagagctacggccacggcagggacacgcggaaccaagacttggaaacttgattgttgtggttcttcttgggggttatgaaatttcattaatcttttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgcggcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacgctcaccccagaggaagaggagaagcgaagggtgcgccgggaacgaaataaactagcagcagctaaatgcaggaaccggcggagggagctgaccgaccgactccaggcggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctcggtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggatgaagggacgggggtgggaggtaggctgtggggtgggctggagtcctctccagagaggctcaacaaggaaaaatgccactccctacccaatgtctcccacacccaccctttttttggggtgcccaggttggtttcccctgcactcccgaccttagcttattgatcccacatttccatggtgtgagatcctctttactctgggcagaagtgagcccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaaggcagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgc

SEQ ID NO: 31 - DNA, 4145 bp, Mus musculus
  misc_feature: FOSB
  misc_feature (4045)..(4045): n is a, c, g, or t
ataaattcttattttgacactcaccaaaatagtcacctggaaaacccgctttttgtgacaaagtacagaaggcttggtcacatttaaatcactgagaactagagagaaatactatcgcaaactgtaatagacattacatccataaaagtttccccagtccttattgtaatattgcacagtgcaattgctacatggcaaactagtgtagcatagaagtcaaagcaaaaacaaaccaaagaaaggagccacaagagtaaaactgttcaacagttaatagttcaaactaagccattgaatctatcattgggatcgttaaaatgaatcttcctacaccttgcagtgtatgatttaacttttacagaacacaagccaagtttaaaatcagcagtagagatattaaaatgaaaaggtttgctaatagagtaacattaaataccctgaaggaaaaaaaacctaaatatcaaaataactgattaaaattcacttgcaaattagcacacgaatatgcaacttggaaatcatgcagtgttttatttaagaaaacataaaacaaaactattaaaatagttttagagggggtaaaatccaggtcctctgccaggatgctaaaattagacttcaggggaattttgaagtcttcaattttgaaacctattaaaaagcccatgattacagttaattaagagcagtgcacgcaacagtgacacgcctttagagagcattactgtgtatgaacatgttggctgctaccagccacagtcaatttaacaaggctgctcagtcatgaacttaatacagagagagcacgcctaggcagcaagcacagcttgctgggccactttcctccctgtcgtgacacaatcaatccgtgtacttggtgtatctgaagcgcacgctgcaccgcggcactgcccggcgggtttctgggcggggagcgatccccgcgtcgccccccgtgaaaccgacagagcctggactttcaggaggtacagcggcggtctgaaggggatctgggatcttgcagagggaacttgcatcgaaacttgggcagttctccgaaccggagactaagcttccccgagcagcgcactttggagacgtgtccggtctactccggactcgcatctcattccactcggccatagccttggcttcccggcgacctcagcgtggtcacaggggcccccctgtgcccagggaaatgtttcaagcttttcccggagactacgactccggctcccggtgtagctcatcaccctccgccgagtctcagtacctgtcttcggtggactccttcggcagtccacccaccgccgccgcctcccaggagtgcgccggtctcggggaaatgcccggctccttcgtgccaacggtcaccgcaatcacaaccagccaggatcttcagtggctcgtgcaacccaccctcatctcttccatggcccagtcccaggggcagccactggcctcccagcctccagctgttgacccttatgacatgccaggaaccagctactcaaccccaggcctgagtgcctacagcactggcggggcaagcggaagtggtgggccttcaaccagcacaaccaccagtggacctgtgtctgcccgtccagccagagccaggcctagaagaccccgagaagagacacttaccccagaagaagaagaaaagcgaagggttcgcagagagcggaacaagctggctgcagctaagtgcaggaaccgtcggagggagctgacagatcgacttcaggcggaaactgatcagcttgaagaggaaaaggcagagctggagtcggagatcgccgagctgcaaaaagagaaggaacgcctggagtttgtcctggtggcccacaaaccgggctgcaagatcccctacgaagaggggccggggccaggcccgctggccgaggtgagagatttgccagggtcaacatccgctaaggaagacggcttcggctggctgctgccgccccctccaccaccccccctgcccttccagagcagccgagacgcaccccccaacctgacggcttctctctttacacacagtgaagttcaagtcctcggcgaccccttccccgttgttagcccttcgtacacttcctcgtttgtcctcacctgcccggaggtctccgcgttcgccggcgcccaacgcaccagcggcagcgagcagccgtccgacccgctgaactcgccctcccttcttgctctgtaaactctttagacaaacaaaacaaacaaacccgcaaggaacaaggaggaggaagatgaggaggagaggggaggaagcagtccgggggtgtgtgtgtggaccctttgactcttctgtctgaccacctgccgcctctgccatcggacatgacggaaggacctcctttgtgttttgtgctccgtctctggttttctgtgccccggcgagaccggagagctggtgactttggggacagggggtggggcggggatggacacccctcctgcatatctttgtcctgttacttcaacccaacttctggggatagatggctggctgggtgggtagggtggggtgcaacgcccacctttggcgtcttgcgtgaggctggaggggaaagggtgctgagtgtggggtgcagggtgggttgaggtcgagctggcatgcacctccagagagacccaacgaggaaatgacagcaccgtcctgtccttcttttcccccacccacccatccaccctcaagggtgcagggtgaccaagatagctctgttttgctccctcgggccttagctgattaacttaacatttccaagaggttacaacctcctcctggacgaattgagcccccgactgagggaagtcgatgccccctttgggagtctgctaaccccacttcccgctgattccaaaatgtgaacccctatctgactgctcagtctttccctcctgggaaaactggctcaggttggatttttttcctcgtctgctacagagccccctcccaactcaggcccgctcccacccctgtgcagtattatgctatgtccctctcaccctcacccccaccccaggcgcccttggccgtcctcgttgggccttactggttttgggcagcagggggcgctgcgacgcccatcttgctggagcgctttatactgtgaatgagtggtcggattgctgggtgcgccggatgggattgacccccagccctccaaaactttccctgggcctccccttcttccacttgcttcctccctccccttgacagggagttagactcgaaaggatgaccacgacgcatcccggtggccttcttgctcaggccccagactttttctctttaagtccttcgccttccccagcctaggacgccaacttctccccaccctgggagccccgcatcctctcacagaggtcgaggcaattttcagagaagttttcagggctgaggctttggctcccctatcctcgatatttgaatccccaaatatttttggactagcatacttaagagggggctgagttcccactatcccactccatccaattccttcagtcccaaagacgagttctgtcccttccctccagctttcacctcgtgagaatcccacgagtcagatttctattttttaatattggggagatgggccctaccgcccgtcccccgtgctgcatggaacattccataccctgtcctgggccctaggttccaaacctaatcccaaaccccacccccagctatttatccctttcctggttcccaaaaagcacttatatctattatgtataaataaatatattatatatgagtgtgcgtgtgtgtgcgtgtgcgtgcgtgcgtgcgtgcgtgcgagcttccttgttttcaagtgtgctgtggagttcaaaatcgcttctggggatttgagtcagactttctggctgtccctttttgtcacctttttgttgttgtctcggctcctctggctgttggagacagtcccggcctctccctttatcctttctcaagtctgtctcgctcagaccacttccaacatgtctccactctcaatgactctgatctccggtntgtctgttaattctggatttgtcggggacatgcaattttacttctgtaagtaagtgtgactgggtggtagattttttacaatctatatcgttgagaattc

SEQ ID NO: 32 - protein, 338 aa, Mus musculus
  misc_feature: FOSB
MFQAFPGDYDSGSRCSSSPSAESQYLSSVDSFGSPPTAAASQECAGLGEMPGSFVPTVTAITTSQDLQWLVQPTLISSMAQSQGQPLASQPPAVDPYDMPGTSYSTPGLSAYSTGGASGSGGPSTSTTTSGPVSARPARARPRRPREETLTPEEEEKRRVRRERNKLAAAKCRNRRRELTDRLQAETDQLEEEKAELESEIAELQKEKERLEFVLVAHKPGCKIPYEEGPGPGPLAEVRDLPGSTSAKEDGFGWLLPPPPPPPLPFQSSRDAPPNLTASLFTHSEVQVLGDPFPVVSPSYTSSFVLTCPEVSAFAGAQRTSGSEQPSDPLNSPSLLAL

SEQ ID NO: 33 - DNA, 2000 bp, Homo sapiens
  misc_feature: DUSP1
tttgggctgtgtgtgcgacgcgggtcggaggggcagtcgggggaaccgcgaagaagccgaggagcccggagccccgcgtgacgctcctctctcagtccaaaagcggcttttggttcggcgcagagagacccgggggtctagcttttcctcgaaaagcgccgccctgcccttggccccgagaacagacaaagagcaccgcagggccgatcacgctgggggcgctgaggccggccatggtcatggaagtgggcaccctggacgctggaggcctgcgggcgctgctgggggagcgagcggcgcaatgcctgctgctggactgccgctccttcttcgctttcaacgccggccacatcgccggctctgtcaacgtgcgcttcagcaccatcgtgcggcgccgggccaagggcgccatgggcctggagcacatcgtgcccaacgccgagctccgcggccgcctgctggccggcgcctaccacgccgtggtgttgctggacgagcgcagcgccgccctggacggcgccaagcgcgacggcaccctggccctggcggccggcgcgctctgccgcgaggcgcgcgccgcgcaagtcttcttcctcaaaggaggatacgaagcgttttcggcttcctgcccggagctgtgcagcaaacagtcgacccccatggggctcagccttcccctgagtactagcgtccctgacagcgcggaatctgggtgcagttcctgcagtaccccactctacgatcagggtggcccggtggaaatcctgccctttctgtacctgggcagtgcgtatcacgcttcccgcaaggacatgctggatgccttgggcataactgccttgatcaacgtctcagccaattgtcccaaccattttgagggtcactaccagtacaagagcatccctgtggaggacaaccacaaggcagacatcagctcctggttcaacgaggccattgacttcatagactccatcaagaatgctggaggaagggtgtttgtccactgccaggcaggcatttcccggtcagccaccatctgccttgcttaccttatgaggactaatcgagtcaagctggacgaggcctttgagtttgtgaagcagaggcgaagcatcatctctcccaacttcagcttcatgggccagctgctgcagtttgagtcccaggtgctggctccgcactgttcggcagaggctgggagccccgccatggctgtgctcgaccgaggcacctccaccaccaccgtgttcaacttccccgtctccatccctgtccactccacgaacagtgcgctgagctaccttcagagccccattacgacctctcccagctgctgaaaggccacgggaggtgaggctcttcacatcccattgggactccatgctccttgagaggagaaatgcaataactctgggaggggctcgagagggctggtccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggtttttttttagttgccaacagttgtatgtttgctgattatttatgacctgaaataatatatttcttcttctaagaagacattttgttacataaggatgacttttttatacaatggaataaattatggcatttctattg

SEQ ID NO: 34 - DNA, 1933 bp, Mus musculus
  misc_feature: DUSP1
cggcgggaggaaagcgcggtgaagccagattaggagcagcgagcacttggggacttagggccacaggacaccgcacaagatcgaccgactttttctggagaaccgcagaacgggcacgctggggtcgctggggctggccatggtgatggaggtgggcatcctggacgccggggggctgcgcgcgctgctgcgagagggcgccgcgcagtgcctgttgttggattgtcgctccttcttcgctttcaacgccggccacatcgcgggctcagtgaacgtgcgcttcagcaccatcgtgcggcgccgcgccaagggcgccatgggcctggagcatatcgtgcccaacgctgaactgcgtggccgcctgctggccggagcctaccacgccgtggtgctgctggacgagcgcagcgcctccctggacggcgccaagcgcgacggcaccctggccctggccgcgggcgcgctctgccgagaggcgcgctccactcaagtcttctttctccaaggaggatatgaagcgttttcggcttcctgccctgagctgtgcagcaaacagtccacccccacggggctcagcctccccctgagtactagtgtgcctgacagtgcagaatccggatgcagctcctgtagtacccctctctacgatcaggggggcccagtggagatcctgtccttcctgtacctgggcagtgcctatcacgcttctcggaaggatatgcttgacgccttgggcatcaccgccttgatcaacgtctcagccaattgtcctaaccactttgagggtcactaccagtacaagagcatccctgtggaggacaaccacaaggcagacatcagctcctggttcaacgaggctattgacttcatagactccatcaaggatgctggagggagagtgtttgttcattgccaggccggcatctcccggtcagccaccatctgccttgcttacctcatgaggactaaccgggtaaagctggacgaggcctttgagtttgtgaagcagaggcggagtatcatctccccgaacttcagcttcatgggccagctgctgcagtttgagtcccaagtgctagcccctcactgctctgctgaagctgggagccctgccatggctgtccttgaccggggcacctctactaccacagtcttcaacttccctgtttccatccccgtccaccccacgaacagtgccctgaactaccttaaaagccccatcaccacctctccaagctgctgaagggcaaggggaggtgtggagtttcacttgccaccgggtcgccactcctcctgtgggaggagcaatgcaataactctgggagaggctcatgggagctggtccttatttatttaacacccccctcaccccccaactcctcctgagttccactgagttcctaagcagtcacaacaatgacttgaccgcaagacatttgctgaactcggcacattcgggaccaatatattgtgggtacatcaagtccctctgacaaaacagggcagaagagaaaggactctgtttgaggcagtttcttcgcttgcctgttttttttttctagaaacttcatgcttgacacacccaccagtattaaccattcccgatgacatgcgcgtatgagagtttttacctttatttatttttgtgtaggtcggtggtttctgccttcacaaatgtcattgtctactcatagaagaaccaaatacctcaattttgtgtttgcgtactgtactatcttgtaaatagacccagagcaggtttgctttcggcactgacagacaaagccagtgtaggtttgtagctttcagttatcgacagttgtatgtttgtttatttatgatctgaagtaatatatttcttcttctgtgaagacattttgttactgggatgactttttttatacaacagaataaattatgacgtttctattga

SEQ ID NO: 35 - protein, 367 aa, Mus musculus
  misc_feature: DUSP1
MVMEVGILDAGGLRALLREGAAQCLLLDCRSFFAFNAGHIAGSVNVRFSTIVRRRAKGAMGLEHIVPNAELRGRLLAGAYHAVVLLDERSASLDGAKRDGTLALAAGALCREARSTQVFFLQGGYEAFSASCPELCSKQSTPTGLSLPLSTSVPDSAESGCSSCSTPLYDQGGPVEILSFLYLGSAYHASRKDMLDALGITALINVSANCPNHFEGHYQYKSIPVEDNHKADISSWFNEAIDFIDSIKDAGGRVFVHCQAGISRSATICLAYLMRTNRVKLDEAFEFVKQRRSIISPNFSFMGQLLQFESQVLAPHCSAEAGSPAMAVLDRGTSTTTVFNFPVSIPVHPTNSALNYLKSPITTSPSC

SEQ ID NO: 36 - DNA, 996 bp, Homo sapiens
  misc_feature: Jun
atgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtgtcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttga

SEQ ID NO: 37 - DNA, 2565 bp, Mus musculus
  misc_feature: Jun
gtgacgactggtcagcaccgccggagagccgctgttgctgggactggtctgcgggctccaaggaaccgctgctccccgagagcgctccgtgagtgaccgcgacttttcaaagctcggcatcgcgcgggagcctaccaacgtgagtgctagcggagtcttaaccctgcgctccctggagcaactggggaggagggctcagggggaagcactgccgtctggagcgcacgctctaaacaaactttgttacagaagcggggacgcgcgggtatccccccgcttcccggcgcgctgttgcggccccgaaacttctgcgcacagcccaggctaaccccgcgtgaagtgacggaccgttctatgactgcaaagatggaaacgaccttctacgacgatgccctcaacgcctcgttcctccagtccgagagcggtgcctacggctacagtaaccctaagatcctaaaacagagcatgaccttgaacctggccgacccggtgggcagtctgaagccgcacctccgcgccaagaactcggaccttctcacgtcgcccgacgtcgggctgctcaagctggcgtcgccggagctggagcgcctgatcatccagtccagcaatgggcacatcaccactacaccgacccccacccagttcttgtgccccaagaacgtgaccgacgagcaggagggcttcgccgagggcttcgtgcgcgccctggctgaactgcatagccagaacacgcttcccagtgtcacctccgcggcacagccggtcagcggggcgggcatggtggctcccgcggtggcctcagtagcaggcgctggcggcggtggtggctacagcgccagcctgcacagtgagcctccggtctacgccaacctcagcaacttcaacccgggtgcgctgagctgcggcggtggggcgccctcctatggcgcggccgggctggcctttccctcgcagccgcagcagcagcagcagccgcctcagccgccgcaccacttgccccaacagatcccggtgcagcacccgcggctgcaagccctgaaggaagagccgcagaccgtgccggagatgccgggagagacgccgcccctgtcccctatcgacatggagtctcaggagcggatcaaggcagagaggaagcgcatgaggaaccgcattgccgcctccaagtgccggaaaaggaagctggagcggatcgctcggctagaggaaaaagtgaaaaccttgaaagcgcaaaactccgagctggcatccacggccaacatgctcagggaacaggtggcacagcttaagcagaaagtcatgaaccacgttaacagtgggtgccaactcatgctaacgcagcagttgcaaacgttttgagaacagactgtcagggctgaggggcaatggaagaaaaaaaataacagagacaaacttgagaacttgactggaagcgacagagaaaaaaaaagtgtccgagtactgaagccaagggtacacaagatggactgggttgcgacctgacggcgcccccagtgtgctggagtgggaaggacgtggcgcgcctggctttggcgtggagccagagagcagaggcctattggccggcagactttgcggacgggctgtgcccgcgcgaccagaacgatggacttttcgttaacattgaccaagaactgcatggacctaacattcgatctcattcagtattaaaggggggtgggaggggttacaaactgcaatagagactgtagattgcttctgtagtgctccttaacacaaagcagggagggctgggaaggggggggaggcttgtaagtgccaggctagactgcagatgaactcccctggcctgcctctctcaactgtgtatgtacatatatttttttttttaatttgatgaaagctgattactgtcaataaacagcttccgcctttgtaagttattccatgtttgtttgggtgtcctgcccagtgtttgtaaataagagatttgaagcattctgagtttaccatttgtaataaagtatataatttttttatgttttgtttctgaaaatttccagaaaggatatttaagaaaaatacaataaactattgaaaagtagcccccaacctctttgctgcattatccatagataatgatagctagatgaagtgacagctgagtgcccaatatactagggtgaaagctgtgtcccctgtctgattgtaggaatagataccctgcatgctatcattggctcatactctctcccccggcaacacacaagtccagactgtacaccagaagatggtgtggtgtttcttaaggctggaagaagggctgttgcaaggggagagggtcagcccgctggaaagcagacactttggttgaaagctgtatgaagtggcatgtgctgtgatcatttataatcataggaaagatttagtaattagctgttgattctcaaagcagggacccatggaagtttttaacaaaaggtgtctccttccaactttgaatctgacaactcctagaaaaagatgacctttgcttgtgcatatttataatagcgttcgttatcacaataaatgtattcaaat

SEQ ID NO: 38 - protein, 334 aa, Mus musculus
  misc_feature: Jun
MTAKMETTFYDDALNASFLQSESGAYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVSGAGMVAPAVASVAGAGGGGGYSASLHSEPPVYANLSNFNPGALSSGGGAPSYGAAGLAFPSQPQQQQQPPQPPHHLPQQIPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 39 - DNA, 2109 bp, Homo sapiens
  misc_feature: Dusp6
ccagcctcggagggagggattagaagccgctagactttttttcctcccctctcagtagcacggagtccgaattaattggatttcattcactggggaggaacaaaaactatctgggcagcttcattgagagagattcattgacactaagagccagcgctgcagctggtgcagagagaacctccggctttgacttctgtctcgtctgccccaaggccgctagcctcggcttgggaaggcgaggcggaattaaaccccgctccgagagcgcacgttcgcgcgcggtgcgtcggccattgcctgccccgaggggcgtctggtaggcaccccgccctctcccgcagctcgacccccatgatagatacgctcagacccgtgcccttcgcgtcggaaatggcgatcagcaagacggtggcgtggctcaacgagcagctggagctgggcaacgagcggctgctgctgatggactgccggccgcaggagctatacgagtcgtcgcacatcgagtcggccatcaacgtggccatcccgggcatcatgctgcggcgcctgcagaagggtaacctgccggtgcgcgcgctcttcacgcgcggcgaggaccgggaccgcttcacccggcgctgtggcaccgacacagtggtgctctacgacgagagcagcagcgactggaacgagaatacgggcggcgagtcgttgctcgggctgctgctcaagaagctcaaggacgagggctgccgggcgttctacctggaaggtggcttcagtaagttccaagccgagttctccctgcattgcgagaccaatctagacggctcgtgtagcagcagctcgccgccgttgccagtgctggggctcgggggcctgcggatcagctctgactcttcctcggacatcgagtctgaccttgaccgagaccccaatagtgcaacagactcggatggtagtccgctgtccaacagccagccttccttcccagtggagatcttgcccttcctctacttgggctgtgccaaagactccaccaacttggacgtgttggaggaattcggcatcaagtacatcttgaacgtcacccccaatttgccgaatctctttgagaacgcaggagagtttaaatacaagcaaatccccatctcggatcactggagccaaaacctgtcccagtttttccctgaggccatttctttcatagatgaagcccggggcaagaactgtggtgtcttggtacattgcttggctggcattagccgctcagtcactgtgactgtggcttaccttatgcagaagctcaatctgtcgatgaacgatgcctatgacattgtcaaaatgaaaaaatccaacatatcccctaacttcaacttcatgggtcagctgctggacttcgagaggacgctgggactcagcagcccatgtgacaacagggttccagcacagcagctgtattttaccaccccttccaaccagaatgtataccaggtggactctctgcaatctacgtgaaagaccccacacccctccttgctggaatgtgtctggcccttcagcagtttctcttggcagcatcagctgggctgctttctttgtgtgtggccccaggtgtcaaaatgacaccagctgtctgtactagacaaggttaccaagtgcggaattggttaatactaacagagagatttgctccattctctttggaataacaggacatgctgtatagatacaggcagtaggtttgctctgtacccatgtgtacagcctacccatgcagggactgggattcgaggacttccaggcgcatagggtagaaccaaatgatagggtaggagcatgtgttctttagggccttgtaaggctgtttccttttgcatctggaactgactatataattgtcttcaatgaagactaattcaattttgcatatagaggagccaaagagagatttcagctctgtatttgtggtatcagtttggaaaaaaaaatctgatactccatttgattattgtaaatatttgatcttgaatcacttgacagtgtttgtttgaattgtgtttgttttttcctttgatgggcttaaaagaaattatccaaagggagaaagagcagtatgccacttcttaa

SEQ ID NO: 40 - DNA, 2793 bp, Mus musculus
  misc_feature: Dusp6
gatccattgaggagctgcctcgcacagggggtgtgctctcgcggagtcctagggactgtgagcaaacccagtcttgaataatccggcgagaaacaccgggttggatccgaggtgcagcctcagagggaaggattaagagccgctagactttttttcttttccctttttctcctctcagtggcacggagtccgaattaattggatttcattcactgggtaggaacaaaactgggcaccttcattcagagagagagattcattgactcggagagtgatctggtgcagagggaccaccgacttgacttctgtgtcgctttccctaaccgctagcctcggcttgggaaaggcgaggcggaatcaaaccccgctccgagagcgggagcttcgcgcagcgtgctcggcctatgcctgcctcgaggggcgtctgctaggcaccccgccttctcctgcagctcgacccccatgatagatacgctcagacccgtgcccttcgcgtcggaaatggcgatctgcaagacggtgtcgtggctcaacgagcagctggagctgggcaacgaacggcttctgctgatggactgccgaccacaggagctgtacgagtcgtcacacatcgaatctgccattaatgtggccatccccggcatcatgctgcggcgtctgcagaagggcaacctgcccgtgcgtgcgctcttcacgcgctgcgaggaccgggaccgctttaccaggcgctgcggcaccgacaccgtggtgctgtacgacgagaatagcagcgactggaatgagaacactggtggagagtcggtcctcgggctgctgctcaagaaactcaaagacgagggctgccgggcgttctacctggaaggtggcttcagtaagttccaggccgagttcgccctgcactgcgagaccaatctagacggctcgtgcagcagcagttccccgcctttgccagtgctggggctcgggggcctgcggatcagctcggactcttcctcggacattgagtctgaccttgaccgagaccccaatagtgcaacggactctgatggcagcccgctgtccaacagccagccttccttcccggtggagattttgcccttcctttacctgggctgtgccaaggactcgaccaacttggacgtgttggaagagtttggcatcaagtacatcttgaatgtcacccccaatttgcccaatctgtttgagaatgcgggcgagttcaaatacaagcaaattcctatctcggatcactggagccaaaacctgtcccagtttttccctgaggccatttctttcatagatgaagcccgaggcaaaaactgtggtgtcctggtgcattgcttggcaggtatcagccgctctgtcaccgtgacagtggcgtacctcatgcagaagctcaacctgtccatgaacgatgcttacgacattgttaagatgaagaagtccaacatctcccccaacttcaacttcatgggccagctgcttgacttcgaaaggaccctgggactgagcagcccttgtgacaaccgtgtccccactccgcagctgtacttcaccacgccctccaaccagaacgtctaccaggtggactccctgcagtctacgtgaaaggcacccacctctcctagccgggagttgtccccattccttcagttcctcttgagcagcatcgaccaggctgctttctttctgtgtgtggccccgggtgtcaaaagtgtcaccagctgtctgtgttagacaaggttgccaagtgcaaaattggttattacggagggagagatttgctccattcattgtttttttggaaggacaggacatgctgtctctagatccagcaataggtttgcttctgtaccccagcctacccaagcagggactggacatccatccagatagagggtagcataggaatagggacaggagcatctgttctttaaggccttgtatggctgtttcctgttgcatctggaactaactatatatattgtcttcagtgaagactgattcaactttgggtatagtggagccaaagagatttttagctctgtatttgcggtatcggtttagaagacaaaaaaaattaaaacctgatacttttatctgattattgtaaatatttgatcttcaatcacttgacagtgtttgtttggcttgtatttgttttttatctttgggcttaaaagagatccaaagagagaaagagcagtatgccacttcttagaacaaaagtataaggaaaaaaatgttctttttaatccaaagggtatatttgcagcatgcttgaccttgatgtaccaattctgacggcattttcgtggatattattatcactaagactttgttatgatgaggtcttcagtctctttcatatatcttccttgtaactttttttttcctcttaatgtagttttgactctgccttacctttgtaaatatttggcttacagtgtctcaaggggtattttggaaagacaccaaaattgtgggttcactttttttttttttttaaataacttcagctgtgctaaacagcatattacctctgtacaaaattcttcagggagtgtcacctcaaatgcaatactttgggttggtttctttccttttaaaaaaaaaatacgaaactggaagtgtgtgtatgtgtgcgagtatgagcgcccatttggtggatgcaacaggttgagaggaagggagaattaacttgctccatgatgttcgtggtgtaaagttttgagctggaatttattataagaatgtaaaaccttaaattattaataaataactattttggct

SEQ ID NO: 41 - protein, 381 aa, Mus musculus
  misc_feature: Dusp6
MIDTLRPVPFASEMAICKTVSWLNEQLELGNERLLLMDCRPQELYESSHIESAINVAIPGIMLRRLQKGNLPVRALFTRCEDRDRFTRRCGTDTVVLYDENSSDWNENTGGESVLGLLLKKLKDEGCRAFYLEGGFSKFQAEFALHCETNLDGSCSSSSPPLPVLGLGGLRISSDSSSDIESDLDRDPNSATDSDGSPLSNSQPSFPVEILPFLYLGCAKDSTNLDVLEEFGIKYILNVTPNLPNLFENAGEFKYKQIPISDHWSQNLSQFFPEAISFIDEARGKNCGVLVHCLAGISRSVTVTVAYLMQKLNLSMNDAYDIVKMKKSNISPNFNFMGQLLDFERTLGLSSPCDNRVPTPQLYFTTPSNQNVYQVDSLQST

SEQ ID NO: 42 - DNA, 1050 bp, Homo sapiens
  misc_feature: Cdk1
ggggggggggggcacttggcttcaaagctggctcttggaaattgagcggagacgagcggcttgttgtagctgccgtgcggccgccgcggaataataagccgggatctaccataccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatg

SEQ ID NO: 43 - DNA, 963 bp, Mus musculus
  misc_feature: Cdk1
tccgtcgtaacctgttgagtaactatggaagactatatcaaaatagagaaaattggagaaggtacttacggtgtggtgtataagggtagacacagagtcactggccagatagtggccatgaagaagatcagacttgaaagcgaggaagaaggagtgcccagtactgcaattcgggaaatctctctattaaaagaacttcgacatccaaatatagtcagcctgcaggatgtgctcatgcaggactccaggctgtatctcatctttgagttcctgtccatggacctcaagaagtacctggactccatccctcctgggcagttcatggattcttcactcgttaagagttacttacaccaaatcctccagggaattgtgttttgccactcccggcgagttcttcacagagacttgaaacctcaaaatctattgattgatgacaaaggaacaatcaaactggctgatttcggccttgccagagcgtttggaataccgatacgagtgtacacacacgaggtagtgacgctgtggtaccgatctccagaagtgttgctgggctcggctcgttactccactccggttgacatctggagtatagggaccatatttgcagaactggccaccaagaagccgcttttccacggcgactcagagattgaccagctcttcaggatcttcagagctctgggcactcctaacaacgaagtgtggccagaagtcgagtccctgcaggactacaagaacacctttcccaagtggaagccggggagcctcgcatcccacgtcaagaacctggacgagaacggcttggatttgctctcaaaaatgctagtctatgatcctgccaaacgaatctctggcaaaatggccctgaagcacccgtactttgatgacttggacaatcagattaagaagatgtagccctctggatggatgtccctgtctgctggtcgtaggggaagatcg

SEQ ID NO: 44 - protein, 297 aa, Mus musculus
  misc_feature: Cdk1
MEDYIKIEKIGEGTYGVVYKGRHRVTGQIVAMKKIRLESEEEGVPSTAIREISLLKELRHPNIVSLQDVLMQDSRLYLIFEFLSMDLKKYLDSIPPGQFMDSSLVKSYLHQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLVYDPAKRISGKMALKHPYFDDLDNQIKKM

SEQ ID NO: 45 - DNA, 2496 bp, Homo sapiens
  misc_feature: Fignl1
gtcagtccccgcgcttttcggaggctgccagcgtcccacaccagccgcaggtgaaaaccggcagaaagacattaagagattttcctgcagtcactgctggcagatgatagagccaggatttgaaagcaggcagcctggctccagaccctgtgctcttaactcccgttttgcatcaagaacagaatcctatgaaaggcttgtacagtgcttggatagcagcatcaaggagcattgtgtacatgcagaagtgcacagtacctggagtgaaactgcttgtgttcgatttctgataccattcataactggctgtgtgatctcaaaacctctaaaatgcagacctccagctctagatctgtgcacctgagtgaatggcagaagaattacttcgcaattacatctggcatatgtaccggaccgaaggcagatgcataccgtgcacagatattacgcattcagtatgcatgggcaaactctgagatttcccaggtctgtgctaccaaactgttcaaaaaatatgcagagaaatattctgcaattattgattctgacaatgttgaatctgggttgaataattatgcagaaaacattttaactttggcaggatctcaacaaacagatagtgacaagtggcagtctggattgtcaataaataatgttttcaaaatgagtagtgtacagaagatgatgcaagctggcaaaaaattcaaagactctctgttggaacctgctcttgcatcagtggtaatccataaggaggccactgtctttgatcttcctaaatttagtgtttgtggtagttctcaagagagtgactcattacctaactcagctcatgatcgagaccggacccaagacttcccggagagcaatcgtttgaaactccttcagaatgcccagccacctatggtgactaacactgctaggacttgtcctacattctcagcacctgtaggtgagtcagctactgcaaaattccatgtcacaccattgtttggaaatgtcaaaaaggaaaatcacagctctgcaaaagaaaacataggacttaatgtgttcttatctaaccagtcttgttttcctgctgcctgtgaaaatccacagaggaagtctttttatggttctggcaccattgatgcactttccaatccaatactgaataaggcttgtagtaaaacagaagataatggcccaaaggaggatagcagcctgcctacatttaaaactgcaaaagaacaattatgggtagatcagcaaaaaaagtaccaccaacctcagcgtgcatcagggtcttcatatggtggtgtaaaaaagtctctaggagctagtagatcccgagggatacttggaaagtttgttcctcctatacccaagcaagatgggggagagcagaatggaggaatgcaatgtaagccttatggggcaggacctacagaaccagcacatccagttgatgagcgtctgaagaacttggagccaaagatgattgaacttattatgaatgagattatggatcatggacctccagtaaattgggaagatattgcaggagtagaatttgctaaagccaccataaaggaaatagttgtgtggcccatgttgaggccagacatctttactggtttaaggggaccccctaaaggaattttgctctttggtcctcctgggactggtaaaactctaattggcaagtgcattgctagtcagtctggggcaacattctttagcatctctgcttcatccttaacttctaaatgggtaggtgagggggagaaaatggtccgtgcattgtttgctgttgcaaggtgtcagcaaccagctgtgatatttattgacgaaattgattccttgttatctcaacggggagatggtgagcatgaatcttctagaaggataaaaacagaatttttagttcaattagatggagcaacaacatcttctgaagatcgtatcctagtggtgggagcaacaaatcggccacaagaaattgatgaggctgcccggagaagattggtgaaaaggctttatattcccctcccagaagcttcagccaggaaacagatagtaattaatctaatgtccaaagagcagtgttgcctcagtgaagaagaaattgaacagattgtacagcagtctgatgcgttttcaggagcagacatgacacagctttgcaggggggcttctcttggtcctattcgcagtttacaaactgctgacattgctaccataacaccggatcaagttcgacccatagcttacattgattttgaaaatgcttttagaactgtgcgacctagtgtttctccaaaagatttagagctttatgaaaactggaacaaaacttttggttgtggaaagtaagtgggatacttggaatcaaggcatctctgtattacagtcttctttattttttagcatagaaagttggggatgtgttaattgtatttttaagaatatattctaaattctgtacttcaaataatagcacagattttacatctg

SEQ ID NO: 46 - DNA, 2333 bp, Mus musculus
  misc_feature: Fignl1
catcgagaagtgttcagtgcctggtaaagtacatagaccttgcttcacttggaactcggccttgatttctgccgttggtcataatcagcagagttctctctaaacctttgacatggagacgtccagctccatgtctgtggagacgactaggtctgtgcaggtggacgaatggcagaagaattactgtgtggttacatccagcatatgtacaccaaagcagaaggccgatgcataccgtgcactactactgcatattcagtatgcatatgccaactccgagatctctcaggtctttgctaccaacctgttcaaaaggtatacagaaaaatactctgcaattattgattctgacaatgttgtaactggcttgaataactatgcagagagcatttttgctttggcaggatctcgacaggctgacagtaacaagtggcagtctggattgtcaatagataatgttttcaaaatgagttgtgtacaggagatgatgcaggctggcaagaaatttgaagagtctctgttggaacctgctgatgcatcagtagtcctgtgtaaagagcccaccgcctttgaggttcctcagcttagtgtttgtggaggttctgaagacgctgacatattatccagttcaggtcatgacacagataagacccaagccattccagggagcagtctgagatgttccccttttcagagtgctcggctgcctaaggaaactaataccactaagacatgcctcacctcctcaacatctttaggtgagtcagccactgcagcatttcacatgacaccattatttggaaacaccgaaaaggacactcaaagctttcctaaaaccagcacaggactaaatatgttcttatctaatctgtcttgtgttccttctggctgtgaaaaccctcaagaaaggaaggcttttaatgactctgacatcattgacatactttccaatccaacactgaacaaggctcctagtaaaacagaagacagaggccgaagggaagataatagcctgcctacctttaaaactgcaaaagaacaattatgggtagatcaaaagaaaaagggccatcaatcccagcatacatctaaatcttctaatggtgttatgaaaaagtctctgggagctgggaggtcgagagggatatttggcaagtttgttcctcctgtatctaataagcaagacggaagtgagcagcatgccaagaagcacaagtctagtagggcagggtctgcagaaccagcacacctcactgatgattgtctgaagaacgtggagccaaggatggttgaacttgttatgaatgaaattatggaccatgggcctccagtacattgggacgatattgctggagtagaatttgccaaagccacaataaaggaaatcgttgtgtggcccatgatgaggccagatatctttactggattgcgagggccccctaaaggaattctactctttggccctccagggactggtaaaactctgattggcaagtgcattgctagccagtctggagcaacattcttcagcatctctgcttcatcgctgacttctaagtgggtaggtgagggagaaaaaatggtccgtgcactgtttgctgttgccaggtgtcagcagccagctgtcatatttattgatgaaattgattctttattgtctcaacgaggagatggtgaacatgaatcttcaagaaggataaaaacggaatttttagttcagttagatggagcaaccacatcttctgaagaccggattcttgtggtgggagctacaaatcggccccaagagattgatgaagctgcccggagaagattggtgaaaagactttatattcccctcccagaagcttcagccaggaaacagatagtaggtaatctaatgtctaaggagcaatgttgtctcagtgatgaagaaactgatctggtagtgcagcagtctgatgggttttctggcgcagatatgacacagctttgcagagaggcttctcttggtcctattcgcagtttgcacgctgctgacattgctaccataagtccagatcaagttcgaccaatagcttatattgattttgaaaatgcttttaaaactgtgcgacctactgtatctccaaaagacttggagctttatgaaaactggaatgaaacatttggttgtggaaagtgaatatagcgattgaaaggagaagctgttatctagtagtcgtctttacctttagcctcggaagcttgctgtgctacttgtattgttttggagtatatcctgaattctgtgcctcagattagaatgataacagcttgactactgactgatatattagtatgttgtatttgcc

SEQ ID NO: 47 - protein, 683 aa, Mus musculus
  misc_feature: Fignl1
METSSSMSVETTRSVQVDEWQKNYCVVTSSICTPKQKADAYRALLLHIQYAYANSEISQVFATNLFKRYTEKYSAIIDSDNVVTGLNNYAESIFALAGSRQADSNKWQSGLSIDNVFKMSCVQEMMQAGKKFEESLLEPADASVVLCKEPTAFEVPQLSVCGGSEDADILSSSGHDTDKTQAIPGSSLRCSPFQSARLPKETNTTKTCLTSSTSLGESATAAFHMTPLFGNTEKDTQSFPKTSTGLNMFLSNLSCVPSGCENPQERKAFNDSDIIDILSNPTLNKAPSKTEDRGRREDNSLPTFKTAKEQLWVDQKKKGHQSQHTSKSSNGVMKKSLGAGRSRGIFGKFVPPVSNKQDGSEQHAKKHKSSRAGSAEPAHLTDDCLKNVEPRMVELIMNEIMDHGPPVHWDDIAGVEFAKATIKEIVVWPMMRPDIFTGLRGPPKGILLFGPPGTGKTLIGKCIASQSGATFFSISASSLTSKWVGEGEKMVRALFAVARCQQPAVIFIDEIDSLLSQRGDGEHESSRRIKTEFLVQLDGATTSSEDRILVVGATNRPQEIDEAARRRLVKRLYIPLPEASARKQIVGNLMSKEQCCLSDEETDLVVQQSDGFSGADMTQLCREASLGPIRSLHAADIATISPDQVRPIAYIDFENAFKTVRPTVSPKDLELYENWNETFGCGK

SEQ ID NO: 48 - DNA, 2972 bp, Homo sapiens
  misc_feature: Plk2
gcgcgcggctccgatgggaagcatgacccgggtggcgggacaagacttgcttcccggccacgcgcgctcggccggccgtggggcggggcataggcgtgacgtggtgtcgcgtatcgagtctccgcccccttcccgcctccccgtatataagacttcgccgagcactctcactcgcacaagtggaccggggtgttgggtgctagtcggcaccagaggcaagggtgcgaggaccacggccggctcggacgtgtgaccgcgcctagggggtggcagcgggcagtgcggggcggcaaggcgaccatggarcttttgcggactatcacctaccagccagccgccagcaccaaaatgtgcgagcaggcgctgggcaagggttgcggaggggactcgaagaagaagcggccgccgcagccccccgaggaatcgcagccacctcagtcccaggcgcaagtgcccccggcggcccctcaccaccatcaccaccattcgcactcggggccggagatctcgcggattatcgtcgaccccacgactgggaagcgctactgccggggcaaagtgctgggaaagggtggctttgcaaaatgttacgagatgacagatttgacaaataacaaagtctacgccgcaaaaattattcctcacagcagagtagctaaacctcatcaaagggaaaagattgacaaagaaatagagcttcacagaattcttcatcataagcatgtagtgcagttttaccactacttcgaggacaaagaaaacatttacattctcttggaatactgcagtagaaggtcaatggctcatattttgaaagcaagaaaggtgttgacagagccagaagttcgatactacctcaggcagattgtgtctggactgaaataccttcatgaacaagaaatcttgcacagagatctcaaactagggaacttttttattaatgaagccatggaactaaaagttggggacttcggtctggcagccaggctagaacccytggaacacagaaggagaacgatatgtggtaccccaaattatctctctcctgaagtcctcaacaaacaaggacatggctgtgaatcagacatttgggccctgggctgtgtaatgtatacaatgttactagggaggcccccatttgaaactacaaatctcaaagaaacttataggtgcataagggaagcaaggtatacaatgccgtcctcattgctggctcctgccaagcacttaattgctagtatgttgtccaaaaacccagaggatcgtcccagtttggatgacatcattcgacatgacttttttttgcagggcttcactccggacagactgtcttctagctgttgtcatacagttccagatttccacttatcaagcccagctaagaatttctttaagaaagcagctgctgctctttttggtggcaaaaaagacaaagcaagatatattgacacacataatagagtgtctaaagaagatgaagacatctacaagcttaggcatgatttgaaaaagacttcaataactcagcaacccagcaaacacaggacagatgaggagctccagccacctaccaccacagttgccaggtctggaacacccgcagtagaaaacaagcagcagattggggatgctattcggatgatagtcagagggactcttggcagctgtagcagcagcagtgaatgccttgaagacagtaccatgggaagtgttgcagacacagtggcaagggttcttcggggatgtctggaaaacatgccggaagctgattgcattcccaaagagcagctgagcacatcatttcagtgggtcaccaaatgggttgattactctaacaaatatggctttgggtaccagctctcagaccacaccgtcggtgtccttttcaacaatggtgctcacatgagcctccttccagacaaaaaaacagttcactattacgcagagcttggccaatgctcagttttcccagcaacagatgctcctgagcaatttattagtcaagtgacggtgctgaaatacttttctcattacatggaggagaacctcatggatggtggagatctgcctagtgttactgatattcgaagacctcggctctacctccttcagtggctaaaatctgataaggccctaatgatgctctttaatgatggcacctttcaggtgaatttctaccatgatcatacaaaaatcatcatctgtagccaaaatgaagaataccttctcacctacatcaatgaggataggatatctacaactttcaggctgacaactctgctgatgtctggctgttcatcagaattaaaaaatcgaatggaatatgccctgaacatgctcttacaaagatgtaactgaaagacttttcgaatggaccctatgggactcctcttttccactgtgagatctacagggaagccaaaagaatgatctagagtatgttgaagaagatggacatgtggtggtacgaaaacaattcccctgtggcctgctggactgggtggaacccagaaccaggctaaggcatacagttcttgactttggacaatcccaagagtgaaccagaatgcagttttccttgagatacctgttttaaaaggtttttcagacaattttgcagaaaggtgcattgattcttaaattctctctgttgagagcatttcagccagaggactttggaactgtgaatatacttcctgaaggggagggagaagggaggaagctcccatgttgtttaaaggctgtaattggagcagcttttggctgcgtaactgtgaactatggccatatataattttttttcattaatttttgaagatacttgtggctggaaaagtgcattccttgttaataaactttttatttattacagcccaaagagcagtatttattatcaaaatgtctttttttttatgttgaccattttaaaccgttggcaataaagagtatgaaaacgcaaaaaaaaaaaaaaa

SEQ ID NO: 49 - DNA, 2772 bp, Mus musculus
  misc_feature: plk2
cgtagggagagagactggtgctcgagggacagggctagcccggacgcgtgtccgcgcctcggaggtggcaagtaggcagtgtcgggtggcgaggcaacgatggagctcctgcggactatcacctaccagccggccgccggcaccaagatgtgcgagcaggctctgggcaaagcttgcggcggggactcaaagaagaagcgaccacagcagccttctgaagatgggcagccccaagcccaggtgaccccggcggccccgcaccaccatcaccaccattcccactcgggacccgagatctcgcggattatagtcgaccccacgacggggaagcgctactgccggggcaaagtgctgggcaagggtggatttgcaaagtgttacgaaatgacagatctgacaaacaacaaagtctacgctgcaaaaattattcctcacagcagagtagctaaacctcatcagagggaaaagatcgacaaagaaatcgagcttcacagactactgcaccataagcatgtcgtgcagttttaccactactttgaagacaaagaaaacatttacattctcttggaatactgcagtagaaggtccatggctcacatcttgaaagcaagaaaggtgttgacagagccagaagtccgatactacctcaggcagattgtgtcaggactcaagtatcttcacgaacaagaaatcttgcacagggatctcaagctagggaacttttttattaatgaagccatggagctgaaggtgggagactttggtttggcagccagactggaaccactggaacacagaaggagaacaatatgtggaaccccaaattatctctcccccgaagtcctcaacaaacaaggacacggctgtgaatcagacatctgggccttaggctgtgtaatgtatacgatgctgctaggaagacctccattcgaaaccacaaatctgaaagaaacgtacaggtgcataagggaagcaaggtataccatgccgtcctcattgctggcccctgctaagcacttgatagctagcatgctgtccaaaaacccagaggaccgccccagtttggatgacatcattcggcatgacttcttcctgcagggtttcactccggacagactctcttccagctgttgccacacagttccagatttccacttgtcaagcccagccaagaatttctttaagaaagccgcagccgctctttttggtggcaagaaggacaaagcaagatataacgacacacacaataaggtgtctaaggaagatgaagacatttacaagcttcggcatgatttgaagaaagtgtcgataacccagcagcctagcaaacacagagcagacgaggagccccagccgcctcccactactgttgccagatctggaacgtccgcagtggaaaacaaacagcagattggggatgcaatccggatgatagtcagggggactctcggcagctgcagcagcagcagcgaatgccttgaagacagcaccatgggaagtgttgcagacacagtggcaagagtccttcgaggatgtctagaaaacatgccggaagctgactgtatccccaaagagcagctgagcacgtcctttcagtgggtcaccaagtgggtcgactactccaacaaatatggctttgggtaccagctctcggaccacactgttggcgtccttttcaacaacggggctcacatgagcctccttccggacaaaaagacagttcactattatgcggaacttggccaatgctctgttttcccagcaacagatgcccctgaacaatttattagtcaagtgacggtgctgaaatacttttctcattacatggaggagaacctcatggatggtggtgatctcccgagtgttactgacattcgaagacctcggctctacctcctgcagtggttaaagtctgataaagccttaatgatgctcttcaatgacggcacatttcaggtgaatttctaccacgatcatacaaaaatcatcatctgtaaccagagtgaagaataccttctcacctacatcaatgaggacaggatctctacaactttcagactgacgactctgctgatgtctggctgttcgttagaattgaaaaatcgaatggaatatgccctgaacatgctcttacagagatgtaactgaaaacattattattattattattataattatttcgagcggacctcatgggactcttttccactgtgagatcaacagggaagccagcggaaagatacagagcatgttagagaagtcggacaggtggtggtacgaatacaattcctctgtggcctgctggactgctggaaccagaccagcctaaggtgtagagttgactttggacaatcctgagtgtggagccgagtgcagttttccctgagatacctgtcgtgaaaaggtttatgggacagtttttcagaaagatgcattgactctgaagttctctctgttgagagcgtcttcagttggaagacttggaactgtgaatacacttcctgaaggggagggagaagggaggttgctcccttgctgtttaaaggctacaatcagagcagcttttggctgcttaactgtgaactatggccatacattttttttttttttggttatttttgaatacacttgtggttggaaaagtgcattccttgttaataaactttttatttattacagccccaagagcagtatttattatcaagatgttctctttttttatgttgaccatttcaaactcttggcaataaagagtatgacatagaaaaaaaa

SEQ ID NO: 50 - protein, 682 aa, Mus musculus
  misc_feature: Plk2
MELLRTITYQPAAGTKMCEQALGKACGGDSKKKRPQQPSEDGQPQAQVTPAAPHHHHHHSHSGPEISRIIVDPTTGKRYCRGKVLGKGGFAKCYEMTDLTNNKVYAAKIIPHSRVAKPHQREKIDKEIELHRLLHHKHVVQFYHYFEDKENIYILLEYCSRRSMAHILKARKVLTEPEVRYYLRQIVSGLKYLHEQEILHRDLKLGNFFINEAMELKVGDFGLAARLEPLEHRRRTICGTPNYLSPEVLNKQGHGCESDIWALGCVMYTMLLGRPPFETTNLKETYRCIREARYTMPSSLLAPAKHLIASMLSKNPEDRPSLDDIIRHDFFLQGFTPDRLSSSCCHTVPDFHLSSPAKNFFKKAAAALFGGKKDKARYNDTHNKVSKEDEDIYKLRHDLKKVSITQQPSKHRADEEPQPPPTTVARSGTSAVENKQQIGDAIRMIVRGTLGSCSSSSECLEDSTMGSVADTVARVLRGCLENMPEADCIPKEQLSTSFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICNQSEEYLLTYINEDRISTTFRLTTLLMSGCSLELKNRMEYALNMLLQRCN

SEQ ID NO: 51 - DNA, 3200 bp, Homo sapiens
  misc_feature: Rsad2
caggaagggccatgaagattaataaagatttggactcagggcaaatatttacttagtagcaataactcaaagaattactgttgaataaataagccaattaagcagccaatcacgtactatgcggatgcacacaaatgaaaccctcacttcaacctgaagacattcgcacatgagttacgtagagggacctgcaggaagcggtagagaaaacataaggcttatgcgtttaatttccacaccaatttcaggatctttgtcactgacagcagcactaagacttgttaactttatatagttaagaagaacaaggctgagcgcgatgactcacgcctgtaagcctagaactttgggaggccaaagcaggcagactgcttgagcccaggagttccagaccagcctgggcaacatggcaacaccccatctctacaaaaaaatacaagaatcagctgggcgtggtgatgtgttcctgtaatctcagctactcgggaggcagaggcaggaggattgcttgaacccgggaggcagaggttgtagttagccgagatctcgccactgcactccagtctggacgacagagtgagactcagtctcaaataaataaataaatacataaatataaggaaaaaaataaagctgctttctcctcttcctcctctttggtctcatctggctctgctccaggcatctgccacaatgtgggtgcttacacctgctgcttttgctgggaagttcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagaatccctttcaagataaattctgtcattaatcgtttcaacgtggaagaggacatgacggaacagatcaaagcactaaaccctgtccgctggaaagtgttccagtgcctcttaattgaaggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaatatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttttgcttttatgccattgcagtcttgtactttttactgtgatgtacagaaatagtcaacagatgtttccaagaacatatgatatgataatcctaccaattttcaagaagtctctagaaagagataacacatggaaagacggcgtggtgcagcccagcccacggtgcctgttccatgaatgctggctacctatgtgtgtggtacctgttgtgtccctttctcttcaaagatccctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatttctgagaagtgacgttacactgggttggcataagatatcctaaaatcacgctggaaccttgggcaaggaagaatgtgagcaagagtagagagagtgcctggatttcatgtcagtgaagccatgtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttaaaaaaaaaaaaaaaaaaaaaaaaaaaaaaaaggcca

SEQ ID NO: 52 - DNA, 1196 bp, Mus musculus
  misc_feature: Rsad2
cctatcaccatggggatgctggtgcccactgctctagctgctcggctgctgagcctgttccagcagcagctgggttccctctggagtggcctggccatcctgttctgctggctgagaatagcattagggtggctagatcccgggaaggaacagccacaggtccggggtgagctggaggagacccaggagacccaggaagatgggaacagcactcagcgcacaacccccgtgagtgtcaactaccacttcactcgtcagtgcaactacaaatgtggcttctgcttccacacagccaagacatccttcgtgctgcccctggaggaggccaagcgaggactgcttctgctcaaacaggctggtttggagaagatcaacttttctggaggagaacccttccttcaggacaggggtgaatacttgggcaagcttgtgagattctgcaaggaggagctagccctgccctctgtgagcatagtgagcaatggcagccttatccaggagagatggttcaaggactatggggagtatttggacattcttgctatctcctgcgacagcttcgatgagcaggttaatgctctgattggccgtggtcaaggaaaaaagaaccacgtggaaaaccttcaaaagctgaggaggtggtgcagggattacaaggtggctttcaagatcaactctgtcattaatcgcttcaacgtggacgaagacatgaatgaacacatcaaggccctgagccctgtgcgctggaaggttttccagtgcctcctaattgagggtgagaactcaggagaagatgccctgagggaagcagaaagatttcttataagcaatgaagaatttgaaacattcttggagcgtcacaaagaggtgtcctgtttggtgcctgaatctaaccagaagatgaaagactcctaccttatcctagatgaatatatgcgctttctgaactgtaccggtggccggaaggacccttccaagtctattctggatgttggcgtggaagaagcaataaagttcagtggatttgatgagaagatgtttctgaagcgtggcggaaagtatgtgtggagtaaagctgacctgaagctggactggtgaggctgagatgggaaggaaactccgaccagctacagggacattcacgcccagctatccttcaacaagctacatcttctggctgtctacagactgttgtt

SEQ ID NO: 53 - protein, 362 aa, Mus musculus
  misc_feature: Rsad2
MGMLVPTALAARLLSLFQQQLGSLWSGLAILFCWLRIALGWLDPGKEQPQVRGEPEDTQETQEDGNSTQPTTPVSVNYHFTRQCNYKCGFCFHTAKTSFVLPLEEAKRGLLLLKQAGLEKINFSGGEPFLQDRGEYLGKLVRFCKEELALPSVSIVSNGSLIRERWFKDYGEYLDILAISCDSFDEQVNALIGRGQGKKNHVENLQKLRRWCRDYKVAFKINSVINRFNVDEDMNEHIKALSPVRWKVFQCLLIEGENSGEDALREAERFLISNEEFETFLERHKEVSCLVPESNQKMKDSYLILDEYMRFLNCTGGRKDPSKSILDVGVEEAIKFSGFDEKMFLKRGGKYVWSKADLKLDW

SEQ ID NO: 54 - DNA, 2370 bp, Homo sapiens
  misc_feature: Sgk1
cacgagggagcgctaacgtctttctgtctccccgcggtggtgatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgatttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgagctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaattttttgaagagcacattctcctcagtgagctcatgaggttttcatttttattcttccttccaacgtggtgctatctctgaaacgagcgttagagtgccgccttagacggaggcaggagtttcgttagaaagcggacctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaataaaaaaaaaaaaaaaaaaaaaaa

SEQ ID NO: 55 - DNA, 2429 bp, Mus musculus
  misc_feature: Sgk1
acccacgcgtccggccggtttcactgctcccctcagtctcttttgggctctttccgggcatcgggacgatgaccgtcaaagccgaggctgctcgaagcacccttacctactccagaatgaggggaatggtagcgattctcatcgcttttatgaaacagagaaggatgggcctgaacgattttattcagaagattgccagcaacacctatgcatgcaaacacgctgaagttcagtccattttgaaaatgtcccatcctcaggagccggagcttatgaacgctaacccctctcctccgccaagtccctctcaacaaatcaacctgggtccgtcctccaaccctcacgccaaaccctccgactttcacttcttgaaagtgatcggaaagggcagttttggaaaggttcttctggctaggcacaaggcagaagaagtattctatgcagtcaaagttttacagaagaaagccatcctgaagaagaaagaggagaagcatattatgtcagagcggaatgttctgttgaagaatgtgaagcaccctttcctggtgggccttcacttctcattccagaccgctgacaaactctactttgtcctggactacattaatggtggagagctgttctaccatctccagagggagcgctgcttcctggaaccacgggctcgattctacgcagctgaaatagccagtgccttgggctatctgcactccctaaacatcgtttatagagacttaaaacctgagaatattctcctagactcccaggggcacatcgtcctcactgactttgggctctgcaaagagaatattgagcataacgggacaacatctaccttctgtggcacgcctgagtatctggctcctgaggtcctccataagcagccgtatgaccggacggtggactggtggtgtcttggggctgtcctgtatgagatgctctacggcctgcccccgttttatagccggaacacggctgagatgtacgacaatattctgaacaagcctctccagttgaaaccaaatattacaaactcggcaaggcacctcctggaaggcctcctgcagaaggaccggaccaagaggctgggtgccaaggatgactttatggagattaagagtcatattttcttctctttaattaactgggatgatctcatcaataagaagattacacccccatttaacccaaatgtgagtgggcccagtgaccttcggcactttgatcccgagtttaccgaggagccggtccccagctccatcggcaggtcccctgacagcatccttgtcacggccagtgtgaaggaagcagcagaagccttcctcggcttctcctatgcacctcctgtggattccttcctctgagtgctcccgggatggttctgaaggacttcctcagcgtttcctaaagtgttttccttaccctttggtggaggttgccagctgacagaacattttaaaagaatttgcacacctggaagcttggcagtctcgcctgcccggcgtggcgcgacgcagcgcgcgctgcttgatgggagctttccgaagagcacaccctcctctcaatgagcttgtgaggtcttcttttcttctcttccttccaacgtggtgctagctccaggcgagcgagcgtgagagtgccgcctgagacagacaccttggtctcagttagaaggaagatgcaggtctaagaggaatccccgcagtctgtctgagctgtgatcaagaatattctgcaatgtgccttttctgagatcgtgttagctccaaagctttttcctatcgcagagtgttcagtttgtgtttgtttgtttttgttttgttttgtttttcccttggcggatttcccgtgtgtgcagtggcgtgagtgtgctatgcctgatcacagacggttttgttgtgagcatcaatgtgacacttgcaggacactacaatgtgggacattgtttgtttcttccacatttggaagataaatttatgtgtagactgttttgtaagatatagttaataactaaaacctattgaaacggtcttgcaatgacgagcattcagatgcttaaggaaagcattgctgctacaaatatttctatttttagaaagggtttttatggaccaatgccccagttgtcagtcaaagccgttggtgttttcattgtttaaaatgtcacctataaaacgggcattatttatgttttttttccctttgttcatattcttttgcattcctgattattgtatgtatcgtgtaaaggaagtctgtacattgggttataacactagatatttaaacttacaggcttatttgtaaaccatcattttaatgtactgtaattaacatgggttataatatgtacaattcctcctccttaccacacaactttttttgtgtgcgataaaccaattttggtttgcaataaaatcttgaaacct

SEQ ID NO: 56 - protein, 431 aa, Mus musculus
  misc_feature: Sgk1
MTVKAEAARSTLTYSRMRGMVAILIAFMKQRRMGLNDFIQKIASNTYACKHAEVQSILKMSHPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNGTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHIFFSLINWDDLINKKITPPFNPNVSGPSDLRHFDPEFTEEPVPSSIGRSPDSILVTASVKEAAEAFLGFSYAPPVDSFL

SEQ ID NO: 57 - DNA, 930 bp, Homo sapiens
  misc_feature: Sdc1
atgagacgcgcggcgctctggctctggctctgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaagctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggtgcctaccagaaacccaccaagcaggaggagttctacgcc

SEQ ID NO: 58 - DNA, 2432 bp, Mus musculus
  misc_feature: Sdc1
actccgcgggagaggtgcgggccagaggagacagagcctaacgcagaggaagggacctggcagtcgggagctgactccagccggcgaaacctacagccctcgctcgagagagcagcgagctgggcaggagcctgggacagcaaagcgcagagcaatcagcagagccggcccggagctccgtgcaaccggcaactcggatccacgaagcccaccgagctcccgccgccggtctgggcagcatgagacgcgcggcgctctggctctggctctgcgcgctggcgctgcgcctgcagcctgccctcccgcaaattgtggctgtaaatgttcctcctgaagatcaggatggctctggggatgactctgacaacttctctggctctggcacaggtgctttgccagatactttgtcacggcagacaccttccacttggaaggacgtgtggctgttgacagccacgcccacagctccagagcccaccagcagcaacaccgagactgcttttacctctgtcctgccagccggagagaagcccgaggagggagagcctgtgctccatgtagaagcagagcctggcttcactgctcgggacaaggaaaaggaggtcaccaccaggcccagggagaccgtgcagctccccatcacccaacgggcctcaacagtcagagtcaccacagcccaggcagctgtcacatctcatccgcacgggggcatgcaacctggcctccatgagacctcggctcccacagcacctggtcaacctgaccatcagcctccacgtgtggagggtggcggcacttctgtcatcaaagaggttgtcgaggatggaactgccaatcagcttcccgcaggagagggctctggagaacaagacttcacctttgaaacatctggggagaacacagctgtggctgccgtagagcccggcctgcggaatcagcccccggtggacgaaggagccacaggtgcttctcagagccttttggacaggaaggaagtgctgggaggtgtcattgccggaggcctagtgggcctcatctttgctgtgtgcctggtggctttcatgctgtaccggatgaagaagaaggacgaaggcagctactccttggaggagcccaaacaagccaatggcggtgcctaccagaaacccaccaagcaggaggagttctacgcctgatggggaaatagttctttctcccccccacagcccctgccactcactaggctcccacttgcctcttctgtgaaaaacttcaagccctggcctccccaccactgggtcatgtcctctgcacccaggcccttccagctgttcctgcccgagcggtcccagggtgtgctgggaactgattcccctcctttgacttctgcctagaagcttgggtgcaaagggtttcttgcatctgatctttctaccacaaccacacctgtcgtccactcttctgacttggtttctccaaatgggaggagacccagctctggacagaaaggggacccgactgctttggacctagatggcctattgcggctggaggatcctgaggacaggagaggggcttcggctgaccagccatagcacttacccatagagaccgctagggttggccgtgctgtggtgggggatggaggcctgagctccttggaatccacttttcattgtggggaggtctactttagacaacttggttttgcacatattttctctaatttctctgttcagagccccagcagaccttattactggggtaaggcaagtctgttgactggtgtccctcacctcgcttccctaatctacattcaggagaccgaatcgggggttaataagactttttttgttttttgtttttgtttttaacctagaagaaccaaatctggacgccaaaacgtaggcttagtttgtgtgttgtctctgagtttgtgctcatgcgtacaacagggtatggactatctgtatggtgccccatttttggcggcccgtaagtaggctaggctagtccaggatactgtggaatagccacctcttgaccagtcatgcctgtgtgcatggactcagggccacggccttggcctgggccaccgtgacattggaagagcctgtgtgagaacttactcgaagttcacagtctaggagtggaggggaggagactgtagagttttgggggaggggtagcaagggtgcccaagcgtctcccacctttggtaccatctctagtcatccttcctcccggaagttgacaagacacatcttgagtatggctggcactggttcctccatcaagaaccaagttcaccttcagctcctgtggccccgcccccaggctggagtcagaaatgtttcccaaagagtgagtcttttgcttttggcaaaacgctacttaatccaatgggttctgtacagtagattttgcagatgtaataaactttaatataaagg

SEQ ID NO: 59 - protein, 311 aa, Mus musculus
  misc_feature: Sdc1
MRRAALWLWLCALALRLQPALPQIVAVNVPPEDQDGSGDDSDNFSGSGTGALPDTLSRQTPSTWKDVWLLTATPTAPEPTSSNTETAFTSVLPAGEKPEEGEPVLHVEAEPGFTARDKEKEVTTRPRETVQLPITQRASTVRVTTAQAAVTSHPHGGMQPGLHETSAPTAPGQPDHQPPRVEGGGTSVIKEVVEDGTANQLPAGEGSGEQDFTFETSGENTAVAAVEPGLRNQPPVDEGATGASQSLLDRKEVLGGVIAGGLVGLIFAVCLVAFMLYRMKKKDEGSYSLEEPKQANGGAYQKPTKQEEFYA

SEQ ID NO: 60 - DNA, 1191 bp, Homo sapiens
  misc_feature: Serpine2
atgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaacgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtccccaagagggtgcaggtgatcctgcccaagttcacagctgtagcacaaacagatttgaaggagccgctgaaagttcttggcattactgacatgtttgattcatcaaaggcaaattttgcaaaaataacaaggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccc

SEQ ID NO: 61 - DNA, 2024 bp, Mus musculus
  misc_feature: Serpine2
agtgcagtggttgcacgggagtgcgggctgcacgcgtcaccgtcaccgccgcctgtcccccaccgccgcgcagcgccgatctccctcccggtttcggccgccacctggggatccaagcgaggacgggctgtccttgttggaaggaaccatgaattggcattttcctttcttcatcttgaccacagtgactttatactctgtgcactcccagttcaactctctgtcactggaggaactaggctccaacacagggatccaggtcttcaatcagatcatcaagtcacggcctcatgagaacgttgttgtctccccacatgggatcgcgtccatcttgggcatgctgcagctcggggctgacggcaagacaaagaagcagctctccacggtgatgcgatataatgtaaacggagttggtaaagtgctgaagaagatcaacaaggctattgtctccaagaaaaataaagacattgtgaccgtggccaatgctgtgtttctcaggaatggctttaaaatggaagtgccttttgcagtaaggaacaaagatgtgtttcagtgtgaagtgcagaatgtgaacttccaggacccagcctctgcctctgagtccatcaatttttgggtcaaaaatgagaccaggggcatgattgataatctgctttccccaaatctgatcgatggtgcccttaccaggctggtcctcgttaatgcagtgtatttcaagggtttgtggaagtctcggtttcaaccagagagcacaaagaaacggacattcgtggcaggtgatgggaaatcctaccaagtacccatgttggctcagctctctgtgttccgctcagggtctaccaggaccccgaatggcttatggtacaacttcattgagctgccctaccatggtgagagcatcagcatgctgatcgccctgccaacagagagctccaccccactgtctgccatcatccctcacatcactaccaagaccattgatagctggatgaacaccatggtacccaagaggatgcagctggtcctacccaagttcacagctgtggcacaaacagatctgaaggagccactgaaagcccttggcattactgagatgtttgagccatcaaaggcaaattttacaaaaataacaaggtcagagagccttcatgtctctcacatcttgcaaaaagcaaaaattgaagtcagtgaagatggaaccaaagcttcagcagcaacaactgcaatcctaattgcaaggtcatcacctccctggtttatagtagacaggcctttcctgttttccatccgacacaatcccacaggtgccatcttgttcctgggccaggtgaacaagccctgaaggacagacaaaggaaagccacgcaaagccaagacgacttggctctgaagagagactccctccccacatctttcatagttctgttaaatatttttatatactgctttcttttttgaaactggttcatagcagcagttaagtgacgcaagtgtttctggtcggggctgtgtcagaagaaagggctggatgcctgggatgctggatgcctgggatgctggatgcctgggatgctggatgcctgggatgctggatgcctgggatgctggatgcctgggatgctgtagtgaaggatgagcaggccggtttcacgatgtctagaagatttctttaaactactgatcagttatctaggttaacaaccctctcgagtatttgctgtctgtcaagttcagcatctttgtttcattcctgttgatatgtgtgactttccaggagaggattaatcagtgtggcaggagaggttaaaaaaaaaaaagacattttatagtagtttttatgtttttatggaaaacaatatcatttgcctttttaattctttttcctctcacttccacccaaaggcttgagggtggcaagggatggagctagcaaaagccgtagcctcttcgtgtgttgtttctgttgctgttgctcttgttgttttatatactgcatgtgttcactaaaataaagttggaaaact

SEQ ID NO: 62 - protein, 397 aa, Mus musculus
  misc_feature: Serpine2
MNWHFPFFILTTVTLYSVHSQFNSLSLEELGSNTGIQVFNQIIKSRPHENVVVSPHGIASILGMLQLGADGKTKKQLSTVMRYNVNGVGKVLKKINKAIVSKKNKDIVTVANAVFLRNGFKMEVPFAVRNKDVFQCEVQNVNFQDPASASESINFWVKNETRGMIDNLLSPNLIDGALTRLVLVNAVYFKGLWKSRFQPESTKKRTFVAGDGKSYQVPMLAQLSVFRSGSTRTPNGLWYNFIELPYHGESISMLIALPTESSTPLSAIIPHITTKTIDSWMNTMVPKRMQLVLPKFTAVAQTDLKEPLKALGITEMFEPSKANFTKITRSESLHVSHILQKAKIEVSEDGTKASAATTAILIARSSPPWFIVDRPFLFSIRHNPTGAILFLGQVNKP

SEQ ID NO: 63 - DNA, 1424 bp, Homo sapiens
  misc_feature: Spp1
gaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataa

SEQ ID NO: 64 - DNA, 1385 bp, Mus musculus
  misc_feature: Spp1
cttgcttgggtttgcagtcttctgcggcaggcattctcggaggaaaccagccaaggactaactacgaccatgagattggcagtgatttgcttttgcctgtttggcattgcctcctccctcccggtgaaagtgactgattctggcagctcagaggagaagctttacagcctgcacccagatcctatagccacatggctggtgcctgacccatctcagaagcagaatctccttgcgccacagaatgctgtgtcctctgaagaaaaggatgactttaagcaagaaactcttccaagcaattccaatgaaagccatgaccacatggacgacgatgatgacgatgatgatgacgatggagaccatgcagggagcgaggattctgtggactcggatgaatctgacgaatctcaccattcggatgagtctgatgagaccgtcactgctagtacacaagcagacactttcactccaatcgtccctacagtcgatgtccccaacggccgaggtgatagcttggcttatggactgaggtcaaagtctaggagtttccaggtttctgatgaacagtatcctgatgccacagatgaggacctcacctctcacatgaagagcggtgagtctaaggagtccctcgatgtcatccctgttgcccagcttctgagcatgccctctgatcaggacaacaacggaaagggcagccatgagtcaagtcagctggatgaaccaagtctggaaacacacagacttgagcattccaaagagagccaggagagtgccgatcagtcggatgtgatcgatagtcaagcaagttccaaagccagcctggaacatcagagccacaagtttcacagccacaaggacaagctagtcctagaccctaagagtaaggaagatgataggtatctgaaattccgaatttctcatgaattagagagttcatcttctgaggtcaactaaagaagaggcaaaaacacagttccttactttgcatttagtaaaaacaagaaaaagtgttagtgaggattaagcaggaatactaactgctcatttctcagttcagtggatatatgtatgtagagaaagagaggtaatattttgggctcttagcttagtctgttgtttcatgcaaacaaccgttgtaaccaaaagcttctgcactttgcttctgttcttcctgtacaagaaatgcaaacggccactgcattttaatgattgttattcttttatgaataaaatgtatgtagaaacaagcaaatttactgaaacaagcagaattaaaagagaaactgtaacagtctatatcactatacccttttagttttataattagcatatattttgttgtgattattttttttgttggtgtgaataaatcttgtaacgaatgt

SEQ ID NO: 65 - protein, 294 aa, Mus musculus
  misc_feature: Spp1
MRLAVICFCLFGIASSLPVKVTDSGSSEEKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 66 - DNA, 2373 bp, Homo sapiens
  misc_feature: Cdca8
ggttgactgtagagccgctctctctcactggcacagcgaggttttgctcagcccttgtctcgggaccgcaggtacgtgtctggcgacttcttcgggtggtccccgtccgccctcctcgtccctacccagtttcttgcttccctgccccatctccgccgctccccgcagcctccgccgagcgccatggctcctaggaagggcagtagtcgggtggccaagaccaactccttacggaggcggaagctcgcctcctttctgaaagacttcgaccgtgaagtggaaatacgaatcaagcaaattgagtcagacaggcagaacctcctcaaggaggtggataacctctacaacatcgagatcctgcggctccccaaggctctgcgcgagatgaactggcttgactacttcgcccttggaggaaacaaacaggccctggaagaggcggcaacagctgacctggatatcaccgaaataaacaaactaacagcagaagctattcagacacccctgaaatctgccaaaacacgaaaggtaatacaggtagatgaaatgatagtggaagagggaagaaggagaaggaaaatttacgtaagaatcttcaaactgcaagagtcaaaaggtgtcctccatccaagaagagaactcagtccatacaaggcaaaggaaaagggaaaaggtcaagccgtgctaacactgttaccccagccgtgggccgattggaggtgtccatggtcaaaccaactccaggcctgacacccaggtttgactcaagggtcttcaagaccctggcctgcgtactccagcagcaggagagcggatttacaacatctcagggaatggcagccctcttgctgacagcaaagagatcttcctcactgtgccagtgggcggcggagagagcctgcgattattggccagtgacttgcagaggcacagtattgcccagctggatccagaggccttgggaaacattaagaagctctccaaccgtctcgcccaaatctgcagcagcatacggacccacaaatgagacaccaaagttgacaggatggacttttaatgggcacttctgggaccctgaagagacttcttcccttcaggcttattgtttgagtgtgaagttccagagcaaggagccatgttcctctaagggaattcaggaattcagacgtgctagtcccacaccagttaggtagagctgtctgttcaccctcccatcccagctgatcccagtcactgcttgctggggccatgccatggaagcttcccatcagtctcccagctgaatcctccctgctctctgagctgctgccttttgcctcctgcaactcaacatcctcttcaccctgccctgcctgcagttgagggggcgaagaagaaccctgtgttctcaggaagactgcctccaccaccgctacccagagaacctctgcatctggcatttctgctctctatgcttgagaccgggaggtttaggctcagataagtgagctctgggccatgagagggtaggtccagaaggtggggggaactgtacagatcagcagagcaggacagttggcagcagtgacctcagtagggaacatgtccgtctaccctctcgcactcatgacacctccccctaccagcctctctctctctcacctcctctgtgggaggtggtcagtgggacttagggatctttcacctgctgtgcccagtagttctgaagtctgcttgtggagcagtgttttatgtttatccctgtttactgaagaccaaatactggtttggagacaacttccatgtcttgctcttctacctccctagttagtggaaatttggataagggaactgtagggcccagattctggaggttttatgtcattggccacagaataactgtctctaagctatccatggtccagtggtccctgccaagtctgtagacttcagagagcacttctctcttatggggttcatgggaacaggggcgggtgtgacttgcttggtggcctcattccatgtgtgcctgtgcctggggcatggactttgttaagcagagtcagcagtgaggtcctcattctccagccagcctctctgccctggagaatcatgtgctatgttctaagaatttgagaactagagtcctcatccccaggcttgaaggcacatggctttctcatgtagggctctctgtggtatttgttattattttgcaacaagaccattttagtaaaacagtcctgttcaagttgtattcttttaagttcttttattctcctttccctgagatttttgtatatattgttctgagtaatggtatctttgagctgattgttctaatcagagctggtacctactttcaataaattctggttttgtgttttcttttgt

SEQ ID NO: 67 - DNA, 1616 bp, Mus musculus
  misc_feature: Cdca8
ggaattgaattgggtggcggttaaccgaggagccgcccgtcccttagttggagctgtgagggttcctcagactgtgttttgggacctgcaggtaggtttcggcagagttctggaaacctagactccaacgactgaactttctcagctctccgaccgctcacaccctctccccgtctcagtcgcggagccggctgcttggcccctcgctcgacgcagccaggcgccatggctcccaagaaacgcagcagccgcggaaccaggaccaacacgctgcggagccggaagctcgcctccttcctgaaggacttcgaccgcgaggtgcaagttcgaaccaagcaaattgagtccgacagacagaccctcctcaaggaggtggaaaatctgtacaacatcgagatccttcggctccccaaggcgctgcaagggatgaagtggcttgactacttcgccctaggaggaaacaagcaggccctggaagaggcagcaaaagctgatcgagacatcacagaaataaacaatttaacagctgaagctattcagacacctttgaaatctgttaaaaagcgaaaggtaatcgaggtggaggaatcgataaaggaagaagaagaagaggaagaagaaggaggaggagaaggaggaagaacaaaaaagagccataagaatcttcgatctgcaaaagtcaaaagatgccttccatccaagaagagaacccagtccatacaaggaagaggcagaagtaaaaggttaagccatgactttgtgacgccagctatgagcaggctggagccgtctctggtgaaaccaaccccaggcatgacacctaggtttgactcccgggtcttcaagactccagggctacgcactccagcagccaaagagcaagtttacaacatctccatcaacggcagccctctcgcagacagcaaagagatctccctcagtgtgcccataggtggcggtgcgagcttgcggttattggccagtgacttgcaaaggattgatattgctcagctgaatccagaggccctgggaaacattagaaagctctcgagccgcctcgcccagatctgcagcagcatacggacgggccgatgagaggacaacaggacacacagtggcagcagggactgtggtagcagagtgcacacatctgtccttcttctgtggggtccttcactgccaacacctgcaacggtgctttgtctctctgacagctatggtgtcttgctgcacacttctagttagtgggaattttagacggggaacacagggctagtcagggcctttgtgtgcttggtgtggagtgactgagaaccgtctatggttcaaggtcccactggggataaactgcttagagcactgtcctagagggcaagtgtagccttcgcctccgggcccaggcaggctatgcagtcagcagtagggtctgtgctccatgcgggtccaggcgcacggctctcctattctgttgtcatttgtgccctctatgggcaggtgtgtttcaagttggttttctgttgctgaggctttcatacacatcagttaccatctcagctgatttgtctactgaaagcttgctgttttcaataaatcttagtttgccatggttttaagtc

SEQ ID NO: 68 - protein, 289 aa, Mus musculus
  misc_feature: Cdca8
MAPKKRSSRGTRTNTLRSRKLASFLKDFDREVQVRTKQIESDRQTLLKEVENLYNIEILRLPKALQGMKWLDYFALGGNKQALEEAAKADRDITEINNLTAEAIQTPLKSVKKRKVIEVEESIKEEEEEEEEGGGGGGRTKKSHKNLRSAKVKRCLPSKKRTQSIQGRGRSKRLSHDFVTPAMSRLEPSLVKPTPGMTPRFDSRVFKTPGLRTPAAKEQVYNISINGSPLADSKEISLSVPIGGGASLRLLASDLQRIDIAQLNPEALGNIRKLSSRLAQICSSIRTGR

SEQ ID NO: 69 - DNA, 2772 bp, Homo sapiens
  misc_feature: Nrp1
atggagagggggctgccgctcctctgcgccgtgctcgccctcgtcctcgccccggccggcgcttttcgcaacgatgaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggagacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaacccatcctcatcaccatcatagccatgagcgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 70 - DNA, 3652 bp, Mus musculus
  misc_feature: Nrp1
tttttttttttttttttttttttttttttttttttcctccttcttcttcttcctgagacatggcccgggcagtggctcctggaagaggaacaagtgtgggaaaagggagaggaaatcggagctaaatgacaggatgcaggcgacttgagacacaaaaagagaagcgcttctcgcgaattcaggcattgcctcgccgctagccttccccgccaagacccgctgaggattttatggttcttaggcggacttaagagcgtttcggattgttaagattatcgtttgctggtttttcgtccgcgcaatcgtgttctcctgcggctgcctggggactggcttggcgaaggaggatggagagggggctgccgttgctgtgcgccacgctcgcccttgccctcgccctggcgggcgctttccgcagcgacaaatgtggcgggaccataaaaatcgaaaacccagggtacctcacatctcccggttaccctcattcttaccatccaagtgagaagtgtgaatggctaatccaagctccggaaccctaccagagaatcataatcaacttcaacccacatttcgatttggaggacagagactgcaagtatgactacgtggaagtaattgatggggagaatgaaggcggccgcctgtgggggaagttctgtgggaagattgcaccttctcctgtggtgtcttcagggccctttctcttcatcaaatttgtctctgactatgagacacatggggcagggttttccatccgctatgaaatcttcaagagagggcccgaatgttctcagaactatacagcacctactggagtgataaagtcccctgggttccctgaaaaataccccaactgcttggagtgcacctacatcatctttgcaccaaagatgtctgagataatcctggagtttgaaagttttgacctggagcaagactcgaatcctcccggaggaatgttctgtcgctatgaccggctggagatctgggatggattccctgaagttggccctcacattgggcgttattgtgggcagaaaactcctggccggatccgctcctcttcaggcgttctatccatggtcttttacactgacagcgcaatagcaaaagaaggtttctcagccaactacagtgtgctacagagcagcatctctgaagattttaagtgtatggaggctctgggcatggaatctggagagatccattctgatcagatcactgcatcttcacagtatggtaccaactggtctgtagagcgctcccgcctgaactaccctgaaaatgggtggactccaggagaagactcctacaaggagtggatccaggtggacttgggcctcctgcgattcgttactgctgtagggacacagggtgccatttccaaggaaaccaagaagaaatattatgtcaagacttacagagtagacatcagctccaacggagaggactggatctccctgaaagagggaaataaagccattatctttcagggaaacaccaaccccacagatgttgtcttaggagttttctccaaaccactgataactcgatttgtccgaatcaaacctgtatcctgggaaactggtatatctatgagatttgaagtttatggctgcaagataacagattatccttgctctggaatgttgggcatggtgtctggacttatttcagactcccagattacagcatccaatcaagccgacaggaattggatgccagaaaacatccgtctggtgaccagtcgtaccggctgggcactgccaccctcaccccacccatacaccaatgaatggctccaagtggacctgggagatgagaagatagtaagaggtgtcatcattcagggtgggaagcaccgagaaaacaaggtgttcatgaggaagttcaagatcgcctatagtaacaatggctctgactggaaaactatcatggatgacagcaagcgcaaggctaagtcgttcgaaggcaacaacaactatgacacacctgagcttcggacgttttcacctctctccacaaggttcatcaggatctaccctgagagagccacacacagtgggcttgggctgaggatggagctactgggctgtgaagtggaagcacctacagctggaccaaccacacccaatgggaacccagtgcatgagtgtgacgacgaccaggccaactgccacagtggcacaggtgatgacttccagctcacaggaggcaccactgtcctggccacagagaagccaaccattatagacagcaccatccaatcagagttcccgacatacggttttaactgcgagtttggctggggctctcacaagacattctgccactgggagcatgacagccatgcacagctcaggtggagtgtgctgaccagcaagacagggccgattcaggaccatacaggagatggcaacttcatctattcccaagctgatgaaaatcagaaaggcaaagtagcccgcctggtgagccctgtggtctattcccagagctctgcccactgtatgaccttctggtatcacatgtccggctctcatgtgggtacactgagggtcaaactacgctaccagaagccagaggaatatgatcaactggtctggatggtggttgggcaccaaggagaccactggaaagaaggacgtgtcttgctgcacaaatctctgaaactatatcaggttatttttgaaggtgaaatcggaaaaggaaaccttggtggaattgctgtggatgatatcagtattaacaaccatatttctcaggaagactgtgcaaaaccaacagacctagataaaaagaacacagaaattaaaattgatgaaacagggagcactccaggatatgaaggagaaggggaaggtgacaagaacatctccaggaagccaggcaatgtgcttaagaccctggatcccatcctgatcaccatcatagccatgagtgccctgggagtactcctgggtgcagtctgtggagttgtgctgtactgtgcctgttggcacaatgggatgtcagaaaggaacctatctgccctggagaactataactttgaacttgtggatggtgtaaagttgaaaaaagataaactgaacccacagagtaattactcagaggcgtgaaggcacggagctggagggaacaagggaggagcacggcaggagaacaggtggaggcatggggactctgttactctgctttcactgtaagctgggaagggcggggactctgttactccgctttcactgtaagctcggaagggcatccacgatgccatgccaggcttttctcaggagcttcaatgagcgtcacctacagacacaagcaggtgactgcggtaacaacaggaatcatgtacaagcctgctttcttctcttggtttcatttgggtaatcagaagccatttgagaccaagtgtgactgacttcatggttcatcctactagcccccttttttcctctctttctccttaccctgtggtggattcttctcggaaactgcaaaatccaagatgctggcactaggcgttattcagtgggcccttttgatggacatgtgacctgtagcccagtgcccagagcatattatcataaccacatttcaggggacgccaacgtccatccacctttgcatcgctacctgcagcgagcacagg

SEQ ID NO: 71 - protein, 923 aa, Mus musculus
  misc_feature: Nrp1
MERGLPLLCATLALALALAGAFRSDKCGGTIKIENPGYLTSPGYPHSYHPSEKCEWLIQAPEPYQRIMINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNSLECTYIIFAPKMSEIILEFESFDLEQDSNPPGGMFCRYDRLEIWDGFPEVGPHIGRYCGQKTPGRIRSSSGVLSMVFYTDSAIAKEGFSANYSVLQSSISEDFKCMEALGMESGEIHSDQITASSQYGTNWSVERSRLNYPENGWTPGEDSYKEWIQVDLGLLRFVTAVGTQGAISKETKKKYYVKTYRVDISSNGEDWISLKEGNKAIIFQGNTNPTDVVLGVFSKPLITRFVRIKPVSWETGISMRFEVYGCKITDYPCSGMLGMVSGLISDSQITASNQADRNWMPENIRLVTSRTGWALPPSPHPYTNEWLQVDLGDEKIVRGVIIQGGKHRENKVFMRKFKIAYSNNGSDWKTIMDDSKRKAKSFEGNNNYDTPELRTFSPLSTRFIRIYPERATHSGLGLRMELLGCEVEAPTAGPTTPNGNPVDECDDDQANCHSGTGDDFQLTGGTTVLATEKPTIIDSTIQSEFPTYGFNCEFGWGSHKTFCHWEHDSHAQLRWSVLTSKTGPIQDHTGDGNFIYSQADENQKGKVARLVSPVVYSQSSAHCMTFWYHMSGSHVGTLRVKLRYQKPEEYDQLVWMVVGHQGDHWKEGRVLLHKSLKLYQVIFEGEIGKGNLGGIAVDDISINNHISQEDCAKPTDLDKKNTEIKIDETGSTPGYEGEGEGDKNISRKPGNVLKTLDPILITIIAMSALGVLLGAVCGVVLYCACWHNGMSERNLSALENYNFELVDGVKLKKDKLNPQSNYSEA

SEQ ID NO: 72 - DNA, 2943 bp, Homo sapiens
  misc_feature: Mcam
gggaagcatggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcaggcctggaacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacagaccaggtgctggaaagggggcctgtgcttcagttgcatgacctgaaacgggaggcaggaggcggctatcgctgcgtggcgtctgtgcccagcatacccggcctgaaccgcacacagctggtcaagctggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatcacgctgcccccgtctcgtaagaccgaacttgtagttgaagttaagtcagataagctcccagaagagatgggcctcctgcagggcagcagcggtgacaagagggctccgggagaccagggagagaaatacatcgatctgaggcattagccccgaatcacttcagctcccttccctgcctggaccattcccagctccctgctcactcttctctcagccaaagctcaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatgctgtaaatacgctcgtcctgccagcagctgagctgggtagcctctctgagctggtttcctgccccaaaggctggcattccaccatccaggtgcaccactgaagtgaggacacaccggagccaggcgcctgctcatgttgaagtgcgctgttcacacccgctccggagagcaccccagcagcatccagaagcagctgcagtgcaagcttgcatgcctgcgtgttgctgcaccaccctcctgtctgcctcttcaaagtctcctgtgacattttttctttggtcagaggccaggaactgtgtcattccttaaagatacgtgccggggccaggtgtggctcacgcctgtaatcccagcactttgggaggccgaggcggcggatcacaaagtcagacgagaccatcctggctaacacggtgaaaccctgtctctactaaaaatacaaaaaaaaattagctaggcgtagtggttggcacctatagtcccagctactcggaaggctgaagcaggagaatggtatgaatccaggaggtggagcttgcagtgagccgagaccgtgccactgcactccagcctgggcaacacagcgagactccgtctcgagccggccggttgcgcgggccctcggaccctcagagaggcgagggttcgagggcacgagttcgaggccaacctggtccacatgggttg

SEQ ID NO: 73 - DNA, 2890 bp, Mus musculus
  misc_feature: Mcam
cgccctccgtcggggaagcatggggctgcccaaactggtgtgcgtcttcttgttcgctgcctgctgctgctgtcgccgtgccgcgggtgtgccaggagaggaaaagcagccagtacccacgcccgacctggtggaggcagaagtgggcagcacagcccttctcaagtgtggcccctcacgggcctcaggcaacttcagccaagtggactggtttttgattcacaaggagaggcagatactgattttccgtgtgcaccaaggcaagggccagcgggaacctggtgaatatgagcaccgccttagcctccaagactcggtggctactctggccctgagtcacgtcactccccatgatgagcgaatgttcctgtgtaagagcaagcgaccacggctccaggatcactacgttgagcttcaggtcttcaaagccccagaggaaccaactattcaagccaatgtcgtgggcatccatgtggacaggcaagagctcagggaggttgctacctgtgtggggagaaacggctaccccattcctcaagtcctatggtacaagaacagtctgcccttgcaagaggaggagaaccgagttcatatccagtcatcacagattgtcgagtccagtggcttgtacaccttgaagagtgttctgagtgcacgcctagttaaggaagacaaagatgcccagttttactgtgaactcagctaccggctacccagtgggaaccacatgaaggaatctaaggaggtcactgtccctgttttctaccctgcagaaaaagtgtgggtggaggtagagcctgtggggctgctgaaggaaggggatcatgtgacaatcaggtgtctgacagatggcaaccctcaaccccacttcactatcaacaagaaggaccccagcactggggagatggaagaggagagcaccgatgaaaatgggctcctgtccttggagcctgccgaaaagcaccatagcgggctctaccagtgtcagagtctggacctggaaactaccatcacactgtcaagtgaccccctggagctgctggtgaactatgtgtctgatgttcaagtgaatccaactgcccctgaagtccaggaaggtgagagcctcacgctgacctgcgaggcagaaagtaaccaggaccttgagtttgagtggctgagagacaagacaggccagctgctgggaaagggtcccgtcctccagctaaacaacgtgagacgggaagcagggggacggtatctctgcatggcatctgtccccagagttcctggcttgaatcgtacccagctggtcagcgtgggcatttttgggtccccatggatggcattaaaggagaggaaggtgtgggtgcaagagaatgcagtgctgaatctgtcttgtgaggcttcaggacatcctcagcccaccatctcctggaatgtcaatggttcggcaactgaatggaacccagatccacagacagtagtgagcaccttgaatgtccttgtgacgccagagcttctggagacaggtgcagagtgtacagcctccaactccctgggctcaaacaccaccaccattgttctgaagctggtcactttaaccaccctcatacctgactccagccaaaccactggcctcagcaccctcacagtcagtcctcacaccagagccaacagcacctccacagagaaaaagctgccacagccagagagcaaaggtgtggtcatcgtggctgtgatagtgtgtaccttggtgcttgctgtgctgggtgctgctctctatttcctctacaagaagggcaagctgccatgtggacgctcgggaaaacaggagatcacgctgcccccgactcgtaagagtgaatttgtagttgaagttaagtcagataagctcccagaagagatggctctccttcagggcagcaacggtgacaagagggctccaggagaccagggagagaaatacatcgatctgaggcattagatggctcccattgcactgctcgcagctccctgctcagacttcaccccaagctgaagcctccagagggacagcagggacgagccacactcaaccccccccctgcacatcaggtctgagagctaggagctgggacaggagtcgtctgcaggagctcagttggccacagaggcctggttttagagaccaagccctcctctgtgtccagtaaataatgcttatcccaaggggcccgtctcccagggcatttccccctcccgtgcacagccattggtggcaaatccttctgccatcagctgtgtgggcttgcctctttgagctcatctcccctcacaggctgtcttcatgatgcaggacctgggcacatggtcacattattccgttcacattggtccttgtgagaacctcacagtctggaggcggctgcttttgtaccttcctgcctgctactaattcagggtctcatttggaacatttttcctttgggtagtggtcaggaactggtgtaagtcctccagacacatccctgtgtaaggaagccagggcactgtttctctgagttttgttgttttgttttctttgaaggctactgagcccaagcttcccgcattcccttagtaacaagagacaggacagagagaaggtctactgttcatggggattaggcttataggaatgttagtaccaaatttctacatgtgagctttgggggccaggtcctagagagcccaagtgggagaatggtatttaggagatgaaaaacctggcctagcaagagcttttgaggtgtgtgtgtgtgtgtgtgtatacatatatgtgtgtatatatatatatatatatataggttttgtctgtaaatttgcaaatttttccttttatatgtgtgttagaaaaataaagtgttattgtcccaaaaaaaaaaaaaaa

SEQ ID NO: 74 - protein, 648 aa, Mus musculus
  misc_feature: Mcam
MGLPKLVCVFLFAACCCCRRAAGVPGEEKQPVPTPDLVEAEVGSTALLKCGPSRASGNFSQVDWFLIHKERQILIFRVHQGKGQREPGEYEHRLSLQDSVATLALSHVTPHDERMFLCKSKRPRLQDHYVELQVFKAPEEPTIQANVVGIHVDRQELREVATCVGRNGYPIPQVLWYKNSLPLQEEENRVHIQSSQIVESSGLYTLKSVLSARLVKEDKDAQFYCELSYRLPSGNHMKESKEVTVPVFYPAEKVWVEVEPVGLLKEGDHVTIRCLTDGNPQPHFTINKKDPSTGEMEEESTDENGLLSLEPAEKHHSGLYQCQSLDLETTITLSSDPLELLVNYVSDVQVNPTAPEVQEGESLTLTCEAESNQDLEFEWLRDKTGQLLGKGPVLQLNNVRREAGGRYLCMASVPRVPGLNRTQLVSVGIFGSPWMALKERKVWVQENAVLNLSCEASGHPQPTISWNVNGSATEWNPDPQTVVSTLNVLVTPELLETGAECTASNSLGSNTTTIVLKLVTLTTLIPDSSQTTGLSTLTVSPHTRANSTSTEKKLPQPESKGVVIVAVIVCTLVLAVLGAALYFFYKKGKLPCGRSGKQEITLPPTRKSEFVVEVKSDKLPEEMALLQGSNGDKRAPGDQGEKYIDLRH

SEQ ID NO: 75 - DNA, 1582 bp, Homo sapiens
  misc_feature: Pbk
gtaagaaagccaggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagccttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacaaaaaaaaaaaaaaaaaa

SEQ ID NO: 76 - DNA, 1666 bp, Mus musculus
  misc_feature: Pbk
gaggggagctgttcctgcattttctggagcgagtcttctgactgcttttagttagaactccagtgcccctcggcgggccgcggcctttgaaaatgcgcgcgccctaaacgctgcggcggttacgctgttggcgggagggagctgagcctgcactttccggactaggtgtccagacagctttgagccagcccgtcactttcacctttttacccgagcgtgcgagcgtggacctaacgtgattgctacaatggaaggaattaataatttcaagacgccaaacaaatctgaaaaaaggaaatctgtattatgttccactccatgtgtaaatatccctgcctctccatttatgcagaagcttggctttgggactggggtcagcgtttacctaatgaaaagatctccaagagggttgtctcattctccttgggccgtgaaaaagataagtcttttatgcgatgatcattatcgaactgtgtatcagaagagactaactgatgaagctaagattttaaaaaaccttaatcacccaaacattataggatatcgtgcttttactgaagccagtgatggtagtctgtgccttgctatggagtatggaggtgaaaagtctctgaatgacttaatagaagagcggaacaaagacagtggaagtccttttccagcagctgtaattctcagagttgctttgcacatggccagagggctaaagtacctgcaccaagaaaagaagctgcttcatggagacataaagtcttcaaatgttgtaattaaaggtgattttgaaacaattaaaatctgtgatgtaggagtctctctgccattggatgaaaatatgactgtgactgatcctgaggcctgttatattggtactgagccatggaaacccaaggaagcgttggaagaaaatggcatcattactgacaaggcagatgtgtttgcttttggccttactctgtgggaaatgatgactttatgtattccacacgtcaatcttccagatgatgatgttgatgaagatgcaacctttgatgagagtgacttcgatgatgaagcatattatgcagctctggggacaaggccatccatcaacatggaagagctggatgactcctaccagaaggccattgaactcttctgtgtgtgcactaatgaggatcctaaagatcgcccgtctgctgcacacatcgttgaagctttggaactagatggccaatgttgtggtctaagctcaaagcattaacttgtatgggaactgttaactagatatatgtagttaatataacttatggtagctagattctagaagtagctttaacactagtgacccctgtctaagatgacttaagaatcaagggaccattgctttgttacagatctttttagatattcttgcttctttagtgggttactaaaaatttcactacgtacatgtggtacagatatctgtctgctcatagtgtcagtccttcagctggcctgtcagcccatgcgccctgggacttgagaagagttcataaacgtagctcctagggtgtcttgcctctctacacttagcttctaatttattactttgtttctactgattgtgtcttaagtcttttaaaataaatgtaagaataaacaataaaagacagttttagtaccaggcaaaaaaaaaaaaaaaaaa

SEQ ID NO: 77 - protein, 330 aa, Mus musculus
  misc_feature: Pbk
MEGINNFKTPNKSEKRKSVLCSTPCVNIPASPFMQKLGFGTGVSVYLMKRSPRGLSHSPWAVKKISLLCDDHYRTVYQKRLTDEAKILKNLNHPNIIGYRAFTEASDGSLCLAMEYGGEKSLNDLIEERNKDSGSPFPAAVILRVALHMARGLKYLHQEKKLLHGDIKSSNVVIKGDFETIKICDVGVSLPLDENMTVTDPEACYIGTEPWKPKEALEENGIITDKADVFAFGLTLWEMMTLCIPHVNLPDDDVDEDATFDESDFDDEAYYAALGTRPSINMEELDDSYQKAIELFCVCTNEDPKDRPSAAHIVEALELDGQCCGLSSKH

SEQ ID NO: 78 - DNA, 1207 bp, Homo sapiens
  misc_feature: Akr1c1
ccagaaatggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctaaaaaa

SEQ ID NO: 79 - protein, 323 aa, Homo sapiens
  misc_feature: Akr1c1
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 80 - DNA, 1356 bp, Mus musculus
  misc_feature: Akr1c1
ttgtcctgactctgttctgcagccctgattgattagtagcagcttggttacaatacatttttgtcatctgcattgacctggtctttaagttatattggatttatgttggatttaagtggacccacaacactttgaggaagaagaagacactcttcttactttggagtacccagtgatatcaggaaagtcagaggcagagcctgcagatgaatcccaagcgctacatggaactaagtgatggccaccacattcctgtgcttggctttggaacctttgtcccaggagaggtttccaagagtatggttgcaaaagccaccaaaatagctatagatgctggattccgccatattgactcagcttatttctaccaaaatgaggaggaagtagggctggccatccgaagcaaggttgctgatggcactgtgaggagagaagatatattctacacttcaaagcttccctgcacatgtcatagaccagagctggtccagccttgcttggaacaatccctgagaaagcttcagctggattatgttgatctgtaccttattcactgcccagtgtccatgaagccaggcaatgatcttattccaacagatgaaaatgggaaattattatttgacacagtggatctctgtgacacatgggaggccatggagaagtgtaaggattcagggttagccaagtccattggtgtgtccaactttaaccggaggcagctggagatgatcctgaacaagccagggctcaggtacaagcctgtgtgcaaccaggtagagtgtcacccttatctgaaccagagcaagctcctggactactgcaagtcaaaagacatcgttctggttgcctatggtgctcttggcagccaacggtgtaagaactggatagaggagaatgccccatatctcttggaagacccaactctgtgtgccatggcggaaaagcacaagcaaactccggccctaatttccctccggtatctgctgcagcgtgggattgtcattgtcaccaagagtttcaatgagaagcggatcaaggagaacctgaaggtctttgagttccacttgccagcagaggacatggcagttatagataggctgaacagaaactaccgatatgctactgctcgtattatttctgctcaccccaattatccatttttggatgaatattaacgcggaagcctttgttgtgacatcgctcagagggagcaatgtgggagatgctgtggatgttgatcagcatcacctctggtcgacgtcgacatcaccgtcaacccacactgaactggatggagaggggtggccatggtgttttgtgatactttgaagacaataaagttttggtctatgaggt

SEQ ID NO: 81 - protein, 322 aa, Mus musculus
  misc_feature: Akr1c1
MNPKRYMELSDGHHIPVLGFGTFVPGEVSKSMVAKATKIAIDAGFRHIDSAYFYQNEEEVGLAIRSKVADGTVRREDIFYTSKLPCTCHRPELVQPCLEQSLRKLQLDYVDLYLIHCPVSMKPGNDLIPTDENGKLLFDTVDLCDTWEAMEKCKDSGLAKSIGVSNFNRRQLEMILNKPGLRYKPVCNQVECHPYLNQSKLLDYCKSKDIVLVAYGALGSQRCKNWIEENAPYLLEDPTLCAMAEKHKQTPALISLRYLLQRGIVIVTKSFNEKRIKENLKVFEFHLPAEDMAVIDRLNRNYRYATARIISAHPNYPFLDEY

SEQ ID NO: 82 - DNA, 1821 bp, Homo sapiens
  misc_feature: Cyp11a1
gggcgctgaagtggagcaggtacagtcacagctgtggggacagcatgctggccaagggtcttcccccacgctcagtcctggtcaaaggctaccagacctttctgagtgcccccagggaggggctggggcgtctcagggtgcccactggcgagggagctggcatctccacccgcagtcctcgccccttcaatgagatcccctctcctggtgacaatggctggctaaacctgtaccatttctggagggagacgggcacacacaaagtccaccttcaccatgtccagaatttccagaagtatggcccgatttacagggagaagctcggcaacgtggagtcggtttatgtcatcgaccctgaagatgtggcccttctctttaagtccgagggccccaacccagaacgattcctcatcccgccctgggtcgcctatcaccagtattaccagagacccataggagtcctgttgaagaagtcggcagcctggaagaaagaccgggtggccctgaaccaggaggtgatggctccagaggccaccaagaactttttgcccctgttggatgcagtgtctcgggacttcgtcagtgtcctgcacaggcgcatcaagaaggcgggctccggaaattactcgggggacatcagtgatgacctgttccgctttgcctttgagtccatcactaacgtcatttttggggagcgccaggggatgctggaggaagtagtgaaccccgaggcccagcgattcattgatgccatctaccagatgttccacaccagcgtccccatgctcaaccttcccccagacctgttccgtctgttcaggaccaagacctggaaggaccatgtggctgcatgggacgtgattttcagtaaagctgacatatacacccagaacttctactgggaattgagacagaaaggaagtgttcaccacgattaccgtggcatgctctacagactcctgggagacagcaagatgtccttcgaggacatcaaggccaacgtcacagagatgctggcaggaggggtggacacgacgtccatgaccctgcagtggcacttgtatgagatggcacgcaacctgaaggtgcaggatatgctgcgggcagaggtcttggctgcgcggcaccaggcccagggagacatggccacgatgctacagctggtccccctcctcaaagccagcatcaaggagacactaagacttcaccccatctccgtgaccctgcagagatatcttgtaaatgacttggttcttcgagattacatgattcctgccaagacactggtgcaagtggccatctatgctctgggccgagagcccaccttcttcttcgacccggaaaattttgacccaacccgatggctgagcaaagacaagaacatcacctacttccggaacttgggctttggctggggtgtgcggcagtgtctgggacggcggatcgctgagctagagatgaccatcttcctcatcaatatgctggagaacttcagagttgaaatccaacacctcagcgatgtgggcaccacattcaacctcattctgatgcctgaaaagcccatctccttcaccttctggccctttaaccaggaagcaacccagcagtgatcagagaggatggcctgcagccacatgggaggaaggcccaggggtggggcccatggggtctctgcatcttcagtcgtctgtcccaagtcctgctcctttctgcccagcctgctcagcaggttgaatgggttctcagtggtcaccttcctcagctcagctgggccactcctcttcacccaccccatggagacaataaacagctgaaccatcg

SEQ ID NO: 83 - DNA, 1774 bp, Mus musculus
  misc_feature: Cyp11a1
aagtggcagtcgtggggacagtatgctggctaaaggactttccctgcgctcagtgctggtcaaaggctgccaacctttcctgagccctacgtggcagggtccagtgctgagtactggaaagggagctggtacctctactagcagtcctaggtccttcaatgagatcccttcccctggcgacaatggttggctaaacctgtaccacttctggagggagagtggcacacagaaaatccattaccatcagatgcagagtttccaaaagtatggccccatttacagggagaagctgggcactttggagtcagtttacatcgtggaccccaaggatgcgtcgatactcttctcatgcgagggtcccaacccggagcggttccttgtgcccccctgggtggcctatcaccagtattatcagaggcccattggggtcctgtttaagagttcagatgcctggaagaaagaccgaatcgtcctaaaccaagaggtgatggcgcctggagccatcaagaacttcgtgcccctgctggaaggtgtagctcaggacttcatcaaagtcttacacagacgcatcaagcagcaaaattctggaaatttctcaggggtcatcagtgatgacctattccgcttttcctttgagtccatcagcagtgttatatttggggagcgcatggggatgctggaggagatcgtggatcccgaggcccagcggttcatcaatgctgtctaccagatgttccacaccagtgtccccatgctcaacctgcctccagacttctttcgactcctcagaactaagacctggaaggaccatgcagctgcctgggatgtgattttcaataaagctgatgagtacacccagaacttctactgggacttaaggcagaagcgagacttcagccagtaccctggtgtcctttatagcctcctggggggcaacaagctgcccttcaagaacatccaggccaacattaccgagatgctggcaggaggggtggacacgacctccatgaccctgcagtggaacctttatgagatggcacacaacttgaaggtacaggagatgctgcgggctgaagtcctggctgcccggcgccaggcccagggagacatggccaagatggtacagttggttccactcctcaaagccagcatcaaggagacactgagactccaccccatctccgtgaccttgcagaggtacactgtgaatgacctggtgcttcgtaattacaagattccagccaagactttggtacaggtggctagctttgccatgggtcgagatccgggcttctttcccaatccaaacaagtttgacccaactcgttggctggaaaaaagccaaaataccacccacttccggtacttgggctttggctggggtgttcggcagtgtctgggccggcggattgcggagctggagatgaccatcctccttatcaatctgctggagaacttcagaattgaagttcaaaatctccgtgatgtggggaccaagttcagcctcatcctgatgcctgagaaccccatcctcttcaacttccagcctctcaagcaggacctgggcccagccgtgaccagaaaagacaacactgtgaactgaaggctggagtcacatggggaggtggcccatggggcatttgagggtggtatctctgtatcttcagaaacagcactctgtgattacctgcccaggttagctgggctctcctctccttcatcctctttccctctttccctacccagggagttaataaacacttgaacactgagg

SEQ ID NO: 84 - protein, 526 aa, Mus musculus
  misc_feature: Cyp11a1
MLAKGLSLRSVLVKGCQPFLSPTWQGPVLSTGKGAGTSTSSPRSFNEIPSPGDNGWLNLYHFWRESGTQKIHYHQMQSFQKYGPIYREKLGTLESVYIVDPKDASILFSCEGPNPERFLVPPWVAYHQYYQRPIGVLFKSSDAWKKDRIVLNQEVMAPGAIKNFVPLLEGVAQDFIKVLHRRIKQQNSGNFSGVISDDLFRFSFESISSVIFGERMGMLEEIVDPEAQRFINAVYQMFHTSVPMLNLPPDFFRLLRTKTWKDHAAAWDVIFNKADEYTQNFYWDLRQKRDFSQYPGVLYSLLGGNKLPFKNIQANITEMLAGGVDTTSMTLQWNLYEMAHNLKVQEMLRAEVLAARRQAQGDMAKMVQLVPLLKASIKETLRLHPISVTLQRYTVNDLVLRNYKIPAKTLVQVASFAMGRDPGFFPNPNKFDPTRWLEKSQNTTHFRYLGFGWGVRQCLGRRIAELEMTILLINLLENFRIEVQNLRDVGTKFSLILMPENPILFNFQPLKQDLGPAVTRKDNTVN

(19) United States
(12) Patent Application Publication
(10) Pub. No.: US 2019/0060366 A1
(43) Pub. Date: Feb. 28, 2019

(54) HIGHLY ENGRAFTABLE HEMATOPOIETIC STEM CELLS

(71) Applicants: President and Fellows of Harvard College, Cambridge, MA (US); The General Hospital Corporation, Boston, MA (US)

(72) Inventors: Jonathan HOGGATT, Somerville, MA (US); David T. SCADDEN, Weston, MA (US)

(21) Appl. No.: 16/080,264

(22) PCT Filed: Feb. 27, 2017
(86) PCT No.: PCT/US17/19778

§ 371 Date: Aug. 27, 2018

Related U.S. Application Data
(60) Provisional application No. 62/300,694, filed on Feb. 26, 2016.
(60) Provisional application No. 62/413,821, filed on Oct. 27, 2016.

(51) Int. Cl. A61K 35/28; A61P 35/02; C12N 5/0797

(52) U.S. Cl. CPC A61K 35/28; A61K 45/06; C12N 5/0623; A61P 35/02

(57) ABSTRACT

The present inventions relates to highly engraftable hematopoietic stem cell (heHSC) and related methods of production and use for the treatment of stem cell and progenitor cell disorders.

### CROSS-REFERENCE TO RELATED APPLICATIONS

This application claims the benefit of U.S. Provisional Application Ser. No. 62/413,821, filed Oct. 27, 2016 and U.S. Provisional Application No. 62/300,694, filed Feb. 26, 2016, the contents of which are incorporated herein by reference in their entireties.

### BACKGROUND OF THE INVENTION

Hematopoietic stem cell (HSC) transplantation is currently the only curative treatment modality for a number of stem cell disorders, including both malignant and non-malignant hematologic conditions. Yet, despite the fact that hematopoietic transplant is the only curative option for patients having such stem cell disorders, transplant-related morbidity and mortality remains high, and only a fraction of the patients that could benefit from an HSC transplant actually receive one.

Sources of HSCs for transplantation include the bone marrow itself, umbilical cord blood, and mobilized peripheral blood. Under steady state conditions, HSCs and hematopoietic progenitor cells (HPCs) normally reside within the bone marrow niches, while the mature cells produced by these populations of HSCs and HPCs ultimately exit the bone marrow and enter the peripheral blood. Considerable evidence over the last several decades, however, clearly demonstrates that HSCs and HPCs (collectively referred to as “HSPCs”) also exit the bone marrow niche and traffic to the peripheral blood and we now know that this natural egress into the periphery can be enhanced, allowing for “mobilization” of these cells from the bone marrow to the peripheral blood. Mobilized adult HSCs and HPCs are widely used for autologous and allogeneic transplantation and have improved patient outcomes when compared to bone marrow grafts.

The hematopoietic growth factor, granulocyte-colony stimulating factor (G-CSF) is widely used clinically to mobilize HSC and HPC for transplantation. G-CSF-mobilized peripheral blood stem cells (PBSCs) are associated with more rapid engraftment, shorter hospital stays, and in some circumstances, superior overall survival compared to bone marrow grafts, though the use of G-CSF-mobilized grafts over bone marrow in some allogeneic settings is under scrutiny.

While successful, G-CSF mobilization regimens involve repeated subcutaneous injections and are often associated with morbidity from bone pain (an often severe and debilitating complication), nausea, headache, and fatigue. These can be lifestyle disruptive in normal volunteers and particularly distressing for patients who are enduring the rigors of cancer chemotherapy. In a small population of normal donors, G-CSF has also been associated with serious toxicity, including enlargement of the spleen and splenic rupture, and the pro-coagulant effects of G-CSF can increase the risk of myocardial infarction and cerebral ischemia in high-risk individuals. Despite its success for most patients and donors, poor mobilization in response to G-CSF occurs in 15% of normal, healthy donors, and often those who do achieve sufficient numbers of CD34+ cells require more than one apheresis procedure. Repeated, prolonged sessions of apheresis are particularly common among autologous donors, which is particularly troubling for them given their ongoing ordeals associated with their underlying cancer and its treatment. Up to 60% of patients that fail to mobilize an optimal CD34+ cell dose for autologous transplantation often requiring tandem cycles of high dose chemotherapy. This is particularly an issue for patients with lymphoma and multiple myeloma, who often require extended aphereses and comprise the largest group of transplant recipients.

The availability of alternative methods for mobilizing HSPC could have high impact on the foregoing obstacles associated with HSC transplantation. Needed are novel therapeutics and methods that are capable of enhancing graft acquisition and hematopoietic recovery and engraftment. Also needed are highly engraftable cells that may be used to treat stem cell and/or progenitor cell disorders, such as malignant and non-malignant hematologic diseases.

### SUMMARY OF THE INVENTION

There remains a need for novel compositions, methods and therapies that are capable of reducing hematopoietic stem cell (HSC) transplant-related morbidity and mortality and enhancing engraftment of transplanted HSCs in subjects in need of a stem cell transplant. The present inventions are directed toward further solutions to address these unmet needs, in addition to having other desirable characteristics. Accordingly, disclosed herein is an isolated, highly engraftable hematopoietic stem cell (heHSC), as well as related methods of preparing such heHSCs and related methods of using such heHSCs for the treatment of stem cell and/or progenitor cell disorders and other diseases for which a stem cell transplant may be indicated.

In certain aspects, the present inventions are directed to an isolated, heHSC, wherein the heHSC is Sca-1+ and c-kit+ and is negative for Lineage markers (e.g., B221−, CD3−, Gr-1−, Mac-1−, TER119−) (e.g., a Sca-1+, c-kit+ and Lin− (SKL) cell). In certain aspects, the isolated heHSC is CD48−. In certain aspects the heHSC is not naturally occurring, i.e., differs from a naturally occurring HSC in one or more ways including but not limited to functionality (e.g., engraftability) and gene expression. In certain aspects, the isolated heHSC is CD150+. In certain aspects, the isolated heHSC is a Signaling lymphocytic activation molecule (SLAM) SKL cell, which is CD150+, CD48−, Sca-1+, c-kit+ and lineage negative. In certain aspects, the isolated heHSC does not express an immunophenotypic means of identifying human hematopoietic stem cells (e.g., the isolated heHSC does not express antigens, markers or other characteristics that may be useful for distinguishing such heHSC from other cell types). In some embodiments, the isolated heHSC comprises a unique transcriptome relative to hematopoietic stem cells contacted with granulocyte colony-stimulating factor (G-CSF), a chemotherapeutic agent, or any combination thereof. For example, in some aspects, the isolated heHSCs disclosed herein are characterized based on their differential expression of one or more of the genes selected from the group consisting of Fos, CD93, Fosb, Dusp1, Jun, Dusp6, Cdk1, Fignl1, Plk2, Rsad2, Sgk1, Sdc1, Serpine2, Spp1, Cdca8, Nrp1, Mcam, Pbk, Akr1cl and Cyp11a1 (e.g., relative to the expression of one or more genes by hematopoietic stem cells mobilized using G-CSF). In some embodiments, the isolated heHSC expresses osteopontin (e.g., the heHSC is OPN+). In some embodiments, the isolated heHSC expresses CD93 (e.g., the heHSC is CD93+) than an HSC obtained from a subject subjected to a conventional mobilization regimen. In some embodiments, the isolated heHSC does not express CD34 or is CD34−. In some embodiments, the isolated heHSC is CD93+ and CD34−. In some embodiments, the heHSC is a non-native or non-naturally occurring cell, i.e., possesses one or more genotypic or phenotypic characteristics not present in native or naturally occurring HSC. In some embodiments, the isolated heHSC is from in a population of cells not present in a non-treated host and/or a host treated with a conventional mobilization regimen (e.g., a cell population with a different gene expression profile or a different phenotype profile). In some embodiments, the heHSC is from in a population of heHSC with a higher proportion of CD93+ cells than a HSC population obtained from a host treated with a conventional mobilization regimen.

Conventional procedures using G-CSF are known in the art. See Schmitt, M et al. “Mobilization of PBSC for Allogeneic Transplantation by the Use of the G-CSF Biosimilar XM02 in Healthy Donors.” Bone Marrow Transplantation 48.7 (2013): 922-925. PMC. Web. 24 Feb. 2017, incorporated herein by reference.

As used herein, “differentially expresses”, when used in reference to a cell population means an expression that is at least 10% higher than or lower than a reference value (e.g., an heHSC population differentially expresses CD93 from an HSC population obtained by a conventional immobilization technique if the heHSC population expresses at least 10% more or less CD93). As used herein, “differentially expresses,” when used in reference to a cell, means that the cell has a different expression pattern of one or more phenotypes than a reference cell.

In certain aspects of the present inventions, the isolated heHSCs disclosed herein may be transformed to express a polynucleotide (e.g., an exogenous polynucleotide). For example, in certain embodiments, an isolated heHSC is transformed with an expression vector to express a polynucleotide (e.g., an exogenous polynucleotide). In some embodiments, the expression vector comprises a viral vector selected from the group consisting of a retrovirus, a herpes simplex, an adenovirus, a lentivirus, and an adeno-associated virus. In some embodiments, the isolated heHSC is transfected with an expression vector that comprises the polynucleotide. In some embodiments, the polynucleotide comprises an exogenous polynucleotide.

Also disclosed herein is the use of isolated heHSCs to deliver an exogenous polynucleotide to a subject in need thereof. For example, the isolated heHSCs disclosed herein may be transformed to express an exogenous polynucleotide and, upon engraftment in the subject's tissues (e.g., bone marrow tissues), the engrafted heHSC expresses the exogenous polynucleotide, thereby delivering the expression product (e.g., a protein, enzyme or amino acid) to the subject.

Also disclosed herein are methods of transforming an isolated heHSC, wherein such methods comprise a step of contacting the heHSC with an expression vector under conditions sufficient for the vector to integrate into the heHSC genome. In yet other embodiments, the isolated heHSC of the present inventions are genetically modified to shut off expression of an endogenous polynucleotide.

In certain embodiments, the isolated heHSC is substantially pure (e.g., at least 10%, 20%, 30%, 40%, 50%, 60%, 70%, 80%, 90%, 95%, 97.5%, 98%, 99% or more pure). In certain aspects, the isolated heHSC is non-quiescent.

Also disclosed herein are methods of preparing an isolated, heHSC. For example, in some embodiments, the isolated heHSC disclosed herein is prepared by contacting a hematopoietic stem cell and/or a progenitor cell with at least one CXCR2 agonist and at least one CXCR4 antagonist, VLA-4 antagonist, α₉β₁ antagonist, α₉β₁ integrin/VLA-4 antagonist or combination thereof. In some embodiments, the isolated heHSC disclosed herein is prepared by contacting a hematopoietic stem cell and/or a progenitor cell with at least one CXCR2 agonist and at least one CXCR4 antagonist. In some embodiments, such contacting is performed in vivo, for example by administering GROβ or an analog or derivative thereof and plerixafor or an analog or derivative thereof to a human subject. In some embodiments, such contacting is performed in vitro. In some in vivo embodiments, such contacting mobilizes an amount of circulating peripheral blood stem cells in the subject sufficient to harvest a cell dose of between about 1×10⁶/kg body weight and 10×10⁶/kg body weight in a single apheresis session. In some in vivo embodiments, such contacting mobilizes an amount of circulating peripheral blood stem cells in the subject sufficient to harvest a cell dose of between about 2×10⁶/kg body weight and 8×10⁶/kg body weight in a single apheresis session. In some in vivo embodiments, such contacting mobilizes an amount of circulating peripheral blood stem cells in the subject sufficient to harvest a cell dose of between about 3×10⁶/kg body weight and 6×10⁶/kg body weight in a single apheresis session. In some in vitro embodiments, isolated HSC are contacted with sufficient amount of at least one CXCR2 agonist and at least one CXCR4 antagonist, VLA-4 antagonist, α₉β₁ antagonist, α₉β₁ integrin/VLA-4 antagonist or combination thereof to obtain between 1×10⁶ and 1.2×10⁹ heHSC cells.

In some embodiments, the at least one CXCR2 agonist comprises GROβ or an analog or derivative thereof. In some embodiments the at least one CXCR2 agonist comprises GROβ-Δ4 or an analog or derivative thereof. In some embodiments, the at least one CXCR4 antagonist comprises plerixafor (AMD-3100) or an analog or derivative thereof. In some embodiments, the at least one CXCR4 antagonist comprises ALT1188, ALT1187, ALT1128, ALT1228, or TG-0054 or an analog or derivative thereof. In some embodiments, the CXCR4 antagonist comprises at least one inhibitor described in Debnath B, et al., “Small Molecule Inhibitors of CXCR4,” Theranostics 2013; 3(1):47-75, incorporated herein by reference. In some embodiments, the α₉β₁ integrin/VLA-4 antagonist is N-(benzenesulfonyl)-L-prolyl-L-O-(1-pyrrolidinylcarbonyl)tyrosine (BOP) or an analog or derivative thereof (e.g., R-BC154). In some embodiments, the VLA-4 antagonist is BIO 5192, Natalizumab, firategrast, or an analog or derivative thereof. In still other embodiments, the at least one CXCR2 agonist is GROβ or an analog or derivative thereof and the at least one CXCR4 antagonist is plerixafor or an analog or derivative thereof. In some embodiments, a Gro-beta analog or derivative is the desamino Gro-beta protein (also known as MIP-2alpha), which comprises the amino acid sequence of mature gro-S protein truncated at its N terminus between amino acid positions 2 and 8, as described in PCT International Application Publication WO/1994/029341, incorporated herein by reference in its entirety. In other embodiments, the Gro-beta analog or derivative is the dimeric modified Gro-beta protein described in U.S. Pat. No. 6,413,510, incorporated herein by reference in its entirety. In some embodiments, the Gro-beta analog or derivative is SB-251353, a Gro-beta analog involved in directing movement of stem cells and other leukocytes, as described by Bensinger et al. (Bone Marrow Transplantation (2009), 43, 181-195, incorporated by reference herein).

The isolated heHSCs disclosed herein are characterized by their enhanced ability to engraft in a target tissue of a subject (e.g., the bone marrow tissue of a subject). Accordingly, in some embodiments upon administration or transplant of the heHSC in a subject such heHSC demonstrates increased engrafting ability, for example, relative to engraftment of the same quantity of hematopoietic stem cells that are contacted or mobilized with granulocyte colony-stimulating factor (G-CSF), chemotherapeutic agents (e.g., mobilizing chemotherapeutic agents), or any combinations thereof. In certain embodiments, such engrafting ability is increased by at least about two-fold, three-fold, four-fold, five-fold, six-fold, or more.

In some embodiments, the heHSC is a non-native cell, i.e., possesses one or more genotypic or phenotypic characteristics not present in native HSC. In some embodiments, the isolated heHSC is from in a population of cells not present in a non-treated host and/or a host treated with a conventional mobilization regimen (e.g., a cell population with a different gene expression profile or a different phenotype profile). In some embodiments, the heHSC is from in a population of heHSC with a higher proportion of CD93+ cells than a HSC population obtained from a host treated with a conventional mobilization regimen.

The isolated heHSCs disclosed herein are also characterized by their ability to produce or cause improved or increased donor chimerism following their engraftment. In some embodiments, upon engraftment of the heHSCs in a subject the heHSCs demonstrate increased donor chimerism, for example, relative to the donor chimerism observed following engraftment of the same quantity of hematopoietic stem cells contacted or mobilized with G-CSF, chemotherapeutic agents (e.g., mobilizing chemotherapeutic agents), or any combinations thereof. In certain embodiments, such donor chimerism is increased by at least about two fold, three-fold, four-fold, five-fold, six-fold, or more. In some embodiments, such donor chimerism is at least about 10%, 20%, 25%, 30%, 40%, 50%, 60%, 70%, 75%, 80%, 85%, 90%, 95%, 97%, 99%, or more.

In certain aspects, the present inventions are directed to methods of treating a stem cell or progenitor cell disorder. Such methods comprise a step of administering an isolated heHSC (e.g., a SLAM SKL heHSC) to a subject in need thereof, wherein the administered heHSC engrafts in the subject's tissues (e.g., the subject's bone marrow compartment), thereby treating the stem cell or progenitor cell disorder. In some embodiments, the methods described herein comprise administering a population of cells comprising at least about 20%, 25%, 30%, 35%, 40%, 45%, 50%, 55%, 60%, 65%, 70%, 75%, 80%, 85%, 90%, or 95% heHSC cells.

In certain aspects, upon engraftment in a subject, the engrafted heHSCs demonstrate enhanced hematopoietic function relative to engraftment of the same quantity of hematopoietic stem cells contacted or mobilized with G-CSF, chemotherapeutic agents (e.g., mobilizing chemotherapeutic agents), or any combinations thereof. In some embodiments, upon engraftment in a subject the engrafted heHSCs demonstrate an enhanced CD34+ number relative to engraftment of the same quantity of hematopoietic stem cells contacted or mobilized with G-CSF, chemotherapeutic agents, or any combinations thereof. In certain embodiments, upon engraftment in a subject the engrafted heHSCs demonstrate enhanced hematopoietic function relative to engraftment of the same quantity of hematopoietic stem cells contacted or mobilized with granulocyte colony-stimulating factor (G-CSF), chemotherapeutic agents, or any combinations thereof.

In some embodiments, the subject (e.g., a human subject) is conditioned for engraftment prior to administering the isolated heHSCs disclosed herein. In some embodiments, the subject (e.g., a human subject) exhibits poor mobilization in response to a conventional mobilization regimen, such as G-CSF.

Also disclosed herein are methods of treating a stem cell and/or progenitor cell disorder in a subject, the method comprising: (a) depleting an endogenous hematopoietic stem cell or progenitor cell population in a bone marrow compartment of the subject; and (b) administering an isolated, non-native heHSC to the subject, wherein the heHSC is Sca-1+, c-kit+ and Lin− (SKL), and where the administered heHSC engrafts in the bone marrow compartment of the subject. In certain embodiments, the heHSC is a SLAM SKL heHSC.

The heHSCs disclosed herein may be used for the treatment of stem cell and/or progenitor cell disorders or any diseases for which a stem cell transplant may be indicted. In some embodiments, such a stem cell or progenitor cell disorder is a malignant hematologic disease. For example, in some embodiments, the malignant hematologic disease may be selected from the group consisting of acute lymphoid leukemia, acute myeloid leukemia, chronic lymphoid leukemia, chronic myeloid leukemia, diffuse large B-cell non-Hodgkin's lymphoma, mantle cell lymphoma, lymphoblastic lymphoma, Burkitt's lymphoma, follicular B-cell non-Hodgkin's lymphoma, lymphocyte predominant nodular Hodgkin's lymphoma, multiple myeloma, and juvenile myelomonocytic leukemia. In some embodiments, the stem cell or progenitor cell disorder is a non-malignant disease. For example, in some embodiments the non-malignant disease may be selected from the group consisting of myelofibrosis, myelodysplastic syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disorder, chronic inflammatory demyelinating polyradiculoneuropathy, human immunodeficiency virus (HIV), Fanconi anemia, sickle cell disorder, beta thalassemia major, Hurler's syndrome (MPS-IH), adrenoleukodystrophy, metachromatic leukodystrophy, familial erythrophagocytic lymphohistiocytosis and other histiocytic disorders, severe combined immunodeficiency (SCID), and Wiskott-Aldrich syndrome.

Also disclosed herein is an isolated, non-native heHSC, wherein the heHSC is Sca-1+, c-kit+ and Lin− (SKL); wherein the heHSC is prepared by mobilizing hematopoietic stem cells and/or progenitor cells from a bone marrow compartment of a subject to a peripheral compartment of the subject by administering at least one CXCR2 agonist and at least one CXCR4 antagonist, VLA-4 antagonist, α₉β₁ antagonist, α₉β₁ integrin/VLA-4 antagonist or combination thereof to the subject, and isolating the mobilized hematopoietic stem cells and/or progenitor cells from the peripheral compartment of the subject. In some embodiments, the isolated heHSC does not express CD48 or is CD48−. In some embodiments, the isolated heHSC expresses CD150 or is CD150+. In some embodiments, the isolated heHSC expresses CD93 or is CD93+. In certain aspects, the isolated heHSC does not express an immunophenotypic means of identifying human hematopoietic stem cells. In some embodiments the heHSC is a SLAM SKL heHSC. In some embodiments, the at least one CXCR2 agonist comprises GROβ or an analog or derivative thereof. In some embodiments the at least one CXCR2 agonist comprises GROβ-Δ4 or an analog or derivative thereof. In some embodiments, the at least one CXCR4 antagonist comprises plerixafor (AMD-3100) or an analog or derivative thereof. In still other embodiments, the at least one CXCR2 agonist is GROβ or an analog or derivative thereof and the at least one CXCR4 antagonist is plerixafor or an analog or derivative thereof. In some embodiments, the at least one CXCR4 antagonist comprises ALT1188, ALT1187, ALT1128, ALT1228, or TG-0054. In some embodiments, the α₉β₁ integrin/VLA-4 antagonist is N-(benzenesulfonyl)-L-prolyl-L-O-(1-pyrrolidinylcarbonyl)tyrosine (BOP) or an analog or derivative thereof (e.g., R-BC154). In some embodiments, the VLA-4 antagonist is BIO 5192 or Natalizumab, or an analog or derivative thereof.

In some embodiments, the isolated heHSC comprises a unique transcriptome relative to hematopoietic stem cells contacted with granulocyte colony-stimulating factor (G-CSF), a chemotherapeutic agent, or any combination thereof. For example, in some aspects, the isolated heHSCs disclosed herein are characterized based on their differential expression of one or more of the genes selected from the group consisting of Fos, CD93, Fosb, Dusp1, Jun, Dusp6, Cdk1, Fignl1, Plk2, Rsad2, Sgk1, Sdc1, Serpine2, Spp1, Cdca8, Nrp1, Mcam, Pbk, Akr1cl and Cyp11a1, relative to, for example the expression of one or more genes in HSCs mobilized using G-CSF. In certain aspects, the isolated heHSC is non-quiescent. In some embodiments, the isolated heHSC is OPN+(e.g., the isolated heHSC express osteopontin). In some embodiments, the isolated heHSC differentially expresses CD93 (e.g., the heHSC is CD93+). In some embodiments, the isolated heHSC does not express CD34 or is CD34−. In some embodiments, the isolated heHSC is CD93+ and CD34−.

In certain aspects of the present inventions, the isolated heHSCs disclosed herein are transformed to express a polynucleotide (e.g., an isolated heHSC may be transformed with an expression vector to express an exogenous polynucleotide). In some embodiments, the expression vector comprises a viral vector selected from the group consisting of a retrovirus, a herpes simplex, a lentivirus, an adenovirus, and an adeno-associated virus. In some embodiments, the isolated heHSC is transfected with an expression vector that comprises the polynucleotide. In some embodiments, the polynucleotide comprises an exogenous polynucleotide.

Also disclosed herein is the use of the isolated heHSC to effect or otherwise facilitate the delivery of an exogenous polynucleotide to a subject in need thereof. For example, the isolated heHSC disclosed herein may be transformed to express an exogenous polynucleotide and, upon engraftment in the subject's tissues (e.g., bone marrow tissues), the engrafted heHSC expresses the exogenous polynucleotide, thereby delivering the expression product of the exogenous polynucleotide (e.g., a protein or amino acid) to the subject.

In some embodiments, also disclosed herein are methods of transforming an isolated heHSC, wherein such methods comprise a step of contacting the heHSC with an expression vector under conditions sufficient for the vector to integrate into the heHSC genome. In yet other embodiments, the isolated heHSC of the present inventions are genetically modified to shut off expression of an endogenous polynucleotide.

In certain embodiments, the isolated heHSC is substantially pure.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates that relative to G-CSF, the combination of the CXCR2 agonist GROβ and the CXCR4 antagonist plerixafor (AMD-3100) mobilized a highly engraftable hematopoietic stem cell (heHSC). As shown in FIG. 1, relative to G-CSF mobilized cells, an increase in donor chimerism was observed following engraftment with the heHSCs that were mobilized with GROβ and AMD-3100. In this demonstration, 195 CD150+, CD48−, SKL cells were transplanted per mouse.

FIG. 2 illustrates that relative to G-CSF, the combination of the CXCR2 agonist GROβ and the CXCR4 antagonist plerixafor (AMD-3100) mobilized a highly engraftable hematopoietic stem cell (heHSC), in a separate, independent demonstration from that shown in FIG. 1. As shown in FIG. 2, relative to G-CSF mobilized cells, an increase in donor chimerism was observed following engraftment of the heHSCs that were mobilized with GROβ and AMD-3100. In this demonstration, 50 CD150+CD48-SKL cells were transplanted per mouse.

FIG. 3 illustrates that certain genes showed higher expression in the heHSCs that were mobilized using the combination of the CXCR2 agonist GROβ and the CXCR4 antagonist plerixafor (AMD-3100), relative to the cells mobilized using G-CSF.

FIG. 4 illustrates a heat map showing the top twenty discriminating genes between hematopoietic stem cells (HSCs) that were mobilized using G-CSF mobilized (the two Tube B replicates), relative to the heHSCs (Tube C) mobilized using the combination of the CXCR2 agonist GROβ and the CXCR4 antagonist plerixafor (AMD-3100). Spp1 corresponds to osteopontin marker I.

### DETAILED DESCRIPTION OF THE INVENTION

The present disclosure relates to a non-native, highly engraftable hematopoietic stem cell (heHSC) that is useful in connection with stem cell transplantation and the treatment of stem cell and/or progenitor cell disorders. Disclosed herein are isolated, non-native heHSCs, methods of their use and manufacture, and kits that comprise such heHSCs for use in connection with stem cell transplantation or the treatment of stem cell and/or progenitor cell disorders. The heHSCs disclosed herein are useful, for example, for transplantation and/or engraftment in a subject in connection with the treatment of any disease requiring stem cell transplantation.

The work described herein relates to the surprising discovery that heHSCs that are prepared by contacting or mobilizing with a combination of a CXCR2 agonist (e.g., GROβ) and a CXCR4 antagonist (e.g., plerixafor) exhibit superior engrafting ability, for example, superior engrafting ability relative to HSCs or peripheral blood stem cells (PBSCs) that are mobilized using traditional mobilizing regimens (e.g., granulocyte-colony stimulating factor (G-CSF) or chemotherapeutic agents). Accordingly, certain aspects of the present inventions relate to non-native, isolated heHSCs that are prepared by contacting or mobilizing hematopoietic stem cells and/or progenitor cells using a combination of one or more CXCR2 agonists (e.g., GROβ) and one or more CXCR4 antagonists (e.g., plerixafor). An exemplary method of mobilizing hematopoietic stem cells and/or progenitor cells in a subject comprises administering to the subject a combination of at least one CXCR2 agonist and at least one CXCR4 antagonist in amounts sufficient to mobilize such hematopoietic stem cells and/or progenitor cells into the subject's peripheral blood. The isolated heHSCs disclosed herein and the related methods of their preparation by mobilizing hematopoietic stem cells and/or progenitor cells have a variety of useful applications, for example for the treatment of stem cell and/or progenitor cell disorders.

In some embodiments, aspects of the present inventions relate to non-native, isolated heHSCs that are prepared by contacting or mobilizing hematopoietic stem cells and/or progenitor cells using a combination of at least one CXCR2 agonist (e.g., GROβ) and at least one CXCR4 antagonist, VLA-4 antagonist, α₉β₁ antagonist, α₉β₁ integrin/VLA-4 antagonist or combination thereof.

As used herein, the term “mobilizing” refers to the act of inducing the migration of hematopoietic stem cells and/or progenitor cells (e.g., heHSCs) from a first location (e.g., the stem cell niche or bone marrow tissues of a subject) to a second location (e.g., the peripheral blood or an organ, such as the spleen, of a subject). For example, in certain embodiments, the non-native, isolated heHSCs disclosed herein may be prepared by mobilizing hematopoietic stem cells and/or progenitor cells from the stem cell niche of a human subject into the subject's peripheral tissue by administering to the subject a combination of one or more CXCR2 agonists (e.g., GROβ) and one or more CXCR4 antagonists (e.g., plerixafor), following which the mobilized heHSCs may be harvested or isolated (e.g., by apheresis), as further described herein. With regard to the heHSCs disclosed herein, the term “isolated” means that the heHSC is substantially free of other cell types or cellular materials with which may be present when the heHSC is isolated from a treated subject. In some embodiments, an isolated heHSC or an isolated population of heHSCs is a substantially pure population of heHSCs, for example, as compared to the heterogeneous population from which the cells were isolated or enriched from (e.g., substantially pure as compared to the population of mobilized cells). In some embodiments, the heHSCs are enriched from a biological sample that is obtained from a subject following treatment with a combination of a CXCR2 agonist (e.g., GROβ) and a CXCR4 antagonist (e.g., plerixafor). In one embodiment, the mobilized and harvested heHSCs disclosed herein may be used in connection with an allogeneic or an autologous transplant. The terms “enriching” or “enriched” are used interchangeably herein and mean that the yield (fraction) of heHSCs is increased by at least about 10%, 15%, 20%, 25%, 30%, 40%, 50%, 60%, 70%, 80%, 90%, 95%, 99% or more over the fraction of mobilized cells.

As used herein with respect to a population of heHSCs, term “substantially pure”, refers to a population of heHSCs that is at least about 75%, preferably at least about 85%, more preferably at least about 90%, and most preferably at least about 95% pure, and still more preferably at least about 99% pure with respect to the cells making up a total population of mobilized cells. Recast, the terms “substantially pure” or “essentially purified”, with regard to a population of heHSCs, refers to a population of cells that contain fewer than about 20%, more preferably fewer than about 15%, 12%, 10%, 8%, 7%, most preferably fewer than about 5%, 4%, 3%, 2%, 1%, or less than 1%, of cells that are not heHSCs as defined by the terms herein. In some embodiments, the present invention encompasses methods to expand a population of heHSCs, wherein the expanded population of heHSCs is a substantially pure population.

While certain embodiments disclosed herein contemplate the in vivo preparation of the heHSCs by mobilizing hematopoietic stem cells and/or progenitor cells, it should be understood that the present inventions are not limited to such in vivo methods. Rather, also contemplated are in vitro methods of preparing heHSCs, for example by contacting hematopoietic stem cells and/or progenitor cells with a combination of a CXCR2 agonist (e.g., GROβ) and a CXCR4 antagonist (e.g., plerixafor), VLA-4 antagonist, α₉β₁ antagonist, α₉β₁ integrin/VLA-4 antagonist or combination thereof. As used herein, the term “contacting” means bringing two or more moieties together, or within close proximity of one another such that the moieties may interact with each other. For example, in one embodiment of the present invention, a hematopoietic stem cell and/or a progenitor cell is contacted with a CXCR2 agonist and/or a CXCR4 antagonist to produce and/or mobilize a heHSC.

Contemplated CXCR2 agonists include any compounds or agents that are capable of activating the CXCR2 receptor (e.g., the human CXCR2 receptor). Exemplary CXCR2 agonists include chemokines, cytokines, biologic agents, antibodies and small organic molecules. For example, contemplated chemokines acting via the CXCR2 receptor include without limitation GROβ, GROα, GROγ, GCP-2 (granulocyte chemo-attractant protein 2), IL-8, NAP-2 (neutrophil activating peptide 2), ENA-78 (epithelial-cell derived neutrophil activating protein 78), and modified forms of any of the foregoing. In some embodiments, the CXCR2 agonist is selected from the group of compounds or agents consisting of small organic or inorganic molecules; oligosaccharides; polysaccharides; biological macromolecules selected from the group consisting of peptides, proteins, peptide analogs and derivatives; peptidomimetics; nucleic acids selected from the group consisting of siRNAs, shRNAs, antisense RNAs, ribozymes, and aptamers; and any combination thereof.

In certain aspects, the CXCR2 agonist comprises GROβ.

In some embodiments, the at least one CXCR2 agonist is the chemokine GROβ or an analog or derivative thereof. An exemplary form of GROβ is the human GROβ polypeptide (GenBank Accession: AAP13104; SEQ ID NO: 1). In certain aspects, an exemplary form of GROβ is the human GROβ (UniProt ID No. P19875; SEQ ID NO: 2).

An exemplary GROβ analog or derivative is the desamino GROβ protein (also known as MIP-2alpha), which comprises the amino acid sequence of mature gro-S protein truncated at its N terminus between amino acid positions 2 and 8, as described in PCT International Application Publication WO/1994/029341, the contents of which are incorporated herein by reference in their entirety. Another GROβ analog or derivative is the dimeric modified GROβ protein described in U.S. Pat. No. 6,413,510, the contents of which are incorporated herein by reference in their entirety. Still another exemplary GROβ analog or derivative is SB-251353, a GROβ analog involved in directing movement of stem cells and other leukocytes, as described by Bensinger, et al., Bone Marrow Transplantation (2009), 43, 181-195, the entire contents of which are incorporated by reference herein.

In some embodiments of the present inventions, the at least one CXCR2 agonist is or comprises GROβ-Δ4 (e.g., SEQ ID NO: 3) or an analog or derivative thereof. In some embodiments, the at least one CXCR2 agonist is selected from the group consisting of GROβ or an analog or derivative thereof and GROβ-Δ4 or an analog or derivative thereof.

Contemplated CXCR4 antagonists include any compounds or agents that are capable of blocking the CXCR4 receptor or preventing its activation. For example, contemplated are compounds and agents that block or otherwise interfere with the binding or interaction of the CXCR4 receptor with such receptor's ligand. Also contemplated are compounds or agents that block the downstream effects of the activated CXCR4 receptor. In some embodiments, the CXCR4 antagonist is selected from the group of compounds or agents consisting of small organic or inorganic molecules; oligosaccharides; polysaccharides; biological macromolecules selected from the group consisting of peptides, proteins, peptide analogs and derivatives; peptidomimetics; nucleic acids selected from the group consisting of siRNAs, shRNAs, antisense RNAs, ribozymes, and aptamers; and any combination thereof.

In some embodiments of the present inventions, the at least one CXCR4 antagonist is plerixafor (formerly known as AMD-3100), the structure of which is depicted below (I), or an analog or derivative thereof.

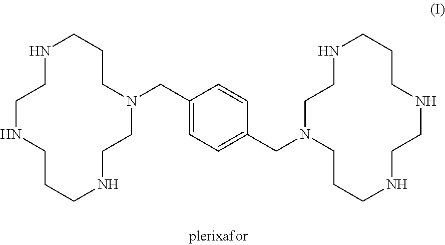 [SMILES: c1cc(CN2CCCNCCNCCCNCC2)ccc1CN1CCCNCCNCCCNCC1]

In some embodiments, the at least one CXCR4 antagonist is MOZOBIL® or an analog or derivative thereof. Exemplary analogs of plerixafor include, but are not limited to, AMD11070, AMD3465, KRH-3955, T-140, and 4F-benzoyl-TN14003, as depicted below (II-VI, respectively) and described by De Clercq, Pharmacol Ther. (2010) 128(3):509-18, the contents of which are incorporated by reference herein in their entirety.

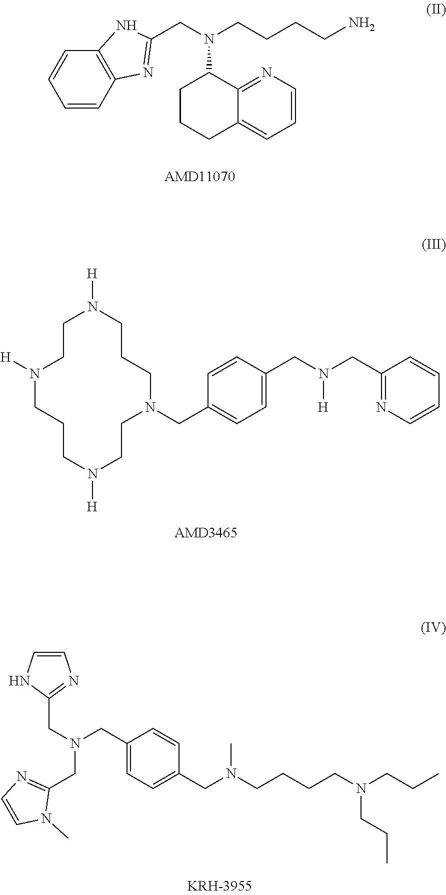

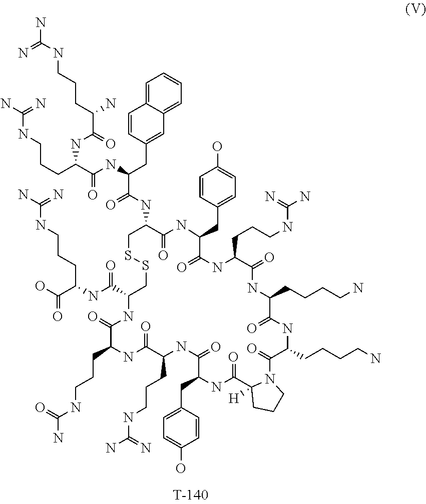 [SMILES: N=C(N)NCCC[C@H](NC(=O)[C@@H]1CSSC[C@H](NC(=O)[C@H](Cc2ccc3ccccc3c2)NC(=O)[C@H](CCCNC(=N)N)NC(=O)[C@@H](N)CCCNC(=N)N)C(=O)N[C@@H](Cc2ccc(O)cc2)C(=O)N[C@@H](CCCNC(=N)N)C(=O)N[C@@H](CCCCN)C(=O)N[C@H](CCCCN)C(=O)N2CCC[C@H]2C(=O)N[C@@H](Cc2ccc(O)cc2)C(=O)N[C@@H](CCCNC(=N)N)C(=O)N[C@@H](CCCNC(N)=O)C(=O)N1)C(=O)O]

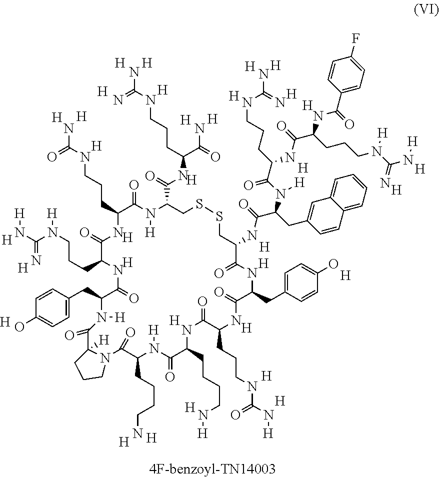

In some embodiments, the at least one CXCR4 antagonist comprises ALT1188, ALT1187, ALT1128, ALT1228, or TG-0054 or an analog or derivative thereof. In some embodiments, the CXCR4 antagonist comprises at least one inhibitor described in Debnath B, et al., “Small Molecule Inhibitors of CXCR4,” Theranostics 2013; 3(1):47-75, incorporated herein by reference.

In some embodiments, non-native, isolated heHSCs are prepared by contacting or mobilizing hematopoietic stem cells and/or progenitor cells using a combination of at least one CXCR2 agonist (e.g., GROβ) and at least one α₉β₁ integrin/VLA-4 antagonist. In some embodiments, the α₉β₁ integrin/VLA-4 antagonist is N-(benzenesulfonyl)-L-prolyl-L-O-(1-pyrrolidinylcarbonyl)tyrosine (BOP) or an analog or derivative thereof (e.g., R-BC154). In some embodiments, non-native, isolated heHSCs are prepared by contacting or mobilizing hematopoietic stem cells and/or progenitor cells using a combination of at least one CXCR2 agonist (e.g., GROβ) and at least one VLA-4 antagonist. In some embodiments, the VLA-4 antagonist is BIO 5192, Natalizumab, or an analog or derivative thereof.

In some embodiments, the at least one CXCR2 agonist is or comprises GROβ or an analog or derivative thereof, and the at least one CXCR4 antagonist is or comprises plerixafor (AMD-3100) or an analog or derivative thereof. In some embodiments, the at least one CXCR2 agonist is selected from the group consisting of GROβ-Δ4 or an analog or derivative thereof and the at least one CXCR4 antagonist is selected from the group consisting of plerixafor or an analog or derivative thereof.

The combination of at least one CXCR2 agonist and at least one CXCR4 antagonist, VLA-4 antagonist, α₉β₁ antagonist, α₉β₁ integrin/VLA-4 antagonist or combination thereof may be administered directly to a subject in combination or, in certain aspects, may be administered independently. For example, the at least one CXCR2 agonist and the at least one CXCR4 antagonist, VLA-4 antagonist, α₉β₁ antagonist, α₉β₁ integrin/VLA-4 antagonist or combination thereof can be, but need not be, administered (e.g., administered intravenously) to a subject at the same time. In one embodiment, the at least one CXCR2 agonist is administered in one or more doses, followed by the administration of the at least one CXCR4 antagonist in one or more doses.

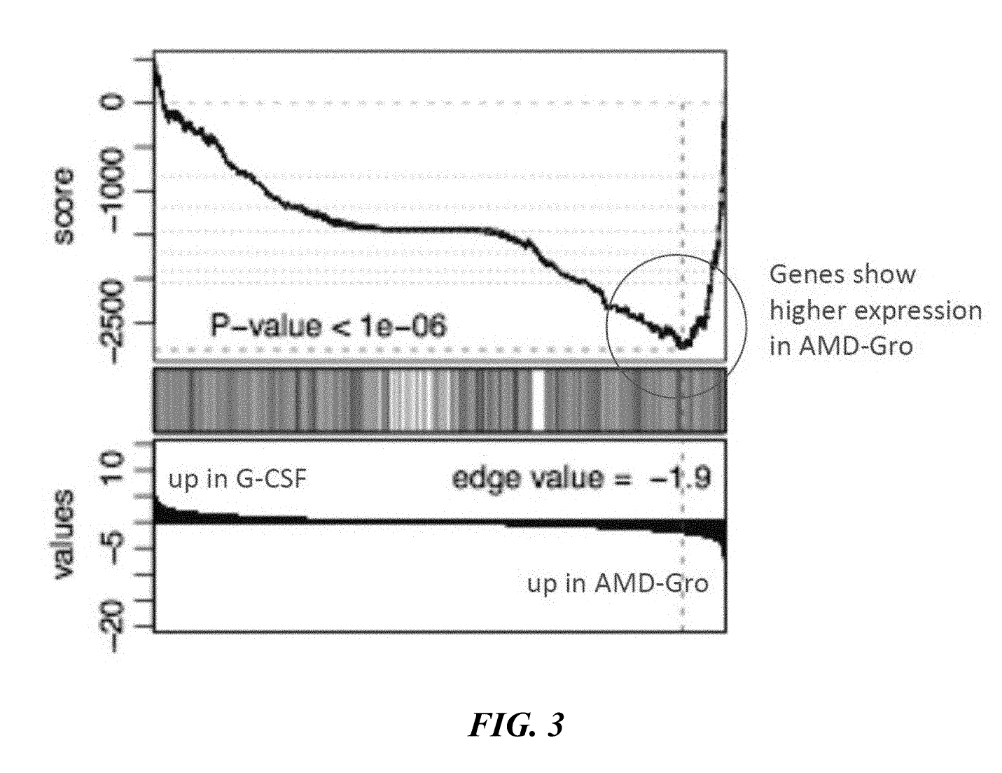

In addition to inducing a faster mobilization (e.g., about two-fold, three-fold, four-fold, five-fold, six-fold, seven-fold, eight-fold, nine-fold, ten-fold, twelve-fold, fifteen-fold, twenty-fold or more faster relative to traditional mobilization regimens that are performed using, for example, G-CSF or, alternatively, within one hour, within 45 minutes, within 30 minutes, within 15 minutes within 10 minutes, within 5 minutes or faster) and producing a greater quantity of mobilized stem cells (e.g., heHSCs), the combination of at least one CXCR2 agonist (e.g., GROB-Δ4 or an analog or derivative thereof) and at least one CXCR4 antagonist (e.g., plerixafor or an analog or derivative thereof), VLA-4 antagonist, α₉β₁ antagonist, α₉β₁ integrin/VLA-4 antagonist or combination thereof mobilizes a non-native stem cell that is characterized by its enhanced engrafting ability and its unique genetic signatures, as illustrated in FIG. 3. As used herein to describe the stem cells that are mobilized using the combination of at least one CXCR2 agonist and at least one CXCR4 antagonist, VLA-4 antagonist, α₉β₁ antagonist, α₉β₁ integrin/VLA-4 antagonist or combination thereof the term “unique” refers to one or more distinguishing characteristics of such mobilized stem cells relative to those cells that are mobilized using traditional mobilization regiments using, for example, G-CSF alone. For example, stem cells that are mobilized using the combination of at least one CXCR2 agonist and at least one CXCR4 antagonist, VLA-4 antagonist, α₉β₁ antagonist, α₉β₁ integrin/VLA-4 antagonist or combination thereof may be characterized by their expression of one or more unique markers or antigens (e.g., CD93+) or by their unique transcriptome.

One such marker, CD93, is expressed in hematopoietic cells at the apex of hematopoiesis. These early hematopoietic CD93 expressing cells in humans may also be negative for CD34. heHSC populations generated upon treatment with combination of at least one CXCR2 agonist and at least one CXCR4 antagonist which also exhibit CD93 expression are indicative of early lineage stem cells and may serve to support improved transplantation and/or engraftment.

Similarly, in certain embodiments, stem cells that are mobilized using the combination of at least one CXCR2 agonist and at least one CXCR4 antagonist, VLA-4 antagonist, α₉β₁ antagonist, α₉β₁ integrin/VLA-4 antagonist or combination thereof may be characterized by improved function. In particular, the engrafting ability of the heHSCs mobilized using the combination of at least one CXCR2 agonist and at least one CXCR4 antagonist, VLA-4 antagonist, α₉β₁ antagonist, α₉β₁ integrin/VLA-4 antagonist or combination thereof is surprisingly increased or enhanced relative to the engrafting ability of stem cells or PBSCs that are mobilized following the contacting of hematopoietic stem cells and/or progenitor cells with traditional mobilizing agents, such as G-CSF.

In certain aspects, the heHSCs are characterized by their increased or enhanced engrafting ability relative to stem cells or PBSCs that are mobilized following the contacting of hematopoietic stem cells and/or progenitor cells with one or more chemotherapeutic agents (e.g., chemotherapeutic mobilization agents). Exemplary chemotherapeutic agents include paclitaxel, etoposide, vinblastine, doxorubicin, bleomycin, methotrexate, 5-fluorouracil, 6-thioguanine, cytarabine, cyclophosphamide, cisplatinum and combinations thereof. In certain aspects, such chemotherapeutic agents mobilize hematopoietic stem cells and/or progenitor cells. For example, such a chemotherapeutic mobilization agent may comprise EPO. In some embodiments, such a chemotherapeutic mobilization agent is or comprises stem cell factor. In some embodiments, such a chemotherapeutic mobilization agent is or comprises TPO. In still other embodiments, such a chemotherapeutic mobilization agent is or comprises parathyroid hormone.

As used herein, the term “hematopoietic stem cells” or “HSC” refers to stem cells that can differentiate into the hematopoietic lineage and give rise to all blood cell types such as white blood cells and red blood cells, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). Stem cells are defined by their ability to form multiple cell types (multipotency) and their ability to self-renew. Hematopoietic stem cells can be identified, for example by cell surface markers such as CD34−, CD133+, CD48−, CD150+, CD244−, cKit+, Sca1+, and lack of lineage markers (negative for B220, CD3, CD4, CD8, Mac1, Gr1, and Ter119, among others).

As used herein, the term “hematopoietic progenitor cells” encompasses pluripotent cells which are committed to the hematopoietic cell lineage, generally do not self-renew, and are capable of differentiating into several cell types of the hematopoietic system, such as granulocytes, monocytes, erythrocytes, megakaryocytes, B-cells and T-cells, including, but not limited to, short term hematopoietic stem cells (ST-HSCs), multi-potent progenitor cells (MPPs), common myeloid progenitor cells (CMPs), granulocyte-monocyte progenitor cells (GMPs), megakaryocyte-erythrocyte progenitor cells (MEPs), and committed lymphoid progenitor cells (CLPs). The presence of hematopoietic progenitor cells can be determined functionally as colony forming unit cells (CFU-Cs) in complete methylcellulose assays, or phenotypically through the detection of cell surface markers (e.g., CD45−, CD34+, Ter119−, CD16/32, CD127, cKit, Sca1) using assays known to those of skill in the art.

In some embodiments, the mobilized hematopoietic stem cells and/or progenitor cells comprise SKL cells. In certain aspects, the mobilized hematopoietic stem cells and/or progenitor cells comprise SKL SLAM cells. In certain aspects, the mobilized hematopoietic stem cells and/or progenitor cells exhibit a SLAM (Signaling lymphocyte activation molecule) expression pattern which is CD150+, CD48−. A SLAM expression pattern (SLAM code) is an expression pattern of specific markers (SLAM markers) that are used to identify subpopulations of hematopoietic stem cells and multipotent progenitors. See Oguro, et al. (2013) “SLAM family markers resolve functionally distinct subpopulations of hematopoietic stem cells and multipotent progenitors,” Cell Stem Cell, 13(1), 102-116, and references cited therein.

In some embodiments, the mobilized hematopoietic stem cells and/or progenitor cells comprise CD34−, CD133+ cells. In some embodiments, the mobilized hematopoietic stem cells and/or progenitor cells comprise common myeloid progenitor cells. In some embodiments, the mobilized hematopoietic stem cells and/or progenitor cells comprise granulocyte/monocyte progenitor cells. In some embodiments, the mobilized hematopoietic stem cells and/or progenitor cells comprise megakaryocyte/erythroid progenitor cells. In some embodiments, the mobilized hematopoietic stem cells and/or progenitor cells comprise committed lymphoid progenitor cells. In some embodiments, the mobilized hematopoietic stem cells and/or progenitor cells comprise a combination of common myeloid progenitor cells, granulocyte/monocyte progenitor cells, megakaryocyte/erythroid progenitor cells. In some embodiments, the mobilized hematopoietic stem cells and/or progenitor cells comprise CD150-, CD48−, CD244+ cells. In some embodiments, the mobilized hematopoietic stem cells and/or progenitor cells comprise CD150-, CD48+, CD244+ cells. In some embodiments, the mobilized hematopoietic stem cells and/or progenitor cells comprise Sca-1−, c-kit+, Lin−, CD34+, CD16/32^{mid} cells. In some embodiments, the mobilized hematopoietic stem cells and/or progenitor cells comprise Sca-1−, c-kit+, Lin−, CD34−, CD16/32^{low} cells. In some embodiments, the isolated heHSC does not express an immunophenotypic means of identifying human hematopoietic stem cells.

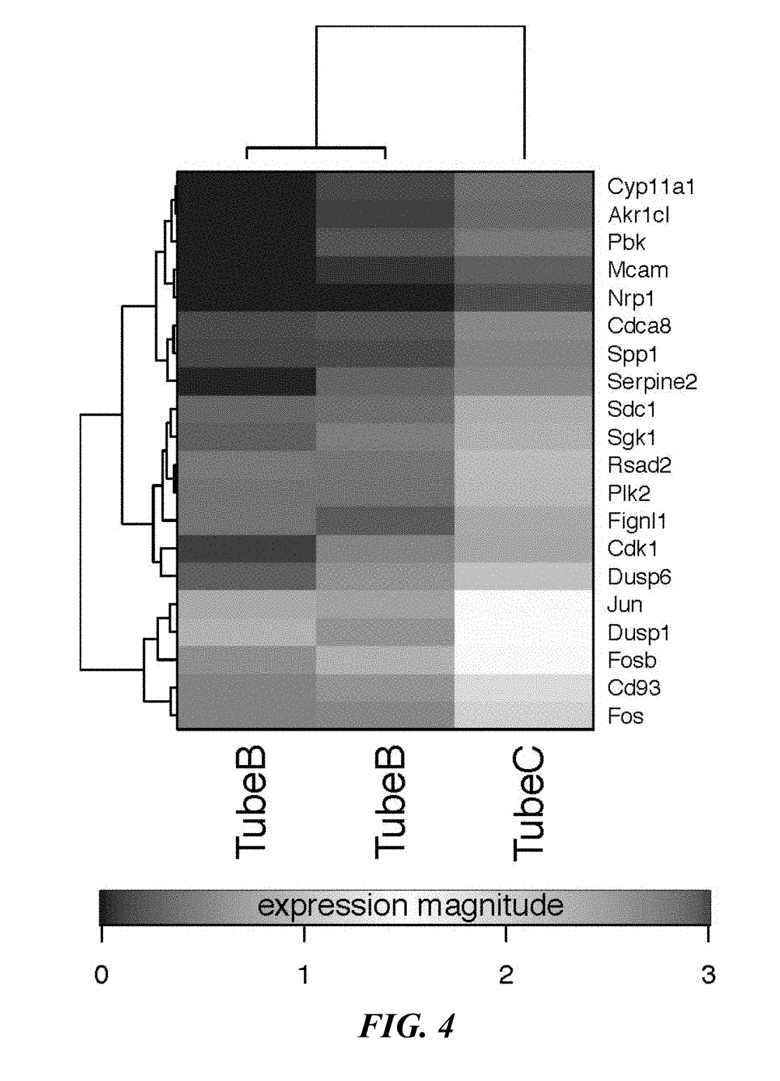

In some embodiments, the isolated heHSCs disclosed herein comprise a unique transcriptome relative to hematopoietic stem cells contacted with G-CSF, a chemotherapeutic agent, or a combination thereof. For example, in certain aspects, the isolated heHSCs disclosed herein are characterized based on their differential expression of one or more of the genes identified in FIG. 4, relative to, for example the expression of one or more genes in hematopoietic stem cells (HSCs) that were mobilized using G-CSF. In some aspects, the isolated heHSCs disclosed herein are characterized based on their differential expression of one or more of the genes selected from the group consisting of Fos (e.g., SEQ ID NO: 4), CD93 (e.g., SEQ ID NO: 5), Fosb (e.g., SEQ ID NO: 6), Dusp1 (e.g., SEQ ID NO: 7), Jun (e.g., SEQ ID NO: 8), Dusp6 (e.g., SEQ ID NO: 9), Cdk1 (e.g., SEQ ID NO: 10), Fignl1 (e.g., SEQ ID NO: 11), Plk2 (e.g., SEQ ID NO: 12), Rsad2 (e.g., SEQ ID NO: 13), Sgk1 (e.g., SEQ ID NO: 14), Sdc1 (e.g., SEQ ID NO: 15), Serpine2 (e.g., SEQ ID NO: 16), Spp1 (e.g., SEQ ID NO: 17), Cdca8 (e.g., SEQ ID NO: 18), Nrp1 (e.g., SEQ ID NO: 19), Mcam (e.g., SEQ ID NO: 20), Pbk (e.g., SEQ ID NO: 21), Akr1cl (e.g., SEQ ID NO: 22) and Cyp11a1 (e.g., SEQ ID NO: 23), relative to, for example the expression of one or more genes by hematopoietic stem cells (HSCs) that were mobilized using G-CSF. In some embodiments, the isolated heHSC is OPN+(e.g., the isolated heHSC express osteopontin). In some embodiments, the isolated heHSC differentially expresses CD93 (e.g., the heHSC is CD93+). In certain aspects, the isolated heHSC disclosed herein is non-quiescent. In some embodiments, the heHSC is CD34−.

The heHSCs disclosed herein are prepared by mobilizing or contacting hematopoietic stem cells and/or progenitor cells with a combination of a CXCR2 agonist and a CXCR4 antagonist, VLA-4 antagonist, α₉β₁ antagonist, α₉β₁ integrin/VLA-4 antagonist or combination thereof. As used herein, the terms “highly engraftable hematopoietic stem cell” and “heHSC” refer to the isolated population or fraction of stem cells or PBSCs that are, for example, mobilized from the stem cell niche or bone marrow of a subject into the peripheral blood or organs of the subject following the administration of one or more CXCR2 agonists (e.g., GROβ or an analog or derivative thereof) and one or more CXCR4 antagonists (e.g., plerixafor or an analog or derivative thereof), VLA-4 antagonist, α₉β₁ antagonist, α₉β₁ integrin/VLA-4 antagonist or combination thereof. In certain aspects, such heHSCs are substantially pure.

In some embodiments, the isolated heHSCs disclosed herein are immunophenotypically unique relative to cells or stem cells mobilized using traditional mobilization regimens (e.g., stem cells mobilized using G-CSF). For example, as illustrated in FIG. 3, certain genes showed higher expression in the heHSCs that were mobilized using the combination of the CXCR2 agonist GROβ and the CXCR4 antagonist plerixafor (AMD-3100), relative to the cells mobilized using G-CSF. In certain aspects, the heHSCs disclosed herein express osteopontin or are osteopontin positive (OPN+). In some embodiments, the isolated heHSC differentially expresses CD93 (e.g., the heHSC is CD93+). In some embodiments, the isolated heHSC does not express CD34 or is CD34−. In some embodiments, the isolated heHSC is CD93+ and CD34−. In some embodiments, the isolated heHSC differentially expresses one or more genes shown in FIG. 3 or FIG. 4 as compared to an isolated HSC mobilized using traditional mobilization regimens (e.g., stem cells mobilized using G-CSF).

In some embodiments, a population of cells (i.e., a cell population comprising or consisting of heHSC) isolated by the methods disclosed herein (e.g., by contacting cells with a combination of at least one CXCR2 agonist (e.g., GROβ) and at least one CXCR4 antagonist, VLA-4 antagonist, α₉β₁ antagonist, α₉β₁ integrin/VLA-4 antagonist or combination thereof) has an increased or decreased proportion of cells exhibiting one or more cell surface markers or one or more expression profiles disclosed herein as compared to cells isolated by conventional methods. The one or more cell surface markers or cell expression profiles may be increased or decreased by about 10%, 15%, 20%, 25%, 30%, 35%, 40%, 45%, 50%, 55%, 60%, 65%, 70%, 75%, 80%, 85%, 90%, 95%, or more. In some embodiments, the one or more cell surface marker is CD93. In some embodiments, after performing the methods disclosed herein, an obtained cell population may be assayed to determine whether the prevalence of one or more cell surface markers or cell expression profiles has increased or decreased to determine whether the obtained cell population is suitable as heHSC for transplantation. In some embodiments, the obtained cell population is assayed to determine if at least about 10%, 15%, 20%, 25%, 30%, 35%, 40%, 45%, 50%, 55%, 60%, 65%, 70%, 75%, 80%, 85%, 90%, 95%, or more of the cells are CD93+. Any suitable assay (e.g., FACS analysis) may be used for the determination.

In some embodiments, the obtained cell population may be further enriched for a desired cell surface marker or gene expression pattern to obtain a desired heHSC population for transplantation. In some embodiments, the obtained cell population may be enriched for CD93+ cells or CD93+ and CD34− cells. In some embodiments, the cell population may be enriched by about 1.5-fold, 2-fold, 2.5-fold, 3-fold, 4-fold, 5-fold or more. In some embodiments, the cell population may be enriched to contain at least 40%, 45%, 50%, 55%, 60%, 65%, 70%, 75%, 80%, 85%, 90%, 95%, or more of cells containing a desired cell surface marker or cell expression pattern (e.g., enriched for CD93+ cells or CD93+/CD34− cells). Any suitable procedure (e.g., FACS sorting) may be used for the enrichment. In some embodiments, the isolated heHSCs disclosed herein are not immunophenotypically unique relative to cells or stem cells mobilized using traditional mobilization regimens (e.g., stem cells mobilized using G-CSF). Such isolated heHSC may be functionally unique relative to cells or stem cells mobilized using traditional mobilization regimens.

Upon mobilization, which in certain instances may occur within 15-30 minutes of having administered a CXCR2 agonist and a CXCR4 antagonist, VLA-4 antagonist, α₉β₁ antagonist, α₉β₁ integrin/VLA-4 antagonist or combination thereof, the mobilized heHSCs can be harvested or isolated (e.g., via apheresis) as disclosed herein and are useful for subsequent transplantation in a subject in need thereof. For example, such mobilized heHSCs may be harvested or isolated for autologous transplantation into a subject or for allogeneic transplantation into a recipient subject. In some instances, the harvesting or isolation of the mobilized hematopoietic stem cells and/or progenitor cells can be initiated within as little as 15 minutes following the administration of the at least one CXCR2 agonist and the at least one CXCR4 antagonist, VLA-4 antagonist, α₉β₁ antagonist, α₉β₁ integrin/VLA-4 antagonist or combination thereof. In some embodiments, the harvesting or isolating procedure can begin in as little as 10 minutes, 12 minutes, 15 minutes, 18 minutes, 20 minutes, 22 minutes, 25 minutes, 30 minutes, 35 minutes, 40 minutes, 47 minutes, 52 minutes, 58 minutes, or an hour after administration of the at least one CXCR2 agonist and the at least one CXCR4 antagonist, VLA-4 antagonist, α₉β₁ antagonist, α₉β₁ integrin/VLA-4 antagonist or combination thereof.

The disclosure contemplates the use of any suitable method of harvesting and/or collecting mobilized hematopoietic stem cells and/or progenitor cells to prepare the isolated heHSCs disclosed herein. In some embodiments harvesting the mobilized hematopoietic stem cells and/or progenitor cells comprises apheresis. In some embodiments, the combination of at least one CXCR2 agonist (e.g., GROβ or GROβ-Δ4) and at least one CXCR4 antagonist (e.g., plerixafor), VLA-4 antagonist, α₉β₁ antagonist, α₉β₁ integrin/VLA-4 antagonist or combination thereof rapidly and efficiently mobilizes mobilized hematopoietic stem cells and/or progenitor cells, and exhibits increased efficiencies compared to traditional mobilizing regimens. As a result, in some embodiments an apheresis procedure may be performed on the same day that the at least one CXCR2 agonist and the at least one CXCR4 antagonist, VLA-4 antagonist, α₉β₁ antagonist, α₉β₁ integrin/VLA-4 antagonist or combination thereof are administered to the subject. In other words, harvesting mobilized heHSCs from a subject (e.g., a donor) via apheresis can be performed on the same day that the mobilization agents are administered to the subject (e.g., during a single visit to a healthcare facility). In some embodiments, an apheresis procedure may be performed on the same day that at least one CXCR2 agonist (e.g., GROβ or GROβ-Δ4) and at least one CXCR4 antagonist, VLA-4 antagonist, α₉β₁ antagonist, α₉β₁ integrin/VLA-4 antagonist or combination thereof is administered to the subject.

In some embodiments, administration of the at least one CXCR2 agonist (e.g., GROβ or GROβ-Δ4) and the at least one CXCR4 antagonist, VLA-4 antagonist, α₉β₁ antagonist, α₉β₁ integrin/VLA-4 antagonist or combination thereof mobilizes an amount of hematopoietic stem cells and/or progenitor cells in the subject to harvest a heHSC cell dose of between about 1×10⁶/kg body weight and 10×10⁶/kg body weight in a single apheresis session. In some embodiments, a single session of apheresis collects enough heHSCs for a cell dose of between about 1×10⁶/kg and 10×10⁶/kg of the recipient's body weight. In some embodiments, administration of the at least one CXCR2 agonist (e.g., GROβ or GROβ-Δ4) and the at least one CXCR4 antagonist, VLA-4 antagonist, α₉β₁ antagonist, α₉β₁ integrin/VLA-4 antagonist or combination thereof mobilizes an amount of hematopoietic stem cells and/or progenitor cells in the subject to harvest enough heHSCs for a cell dose of between about 2×10⁶/kg body weight and 8×10⁶/kg body weight in a single apheresis session. In some embodiments, a single session of apheresis collects enough heHSCs for a cell dose of between about 2×10⁶/kg and 8×10⁶/kg of the recipient's body weight. In some embodiments, administration of the at least one CXCR2 agonist (e.g., GROβ or GROβ-Δ4) and the at least one CXCR4 antagonist, VLA-4 antagonist, α₉β₁ antagonist, α₉β₁ integrin/VLA-4 antagonist or combination thereof mobilizes an amount of hematopoietic stem cells and/or progenitor cells in the subject to harvest a heHSC cell dose of between about 3×10⁶/kg body weight and 6×10⁶/kg body weight in a single apheresis session. In some embodiments, a single session of apheresis collects enough heHSCs for a cell dose of between about 1×10⁶/kg and 10×10⁶/kg of the recipient's body weight.

Following harvesting, the isolated heHSCs disclosed herein may be administered to or transplanted in the donor subject (e.g., an autologous transplant), or alternatively may be donated to a different subject in need thereof (e.g., allogeneic transplant). In certain aspects, the administration or transplant of the isolated heHsCs occurs following or in combination with radiation or chemotherapy.

The mobilized heHSC disclosed herein are characterized by their increased engrafting ability (e.g., a two-fold increased engrafting ability), which makes such heHSCs suitable for use in connection with gene therapy. For example, where genetic manipulation of cells is associated with a corresponding reduction in their engrafting ability and, due to the improved or enhanced engrafting ability of the heHSCs disclosed herein, such heHSCs are rendered more tolerant to genetic manipulation, following which only limited reductions in their engrafting ability may be observed.

Gene therapy can be used to transform a heHSC, modify a heHSC to replace a gene product, to treat disease, or to improve engraftment of the heHSC following implantation into a subject. For example, in certain embodiments, the heHSCs disclosed herein may be transformed with an expression vector (e.g., a viral vector selected from the group consisting of a retrovirus, a herpes simplex, a lentivirus, an adenovirus, and an adeno-associated virus). In some embodiments, the isolated heHSC is transformed or transfected with an expression vector that comprises a polynucleotide. In some embodiments, the polynucleotide comprises an exogenous polynucleotide. In some embodiments, the expression product of a polynucleotide is a protein that is not endogenously expressed or is under expressed by the subject's cells.

As used herein, the term “transform” means to introduce into a heHSC an exogenous polynucleotide (e.g., a nucleic acid or nucleic acid analog) which replicates within that heHSC, that encodes a gene product (e.g., an amino acid, polypeptide sequence, protein or enzyme) which is expressed in that heHSC, and/or that is integrated into the genome of that heHSC so as to affect the expression of a genetic locus within the genome. The term “transform” is used to embrace all of the various methods of introducing such polynucleotides (e.g., nucleic acids or nucleic acid analogs), including, but not limited to the methods referred to in the art as transformation, transfection, transduction, or gene transfer, and including techniques such as microinjection, DEAE-dextran-mediated endocytosis, calcium phosphate coprecipitation, electroporation, liposome-mediated transfection, ballistic injection, viral-mediated transfection, and the like.

In some embodiments, also disclosed herein are methods of transforming an isolated heHSC, wherein such methods comprise a step of contacting the heHSC with an expression vector under conditions sufficient for the vector to integrate into the heHSC genome. In yet other embodiments, the isolated heHSC of the present inventions are genetically modified to shut off expression of an endogenous polynucleotide.

As used herein, the term “vector” means any genetic construct, such as for example, a plasmid, phage, transposon, cosmid, chromosome, virus and/or virion, which is capable transferring nucleic acids between cells. Vectors may be capable of one or more of replication, expression, and insertion or integration, but need not possess each of these capabilities. Thus, the term includes cloning, expression, homologous recombination, and knock-out vectors.

In certain aspects, prior to engraftment, a mobilized hematopoietic stem cell and/or progenitor cell can be manipulated to express one or more desired polynucleotides or gene products (e.g., one or more of a polypeptide, amino acid sequence protein and/or enzyme). Gene therapy can be used to either modify a mobilized hematopoietic stem cell and/or progenitor cell to replace a polynucleotide or gene product or to add or knockdown a gene product. In some embodiments the genetic engineering is done, for example, to treat disease, following which the genetically engineered heHSC would be transplanted and engraft into a subject. For example, a mobilized heHSC may be manipulated to express one or more polynucleotides or genes that would enhance the engrafting ability of the transplanted heHSC.

Techniques for transfecting cells are known in the art. In an exemplary embodiment, gene therapy can be used to insert a polynucleotide (e.g., DNA) into a mobilized hematopoietic stem cell from a patient or subject with a genetic defect to correct such genetic defect, following which the corrected or genetically engineered mobilized hematopoietic stem cell may be transplanted into a subject.

In some other embodiments, the heHSCs disclosed herein can be used as carriers for gene therapy.

In some embodiments, the isolated heHSCs and the related methods of mobilizing such heHSCs are useful for treating subjects that have demonstrated poor mobilization in response to a conventional hematopoietic stem cell and/or progenitor cell mobilization regimen (e.g., subjects that have failed to mobilize a sufficient numbers of stem cells following a mobilization regimen comprising or consisting of G-CSF). For example, such heHSCs and the related methods disclosed herein may be used to enhance hematopoietic stem cell and/or progenitor cell mobilization in individuals exhibiting stem cell and/or progenitor cell mobilopathy. Accordingly, in certain embodiments, any of the methods and compositions disclosed herein may be suitable for use in mobilizing hematopoietic stem cell and/or progenitor stem cells in a subject having an underlying disease that impairs egress of such hematopoietic stem cells and/or progenitor stem cells from bone marrow and into the peripheral circulation, including, for example, subjects that have or are at risk of developing diabetic stem cell mobilopathy. In certain aspects, subjects that have failed to mobilize a sufficient number of hematopoietic stem cells and/or progenitor cells in response to a mobilization regimen comprising G-CSF (e.g., subjects that have failed to mobilize a sufficient number of stem cells about five days after receiving a G-CSF mobilization regimen) are candidates for mobilization using the methods and compositions disclosed herein. In certain embodiments, the isolated heHSCs may be administered to a subject exhibiting mobilopathy for the treatment of a stem cell or progenitor cell disorder.

As used herein to describe a mobilization regimen, the term “conventional” generally refers to those mobilization regimens that have traditionally been used to mobilize stem cells. For example, conventional mobilization regimens include those comprising or consisting of G-CSF and that have historically been used to mobilize stem cells from the bone marrow compartment. Such convention mobilization regimens are frequently associated with poor mobilization results, which may often occur over an extended period of time (e.g., over about 5 days), and subjecting the patient to repeated and prolonged apheresis procedures.

In addition to being phenotypically unique relative to stem cells mobilized using traditional mobilization regimens, the heHSCs disclosed herein are characterized by their improved functional properties. For example, in certain embodiments, the heHSCs disclosed herein are characterized by their improved engrafting ability. Accordingly, certain aspects of the methods disclosed herein comprise administering or otherwise transplanting the isolated, non-native heHSCs to a subject in need, such that the administered heHSCs engraft in the tissues (e.g., the bone marrow tissue) of the recipient subject. As used herein, the terms “engrafting” and “engraftment” refer to placing or administration of the heHSCs into an animal (e.g., by injection), wherein following such placement or administration, the heHSCs persist in vivo. Engraftment may be readily measured by the ability of the transplanted heHSCs to, for example, contribute to the ongoing blood cell formation or by assessing donor chimerism following the transplant of such heHSCs.

Successful stem cell transplantation depends on the ability to engraft sufficient quantities of transplanted stem cells in the tissues of the subject (e.g., the bone marrow tissues of the subject). The heHSCs disclosed herein are characterized by their improved engrafting ability and accordingly, certain aspects of the present invention relate to methods of treating stem cell and/or progenitor cell disorders or other diseases requiring transplantation of hematopoietic stem cells and/or progenitor cells by administering to a subject the non-native, isolated heHSCs disclosed herein.

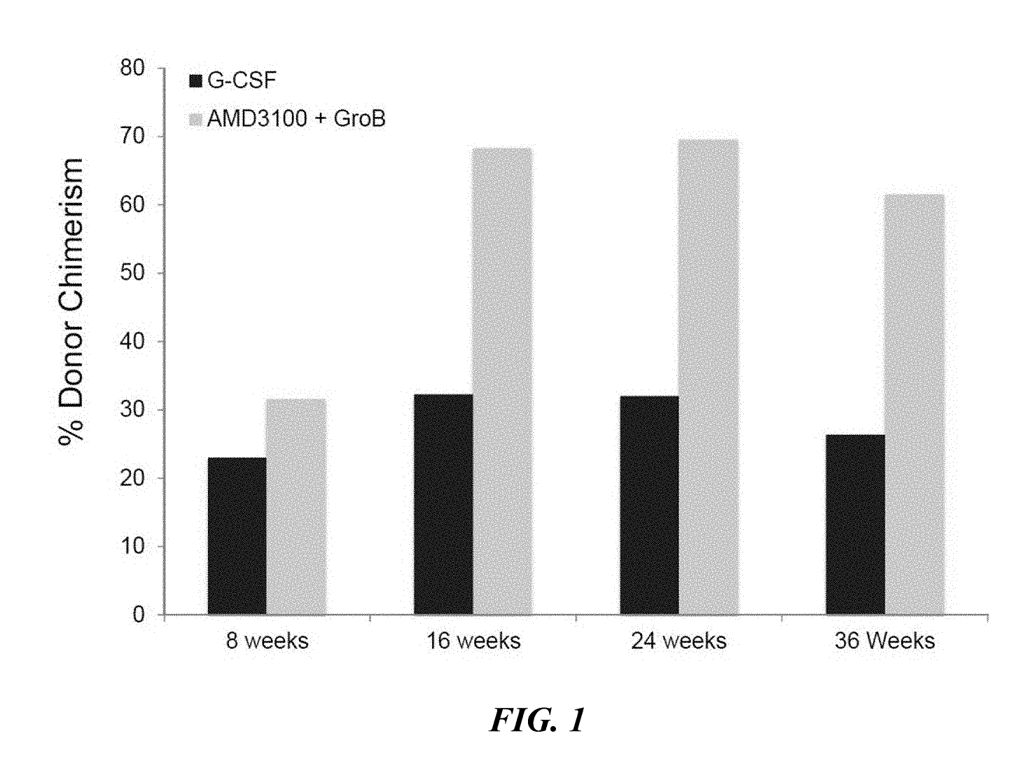

The heHSCs disclosed herein are also characterized by their ability to achieve enhanced or improved donor chimerism following their engraftment in the tissues of a subject. For example, as illustrated in FIG. 1, relative to G-CSF-mobilized stem cells, in certain embodiments, an increase in donor chimerism is observed following engraftment of heHSCs that were mobilized with the combination of one or more CXCR2 agonists (e.g., GROβ and analogs or derivatives thereof) and one or more CXCR4 antagonist (e.g., AMD-3100 and analogs or derivatives thereof). As used herein, the term “donor chimerism” refers to the fraction or percentage of bone marrow cells that originate from the donor heHSCs following engraftment of such heHSCs in a subject. In certain embodiments, donor chimerism following engraftment of the heHSCs is increased relative to, for example, donor chimerism observed following engraftment of the same or a similar quantity of stem cells that are mobilized using conventional mobilization regimens (e.g., conventional mobilization regimens comprising or consisting of G-CSF or other chemotherapeutic agents). In certain embodiments, donor chimerism following engraftment of the heHSCs is increased by at least about two fold, three-fold, four-fold, five-fold, six-fold, or more. In some embodiments, such donor chimerism is at least about 10%, 20%, 25%, 30%, 40%, 50%, 60%, 70%, 75%, 80%, 85%, 90%, 95%, 97%, 99%, or more.

In certain aspects, the heHSCs disclosed herein are also characterized by their ability to achieve an enhanced or improved CD34+ number upon engraftment in a subject. For example, such engrafted heHSCs demonstrate an enhanced or improved CD34+ number relative to an engraftment of the same quantity of hematopoietic stem cells contacted with G-CSF or one or more chemotherapeutic agents described herein. In some embodiments, such CD34+ number is increased by at least about 10%, 20%, 25%, 30%, 40%, 50%, 60%, 70%, 75%, 80%, 85%, 90%, 95%, 97%, 99%, 100%, 150%, 200%, 300%, or more relative to, for example, the CD34+ number observed following engraftment of a G-CSF-mobilized stem cell. In some embodiments, such CD34+ number is increased by at least about 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, or more relative to, for example, the CD34+ number observed following engraftment of a G-CSF-mobilized stem cell.

In some embodiments, also disclosed herein are methods of treating a stem cell or progenitor cell disorder or a disease requiring transplantation of stem cells, the methods comprising administering the isolated, non-native heHSCs to a subject, wherein the administered heHSCs engrafts in the subject's tissues (e.g., the subject's bone marrow compartment), thereby treating the stem cell or progenitor cell disorder.

As used herein, the terms “treat,” “treatment,” “treating,” or “amelioration” when used in reference to a stem cell disorder, progenitor cell disorder or any disease requiring stem cell transplantation, generally refer to therapeutic treatments for a condition, wherein the object is to reverse, alleviate, ameliorate, inhibit, slow down or stop the progression or severity of a symptom or condition. The term “treating” also includes reducing or alleviating at least one adverse effect or symptom of a condition, disease or disorder. Treatment is generally effective if one or more symptoms or clinical markers of the condition or disease are reduced. Alternatively, treatment is effective if the progression of a condition is reduced or halted. That is, treatment includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized state of, for example, a condition, disease, or disorder described herein, or delaying or slowing onset of a condition, disease, or disorder described herein, and an increased lifespan as compared to that expected in the absence of treatment.

As used herein, the term “administering,” generally refers to the placement of the heHSCs described herein into a subject (e.g., the parenteral placement of heHSCs into a subject) by a method or route which results in delivery of such heHSCs to an intended target tissue or site of action (e.g., the bone marrow tissue of a subject). In certain aspects, the term “administering” refers to the placement of at least one CXCR2 agonist and at least one CXCR4 antagonist, VLA-4 antagonist, α₉β₁ antagonist, α₉β₁ integrin/VLA-4 antagonist or combination thereof to a subject to mobilize hematopoietic stem cells and/or progenitor cells from, for example, the subject's bone marrow tissues and into the subject's peripheral tissues (e.g., mobilizing such hematopoietic stem cells and/or progenitor cells out of the bone marrow compartment and into one or more of the peripheral compartments, such as the peripheral blood compartment).

The isolated, non-native heHSCs disclosed herein are useful for the treatment of any disease, disorder, condition, or complication associated with a disease, disorder, or condition, in which transplantation of hematopoietic stem cells and/or progenitor cells is desirable. In some embodiments, the present inventions relate to methods of treating diseases that require peripheral blood stem cell transplantation. In some embodiments, the disclosure provides method of treating stem cell disorders and progenitor cell disorders in a subject in need of such treatment. Examples of such stem cell and progenitor disorders include hematological malignancies and non-malignant hematological diseases.

In some embodiments, the disease, stem cell disorder or progenitor cell disorder is a hematological malignancy. Exemplary hematological malignancies which can be treated with the heHSCs and methods described herein include, but are not limited to, acute lymphoid leukemia, acute myeloid leukemia, chronic lymphoid leukemia, chronic myeloid leukemia, diffuse large B-cell non-Hodgkin's lymphoma, mantle cell lymphoma, lymphoblastic lymphoma, Burkitt's lymphoma, follicular B-cell non-Hodgkin's lymphoma, T-cell non-Hodgkin's lymphoma, lymphocyte predominant nodular Hodgkin's lymphoma, multiple myeloma, and juvenile myelomonocytic leukemia.

In some embodiments, the disease, stem cell disorder or progenitor cell disorder is a non-malignant disorder. Exemplary non-malignant diseases which can be treated with the methods and heHSCs described herein include, but are not limited to, myelofibrosis, myelodysplastic syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disease, chronic inflammatory demyelinating polyradiculoneuropathy, human immunodeficiency virus (HIV), Fanconi anemia, sickle cell disease, beta thalassemia major, Hurler's syndrome (MPS-IH), adrenoleukodystrophy, metachromatic leukodystrophy, familial erythrophagocytic lymphohistiocytosis and other histiocytic disorders, severe combined immunodeficiency (SCID), and Wiskott-Aldrich syndrome.

As used herein, the term “subject” means any human or animal. In certain aspects, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing (e.g., all of the above), but excluding one or more groups or species such as humans, primates or rodents. In certain embodiments, the subject is a mammal (e.g., a primate or human). In some embodiments, the subject is a mammal. In some embodiments, the mammal is a human, a non-human primate, a mouse, a rat, a dog, a cat, a horse, or a cow, and is not limited to these examples. Mammals other than humans can be advantageously used, for example, as subjects that represent animal models of, for example, a hematological malignancy. In addition, the methods described herein can be used to treat domesticated animals and/or pets. A subject can be male or female.

In certain embodiments, a subject can be one who has been previously diagnosed with or otherwise identified as suffering from or having a condition, disease, stem cell disorder or progenitor cell disorder described herein in need of treatment (e.g., of a hematological malignancy or non-malignant disease described herein) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors for a condition or one or more complications related to a condition.

A “subject in need” of treatment for a particular condition (e.g., a stem cell or progenitor cell disorder) can be a subject having that condition, diagnosed as having that condition, or at increased risk of developing that condition relative to a given reference population. In some embodiments, the methods of treatment described herein comprise selecting a subject diagnosed with, suspected of having, or at risk of developing a hematological malignancy, for example a hematological malignancy described herein. In some embodiments, the methods described herein comprise selecting a subject diagnosed with, suspected of having, or at risk of developing a non-malignant disease, for example a non-malignant disease described herein.

In other aspects of the invention, heHSC described herein may be produced by obtaining a HSC cell population by any conventional method disclosed in the art and enriching the HSC cell population for one or more cell surface markers or gene expression profiles for heHSC disclosed herein. In some embodiments, the obtained HSC cell population is enriched for CD93+ cells. In some embodiments, the HSC cell population is enriched for CD93+/CD34− cells. In some embodiments, the HSC cell population is enriched by about 1.5-fold, 2-fold, 2.5-fold, 3-fold, 4-fold, 5-fold or more. In some embodiments, the cell population may be enriched to contain at least 40%, 45%, 50%, 55%, 60%, 65%, 70%, 75%, 80%, 85%, 90%, 95%, or more of cells containing a desired cell surface marker or cell expression pattern (e.g., enriched for CD93+ cells or CD93+/CD34− cells). Any suitable procedure (e.g., FACS sorting) may be used for the enrichment.

Some aspects of the invention are directed towards a method of making an HSC product comprising: i) contacting hematopoietic stem cells and/or progenitor cells with at least one CXCR2 agonist and at least one CXCR4 antagonist, VLA-4 antagonist, α9β1 antagonist, α9β1 integrin/VLA-4 antagonist or combination thereof to produce a candidate product; ii) providing a target expression profile for an heHSC product; iii) determining whether the candidate product meets the target expression profile of an heHSC product; and iv) releasing the candidate product as an heHSC product if the candidate product meets the target expression profile of an heHSC product.

In some embodiments, the target expression profile comprises Sca-1+, c-kit+ and Lin− (SKL) cells. In some embodiments, the target expression profile comprises CD48− cells. In some embodiments, the target expression profile comprises CD150+ cells. In some embodiments, the target expression profile comprises CD93+ cells. In some embodiments, the target expression profile comprises CD34− cells. In some embodiments, the target expression profile comprises OPN+ cells.

“The target expression profile” refers to a transcriptome and/or cell surface marker profile indicating the presence of heHSC cells or a certain percentage of heHSC cells in a cell population. In some embodiments, the target expression profile comprises at least 40%, 45%, 50%, 55%, 60%, 65%, 70%, 75%, 80%, 85%, 90%, 95%, or more of cells in the candidate product or enriched candidate product having one or more cell surface markers. In some embodiments, the target expression profile can be a transcriptome profile of the candidate product or enriched candidate product indicating an heHSC product. In some embodiments, the transcriptome profile can be similar or substantially similar to the profiles shown in FIG. 3 or FIG. 4.

In some embodiments, the contacting of the hematopoietic stem cells and/or progenitor cells with at least one CXCR2 agonist and at least one CXCR4 antagonist, VLA-4 antagonist, α9β1 antagonist, α9β1 integrin/VLA-4 antagonist or combination thereof is performed in vivo. In some embodiments, the contacting is performed in vitro.

In some embodiments, the at least one CXCR2 agonist comprises GROβ or an analog or derivative thereof. In some embodiments, the at least one CXCR2 agonist comprises GROβ-Δ4 or an analog or derivative thereof. In some embodiments, the at least one CXCR4 antagonist comprises plerixafor or an analog or derivative thereof. In some embodiments, the at least one CXCR2 agonist is GROβ or an analog or derivative thereof, and wherein the at least one CXCR4 antagonist is plerixafor or an analog or derivative thereof.

In some embodiments of the invention, the heHSC product, upon transplant into a subject, demonstrates increased engrafting ability relative to engraftment of the same quantity of hematopoietic stem cells contacted with granulocyte colony-stimulating factor (G-CSF), a chemotherapeutic agent, or a combination thereof. In some embodiments, the engrafting ability is increased by at least about two-fold. In certain embodiments, such engrafting ability is increased by at least about two-fold, three-fold, four-fold, five-fold, six-fold, or more.

In some embodiments of the invention, upon engraftment in a subject the heHSC product demonstrates increased donor chimerism relative to engraftment of the same quantity of hematopoietic stem cells contacted with G-CSF, a chemotherapeutic agent, or a combination thereof. In some embodiments, the donor chimerism is increased by at least about two fold. In certain embodiments, such donor chimerism is increased by at least about two-fold, three-fold, four-fold, five-fold, six-fold, or more. In some embodiments, donor chimerism is increased by at least about 50%.

In some embodiments, the heHSC product is non-quiescent.

In some embodiments, the method of making an HSC product additionally comprises a step of enriching the candidate product for one or more cell surface markers and/or one or more gene expression profiles. Any suitable method of enrichment may be employed. In some embodiments, the method is FACS.

In some embodiments, the heHSC product comprises a unique transcriptome relative to hematopoietic stem cells contacted with granulocyte colony-stimulating factor (G-CSF), a chemotherapeutic agent, or a combination thereof. In some embodiments, the heHSC product differentially express one or more of genes selected from the group consisting of Fos, CD93, Fosb, Dusp1, Jun, Dusp6, Cdk1, Fignl1, Plk2, Rsad2, Sgk1, Sdc1, Serpine2, Spp1, Cdca8, Nrp1, Mcam, Pbk, Akr1cl and Cyp11a1, relative to one or more genes expressed by hematopoietic stem cells mobilized using G-CSF. In some embodiments, the heHSC product comprises at least a unique transcriptome or a unique phenotype as compared to a naturally occurring HSC.

In some aspects of the invention, the heHSC product is transformed to express a polynucleotide. In some embodiments, the heHSC product is transformed with an expression vector to express a polynucleotide. In some embodiments, the expression vector comprises a viral vector selected from the group consisting of a retrovirus, a herpes simplex, a lentivirus, an adenovirus, and an adeno-associated virus. In some embodiments, the heHSC product is transfected with an expression vector that comprises the polynucleotide. In some embodiments, polynucleotide comprises an exogenous polynucleotide.

In some embodiments, the heHSC product comprises at least 40% CD93+ cells. In some embodiments, the heHSC product comprises at least about 2×106 cells. In some embodiments, the hematopoietic stem cells and/or progenitor cells are human or mouse cells.

Another aspect of the invention is directed to a method of treating a stem cell or progenitor cell disorder comprising: i) contacting hematopoietic stem cells and/or progenitor cells with at least one CXCR2 agonist and at least one CXCR4 antagonist, VLA-4 antagonist, α9β1 antagonist, α9β1 integrin/VLA-4 antagonist or combination thereof to produce a candidate product; ii) providing a target expression profile for an heHSC product; iii) determining whether the candidate product meets the target expression profile of an heHSC product; and iv) administering the candidate product to a subject in need thereof if the candidate product meets the target expression profile of an heHSC product.

In some embodiments, the target expression profile comprises Sca-1+, c-kit+ and Lin− (SKL) cells. In some embodiments, the target expression profile comprises CD48− cells. In some embodiments, the target expression profile comprises CD150+ cells. In some embodiments, the target expression profile comprises CD93+ cells. In some embodiments, the target expression profile comprises CD34− cells. In some embodiments, the target expression profile comprises OPN+ cells.

“The target expression profile” refers to a transcriptome and/or cell surface marker profile indicating the presence of heHSC cells or a certain percentage of heHSC cells in a cell population. In some embodiments, the target expression profile comprises at least 40%, 45%, 50%, 55%, 60%, 65%, 70%, 75%, 80%, 85%, 90%, 95%, or more of cells in the candidate product or enriched candidate product having one or more cell surface markers. In some embodiments, the target expression profile can be a transcriptome profile of the candidate product or enriched candidate product indicating an heHSC product. In some embodiments, the transcriptome profile can be similar or substantially similar to the profiles shown in FIG. 3 or FIG. 4.

In some embodiments, the contacting of the hematopoietic stem cells and/or progenitor cells with at least one CXCR2 agonist and at least one CXCR4 antagonist, VLA-4 antagonist, α9β1 antagonist, α9β1 integrin/VLA-4 antagonist or combination thereof is performed in vivo. In some embodiments, the contacting is performed in vitro.

In some embodiments, the at least one CXCR2 agonist comprises GROβ or an analog or derivative thereof. In some embodiments, the at least one CXCR2 agonist comprises GROβ-Δ4 or an analog or derivative thereof. In some embodiments, the at least one CXCR4 antagonist comprises plerixafor or an analog or derivative thereof. In some embodiments, the at least one CXCR2 agonist is GROβ or an analog or derivative thereof, and wherein the at least one CXCR4 antagonist is plerixafor or an analog or derivative thereof.

In some embodiments of the invention, the heHSC product, upon transplant into a subject, demonstrates increased engrafting ability relative to engraftment of the same quantity of hematopoietic stem cells contacted with granulocyte colony-stimulating factor (G-CSF), a chemotherapeutic agent, or a combination thereof. In some embodiments, the engrafting ability is increased by at least about two-fold. In certain embodiments, such engrafting ability is increased by at least about two-fold, three-fold, four-fold, five-fold, six-fold, or more.

In some embodiments of the invention, upon engraftment in a subject the heHSC product demonstrates increased donor chimerism relative to engraftment of the same quantity of hematopoietic stem cells contacted with G-CSF, a chemotherapeutic agent, or a combination thereof. In some embodiments, the donor chimerism is increased by at least about two fold. In certain embodiments, such donor chimerism is increased by at least about two-fold, three-fold, four-fold, five-fold, six-fold, or more. In some embodiments, donor chimerism is increased by at least about 50%.

In some embodiments, the heHSC product is non-quiescent.

In some embodiments, the method of making an HSC product additionally comprises a step of enriching the candidate product for one or more cell surface markers and/or one or more gene expression profiles. Any suitable method of enrichment may be employed. In some embodiments, the method is FACS.

In some embodiments, the heHSC product comprises a unique transcriptome relative to hematopoietic stem cells contacted with granulocyte colony-stimulating factor (G-CSF), a chemotherapeutic agent, or a combination thereof. In some embodiments, the heHSC product differentially express one or more of genes selected from the group consisting of Fos, CD93, Fosb, Dusp1, Jun, Dusp6, Cdk1, Fignl1, Plk2, Rsad2, Sgk1, Sdc1, Serpine2, Spp1, Cdca8, Nrp1, Mcam, Pbk, Akr1cl and Cyp11a1, relative to one or more genes expressed by hematopoietic stem cells mobilized using G-CSF. In some embodiments, the heHSC product comprises at least a unique transcriptome or a unique phenotype as compared to a naturally occurring HSC.

In some aspects of the invention, the heHSC product is transformed to express a polynucleotide. In some embodiments, the heHSC product is transformed with an expression vector to express a polynucleotide. In some embodiments, the expression vector comprises a viral vector selected from the group consisting of a retrovirus, a herpes simplex, a lentivirus, an adenovirus, and an adeno-associated virus. In some embodiments, the heHSC product is transfected with an expression vector that comprises the polynucleotide. In some embodiments, polynucleotide comprises an exogenous polynucleotide.

In some embodiments, the heHSC product comprises at least 40% CD93+ cells. In some embodiments, the heHSC product comprises at least about 2×106 cells. In some embodiments, the hematopoietic stem cells and/or progenitor cells are human or mouse cells.

In some embodiments, the stem cell or progenitor cell disorder is a malignant hematologic disease. In some embodiments, the malignant hematologic disease is selected from the group consisting of acute lymphoid leukemia, acute myeloid leukemia, chronic lymphoid leukemia, chronic myeloid leukemia, diffuse large B-cell non-Hodgkin's lymphoma, mantle cell lymphoma, lymphoblastic lymphoma, Burkitt's lymphoma, follicular B-cell non-Hodgkin's lymphoma, lymphocyte predominant nodular Hodgkin's lymphoma, multiple myeloma, and juvenile myelomonocytic leukemia. In some embodiments, the stem cell or progenitor cell disorder is a non-malignant disease. In some embodiments, the non-malignant disease is selected from the group consisting of myelofibrosis, myelodysplastic syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disorder, chronic inflammatory demyelinating polyradiculoneuropathy, human immunodeficiency virus (HIV), Fanconi anemia, sickle cell disorder, beta thalassemia major, Hurler's syndrome (MPS-IH), adrenoleukodystrophy, metachromatic leukodystrophy, familial erythrophagocytic lymphohistiocytosis and other histiocytic disorders, severe combined immunodeficiency (SCID), and Wiskott-Aldrich syndrome.

In certain aspects, the heHSCs described herein can be provided in the form of a kit. For example, the kit may comprise one or more isolated, non-native heHSCs and informational or instructional materials relating to the use or administration of such heHSCs to a subject in need. In some embodiments, such kits may comprise at least one CXCR2 agonist, at least one CXCR4 antagonist and instructions for their administration to a subject to mobilize and/or harvest the hematopoietic stem cells and/or progenitor cells, thereby preparing the isolated heHSCs disclosed herein.

It is to be understood that the invention is not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain agents, compounds, compositions and methods of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXAMPLES

### Example 1 Rapid Regimen

To address the still remaining deficiencies in hematopoietic mobilization, the present inventors believe an effective alternative method is the use of rapid mobilizing agents that do not require multiple injections, that are more predictable in their peak mobilization kinetics, and that result in an enhanced CD34+ number and hematopoietic function upon transplant. One agent with potential is the CXCR2 agonist, GROβ. GROβ and GROβ-Δ4 (collectively referred to herein as “GROβ”) rapidly mobilize hematopoietic stem cells (HSC), including all classes of short-term progenitor cells as well as long-term repopulating cells. In mice, peak GROβ-induced mobilization occurs within 15-30 minutes of administration. Moreover, not only was the observed mobilization faster following GROβ administration, the present inventors believe that the stem cell quality was also greater, at least in view of the improved engrafting ability of the mobilized stem cells (e.g., the two-fold greater engrafting ability of the stem cells mobilized from the bone marrow compartment, relative to stem cells mobilized using, for example, a mobilization regimen comprising C-GSF) and the donor chimerism observed following engraftment of such mobilized stem cells.

To assess this, the present inventors mobilized large cohorts of mice (15-20 per group) with either G-CSF (125 ug/kg/day, five days) or with a combination of GROβ (2.5 mg/kg) and plerixafor (AMD-3100) (5 mg/kg), and then sorted the peripheral blood for highly purified SLAM SKL cells (CD150+, CD48−, Sca-1+, c-kit+, lineage negative)

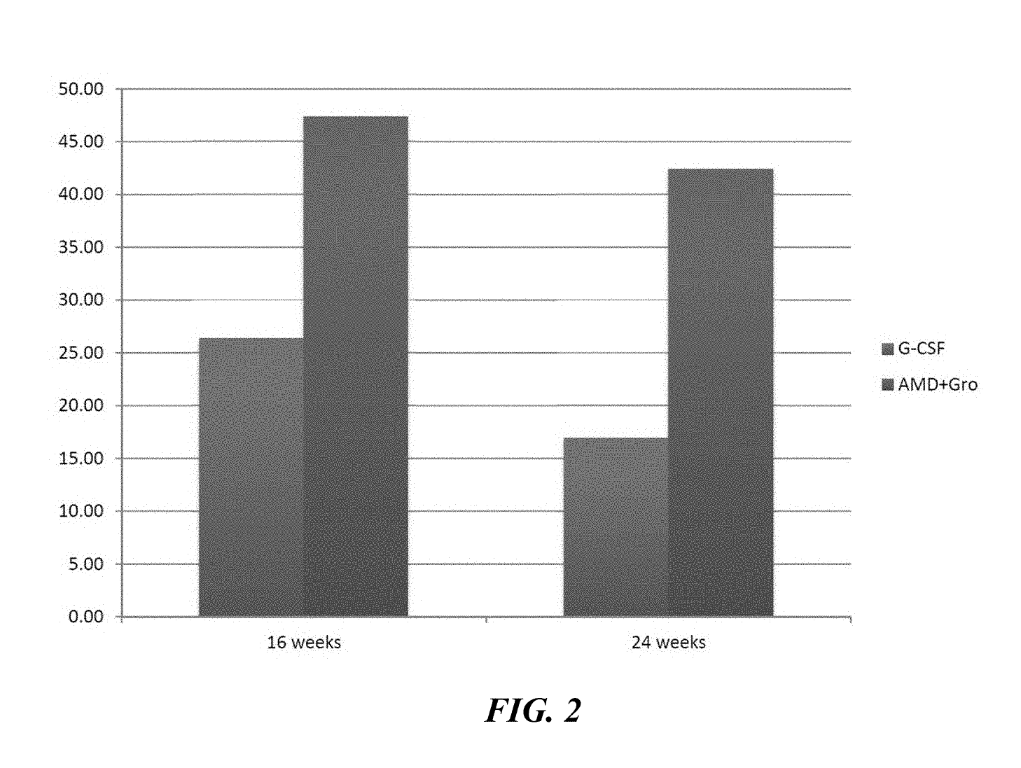

In two separate experiments, the present inventors then competitively transplanted either (a) 190 SLAM SKL cells against 300,000 whole bone marrow competitors, or (b) 50 SLAM SKL cells against 300,000 whole bone marrow competitors. This experimental design allowed for a direct assessment of the engrafting ability of the mobilized SLAM SKL cells, independent of accessory cell populations (e.g., non-CD150+, CD48−, Sca-1+, c-kit+, lineage negative cells) that may have been mobilized, as well as normalized the HSC content so that the same number of HSCs from either the G-CSF-mobilized donors, or the GROβ plus plerixafor-mobilized donors, went into the irradiated recipients. As illustrated in FIGS. 1 and 2 in both sets of experiments, the SLAM SKL cells that were mobilized by the combination of GROβ plus plerixafor demonstrated superior engrafting ability (2 fold greater) relative to the cells that were mobilized by G-CSF. This was evident even when the exact same numbers of phenotypically defined (SLAM SKL) HSCs were transplanted.

### Example 2 Transcriptome Signatures

Over the last decade, there has been increasing evidence that the hematopoietic stem cell (HSC) pool is heterogeneous in function, with identification of HSCs with differing lineage outputs, kinetics of repopulation, length of life-span, and perhaps differences amongst HSCs contributing to homeostatic blood production from those that are the engraftable units in transplantation. To date, however, there are no reliable methods for prospectively isolating differing HSC populations to study heterogeneity. Rather, much of the available data has been acquired based on clonal tracking, single cell transplantation, etc.

Much like panning for gold, the present inventors can now use the differential mobilization properties of the mobilization regimen using GROβ and plerixafor and the regimen using G-CSF as a “biologic sieve” to isolate the heterogeneous HSC populations from the blood. These differential mobilization properties enabled the present inventors, and without destroying the cell, to prospectively isolate what is referred to herein as a highly engraftable HSC (heHSC) population for further functional analysis, and to prospectively isolate a differing HSC population with known, predictable function (the heHSCs) for further molecular characterization.

As a preliminary proof of concept and to demonstrate the feasibility of the approach described herein, SLAM SKL cells were sorted from large cohorts of mice that were treated or mobilized with either G-CSF, or with the combination of GROβ and plerixafor (AMD-3100), as described in Example 1.

In the present study, 200 cells were directly sorted into 5 uL TCL lysis buffer (Qiagen, #1031576). Library preparation was performed by the Smart-Seq2 protocol (Picelli et al., 2013) with subsequent RNA sequencing by Illumina NextSeq500. In addition to SLAM SKL cells from the G-CSF mobilized blood and the GROβ plus plerixafor mobilized blood, additional control samples were sequenced, including steady state bone marrow, bone marrow from the G-CSF-treated mice group, bone marrow from the GROβ plus plerixafor-treated mice, and a “drug spike” control, which consisted of G-CSF mobilized blood spiked with GROβ (350 ng/ml) plus AMD-3100 (10 ug/ml), concentrations based on prior PK data, for 15 minutes, with subsequent downstream processing for FACS sorting. This enabled the present inventors to directly compare the heHSCs from those that were isolated from G-CSF mobilized HSCs, HSCs from the bone marrow of treated and untreated mice, and a drug control to account for any direct effects the GROβ plus plerixafor may have had on the gene signatures that are not due to specific, differential mobilization effects. The RNASeq data was subsequently analyzed, as illustrated in FIG. 3.

Surprisingly, as illustrated in FIG. 4, the highly purified SLAM SKL cells from the GROβ plus plerixafor-mobilized peripheral blood demonstrated a unique transcriptomic signature, including, for example, the expression of CD93 a marker of early lineage stem cells, relative to those HSCs mobilized by G-CSF, as well as from the treated or untreated bone marrow and from the drug spike control. The present inventors believe that the foregoing studies represent the first demonstration of predictable, differential HSC mobilization and provide a novel method to isolate the heHSC cells which have superior clinical utility.

### Example 3 Generation of Unique Stem Cell Populations

Hematopoietic stem cells (HSCs) are at the apex of lifelong blood cell production. Recent clonal analysis studies suggest that HSCs are heterogeneous in function and those that contribute to homeostatic production may be distinct from those that engraft during transplant. The present inventors developed a rapid mobilization regimen utilizing a unique CXCR2 agonist (an N-terminal truncated MIP-2a) and the CXCR4 antagonist AMD-3100. A single subcutaneous injection of both agents together resulted in rapid mobilization in mice with a peak progenitor cell content in blood reached within 15 minutes.

The observed mobilization was equivalent to a 5-day regimen of G-CSF and is the result of synergistic signaling, and was blocked in CXCR4 or CXCR2 knockout mice, confirming receptor and mechanism specificity and is caused by synergistic release of MMP-9 from neutrophils that was blocked in MMP-9 knockout mice, mice treated with an anti-MMP-9 antibody, TIMP-1 transgenic mice, or mice where neutrophils were depleted in vivo using anti-GR-1 antibody. In vivo confocal imaging of mice demonstrated that the mobilization regimen caused a rapid and transient increase in bone marrow vascular permeability, “opening the doorway” for hematopoietic egress to the peripheral blood.

Transplantation of 2×10⁶ peripheral blood mononuclear cells (PBMCs) from the rapid regimen resulted in a 4 or 6 day quicker recovery of neutrophils and platelets, respectively, compared to a G-CSF mobilized graft (n=12 mice per group, P<0.01). In limiting dilution competitive transplants, the rapid regimen demonstrated a greater than 2-fold enhancement in competitiveness (n=30 mice/treatment group, 2 individual experiments, P<0.001). Additionally, in secondarily transplanted mice, competitiveness of the rapidly mobilized graft increased as measured by contribution to chimerism, while G-CSF mobilized grafts remained static (n=16 mice/group, P<0.01). Surprisingly, despite robust enhancement in both short and long-term engraftment by the rapidly mobilized graft, phenotypic analysis of the blood of mobilized mice for CD150+CD48− Sca-1+c-kit+ Lineage neg (SLAM SKL) cells, a highly purified HSC population, showed lower numbers of phenotypically defined HSCs than in the G-CSF group.

The foregoing data suggest that a unique subset of “highly engraftable” HSCs (heHSCs) are mobilized by the rapid regimen comprising an N-terminal truncated MIP-2a and AMD-3100, compared to G-CSF. However, as our earlier studies were performed using grafts that contained the total PBMC fraction (similar to the clinical apheresis product) the present inventors could not rule out the potential contribution of accessory cells to the enhanced engrafting ability of the heHSCs.

### Example 4 Long Term Effects

Following the conclusions set out in Example 3, in 3 independent experiments, the present inventors mobilized large cohorts of mice with the rapid regimen comprising an N-terminal truncated MIP-2a (2.5 mg/kg) and AMD-3100 (5 mg/kg), or G-CSF (125 ug/kg/day, fice days) and sorted SLAM SKL cells from the PBMC fraction and competitively transplanted equal numbers of SLAM SKL cells (190, or 50) from either the rapid regimen or G-CSF and tracked contribution to chimerism over 36 weeks. Remarkably, the heHSCs from the rapid regimen demonstrated a 2-fold enhancement in competitiveness compared to SLAM SKL cells from the G-CSF group (n=11 mice/group, P<0.0004). See FIG. 1.

### Example 5 Molecular Cell Sorting and Signature Determination

While appreciation for HSC heterogeneity has grown, methods are lacking for prospectively isolating differing HSC populations with known biologic function, to study molecular heterogeneity. The present inventors sought to use the differential mobilization properties of our rapid regimen and G-CSF to isolate the heterogeneous HSC populations from the blood. The present inventors again flow sorted SLAM SKL cells from mice mobilized with the rapid regimen or G-CSF and performed RNASeq analysis of the purified populations. The heHSCs mobilized by the rapid regimen had a unique transcriptomic signature compared to G-CSF mobilized or random HSCs acquired from bone marrow (P<0.000001). Strikingly, gene set enrichment analysis (GSEA) demonstrated that the heHSCs had a gene signature highly significantly clustered to that of fetal liver HSCs, further demonstrating the selective harvesting of a subset of highly engraftable stem cells. Our results mechanistically define a new mobilization strategy, that in a single day can mobilize a graft with superior engraftment properties compared to G-CSF, and selectively mobilize a novel population of heHSCs with an immature molecular phenotype capable of robust long-term engraftment.

SEQUENCE LISTING

```
<120> HIGHLY ENGRAFTABLE HEMATOPOIETIC STEM CELLS
<130> HRVY-078-WO1
<150> 62/300,694
<151> 2016 Feb. 26
<150> 62/413,821
<151> 2016 Oct. 27
<160>  23
<210>  1
<211>  73
<212> PRT
<213> Homo sapiens
<220>
<221> MISC_FEATURE
<223> Human Gro-beta
<400> 1
Ala Pro Leu Ala Thr Glu Leu Arg Cys Gln Cys Leu Gln Thr Leu Gln
1 5 10 15
Gly Ile His Leu Lys Asn Ile Gln Ser Val Lys Val Lys Ser Pro Gly
                20 25 30
Pro His Cys Ala Gln Thr Glu Val Ile Ala Thr Leu Lys Asn Gly Gln
        35 40 45
Lys Ala Cys Leu Asn Pro Ala Ser Pro Met Val Lys Lys Ile Ile Glu
    50 55 60
Lys Met Leu Lys Asn Gly Lys Ser Asn
65 70
<210>  2
<211> 107
<212> PRT
<213> Homo sapiens
<220>
<221> MISC_FEATURE
<223> UniProt ID No. P19875- human GRO-beta
<400>  2
Met Ala Arg Ala Thr Leu Ser Ala Ala Pro Ser Asn Pro Arg Leu Leu
1 5 10 15
Arg Val Ala Leu Leu Leu Leu Leu Leu Val Ala Ala Ser Arg Arg Ala
              20 25 30
Ala Gly Ala Pro Leu Ala Thr Glu Leu Arg Cys Gln Cys Leu Gln Thr
          35 40 45
Leu Gln Gly Ile His Leu Lys Asn Ile Gln Ser Val Lys Val Lys Ser
    50 55 60
Pro Gly Pro His Cys Ala Gln Thr Glu Val Ile Ala Thr Leu Lys Asn
65 70 75 80
Gly Gln Lys Ala Cys Leu Asn Pro Ala Ser Pro Met Val Lys Lys Ile
                  85 90 95
Ile Glu Lys Met Leu Lys Asn Gly Lys Ser Asn
                100 105
<210>  3
<211>  69
<212> PRT
<213> Homo sapiens
<220>
<221> MISC_FEATURE
<223> GRO-beta-delta-4
<400>  3
Thr Glu Leu Arg Cys Gln Cys Leu Gln Thr Leu Gln Gly Ile His Leu
1 5 10 15
Lys Asn Ile Gln Ser Val Lys Val Lys Ser Pro Gly Pro His Cys Ala
              20 25 30
Gln Thr Glu Val Ile Ala Thr Leu Lys Asn Gly Gln Lys Ala Cys Leu
        35 40 45
Asn Pro Ala Ser Pro Met Val Lys Lys Ile Ile Glu Lys Met Leu Lys
    50 55 60
Asn Gly Lys Ser Asn
65
<210>  4
<211> 380
<212> PRT
<213> Homo sapiens
<220>
<221> MISC_FEATURE
<223> FOS
<400>  4
Met Met Phe Ser Gly Phe Asn Ala Asp Tyr Glu Ala Ser Ser Ser Arg
1 5 10 15
Cys Ser Ser Ala Ser Pro Ala Gly Asp Ser Leu Ser Tyr Tyr His Ser
              20 25 30
Pro Ala Asp Ser Phe Ser Ser Met Gly Ser Pro Val Asn Ala Gln Asp
          35 40 45
Phe Cys Thr Asp Leu Ala Val Ser Ser Ala Asn Phe Ile Pro Thr Val
    50 55 60
Thr Ala Ile Ser Thr Ser Pro Asp Leu Gln Trp Leu Val Gln Pro Ala
65 70 75 80
Leu Val Ser Ser Val Ala Pro Ser Gln Thr Arg Ala Pro His Pro Phe
                  85 90 95
Gly Val Pro Ala Pro Ser Ala Gly Ala Tyr Ser Arg Ala Gly Val Val
              100 105 110
Lys Thr Met Thr Gly Gly Arg Ala Gln Ser Ile Gly Arg Arg Gly Lys
        115 120 125
Val Glu Gln Leu Ser Pro Glu Glu Glu Glu Lys Arg Arg Ile Arg Arg
    130 135 140
Glu Arg Asn Lys Met Ala Ala Ala Lys Cys Arg Asn Arg Arg Arg Glu
145 150 155 160
Leu Thr Asp Thr Leu Gln Ala Glu Thr Asp Gln Leu Glu Asp Glu Lys
                  165 170 175
Ser Ala Leu Gln Thr Glu Ile Ala Asn Leu Leu Lys Glu Lys Glu Lys
              180 185 190
Leu Glu Phe Ile Leu Ala Ala His Arg Pro Ala Cys Lys Ile Pro Asp
        195 200 205
Asp Leu Gly Phe Pro Glu Glu Met Ser Val Ala Ser Leu Asp Leu Thr
    210 215 220
Gly Gly Leu Pro Glu Val Ala Thr Pro Glu Ser Glu Glu Ala Phe Thr
225 230 235 240
Leu Pro Leu Leu Asn Asp Pro Glu Pro Lys Pro Ser Val Glu Pro Val
                  245 250 255
Lys Ser Ile Ser Ser Met Glu Leu Lys Thr Glu Pro Phe Asp Asp Phe
              260 265 270
Leu Phe Pro Ala Ser Ser Arg Pro Ser Gly Ser Glu Thr Ala Arg Ser
        275 280 285
Val Pro Asp Met Asp Leu Ser Gly Ser Phe Tyr Ala Ala Asp Trp Glu
    290 295 300
Pro Leu His Ser Gly Ser Leu Gly Met Gly Pro Met Ala Thr Glu Leu
305 310 315 320
Glu Pro Leu Cys Thr Pro Val Val Thr Cys Thr Pro Ser Cys Thr Ala
                  325 330 335
Tyr Thr Ser Ser Phe Val Phe Thr Tyr Pro Glu Ala Asp Ser Phe Pro
              340 345 350
Ser Cys Ala Ala Ala His Arg Lys Gly Ser Ser Ser Asn Glu Pro Ser
          355 360 365
Ser Asp Ser Leu Ser Ser Pro Thr Leu Leu Ala Leu
    370 375 380
<210>  5
<211> 652
<212> PRT
<213> Homo sapiens
<220>
<221> MISC_FEATURE
<223> CD93
<400>  5
Met Ala Thr Ser Met Gly Leu Leu Leu Leu Leu Leu Leu Leu Leu Thr
1 5 10 15
Gln Pro Gly Ala Gly Thr Gly Ala Asp Thr Glu Ala Val Val Cys Val
              20 25 30
Gly Thr Ala Cys Tyr Thr Ala His Ser Gly Lys Leu Ser Ala Ala Glu
        35 40 45
Ala Gln Asn His Cys Asn Gln Asn Gly Gly Asn Leu Ala Thr Val Lys
    50 55 60
Ser Lys Glu Glu Ala Gln His Val Gln Arg Val Leu Ala Gln Leu Leu
65 70 75 80
Arg Arg Glu Ala Ala Leu Thr Ala Arg Met Ser Lys Phe Trp Ile Gly
                  85 90 95
Leu Gln Arg Glu Lys Gly Lys Cys Leu Asp Pro Ser Leu Pro Leu Lys
              100 105 110
Gly Phe Ser Trp Val Gly Gly Gly Glu Asp Thr Pro Tyr Ser Asn Trp
        115 120 125
His Lys Glu Leu Arg Asn Ser Cys Ile Ser Lys Arg Cys Val Ser Leu
      130 135 140
Leu Leu Asp Leu Ser Gln Pro Leu Leu Pro Ser Arg Leu Pro Lys Trp
145 150 155 160
Ser Glu Gly Pro Cys Gly Ser Pro Gly Ser Pro Gly Ser Asn Ile Glu
                    165 170 175
Gly Phe Val Cys Lys Phe Ser Phe Lys Gly Met Cys Arg Pro Leu Ala
              180 185 190
Leu Gly Gly Pro Gly Gln Val Thr Tyr Thr Thr Pro Phe Gln Thr Thr
        195 200 205
Ser Ser Ser Leu Glu Ala Val Pro Phe Ala Ser Ala Ala Asn Val Ala
    210 215 220
Cys Gly Glu Gly Asp Lys Asp Glu Thr Gln Ser His Tyr Phe Leu Cys
225 230 235 240
Lys Glu Lys Ala Pro Asp Val Phe Asp Trp Gly Ser Ser Gly Pro Leu
                  245 250 255
Cys Val Ser Pro Lys Tyr Gly Cys Asn Phe Asn Asn Gly Gly Cys His
            260 265 270
Gln Asp Cys Phe Glu Gly Gly Asp Gly Ser Phe Leu Cys Gly Cys Arg
        275 280 285
Pro Gly Phe Arg Leu Leu Asp Asp Leu Val Thr Cys Ala Ser Arg Asn
    290 295 300
Pro Cys Ser Ser Ser Pro Cys Arg Gly Gly Ala Thr Cys Val Leu Gly
305 310 315 320
Pro His Gly Lys Asn Tyr Thr Cys Arg Cys Pro Gln Gly Tyr Gln Leu
                    325 330 335
Asp Ser Ser Gln Leu Asp Cys Val Asp Val Asp Glu Cys Gln Asp Ser
              340 345 350
Pro Cys Ala Gln Glu Cys Val Asn Thr Pro Gly Gly Phe Arg Cys Glu
        355 360 365
Cys Trp Val Gly Tyr Glu Pro Gly Gly Pro Gly Glu Gly Ala Cys Gln
    370 375 380
Asp Val Asp Glu Cys Ala Leu Gly Arg Ser Pro Cys Ala Gln Gly Cys
385 390 395 400
Thr Asn Thr Asp Gly Ser Phe His Cys Ser Cys Glu Glu Gly Tyr Val
                405 410 415
Leu Ala Gly Glu Asp Gly Thr Gln Cys Gln Asp Val Asp Glu Cys Val
              420 425 430
Gly Pro Gly Gly Pro Leu Cys Asp Ser Leu Cys Phe Asn Thr Gln Gly
        435 440 445
Ser Phe His Cys Gly Cys Leu Pro Gly Trp Val Leu Ala Pro Asn Gly
    450 455 460
Val Ser Cys Thr Met Gly Pro Val Ser Leu Gly Pro Pro Ser Gly Pro
465 470 475 480
Pro Asp Glu Glu Asp Lys Gly Glu Lys Glu Gly Ser Thr Val Pro Arg
                485 490 495
Ala Ala Thr Ala Ser Pro Thr Arg Gly Pro Glu Gly Thr Pro Lys Ala
              500 505 510
Thr Pro Thr Thr Ser Arg Pro Ser Leu Ser Ser Asp Ala Pro Ile Thr
        515 520 525
Ser Ala Pro Leu Lys Met Leu Ala Pro Ser Gly Ser Pro Gly Val Trp
    530 535 540
Arg Glu Pro Ser Ile His His Ala Thr Ala Ala Ser Gly Pro Gln Glu
545 550 555 560
Pro Ala Gly Gly Asp Ser Ser Val Ala Thr Gln Asn Asn Asp Gly Thr
                  565 570 575
Asp Gly Gln Lys Leu Leu Leu Phe Tyr Ile Leu Gly Thr Val Val Ala
            580 585 590
Ile Leu Leu Leu Leu Ala Leu Ala Leu Gly Leu Leu Val Tyr Arg Lys
            595 600 605
Arg Arg Ala Lys Arg Glu Glu Lys Lys Glu Lys Lys Pro Gln Asn Ala
    610 615 620
Ala Asp Ser Tyr Ser Trp Val Pro Glu Arg Ala Glu Ser Arg Ala Met
625 630 635 640
Glu Asn Gln Tyr Ser Pro Thr Pro Gly Thr Asp Cys
                645 650
<210>  6
<211> 338
<212> PRT
<213> Homo sapiens
<220>
<221> MISC_FEATURE
<223> FOSB
<400>  6
Met Phe Gln Ala Phe Pro Gly Asp Tyr Asp Ser Gly Ser Arg Cys Ser
1 5 10 15
Ser Ser Pro Ser Ala Glu Ser Gln Tyr Leu Ser Ser Val Asp Ser Phe
              20 25 30
Gly Ser Pro Pro Thr Ala Ala Ala Ser Gln Glu Cys Ala Gly Leu Gly
        35 40 45
Glu Met Pro Gly Ser Phe Val Pro Thr Val Thr Ala Ile Thr Thr Ser
    50 55 60
Gln Asp Leu Gln Trp Leu Val Gln Pro Thr Leu Ile Ser Ser Met Ala
65 70 75 80
Gln Ser Gln Gly Gln Pro Leu Ala Ser Gln Pro Pro Val Val Asp Pro
                  85 90 95
Tyr Asp Met Pro Gly Thr Ser Tyr Ser Thr Pro Gly Met Ser Gly Tyr
            100 105 110
Ser Ser Gly Gly Ala Ser Gly Ser Gly Gly Pro Ser Thr Ser Gly Thr
        115 120 125
Thr Ser Gly Pro Gly Pro Ala Arg Pro Ala Arg Ala Arg Pro Arg Arg
    130 135 140
Pro Arg Glu Glu Thr Leu Thr Pro Glu Glu Glu Glu Lys Arg Arg Val
145 150 155 160
Arg Arg Glu Arg Asn Lys Leu Ala Ala Ala Lys Cys Arg Asn Arg Arg
                  165 170 175
Arg Glu Leu Thr Asp Arg Leu Gln Ala Glu Thr Asp Gln Leu Glu Glu
              180 185 190
Glu Lys Ala Glu Leu Glu Ser Glu Ile Ala Glu Leu Gln Lys Glu Lys
        195 200 205
Glu Arg Leu Glu Phe Val Leu Val Ala His Lys Pro Gly Cys Lys Ile
    210 215 220
Pro Tyr Glu Glu Gly Pro Gly Pro Gly Pro Leu Ala Glu Val Arg Asp
225 230 235 240
Leu Pro Gly Ser Ala Pro Ala Lys Glu Asp Gly Phe Ser Trp Leu Leu
                245 250 255
Pro Pro Pro Pro Pro Pro Pro Leu Pro Phe Gln Thr Ser Gln Asp Ala
              260 265 270
Pro Pro Asn Leu Thr Ala Ser Leu Phe Thr His Ser Glu Val Gln Val
        275 280 285
Leu Gly Asp Pro Phe Pro Val Val Asn Pro Ser Tyr Thr Ser Ser Phe
    290 295 300
Val Leu Thr Cys Pro Glu Val Ser Ala Phe Ala Gly Ala Gln Arg Thr
305 310 315 320
Ser Gly Ser Asp Gln Pro Ser Asp Pro Leu Asn Ser Pro Ser Leu Leu
                  325 330 335
Ala Leu
<210>  7
<211> 367
<212> PRT
<213> Homo sapiens
<220>
<221> MISC_FEATURE
<223> Dusp1
<400>  7
Met Val Met Glu Val Gly Thr Leu Asp Ala Gly Gly Leu Arg Ala Leu
1 5 10 15
Leu Gly Glu Arg Ala Ala Gln Cys Leu Leu Leu Asp Cys Arg Ser Phe
            20 25 30
Phe Ala Phe Asn Ala Gly His Ile Ala Gly Ser Val Asn Val Arg Phe
        35 40 45
Ser Thr Ile Val Arg Arg Arg Ala Lys Gly Ala Met Gly Leu Glu His
    50 55 60
Ile Val Pro Asn Ala Glu Leu Arg Gly Arg Leu Leu Ala Gly Ala Tyr
65 70 75 80
His Ala Val Val Leu Leu Asp Glu Arg Ser Ala Ala Leu Asp Gly Ala
                  85 90 95
Lys Arg Asp Gly Thr Leu Ala Leu Ala Ala Gly Ala Leu Cys Arg Glu
            100 105 110
Ala Arg Ala Ala Gln Val Phe Phe Leu Lys Gly Gly Tyr Glu Ala Phe
        115 120 125
Ser Ala Ser Cys Pro Glu Leu Cys Ser Lys Gln Ser Thr Pro Met Gly
    130 135 140
Leu Ser Leu Pro Leu Ser Thr Ser Val Pro Asp Ser Ala Glu Ser Gly
145 150 155 160
Cys Ser Ser Cys Ser Thr Pro Leu Tyr Asp Gln Gly Gly Pro Val Glu
                    165 170 175
Ile Leu Pro Phe Leu Tyr Leu Gly Ser Ala Tyr His Ala Ser Arg Lys
                180 185 190
Asp Met Leu Asp Ala Leu Gly Ile Thr Ala Leu Ile Asn Val Ser Ala
        195 200 205
Asn Cys Pro Asn His Phe Glu Gly His Tyr Gln Tyr Lys Ser Ile Pro
    210 215 220
Val Glu Asp Asn His Lys Ala Asp Ile Ser Ser Trp Phe Asn Glu Ala
225 230 235 240
Ile Asp Phe Ile Asp Ser Ile Lys Asn Ala Gly Gly Arg Val Phe Val
                  245 250 255
His Cys Gln Ala Gly Ile Ser Arg Ser Ala Thr Ile Cys Leu Ala Tyr
              260 265 270
Leu Met Arg Thr Asn Arg Val Lys Leu Asp Glu Ala Phe Glu Phe Val
        275 280 285
Lys Gln Arg Arg Ser Ile Ile Ser Pro Asn Phe Ser Phe Met Gly Gln
    290 295 300
Leu Leu Gln Phe Glu Ser Gln Val Leu Ala Pro His Cys Ser Ala Glu
305 310 315 320
Ala Gly Ser Pro Ala Met Ala Val Leu Asp Arg Gly Thr Ser Thr Thr
                  325 330 335
Thr Val Phe Asn Phe Pro Val Ser Ile Pro Val His Ser Thr Asn Ser
              340 345 350
Ala Leu Ser Tyr Leu Gln Ser Pro Ile Thr Thr Ser Pro Ser Cys
        355 360 365
<210>  8
<211> 331
<212> PRT
<213> Homo sapiens
<220>
<221> MISC_FEATURE
<223> Jun
<400>  8
Met Thr Ala Lys Met Glu Thr Thr Phe Tyr Asp Asp Ala Leu Asn Ala
1 5 10 15
Ser Phe Leu Pro Ser Glu Ser Gly Pro Tyr Gly Tyr Ser Asn Pro Lys
              20 25 30
Ile Leu Lys Gln Ser Met Thr Leu Asn Leu Ala Asp Pro Val Gly Ser
          35 40 45
Leu Lys Pro His Leu Arg Ala Lys Asn Ser Asp Leu Leu Thr Ser Pro
    50 55 60
Asp Val Gly Leu Leu Lys Leu Ala Ser Pro Glu Leu Glu Arg Leu Ile
65 70 75 80
Ile Gln Ser Ser Asn Gly His Ile Thr Thr Thr Pro Thr Pro Thr Gln
                    85 90 95
Phe Leu Cys Pro Lys Asn Val Thr Asp Glu Gln Glu Gly Phe Ala Glu
              100 105 110
Gly Phe Val Arg Ala Leu Ala Glu Leu His Ser Gln Asn Thr Leu Pro
        115 120 125
Ser Val Thr Ser Ala Ala Gln Pro Val Asn Gly Ala Gly Met Val Ala
      130 135 140
Pro Ala Val Ala Ser Val Ala Gly Gly Ser Gly Ser Gly Gly Phe Ser
145 150 155 160
Ala Ser Leu His Ser Glu Pro Pro Val Tyr Ala Asn Leu Ser Asn Phe
                  165 170 175
Asn Pro Gly Ala Leu Ser Ser Gly Gly Gly Ala Pro Ser Tyr Gly Ala
              180 185 190
Ala Gly Leu Ala Phe Pro Ala Gln Pro Gln Gln Gln Gln Gln Pro Pro
        195 200 205
His His Leu Pro Gln Gln Met Pro Val Gln His Pro Arg Leu Gln Ala
    210 215 220
Leu Lys Glu Glu Pro Gln Thr Val Pro Glu Met Pro Gly Glu Thr Pro
225 230 235 240
Pro Leu Ser Pro Ile Asp Met Glu Ser Gln Glu Arg Ile Lys Ala Glu
                  245 250 255
Arg Lys Arg Met Arg Asn Arg Ile Ala Ala Ser Lys Cys Arg Lys Arg
              260 265 270
Lys Leu Glu Arg Ile Ala Arg Leu Glu Glu Lys Val Lys Thr Leu Lys
        275 280 285
Ala Gln Asn Ser Glu Leu Ala Ser Thr Ala Asn Met Leu Arg Glu Gln
    290 295 300
Val Ala Gln Leu Lys Gln Lys Val Met Asn His Val Asn Ser Gly Cys
305 310 315 320
Gln Leu Met Leu Thr Gln Gln Leu Gln Thr Phe
                325 330
<210>  9
<211> 381
<212> PRT
<213> Homo sapiens
<220>
<221> MISC_FEATURE
<223> DUSP6
<400>  9
Met Ile Asp Thr Leu Arg Pro Val Pro Phe Ala Ser Glu Met Ala Ile
1 5 10 15
Ser Lys Thr Val Ala Trp Leu Asn Glu Gln Leu Glu Leu Gly Asn Glu
              20 25 30
Arg Leu Leu Leu Met Asp Cys Arg Pro Gln Glu Leu Tyr Glu Ser Ser
        35 40 45
His Ile Glu Ser Ala Ile Asn Val Ala Ile Pro Gly Ile Met Leu Arg
    50 55 60
Arg Leu Gln Lys Gly Asn Leu Pro Val Arg Ala Leu Phe Thr Arg Gly
65 70 75 80
Glu Asp Arg Asp Arg Phe Thr Arg Arg Cys Gly Thr Asp Thr Val Val
                85 90 95
Leu Tyr Asp Glu Ser Ser Ser Asp Trp Asn Glu Asn Thr Gly Gly Glu
              100 105 110
Ser Val Leu Gly Leu Leu Leu Lys Lys Leu Lys Asp Glu Gly Cys Arg
        115 120 125
Ala Phe Tyr Leu Glu Gly Gly Phe Ser Lys Phe Gln Ala Glu Phe Ser
    130 135 140
Leu His Cys Glu Thr Asn Leu Asp Gly Ser Cys Ser Ser Ser Ser Pro
145 150 155 160
Pro Leu Pro Val Leu Gly Leu Gly Gly Leu Arg Ile Ser Ser Asp Ser
                  165 170 175
Ser Ser Asp Ile Glu Ser Asp Leu Asp Arg Asp Pro Asn Ser Ala Thr
                180 185 190
Asp Ser Asp Gly Ser Pro Leu Ser Asn Ser Gln Pro Ser Phe Pro Val
        195 200 205
Glu Ile Leu Pro Phe Leu Tyr Leu Gly Cys Ala Lys Asp Ser Thr Asn
    210 215 220
Leu Asp Val Leu Glu Glu Phe Gly Ile Lys Tyr Ile Leu Asn Val Thr
225 230 235 240
Pro Asn Leu Pro Asn Leu Phe Glu Asn Ala Gly Glu Phe Lys Tyr Lys
                245 250 255
Gln Ile Pro Ile Ser Asp His Trp Ser Gln Asn Leu Ser Gln Phe Phe
                260 265 270
Pro Glu Ala Ile Ser Phe Ile Asp Glu Ala Arg Gly Lys Asn Cys Gly
        275 280 285
Val Leu Val His Cys Leu Ala Gly Ile Ser Arg Ser Val Thr Val Thr
    290 295 300
Val Ala Tyr Leu Met Gln Lys Leu Asn Leu Ser Met Asn Asp Ala Tyr
305 310 315 320
Asp Ile Val Lys Met Lys Lys Ser Asn Ile Ser Pro Asn Phe Asn Phe
                    325 330 335
Met Gly Gln Leu Leu Asp Phe Glu Arg Thr Leu Gly Leu Ser Ser Pro
              340 345 350
Cys Asp Asn Arg Val Pro Ala Gln Gln Leu Tyr Phe Thr Thr Pro Ser
        355 360 365
Asn Gln Asn Val Tyr Gln Val Asp Ser Leu Gln Ser Thr
    370 375 380
<210>  10
<211> 297
<212> PRT
<213> Homo sapiens
<220>
<221> MISC_FEATURE
<223> CDK1
<400>  10
Met Glu Asp Tyr Thr Lys Ile Glu Lys Ile Gly Glu Gly Thr Tyr Gly
1 5 10 15
Val Val Tyr Lys Gly Arg His Lys Thr Thr Gly Gln Val Val Ala Met
              20 25 30
Lys Lys Ile Arg Leu Glu Ser Glu Glu Glu Gly Val Pro Ser Thr Ala
        35 40 45
Ile Arg Glu Ile Ser Leu Leu Lys Glu Leu Arg His Pro Asn Ile Val
      50 55 60
Ser Leu Gln Asp Val Leu Met Gln Asp Ser Arg Leu Tyr Leu Ile Phe
65 70 75 80
Glu Phe Leu Ser Met Asp Leu Lys Lys Tyr Leu Asp Ser Ile Pro Pro
                  85 90 95
Gly Gln Tyr Met Asp Ser Ser Leu Val Lys Ser Tyr Leu Tyr Gln Ile
            100 105 110
Leu Gln Gly Ile Val Phe Cys His Ser Arg Arg Val Leu His Arg Asp
        115 120 125
Leu Lys Pro Gln Asn Leu Leu Ile Asp Asp Lys Gly Thr Ile Lys Leu
    130 135 140
Ala Asp Phe Gly Leu Ala Arg Ala Phe Gly Ile Pro Ile Arg Val Tyr
145 150 155 160
Thr His Glu Val Val Thr Leu Trp Tyr Arg Ser Pro Glu Val Leu Leu
                  165 170 175
Gly Ser Ala Arg Tyr Ser Thr Pro Val Asp Ile Trp Ser Ile Gly Thr
              180 185 190
Ile Phe Ala Glu Leu Ala Thr Lys Lys Pro Leu Phe His Gly Asp Ser
          195 200 205
Glu Ile Asp Gln Leu Phe Arg Ile Phe Arg Ala Leu Gly Thr Pro Asn
    210 215 220
Asn Glu Val Trp Pro Glu Val Glu Ser Leu Gln Asp Tyr Lys Asn Thr
225 230 235 240
Phe Pro Lys Trp Lys Pro Gly Ser Leu Ala Ser His Val Lys Asn Leu
                  245 250 255
Asp Glu Asn Gly Leu Asp Leu Leu Ser Lys Met Leu Ile Tyr Asp Pro
              260 265 270
Ala Lys Arg Ile Ser Gly Lys Met Ala Leu Asn His Pro Tyr Phe Asn
        275 280 285
Asp Leu Asp Asn Gln Ile Lys Lys Met
    290 295
<210>  11
<211> 674
<212> PRT
<213> Homo sapiens
<220>
<221> MISC_FEATURE
<223> Fignl1
<400>  11
Met Gln Thr Ser Ser Ser Arg Ser Val His Leu Ser Glu Trp Gln Lys
1 5 10 15
Asn Tyr Phe Ala Ile Thr Ser Gly Ile Cys Thr Gly Pro Lys Ala Asp
              20 25 30
Ala Tyr Arg Ala Gln Ile Leu Arg Ile Gln Tyr Ala Trp Ala Asn Ser
          35 40 45
Glu Ile Ser Gln Val Cys Ala Thr Lys Leu Phe Lys Lys Tyr Ala Glu
    50 55 60
Lys Tyr Ser Ala Ile Ile Asp Ser Asp Asn Val Glu Ser Gly Leu Asn
65 70 75 80
Asn Tyr Ala Glu Asn Ile Leu Thr Leu Ala Gly Ser Gln Gln Thr Asp
                85 90 95
Ser Asp Lys Trp Gln Ser Gly Leu Ser Ile Asn Asn Val Phe Lys Met
              100 105 110
Ser Ser Val Gln Lys Met Met Gln Ala Gly Lys Lys Phe Lys Asp Ser
          115 120 125
Leu Leu Glu Pro Ala Leu Ala Ser Val Val Ile His Lys Glu Ala Thr
    130 135 140
Val Phe Asp Leu Pro Lys Phe Ser Val Cys Gly Ser Ser Gln Glu Ser
145 150 155 160
Asp Ser Leu Pro Asn Ser Ala His Asp Arg Asp Arg Thr Gln Asp Phe
                  165 170 175
Pro Glu Ser Asn Arg Leu Lys Leu Leu Gln Asn Ala Gln Pro Pro Met
              180 185 190
Val Thr Asn Thr Ala Arg Thr Cys Pro Thr Phe Ser Ala Pro Val Gly
        195 200 205
Glu Ser Ala Thr Ala Lys Phe His Val Thr Pro Leu Phe Gly Asn Val
    210 215 220
Lys Lys Glu Asn His Ser Ser Ala Lys Glu Asn Ile Gly Leu Asn Val
225 230 235 240
Phe Leu Ser Asn Gln Ser Cys Phe Pro Ala Ala Cys Glu Asn Pro Gln
                245 250 255
Arg Lys Ser Phe Tyr Gly Ser Gly Thr Ile Asp Ala Leu Ser Asn Pro
            260 265 270
Ile Leu Asn Lys Ala Cys Ser Lys Thr Glu Asp Asn Gly Pro Lys Glu
          275 280 285
Asp Ser Ser Leu Pro Thr Phe Lys Thr Ala Lys Glu Gln Leu Trp Val
    290 295 300
Asp Gln Gln Lys Lys Tyr His Gln Pro Gln Arg Ala Ser Gly Ser Ser
305 310 315 320
Tyr Gly Gly Val Lys Lys Ser Leu Gly Ala Ser Arg Ser Arg Gly Ile
                  325 330 335
Leu Gly Lys Phe Val Pro Pro Ile Pro Lys Gln Asp Gly Gly Glu Gln
            340 345 350
Asn Gly Gly Met Gln Cys Lys Pro Tyr Gly Ala Gly Pro Thr Glu Pro
        355 360 365
Ala His Pro Val Asp Glu Arg Leu Lys Asn Leu Glu Pro Lys Met Ile
    370 375 380
Glu Leu Ile Met Asn Glu Ile Met Asp His Gly Pro Pro Val Asn Trp
385 390 395 400
Glu Asp Ile Ala Gly Val Glu Phe Ala Lys Ala Thr Ile Lys Glu Ile
                  405 410 415
Val Val Trp Pro Met Leu Arg Pro Asp Ile Phe Thr Gly Leu Arg Gly
              420 425 430
Pro Pro Lys Gly Ile Leu Leu Phe Gly Pro Pro Gly Thr Gly Lys Thr
        435 440 445
Leu Ile Gly Lys Cys Ile Ala Ser Gln Ser Gly Ala Thr Phe Phe Ser
    450 455 460
Ile Ser Ala Ser Ser Leu Thr Ser Lys Trp Val Gly Glu Gly Glu Lys
465 470 475 480
Met Val Arg Ala Leu Phe Ala Val Ala Arg Cys Gln Gln Pro Ala Val
                485 490 495
Ile Phe Ile Asp Glu Ile Asp Ser Leu Leu Ser Gln Arg Gly Asp Gly
                500 505 510
Glu His Glu Ser Ser Arg Arg Ile Lys Thr Glu Phe Leu Val Gln Leu
        515 520 525
Asp Gly Ala Thr Thr Ser Ser Glu Asp Arg Ile Leu Val Val Gly Ala
    530 535 540
Thr Asn Arg Pro Gln Glu Ile Asp Glu Ala Ala Arg Arg Arg Leu Val
545 550 555 560
Lys Arg Leu Tyr Ile Pro Leu Pro Glu Ala Ser Ala Arg Lys Gln Ile
                565 570 575
Val Ile Asn Leu Met Ser Lys Glu Gln Cys Cys Leu Ser Glu Glu Glu
                580 585 590
Ile Glu Gln Ile Val Gln Gln Ser Asp Ala Phe Ser Gly Ala Asp Met
          595 600 605
Thr Gln Leu Cys Arg Glu Ala Ser Leu Gly Pro Ile Arg Ser Leu Gln
    610 615 620
Thr Ala Asp Ile Ala Thr Ile Thr Pro Asp Gln Val Arg Pro Ile Ala
625 630 635 640
Tyr Ile Asp Phe Glu Asn Ala Phe Arg Thr Val Arg Pro Ser Val Ser
                    645 650 655
Pro Lys Asp Leu Glu Leu Tyr Glu Asn Trp Asn Lys Thr Phe Gly Cys
              660 665 670
Gly Lys
<210>  12
<211> 685
<212> PRT
<213> Homo sapiens
<220>
<221> MISC_FEATURE
<223> Plk2
<400>  12
Met Glu Leu Leu Arg Thr Ile Thr Tyr Gln Pro Ala Ala Ser Thr Lys
1 5 10 15
Met Cys Glu Gln Ala Leu Gly Lys Gly Cys Gly Ala Asp Ser Lys Lys
            20 25 30
Lys Arg Pro Pro Gln Pro Pro Glu Glu Ser Gln Pro Pro Gln Ser Gln
        35 40 45
Ala Gln Val Pro Pro Ala Ala Pro His His His His His His Ser His
    50 55 60
Ser Gly Pro Glu Ile Ser Arg Ile Ile Val Asp Pro Thr Thr Gly Lys
65 70 75 80
Arg Tyr Cys Arg Gly Lys Val Leu Gly Lys Gly Gly Phe Ala Lys Cys
                85 90 95
Tyr Glu Met Thr Asp Leu Thr Asn Asn Lys Val Tyr Ala Ala Lys Ile
              100 105 110
Ile Pro His Ser Arg Val Ala Lys Pro His Gln Arg Glu Lys Ile Asp
          115 120 125
Lys Glu Ile Glu Leu His Arg Ile Leu His His Lys His Val Val Gln
    130 135 140
Phe Tyr His Tyr Phe Glu Asp Lys Glu Asn Ile Tyr Ile Leu Leu Glu
145 150 155 160
Tyr Cys Ser Arg Arg Ser Met Ala His Ile Leu Lys Ala Arg Lys Val
                  165 170 175
Leu Thr Glu Pro Glu Val Arg Tyr Tyr Leu Arg Gln Ile Val Ser Gly
              180 185 190
Leu Lys Tyr Leu His Glu Gln Glu Ile Leu His Arg Asp Leu Lys Leu
        195 200 205
Gly Asn Phe Phe Ile Asn Glu Ala Met Glu Leu Lys Val Gly Asp Phe
    210 215 220
Gly Leu Ala Ala Arg Leu Glu Pro Leu Glu His Arg Arg Arg Thr Ile
225 230 235 240
Cys Gly Thr Pro Asn Tyr Leu Ser Pro Glu Val Leu Asn Lys Gln Gly
                245 250 255
His Gly Cys Glu Ser Asp Ile Trp Ala Leu Gly Cys Val Met Tyr Thr
              260 265 270
Met Leu Leu Gly Arg Pro Pro Phe Glu Thr Thr Asn Leu Lys Glu Thr
        275 280 285
Tyr Arg Cys Ile Arg Glu Ala Arg Tyr Thr Met Pro Ser Ser Leu Leu
    290 295 300
Ala Pro Ala Lys His Leu Ile Ala Ser Met Leu Ser Lys Asn Pro Glu
305 310 315 320
Asp Arg Pro Ser Leu Asp Asp Ile Ile Arg His Asp Phe Phe Leu Gln
                325 330 335
Gly Phe Thr Pro Asp Arg Leu Ser Ser Ser Cys Cys His Thr Val Pro
              340 345 350
Asp Phe His Leu Ser Ser Pro Ala Lys Asn Phe Phe Lys Lys Ala Ala
        355 360 365
Ala Ala Leu Phe Gly Gly Lys Lys Asp Lys Ala Arg Tyr Ile Asp Thr
    370 375 380
His Asn Arg Val Ser Lys Glu Asp Glu Asp Ile Tyr Lys Leu Arg His
385 390 395 400
Asp Leu Lys Lys Thr Ser Ile Thr Gln Gln Pro Ser Lys His Arg Thr
                  405 410 415
Asp Glu Glu Leu Gln Pro Pro Thr Thr Thr Val Ala Arg Ser Gly Thr
            420 425 430
Pro Ala Val Glu Asn Lys Gln Gln Ile Gly Asp Ala Ile Arg Met Ile
        435 440 445
Val Arg Gly Thr Leu Gly Ser Cys Ser Ser Ser Ser Glu Cys Leu Glu
    450 455 460
Asp Ser Thr Met Gly Ser Val Ala Asp Thr Val Ala Arg Val Leu Arg
465 470 475 480
Gly Cys Leu Glu Asn Met Pro Glu Ala Asp Cys Ile Pro Lys Glu Gln
                485 490 495
Leu Ser Thr Ser Phe Gln Trp Val Thr Lys Trp Val Asp Tyr Ser Asn
              500 505 510
Lys Tyr Gly Phe Gly Tyr Gln Leu Ser Asp His Thr Val Gly Val Leu
        515 520 525
Phe Asn Asn Gly Ala His Met Ser Leu Leu Pro Asp Lys Lys Thr Val
    530 535 540
His Tyr Tyr Ala Glu Leu Gly Gln Cys Ser Val Phe Pro Ala Thr Asp
545 550 555 560
Ala Pro Glu Gln Phe Ile Ser Gln Val Thr Val Leu Lys Tyr Phe Ser
                565 570 575
His Tyr Met Glu Glu Asn Leu Met Asp Gly Gly Asp Leu Pro Ser Val
                580 585 590
Thr Asp Ile Arg Arg Pro Arg Leu Tyr Leu Leu Gln Trp Leu Lys Ser
        595 600 605
Asp Lys Ala Leu Met Met Leu Phe Asn Asp Gly Thr Phe Gln Val Asn
    610 615 620
Phe Tyr His Asp His Thr Lys Ile Ile Ile Cys Ser Gln Asn Glu Glu
625 630 635 640
Tyr Leu Leu Thr Tyr Ile Asn Glu Asp Arg Ile Ser Thr Thr Phe Arg
                645 650 655
Leu Thr Thr Leu Leu Met Ser Gly Cys Ser Ser Glu Leu Lys Asn Arg
              660 665 670
Met Glu Tyr Ala Leu Asn Met Leu Leu Gln Arg Cys Asn
        675 680 685
<210>  13
<211> 361
<212> PRT
<213> Homo sapiens
<220>
<221> MISC_FEATURE
<223> RSAD2
<400>  13
Met Trp Val Leu Thr Pro Ala Ala Phe Ala Gly Lys Leu Leu Ser Val
1 5 10 15
Phe Arg Gln Pro Leu Ser Ser Leu Trp Arg Ser Leu Val Pro Leu Phe
            20 25 30
Cys Trp Leu Arg Ala Thr Phe Trp Leu Leu Ala Thr Lys Arg Arg Lys
        35 40 45
Gln Gln Leu Val Leu Arg Gly Pro Asp Glu Thr Lys Glu Glu Glu Glu
    50 55 60
Asp Pro Pro Leu Pro Thr Thr Pro Thr Ser Val Asn Tyr His Phe Thr
65 70 75 80
Arg Gln Cys Asn Tyr Lys Cys Gly Phe Cys Phe His Thr Ala Lys Thr
                  85 90 95
Ser Phe Val Leu Pro Leu Glu Glu Ala Lys Arg Gly Leu Leu Leu Leu
              100 105 110
Lys Glu Ala Gly Met Glu Lys Ile Asn Phe Ser Gly Gly Glu Pro Phe
        115 120 125
Leu Gln Asp Arg Gly Glu Tyr Leu Gly Lys Leu Val Arg Phe Cys Lys
    130 135 140
Val Glu Leu Arg Leu Pro Ser Val Ser Ile Val Ser Asn Gly Ser Leu
145 150 155 160
Ile Arg Glu Arg Trp Phe Gln Asn Tyr Gly Glu Tyr Leu Asp Ile Leu
                    165 170 175
Ala Ile Ser Cys Asp Ser Phe Asp Glu Glu Val Asn Val Leu Ile Gly
                180 185 190
Arg Gly Gln Gly Lys Lys Asn His Val Glu Asn Leu Gln Lys Leu Arg
        195 200 205
Arg Trp Cys Arg Asp Tyr Arg Val Ala Phe Lys Ile Asn Ser Val Ile
    210 215 220
Asn Arg Phe Asn Val Glu Glu Asp Met Thr Glu Gln Ile Lys Ala Leu
225 230 235 240
Asn Pro Val Arg Trp Lys Val Phe Gln Cys Leu Leu Ile Glu Gly Glu
                245 250 255
Asn Cys Gly Glu Asp Ala Leu Arg Glu Ala Glu Arg Phe Val Ile Gly
            260 265 270
Asp Glu Glu Phe Glu Arg Phe Leu Glu Arg His Lys Glu Val Ser Cys
        275 280 285
Leu Val Pro Glu Ser Asn Gln Lys Met Lys Asp Ser Tyr Leu Ile Leu
    290 295 300
Asp Glu Tyr Met Arg Phe Leu Asn Cys Arg Lys Gly Arg Lys Asp Pro
305 310 315 320
Ser Lys Ser Ile Leu Asp Val Gly Val Glu Glu Ala Ile Lys Phe Ser
                  325 330 335
Gly Phe Asp Glu Lys Met Phe Leu Lys Arg Gly Gly Lys Tyr Ile Trp
              340 345 350
Ser Lys Ala Asp Leu Lys Leu Asp Trp
          355 360
<210>  14
<211> 431
<212> PRT
<213> Homo sapiens
<220>
<221> MISC_FEATURE
<223> SGK1
<400>  14
Met Thr Val Lys Thr Glu Ala Ala Lys Gly Thr Leu Thr Tyr Ser Arg
1 5 10 15
Met Arg Gly Met Val Ala Ile Leu Ile Ala Phe Met Lys Gln Arg Arg
            20 25 30
Met Gly Leu Asn Asp Phe Ile Gln Lys Ile Ala Asn Asn Ser Tyr Ala
        35 40 45
Cys Lys His Pro Glu Val Gln Ser Ile Leu Lys Ile Ser Gln Pro Gln
    50 55 60
Glu Pro Glu Leu Met Asn Ala Asn Pro Ser Pro Pro Pro Ser Pro Ser
65 70 75 80
Gln Gln Ile Asn Leu Gly Pro Ser Ser Asn Pro His Ala Lys Pro Ser
                  85 90 95
Asp Phe His Phe Leu Lys Val Ile Gly Lys Gly Ser Phe Gly Lys Val
            100 105 110
Leu Leu Ala Arg His Lys Ala Glu Glu Val Phe Tyr Ala Val Lys Val
        115 120 125
Leu Gln Lys Lys Ala Ile Leu Lys Lys Lys Glu Glu Lys His Ile Met
    130 135 140
Ser Glu Arg Asn Val Leu Leu Lys Asn Val Lys His Pro Phe Leu Val
145 150 155 160
Gly Leu His Phe Ser Phe Gln Thr Ala Asp Lys Leu Tyr Phe Val Leu
                  165 170 175
Asp Tyr Ile Asn Gly Gly Glu Leu Phe Tyr His Leu Gln Arg Glu Arg
              180 185 190
Cys Phe Leu Glu Pro Arg Ala Arg Phe Tyr Ala Ala Glu Ile Ala Ser
        195 200 205
Ala Leu Gly Tyr Leu His Ser Leu Asn Ile Val Tyr Arg Asp Leu Lys
    210 215 220
Pro Glu Asn Ile Leu Leu Asp Ser Gln Gly His Ile Val Leu Thr Asp
225 230 235 240
Phe Gly Leu Cys Lys Glu Asn Ile Glu His Asn Ser Thr Thr Ser Thr
                245 250 255
Phe Cys Gly Thr Pro Glu Tyr Leu Ala Pro Glu Val Leu His Lys Gln
              260 265 270
Pro Tyr Asp Arg Thr Val Asp Trp Trp Cys Leu Gly Ala Val Leu Tyr
        275 280 285
Glu Met Leu Tyr Gly Leu Pro Pro Phe Tyr Ser Arg Asn Thr Ala Glu
    290 295 300
Met Tyr Asp Asn Ile Leu Asn Lys Pro Leu Gln Leu Lys Pro Asn Ile
305 310 315 320
Thr Asn Ser Ala Arg His Leu Leu Glu Gly Leu Leu Gln Lys Asp Arg
                  325 330 335
Thr Lys Arg Leu Gly Ala Lys Asp Asp Phe Met Glu Ile Lys Ser His
              340 345 350
Val Phe Phe Ser Leu Ile Asn Trp Asp Asp Leu Ile Asn Lys Lys Ile
        355 360 365
Thr Pro Pro Phe Asn Pro Asn Val Ser Gly Pro Asn Asp Leu Arg His
    370 375 380
Phe Asp Pro Glu Phe Thr Glu Glu Pro Val Pro Asn Ser Ile Gly Lys
385 390 395 400
Ser Pro Asp Ser Val Leu Val Thr Ala Ser Val Lys Glu Ala Ala Glu
                  405 410 415
Ala Phe Leu Gly Phe Ser Tyr Ala Pro Pro Thr Asp Ser Phe Leu
            420 425 430
<210>  15
<211> 310
<212> PRT
<213> Homo sapiens
<220>
<221> MISC_FEATURE
<223> Sdc1
<400>  15
Met Arg Arg Ala Ala Leu Trp Leu Trp Leu Cys Ala Leu Ala Leu Ser
1 5 10 15
Leu Gln Pro Ala Leu Pro Gln Ile Val Ala Thr Asn Leu Pro Pro Glu
            20 25 30
Asp Gln Asp Gly Ser Gly Asp Asp Ser Asp Asn Phe Ser Gly Ser Gly
        35 40 45
Ala Gly Ala Leu Gln Asp Ile Thr Leu Ser Gln Gln Thr Pro Ser Thr
    50 55 60
Trp Lys Asp Thr Gln Leu Leu Thr Ala Ile Pro Thr Ser Pro Glu Pro
65 70 75 80
Thr Gly Leu Glu Ala Thr Ala Ala Ser Thr Ser Thr Leu Pro Ala Gly
                  85 90 95
Glu Gly Pro Lys Glu Gly Glu Ala Val Val Leu Pro Glu Val Glu Pro
              100 105 110
Gly Leu Thr Ala Arg Glu Gln Glu Ala Thr Pro Arg Pro Arg Glu Thr
        115 120 125
Thr Gln Leu Pro Thr Thr His Leu Ala Ser Thr Thr Thr Ala Thr Thr
    130 135 140
Ala Gln Glu Pro Ala Thr Ser His Pro His Arg Asp Met Gln Pro Gly
145 150 155 160
His His Glu Thr Ser Thr Pro Ala Gly Pro Ser Gln Ala Asp Leu His
                  165 170 175
Thr Pro His Thr Glu Asp Gly Gly Pro Ser Ala Thr Glu Arg Ala Ala
              180 185 190
Glu Asp Gly Ala Ser Ser Gln Leu Pro Ala Ala Glu Gly Ser Gly Glu
        195 200 205
Gln Asp Phe Thr Phe Glu Thr Ser Gly Glu Asn Thr Ala Val Val Ala
    210 215 220
Val Glu Pro Asp Arg Arg Asn Gln Ser Pro Val Asp Gln Gly Ala Thr
225 230 235 240
Gly Ala Ser Gln Gly Leu Leu Asp Arg Lys Glu Val Leu Gly Gly Val
                  245 250 255
Ile Ala Gly Gly Leu Val Gly Leu Ile Phe Ala Val Cys Leu Val Gly
                260 265 270
Phe Met Leu Tyr Arg Met Lys Lys Lys Asp Glu Gly Ser Tyr Ser Leu
        275 280 285
Glu Glu Pro Lys Gln Ala Asn Gly Gly Ala Tyr Gln Lys Pro Thr Lys
    290 295 300
Gln Glu Glu Phe Tyr Ala
305 310
<210>  16
<211> 398
<212> PRT
<213> Homo sapiens
<220>
<221> MISC_FEATURE
<223> Serpine2
<400>  16
Met Asn Trp His Leu Pro Leu Phe Leu Leu Ala Ser Val Thr Leu Pro
1 5 10 15
Ser Ile Cys Ser His Phe Asn Pro Leu Ser Leu Glu Glu Leu Gly Ser
                20 25 30
Asn Thr Gly Ile Gln Val Phe Asn Gln Ile Val Lys Ser Arg Pro His
        35 40 45
Asp Asn Ile Val Ile Ser Pro His Gly Ile Ala Ser Val Leu Gly Met
    50 55 60
Leu Gln Leu Gly Ala Asp Gly Arg Thr Lys Lys Gln Leu Ala Met Val
65 70 75 80
Met Arg Tyr Gly Val Asn Gly Val Gly Lys Ile Leu Lys Lys Ile Asn
                85 90 95
Lys Ala Ile Val Ser Lys Lys Asn Lys Asp Ile Val Thr Val Ala Asn
              100 105 110
Ala Val Phe Val Lys Asn Ala Ser Glu Ile Glu Val Pro Phe Val Thr
        115 120 125
Arg Asn Lys Asp Val Phe Gln Cys Glu Val Arg Asn Val Asn Phe Glu
    130 135 140
Asp Pro Ala Ser Ala Cys Asp Ser Ile Asn Ala Trp Val Lys Asn Glu
145 150 155 160
Thr Arg Asp Met Ile Asp Asn Leu Leu Ser Pro Asp Leu Ile Asp Gly
                  165 170 175
Val Leu Thr Arg Leu Val Leu Val Asn Ala Val Tyr Phe Lys Gly Leu
              180 185 190
Trp Lys Ser Arg Phe Gln Pro Glu Asn Thr Lys Lys Arg Thr Phe Val
        195 200 205
Ala Ala Asp Gly Lys Ser Tyr Gln Val Pro Met Leu Ala Gln Leu Ser
    210 215 220
Val Phe Arg Cys Gly Ser Thr Ser Ala Pro Asn Asp Leu Trp Tyr Asn
225 230 235 240
Phe Ile Glu Leu Pro Tyr His Gly Glu Ser Ile Ser Met Leu Ile Ala
                    245 250 255
Leu Pro Thr Glu Ser Ser Thr Pro Leu Ser Ala Ile Ile Pro His Ile
            260 265 270
Ser Thr Lys Thr Ile Asp Ser Trp Met Ser Ile Met Val Pro Lys Arg
        275 280 285
Val Gln Val Ile Leu Pro Lys Phe Thr Ala Val Ala Gln Thr Asp Leu
    290 295 300
Lys Glu Pro Leu Lys Val Leu Gly Ile Thr Asp Met Phe Asp Ser Ser
305 310 315 320
Lys Ala Asn Phe Ala Lys Ile Thr Thr Gly Ser Glu Asn Leu His Val
                  325 330 335
Ser His Ile Leu Gln Lys Ala Lys Ile Glu Val Ser Glu Asp Gly Thr
                340 345 350
Lys Ala Ser Ala Ala Thr Thr Ala Ile Leu Ile Ala Arg Ser Ser Pro
        355 360 365
Pro Trp Phe Ile Val Asp Arg Pro Phe Leu Phe Phe Ile Arg His Asn
    370 375 380
Pro Thr Gly Ala Val Leu Phe Met Gly Gln Ile Asn Lys Pro
385 390 395
<210>  17
<211> 314
<212> PRT
<213> Homo sapiens
<220>
<221> MISC_FEATURE
<223> Spp1
<400>  17
Met Arg Ile Ala Val Ile Cys Phe Cys Leu Leu Gly Ile Thr Cys Ala
1 5 10 15
Ile Pro Val Lys Gln Ala Asp Ser Gly Ser Ser Glu Glu Lys Gln Leu
                20 25 30
Tyr Asn Lys Tyr Pro Asp Ala Val Ala Thr Trp Leu Asn Pro Asp Pro
          35 40 45
Ser Gln Lys Gln Asn Leu Leu Ala Pro Gln Asn Ala Val Ser Ser Glu
    50 55 60
Glu Thr Asn Asp Phe Lys Gln Glu Thr Leu Pro Ser Lys Ser Asn Glu
65 70 75 80
Ser His Asp His Met Asp Asp Met Asp Asp Glu Asp Asp Asp Asp His
                  85 90 95
Val Asp Ser Gln Asp Ser Ile Asp Ser Asn Asp Ser Asp Asp Val Asp
            100 105 110
Asp Thr Asp Asp Ser His Gln Ser Asp Glu Ser His His Ser Asp Glu
        115 120 125
Ser Asp Glu Leu Val Thr Asp Phe Pro Thr Asp Leu Pro Ala Thr Glu
    130 135 140
Val Phe Thr Pro Val Val Pro Thr Val Asp Thr Tyr Asp Gly Arg Gly
145 150 155 160
Asp Ser Val Val Tyr Gly Leu Arg Ser Lys Ser Lys Lys Phe Arg Arg
                165 170 175
Pro Asp Ile Gln Tyr Pro Asp Ala Thr Asp Glu Asp Ile Thr Ser His
              180 185 190
Met Glu Ser Glu Glu Leu Asn Gly Ala Tyr Lys Ala Ile Pro Val Ala
        195 200 205
Gln Asp Leu Asn Ala Pro Ser Asp Trp Asp Ser Arg Gly Lys Asp Ser
    210 215 220
Tyr Glu Thr Ser Gln Leu Asp Asp Gln Ser Ala Glu Thr His Ser His
225 230 235 240
Lys Gln Ser Arg Leu Tyr Lys Arg Lys Ala Asn Asp Glu Ser Asn Glu
                  245 250 255
His Ser Asp Val Ile Asp Ser Gln Glu Leu Ser Lys Val Ser Arg Glu
                260 265 270
Phe His Ser His Glu Phe His Ser His Glu Asp Met Leu Val Val Asp
        275 280 285
Pro Lys Ser Lys Glu Glu Asp Lys His Leu Lys Phe Arg Ile Ser His
    290 295 300
Glu Leu Asp Ser Ala Ser Ser Glu Val Asn
305 310
<210>  18
<211> 280
<212> PRT
<213> Homo sapiens
<220>
<221> MISC_FEATURE
<223> Cdca8
<400>  18
Met Ala Pro Arg Lys Gly Ser Ser Arg Val Ala Lys Thr Asn Ser Leu
1 5 10 15
Arg Arg Arg Lys Leu Ala Ser Phe Leu Lys Asp Phe Asp Arg Glu Val
            20 25 30
Glu Ile Arg Ile Lys Gln Ile Glu Ser Asp Arg Gln Asn Leu Leu Lys
        35 40 45
Glu Val Asp Asn Leu Tyr Asn Ile Glu Ile Leu Arg Leu Pro Lys Ala
    50 55 60
Leu Arg Glu Met Asn Trp Leu Asp Tyr Phe Ala Leu Gly Gly Asn Lys
65 70 75 80
Gln Ala Leu Glu Glu Ala Ala Thr Ala Asp Leu Asp Ile Thr Glu Ile
                  85 90 95
Asn Lys Leu Thr Ala Glu Ala Ile Gln Thr Pro Leu Lys Ser Ala Lys
              100 105 110
Thr Arg Lys Val Ile Gln Val Asp Glu Met Ile Val Glu Glu Glu Glu
        115 120 125
Glu Glu Glu Asn Glu Arg Lys Asn Leu Gln Thr Ala Arg Val Lys Arg
    130 135 140
Cys Pro Pro Ser Lys Lys Arg Thr Gln Ser Ile Gln Gly Lys Gly Lys
145 150 155 160
Gly Lys Arg Ser Ser Arg Ala Asn Thr Val Thr Pro Ala Val Gly Arg
                165 170 175
Leu Glu Val Ser Met Val Lys Pro Thr Pro Gly Leu Thr Pro Arg Phe
            180 185 190
Asp Ser Arg Val Phe Lys Thr Pro Gly Leu Arg Thr Pro Ala Ala Gly
        195 200 205
Glu Arg Ile Tyr Asn Ile Ser Gly Asn Gly Ser Pro Leu Ala Asp Ser
    210 215 220
Lys Glu Ile Phe Leu Thr Val Pro Val Gly Gly Gly Glu Ser Leu Arg
225 230 235 240
Leu Leu Ala Ser Asp Leu Gln Arg His Ser Ile Ala Gln Leu Asp Pro
                245 250 255
Glu Ala Leu Gly Asn Ile Lys Lys Leu Ser Asn Arg Leu Ala Gln Ile
              260 265 270
Cys Ser Ser Ile Arg Thr His Lys
        275 280
<210>  19
<211> 923
<212> PRT
<213> Homo sapiens
<220>
<221> MISC_FEATURE
<223> Nrp1
<400>  19
Met Glu Arg Gly Leu Pro Leu Leu Cys Ala Val Leu Ala Leu Val Leu
1 5 10 15
Ala Pro Ala Gly Ala Phe Arg Asn Asp Lys Cys Gly Asp Thr Ile Lys
              20 25 30
Ile Glu Ser Pro Gly Tyr Leu Thr Ser Pro Gly Tyr Pro His Ser Tyr
          35 40 45
His Pro Ser Glu Lys Cys Glu Trp Leu Ile Gln Ala Pro Asp Pro Tyr
    50 55 60
Gln Arg Ile Met Ile Asn Phe Asn Pro His Phe Asp Leu Glu Asp Arg
65 70 75 80
Asp Cys Lys Tyr Asp Tyr Val Glu Val Phe Asp Gly Glu Asn Glu Asn
                85 90 95
Gly His Phe Arg Gly Lys Phe Cys Gly Lys Ile Ala Pro Pro Pro Val
              100 105 110
Val Ser Ser Gly Pro Phe Leu Phe Ile Lys Phe Val Ser Asp Tyr Glu
        115 120 125
Thr His Gly Ala Gly Phe Ser Ile Arg Tyr Glu Ile Phe Lys Arg Gly
    130 135 140
Pro Glu Cys Ser Gln Asn Tyr Thr Thr Pro Ser Gly Val Ile Lys Ser
145 150 155 160
Pro Gly Phe Pro Glu Lys Tyr Pro Asn Ser Leu Glu Cys Thr Tyr Ile
                  165 170 175
Val Phe Val Pro Lys Met Ser Glu Ile Ile Leu Glu Phe Glu Ser Phe
              180 185 190
Asp Leu Glu Pro Asp Ser Asn Pro Pro Gly Gly Met Phe Cys Arg Tyr
        195 200 205
Asp Arg Leu Glu Ile Trp Asp Gly Phe Pro Asp Val Gly Pro His Ile
    210 215 220
Gly Arg Tyr Cys Gly Gln Lys Thr Pro Gly Arg Ile Arg Ser Ser Ser
225 230 235 240
Gly Ile Leu Ser Met Val Phe Tyr Thr Asp Ser Ala Ile Ala Lys Glu
                    245 250 255
Gly Phe Ser Ala Asn Tyr Ser Val Leu Gln Ser Ser Val Ser Glu Asp
              260 265 270
Phe Lys Cys Met Glu Ala Leu Gly Met Glu Ser Gly Glu Ile His Ser
        275 280 285
Asp Gln Ile Thr Ala Ser Ser Gln Tyr Ser Thr Asn Trp Ser Ala Glu
    290 295 300
Arg Ser Arg Leu Asn Tyr Pro Glu Asn Gly Trp Thr Pro Gly Glu Asp
305 310 315 320
Ser Tyr Arg Glu Trp Ile Gln Val Asp Leu Gly Leu Leu Arg Phe Val
                  325 330 335
Thr Ala Val Gly Thr Gln Gly Ala Ile Ser Lys Glu Thr Lys Lys Lys
            340 345 350
Tyr Tyr Val Lys Thr Tyr Lys Ile Asp Val Ser Ser Asn Gly Glu Asp
        355 360 365
Trp Ile Thr Ile Lys Glu Gly Asn Lys Pro Val Leu Phe Gln Gly Asn
    370 375 380
Thr Asn Pro Thr Asp Val Val Val Ala Val Phe Pro Lys Pro Leu Ile
385 390 395 400
Thr Arg Phe Val Arg Ile Lys Pro Ala Thr Trp Glu Thr Gly Ile Ser
                  405 410 415
Met Arg Phe Glu Val Tyr Gly Cys Lys Ile Thr Asp Tyr Pro Cys Ser
            420 425 430
Gly Met Leu Gly Met Val Ser Gly Leu Ile Ser Asp Ser Gln Ile Thr
        435 440 445
Ser Ser Asn Gln Gly Asp Arg Asn Trp Met Pro Glu Asn Ile Arg Leu
    450 455 460
Val Thr Ser Arg Ser Gly Trp Ala Leu Pro Pro Ala Pro His Ser Tyr
465 470 475 480
Ile Asn Glu Trp Leu Gln Ile Asp Leu Gly Glu Glu Lys Ile Val Arg
                    485 490 495
Gly Ile Ile Ile Gln Gly Gly Lys His Arg Glu Asn Lys Val Phe Met
                500 505 510
Arg Lys Phe Lys Ile Gly Tyr Ser Asn Asn Gly Ser Asp Trp Lys Met
        515 520 525
Ile Met Asp Asp Ser Lys Arg Lys Ala Lys Ser Phe Glu Gly Asn Asn
      530 535 540
Asn Tyr Asp Thr Pro Glu Leu Arg Thr Phe Pro Ala Leu Ser Thr Arg
545 550 555 560
Phe Ile Arg Ile Tyr Pro Glu Arg Ala Thr His Gly Gly Leu Gly Leu
                    565 570 575
Arg Met Glu Leu Leu Gly Cys Glu Val Glu Ala Pro Thr Ala Gly Pro
            580 585 590
Thr Thr Pro Asn Gly Asn Leu Val Asp Glu Cys Asp Asp Asp Gln Ala
        595 600 605
Asn Cys His Ser Gly Thr Gly Asp Asp Phe Gln Leu Thr Gly Gly Thr
    610 615 620
Thr Val Leu Ala Thr Glu Lys Pro Thr Val Ile Asp Ser Thr Ile Gln
625 630 635 640
Ser Glu Phe Pro Thr Tyr Gly Phe Asn Cys Glu Phe Gly Trp Gly Ser
                  645 650 655
His Lys Thr Phe Cys His Trp Glu His Asp Asn His Val Gln Leu Lys
              660 665 670
Trp Ser Val Leu Thr Ser Lys Thr Gly Pro Ile Gln Asp His Thr Gly
        675 680 685
Asp Gly Asn Phe Ile Tyr Ser Gln Ala Asp Glu Asn Gln Lys Gly Lys
    690 695 700
Val Ala Arg Leu Val Ser Pro Val Val Tyr Ser Gln Asn Ser Ala His
705 710 715 720
Cys Met Thr Phe Trp Tyr His Met Ser Gly Ser His Val Gly Thr Leu
                725 730 735
Arg Val Lys Leu Arg Tyr Gln Lys Pro Glu Glu Tyr Asp Gln Leu Val
            740 745 750
Trp Met Ala Ile Gly His Gln Gly Asp His Trp Lys Glu Gly Arg Val
        755 760 765
Leu Leu His Lys Ser Leu Lys Leu Tyr Gln Val Ile Phe Glu Gly Glu
    770 775 780
Ile Gly Lys Gly Asn Leu Gly Gly Ile Ala Val Asp Asp Ile Ser Ile
785 790 795 800
Asn Asn His Ile Ser Gln Glu Asp Cys Ala Lys Pro Ala Asp Leu Asp
                805 810 815
Lys Lys Asn Pro Glu Ile Lys Ile Asp Glu Thr Gly Ser Thr Pro Gly
              820 825 830
Tyr Glu Gly Glu Gly Glu Gly Asp Lys Asn Ile Ser Arg Lys Pro Gly
        835 840 845
Asn Val Leu Lys Thr Leu Asp Pro Ile Leu Ile Thr Ile Ile Ala Met
    850 855 860
Ser Ala Leu Gly Val Leu Leu Gly Ala Val Cys Gly Val Val Leu Tyr
865 870 875 880
Cys Ala Cys Trp His Asn Gly Met Ser Glu Arg Asn Leu Ser Ala Leu
                885 890 895
Glu Asn Tyr Asn Phe Glu Leu Val Asp Gly Val Lys Leu Lys Lys Asp
              900 905 910
Lys Leu Asn Thr Gln Ser Thr Tyr Ser Glu Ala
        915 920
<210>  20
<211> 646
<212> PRT
<213>Homo sapiens
<220>
<221> MISC_FEATURE
<223> Mcam
<400>  20
Met Gly Leu Pro Arg Leu Val Cys Ala Phe Leu Leu Ala Ala Cys Cys
1 5 10 15
Cys Cys Pro Arg Val Ala Gly Val Pro Gly Glu Ala Glu Gln Pro Ala
            20 25 30
Pro Glu Leu Val Glu Val Glu Val Gly Ser Thr Ala Leu Leu Lys Cys
        35 40 45
Gly Leu Ser Gln Ser Gln Gly Asn Leu Ser His Val Asp Trp Phe Ser
    50 55 60
Val His Lys Glu Lys Arg Thr Leu Ile Phe Arg Val Arg Gln Gly Gln
65 70 75 80
Gly Gln Ser Glu Pro Gly Glu Tyr Glu Gln Arg Leu Ser Leu Gln Asp
                  85 90 95
Arg Gly Ala Thr Leu Ala Leu Thr Gln Val Thr Pro Gln Asp Glu Arg
              100 105 110
Ile Phe Leu Cys Gln Gly Lys Arg Pro Arg Ser Gln Glu Tyr Arg Ile
          115 120 125
Gln Leu Arg Val Tyr Lys Ala Pro Glu Glu Pro Asn Ile Gln Val Asn
    130 135 140
Pro Leu Gly Ile Pro Val Asn Ser Lys Glu Pro Glu Glu Val Ala Thr
145 150 155 160
Cys Val Gly Arg Asn Gly Tyr Pro Ile Pro Gln Val Ile Trp Tyr Lys
                  165 170 175
Asn Gly Arg Pro Leu Lys Glu Glu Lys Asn Arg Val His Ile Gln Ser
              180 185 190
Ser Gln Thr Val Glu Ser Ser Gly Leu Tyr Thr Leu Gln Ser Ile Leu
        195 200 205
Lys Ala Gln Leu Val Lys Glu Asp Lys Asp Ala Gln Phe Tyr Cys Glu
    210 215 220
Leu Asn Tyr Arg Leu Pro Ser Gly Asn His Met Lys Glu Ser Arg Glu
225 230 235 240
Val Thr Val Pro Val Phe Tyr Pro Thr Glu Lys Val Trp Leu Glu Val
                  245 250 255
Glu Pro Val Gly Met Leu Lys Glu Gly Asp Arg Val Glu Ile Arg Cys
            260 265 270
Leu Ala Asp Gly Asn Pro Pro Pro His Phe Ser Ile Ser Lys Gln Asn
        275 280 285
Pro Ser Thr Arg Glu Ala Glu Glu Glu Thr Thr Asn Asp Asn Gly Val
    290 295 300
Leu Val Leu Glu Pro Ala Arg Lys Glu His Ser Gly Arg Tyr Glu Cys
305 310 315 320
Gln Gly Leu Asp Leu Asp Thr Met Ile Ser Leu Leu Ser Glu Pro Gln
                  325 330 335
Glu Leu Leu Val Asn Tyr Val Ser Asp Val Arg Val Ser Pro Ala Ala
            340 345 350
Pro Glu Arg Gln Glu Gly Ser Ser Leu Thr Leu Thr Cys Glu Ala Glu
        355 360 365
Ser Ser Gln Asp Leu Glu Phe Gln Trp Leu Arg Glu Glu Thr Gly Gln
    370 375 380
Val Leu Glu Arg Gly Pro Val Leu Gln Leu His Asp Leu Lys Arg Glu
385 390 395 400
Ala Gly Gly Gly Tyr Arg Cys Val Ala Ser Val Pro Ser Ile Pro Gly
                  405 410 415
Leu Asn Arg Thr Gln Leu Val Asn Val Ala Ile Phe Gly Pro Pro Trp
            420 425 430
Met Ala Phe Lys Glu Arg Lys Val Trp Val Lys Glu Asn Met Val Leu
        435 440 445
Asn Leu Ser Cys Glu Ala Ser Gly His Pro Arg Pro Thr Ile Ser Trp
    450 455 460
Asn Val Asn Gly Thr Ala Ser Glu Gln Asp Gln Asp Pro Gln Arg Val
465 470 475 480
Leu Ser Thr Leu Asn Val Leu Val Thr Pro Glu Leu Leu Glu Thr Gly
                485 490 495
Val Glu Cys Thr Ala Ser Asn Asp Leu Gly Lys Asn Thr Ser Ile Leu
              500 505 510
Phe Leu Glu Leu Val Asn Leu Thr Thr Leu Thr Pro Asp Ser Asn Thr
        515 520 525
Thr Thr Gly Leu Ser Thr Ser Thr Ala Ser Pro His Thr Arg Ala Asn
    530 535 540
Ser Thr Ser Thr Glu Arg Lys Leu Pro Glu Pro Glu Ser Arg Gly Val
545 550 555 560
Val Ile Val Ala Val Ile Val Cys Ile Leu Val Leu Ala Val Leu Gly
                    565 570 575
Ala Val Leu Tyr Phe Leu Tyr Lys Lys Gly Lys Leu Pro Cys Arg Arg
              580 585 590
Ser Gly Lys Gln Glu Ile Thr Leu Pro Pro Ser Arg Lys Ser Glu Leu
          595 600 605
Val Val Glu Val Lys Ser Asp Lys Leu Pro Glu Glu Met Gly Leu Leu
    610 615 620
Gln Gly Ser Ser Gly Asp Lys Arg Ala Pro Gly Asp Gln Gly Glu Lys
625 630 635 640
Tyr Ile Asp Leu Arg His
                    645
<210>  21
<211> 322
<212> PRT
<213>Homo sapiens
<220>
<221> MISC_FEATURE
<223> Pbk
<400>  21
Met Glu Gly Ile Ser Asn Phe Lys Thr Pro Ser Lys Leu Ser Glu Lys
1 5 10 15
Lys Lys Ser Val Leu Cys Ser Thr Pro Thr Ile Asn Ile Pro Ala Ser
            20 25 30
Pro Phe Met Gln Lys Leu Gly Phe Gly Thr Gly Val Asn Val Tyr Leu
        35 40 45
Met Lys Arg Ser Pro Arg Gly Leu Ser His Ser Pro Trp Ala Val Lys
    50 55 60
Lys Ile Asn Pro Ile Cys Asn Asp His Tyr Arg Ser Val Tyr Gln Lys
65 70 75 80
Arg Leu Met Asp Glu Ala Lys Ile Leu Lys Ser Leu His His Pro Asn
                85 90 95
Ile Val Gly Tyr Arg Ala Phe Thr Glu Ala Asn Asp Gly Ser Leu Cys
                100 105 110
Leu Ala Met Glu Tyr Gly Gly Glu Lys Ser Leu Asn Asp Leu Ile Glu
        115 120 125
Glu Arg Tyr Lys Ala Ser Gln Asp Pro Phe Pro Ala Ala Ile Ile Leu
    130 135 140
Lys Val Ala Leu Asn Met Ala Arg Gly Leu Lys Tyr Leu His Gln Glu
145 150 155 160
Lys Lys Leu Leu His Gly Asp Ile Lys Ser Ser Asn Val Val Ile Lys
                  165 170 175
Gly Asp Phe Glu Thr Ile Lys Ile Cys Asp Val Gly Val Ser Leu Pro
            180 185 190
Leu Asp Glu Asn Met Thr Val Thr Asp Pro Glu Ala Cys Tyr Ile Gly
        195 200 205
Thr Glu Pro Trp Lys Pro Lys Glu Ala Val Glu Glu Asn Gly Val Ile
    210 215 220
Thr Asp Lys Ala Asp Ile Phe Ala Phe Gly Leu Thr Leu Trp Glu Met
225 230 235 240
Met Thr Leu Ser Ile Pro His Ile Asn Leu Ser Asn Asp Asp Asp Asp
                245 250 255
Glu Asp Lys Thr Phe Asp Glu Ser Asp Phe Asp Asp Glu Ala Tyr Tyr
            260 265 270
Ala Ala Leu Gly Thr Arg Pro Pro Ile Asn Met Glu Glu Leu Asp Glu
        275 280 285
Ser Tyr Gln Lys Val Ile Glu Leu Phe Ser Val Cys Thr Asn Glu Asp
    290 295 300
Pro Lys Asp Arg Pro Ser Ala Ala His Ile Val Glu Ala Leu Glu Thr
305 310 315 320
Asp Val
<210>  22
<211> 262
<212> PRT
<213> Mus musculus
<220>
<221> MISC_FEATURE
<223> Akr1c1
<400>  22
Gly Leu Ala Ile Arg Ser Lys Val Ala Asp Gly Thr Val Arg Arg Glu
1 5 10 15
Asp Ile Phe Tyr Thr Ser Lys Leu Pro Cys Thr Cys His Arg Pro Glu
                20 25 30
Leu Val Gln Pro Cys Leu Glu Gln Ser Leu Arg Lys Leu Gln Leu Asp
        35 40 45
Tyr Val Asp Leu Tyr Leu Ile His Cys Pro Val Ser Met Lys Pro Gly
    50 55 60
Asn Asp Leu Ile Pro Thr Asp Glu Asn Gly Lys Leu Leu Phe Asp Thr
65 70 75 80
Val Asp Leu Cys Asp Thr Trp Glu Ala Met Glu Lys Cys Lys Asp Ser
                  85 90 95
Gly Leu Ala Lys Ser Ile Gly Val Ser Asn Phe Asn Arg Arg Gln Leu
              100 105 110
Glu Met Ile Leu Asn Lys Pro Gly Leu Arg Tyr Lys Pro Val Cys Asn
        115 120 125
Gln Val Glu Cys His Pro Tyr Leu Asn Gln Ser Lys Leu Leu Asp Tyr
    130 135 140
Cys Lys Ser Lys Asp Ile Val Leu Val Ala Tyr Gly Ala Leu Gly Ser
145 150 155 160
Gln Arg Cys Lys Asn Trp Ile Glu Glu Asn Ala Pro Tyr Leu Leu Glu
                165 170 175
Asp Pro Thr Leu Cys Ala Met Ala Glu Lys His Lys Gln Thr Pro Ala
              180 185 190
Leu Ile Ser Leu Arg Tyr Leu Leu Gln Arg Gly Ile Val Ile Val Thr
        195 200 205
Lys Ser Phe Asn Glu Lys Arg Ile Lys Glu Asn Leu Lys Val Phe Glu
    210 215 220
Phe His Leu Pro Ala Glu Asp Met Ala Val Ile Asp Arg Leu Asn Arg
225 230 235 240
Asn Tyr Arg Tyr Ala Thr Ala Arg Ile Ile Ser Ala His Pro Asn Tyr
                245 250 255
Pro Phe Leu Asp Glu Tyr
              260
<210>  23
<211> 521
<212> PRT
<213> Homo sapiens
<220>
<221> MISC_FEATURE
<223> Cypl1a1
<400>  23
Met Leu Ala Lys Gly Leu Pro Pro Arg Ser Val Leu Val Lys Gly Cys
1 5 10 15
Gln Thr Phe Leu Ser Ala Pro Arg Glu Gly Leu Gly Arg Leu Arg Val
            20 25 30
Pro Thr Gly Glu Gly Ala Gly Ile Ser Thr Arg Ser Pro Arg Pro Phe
        35 40 45
Asn Glu Ile Pro Ser Pro Gly Asp Asn Gly Trp Leu Asn Leu Tyr His
    50 55 60
Phe Trp Arg Glu Thr Gly Thr His Lys Val His Leu His His Val Gln
65 70 75 80
Asn Phe Gln Lys Tyr Gly Pro Ile Tyr Arg Glu Lys Leu Gly Asn Val
                  85 90 95
Glu Ser Val Tyr Val Ile Asp Pro Glu Asp Val Ala Leu Leu Phe Lys
              100 105 110
Ser Glu Gly Pro Asn Pro Glu Arg Phe Leu Ile Pro Pro Trp Val Ala
          115 120 125
Tyr His Gln Tyr Tyr Gln Arg Pro Ile Gly Val Leu Leu Lys Lys Ser
    130 135 140
Ala Ala Trp Lys Lys Asp Arg Val Ala Leu Asn Gln Glu Val Met Ala
145 150 155 160
Pro Glu Ala Thr Lys Asn Phe Leu Pro Leu Leu Asp Ala Val Ser Arg
                  165 170 175
Asp Phe Val Ser Val Leu His Arg Arg Ile Lys Lys Ala Gly Ser Gly
            180 185 190
Asn Tyr Ser Gly Asp Ile Ser Asp Asp Leu Phe Arg Phe Ala Phe Glu
        195 200 205
Ser Ile Thr Asn Val Ile Phe Gly Glu Arg Gln Gly Met Leu Glu Glu
    210 215 220
Val Val Asn Pro Glu Ala Gln Arg Phe Ile Asp Ala Ile Tyr Gln Met
225 230 235 240
Phe His Thr Ser Val Pro Met Leu Asn Leu Pro Pro Asp Leu Phe Arg
                  245 250 255
Leu Phe Arg Thr Lys Thr Trp Lys Asp His Val Ala Ala Trp Asp Val
              260 265 270
Ile Phe Ser Lys Ala Asp Ile Tyr Thr Gln Asn Phe Tyr Trp Glu Leu
          275 280 285
Arg Gln Lys Gly Ser Val His His Asp Tyr Arg Gly Ile Leu Tyr Arg
    290 295 300
Leu Leu Gly Asp Ser Lys Met Ser Phe Glu Asp Ile Lys Ala Asn Val
305 310 315 320
Thr Glu Met Leu Ala Gly Gly Val Asp Thr Thr Ser Met Thr Leu Gln
                  325 330 335
Trp His Leu Tyr Glu Met Ala Arg Asn Leu Lys Val Gln Asp Met Leu
              340 345 350
Arg Ala Glu Val Leu Ala Ala Arg His Gln Ala Gln Gly Asp Met Ala
        355 360 365
Thr Met Leu Gln Leu Val Pro Leu Leu Lys Ala Ser Ile Lys Glu Thr
    370 375 380
Leu Arg Leu His Pro Ile Ser Val Thr Leu Gln Arg Tyr Leu Val Asn
385 390 395 400
Asp Leu Val Leu Arg Asp Tyr Met Ile Pro Ala Lys Thr Leu Val Gln
                405 410 415
Val Ala Ile Tyr Ala Leu Gly Arg Glu Pro Thr Phe Phe Phe Asp Pro
              420 425 430
Glu Asn Phe Asp Pro Thr Arg Trp Leu Ser Lys Asp Lys Asn Ile Thr
        435 440 445
Tyr Phe Arg Asn Leu Gly Phe Gly Trp Gly Val Arg Gln Cys Leu Gly
    450 455 460
Arg Arg Ile Ala Glu Leu Glu Met Thr Ile Phe Leu Ile Asn Met Leu
465 470 475 480
Glu Asn Phe Arg Val Glu Ile Gln His Leu Ser Asp Val Gly Thr Thr
                485 490 495
Phe Asn Leu Ile Leu Met Pro Glu Lys Pro Ile Ser Phe Thr Phe Trp
            500 505 510
Pro Phe Asn Gln Glu Ala Thr Gln Gln
        515 520
The following “DNA” are from mRNA
FOS Human DNA
AACCGCATCTGCAGCGAGCAACTGAGAAGCCAAGACTGAGCCGGCGGCCGCGGCGCAGCG
AACGAGCAGTGACCGTGCTCCTACCCAGCTCTGCTTCACAGCGCCCACCTGTCTCCGCCC
CTCGGCCCCTCGCCCGGCTTTGCCTAACCGCCACGATGATGTTCTCGGGCTTCAACGCAG
ACTACGAGGCGTCATCCTCCCGCTGCAGCAGCGCGTCCCCGGCCGGGGATAGCCTCTCTT
ACTACCACTCACCCTTTCGGAGTCCCCGCCCCCTCCGCTGGGGCTTACTCCAGGGCTGGC
GTTGTGAAGACCATGACAGGAGGCCGAGCGCAGAGCATTGGCAGGAGGGGCAAGGTGGAA
CAGTTATCTCCTGAAGAAGAAGAGAAAAGGAGAATCCGAAGGGAAAGGAATAAGATGGCT
GCAGCCAAATGCCGCAACCGGAGGAGGGAGCTGACTGATACACTCCAAGCGGAGACAGAC
CAACTAGAAGATGAGAAGTCTGCTTTGCAGACCGAGATTGCCAACCTGCTGAAGGAGAAG
GAAAAACTAGAGTTCATCCTGGCAGCTCACCGACCTGCCTGCAAGATCCCTGATGACCTG
GGCTTCCCAGAAGAGATGTCTGTGGCTTCCCTTGATCTGACTGGGGGCCTGCCAGAGGTT
GCCACCCCGGAGTCTGAGGAGGCCTTCACCCTGCCTCTCCTCAATGACCCTGAGCCCAAG
CCCTCAGTGGAACCTGTCAAGAGCATCAGCAGCATGGAGCTGAAGACCGAGCCCTTTGAT
GACTTCCTGTTCCCAGCATCATCCAGGCCCAGTGGCTCTGAGACAGCCCGCTCCGTGCCA
GACATGGACCTATCTGGGTCCTTCTATGCAGCAGACTGGGAGCCTCTGCACAGTGGCTCC
CTGGGGATGGGGCCCATGGCCACAGAGCTGGAGCCCCTGTGCACTCCGGTGGTCACCTGT
ACTCCCAGCTGCACTGCTTACACGTCTTCCTTCGTCTTCACCTACCCCGAGGCTGACTCC
TTCCCCAGCTGTGCAGCTGCCCACCGCAAGGGCAGCAGCAGCAATGAGCCTTCCTCTGAC
TCGCTCAGCTCACCCACGCTGCTGGCCCTGTGAGGGGGCAGGGAAGGGGAGGCAGCCGGC
ACCCACAAGTGCCACTGCCCGAGCTGGTGCATTACAGAGAGGAGAAACACATCTTCCCTA
GAGGGTTCCTGTAGACCTAGGGAGGACCTTATCTGTGCGTGAAACACACCAGGCTGTGGG
CCTCAAGGACTTGAAAGCATCCATGTGTGGACTCAAGTCCTTACCTCTTCCGGAGATGTA
GCAAAACGCATGGAGTGTGTATTGTTCCCAGTGACACTTCAGAGAGCTGGTAGTTAGTAG
CATGTTGAGCCAGGCCTGGGTCTGTGTCTCTTTTCTCTTTCTCCTTAGTCTTCTCATAGC
ATTAACTAATCTATTGGGTTCATTATTGGAATTAACCTGGTGCTGGATATTTTCAAATTG
TATCTAGTGCAGCTGATTTTAACAATAACTACTGTGTTCCTGGCAATAGTGTGTTCTGAT
TAGAAATGACCAATATTATACTAAGAAAAGATACGACTTTATTTTCTGGTAGATAGAAAT
AAATAGCTATATCCATGTACTGTAGTTTTTCTTCAACATCAATGTTCATTGTAATGTTAC
TGATCATGCATTGTTGAGGTGGTCTGAATGTTCTGACATTAACAGTTTTCCATGAAAACG
TTTTATTGTGTTTTTAATTTATTTATTAAGATGGATTCTCAGATATTTATATTTTTATTT
TATTTTTTTCTACCTTGAGGTCTTTTGACATGTGGAAAGTGAATTTGAATGAAAAATTTA
AGCATTGTTTGCTTATTGTTCCAAGACATTGTCAATAAAAGCATTTAAGTT
GAATGCG
FOS Mouse Protein
MMFSGFNADYEASSSRCSSASPAGDSLSYYHSPADSFSSMGSPVNTQDFCADLSVSSANF
IPTVTAISTSPDLQWLVQPTLVSSVAPSQTRAPHPYGLPTQSAGAYARAGMVKTVSGGRA
QSIGRRGKVEQLSPEEEEKRRIRRERNKMAAAKCRNRRRELTDTLQAETDQLEDEKSALQ
TEIANLLKEKEKLEFILAAHRPACKIPDDLGFPEEMSVASLDLTGGLPEASTPESEEAFT
LPLLNDPEPKPSLEPVKSISNVELKAEPFDDFLFPASSRPSGSETSRSVPDVDLSGSFYA
ADWEPLHSNSLGMGPMVTELEPLCTPVVTCTPGCTTYTSSFVFTYPEADSFPSCAAAHRK
GSSSNEPSSDSLSSPTLLAL
FOS Mouse DNA
CAGCGAGCAACTGAGAAGACTGGATAGAGCCGGCGGTTCCGCGAACGAGCAGTGACCGCG
CTCCCACCCAGCTCTGCTCTGCAGCTCCCACCAGTGTCTACCCCTGGACCCCTTGCCGGG
CTTTCCCCAAACTTCGACCATGATGTTCTCGGGTTTCAACGCCGACTACGAGGCGTCATC
CTCCCGCTGCAGTAGCGCCTCCCCGGCCGGGGACAGCCTTTCCTACTACCATTCCCCAGC
CGACTCCTTCTCCAGCATGGGCTCTCCTGTCAACACACAGGACTTTTGCGCAGATCTGTC
CGTCTCTAGTGCCAACTTTATCCCCACGGTGACAGCCATCTCCACCAGCCCAGACCTGCA
GTGGCTGGTGCAGCCCACTCTGGTCTCCTCCGTGGCCCCATCGCAGACCAGAGCGCCCCA
TCCTTACGGACTCCCCACCCAGTCTGCTGGGGCTTACGCCAGAGCGGGAATGGTGAAGAC
CGTGTCAGGAGGCAGAGCGCAGAGCATCGGCAGAAGGGGCAAAGTAGAGCAGCTATCTCC
TGAAGAGGAAGAGAAACGGAGAATCCGAAGGGAACGGAATAAGATGGCTGCAGCCAAGTG
CCGGAATCGGAGGAGGGAGCTGACAGATACACTCCAAGCGGAGACAGATCAACTTGAAGA
TGAGAAGTCTGCGTTGCAGACTGAGATTGCCAATCTGCTGAAAGAGAAGGAAAAACTGGA
GTTTATTTTGGCAGCCCACCGACCTGCCTGCAAGATCCCCGATGACCTTGGCTTCCCAGA
GGAGATGTCTGTGGCCTCCCTGGATTTGACTGGAGGTCTGCCTGAGGCTTCCACCCCAGA
GTCTGAGGAGGCCTTCACCCTGCCCCTTCTCAACGACCCTGAGCCCAAGCCATCCTTGGA
GCCAGTCAAGAGCATCAGCAACGTGGAGCTGAAGGCAGAACCCTTTGATGACTTCTTGTT
TCCGGCATCATCTAGGCCCAGTGGCTCAGAGACCTCCCGCTCTGTGCCAGATGTGGACCT
GTCCGGTTCCTTCTATGCAGCAGACTGGGAGCCTCTGCACAGCAATTCCTTGGGGATGGG
GCCCATGGTCACAGAGCTGGAGCCCCTGTGTACTCCCGTGGTCACCTGTACTCCGGGCTG
CACTACTTACACGTCTTCCTTTGTCTTCACCTACCCTGAAGCTGACTCCTTCCCAAGCTG
TGCCGCTGCCCACCGAAAGGGCAGCAGCAGCAACGAGCCCTCCTCCGACTCCCTGAGCTC
ACCCACGCTGCTGGCCCTGTGAGCAGTCAGAGAAGGCAAGGCAGCCGGCATCCAGACGTG
CCACTGCCCGAGCTGGTGCATTACAGAGAGGAGAAACACGTCTTCCCTCGAAGGTTCCCG
TCGACCTAGGGAGGACCTTACCTGTTCGTGAAACACACCAGGCTGTGGGCCTCAAGGACT
TGCAAGCATCCACATCTGGCCTCCAGTCCTCACCTCTTCCAGAGATGTAGCAAAAACAAA
ACAAAACAAAACAAAAAACCGCATGGAGTGTGTTGTTCCTAGTGACACCTGAGAGCTGGT
AGTTAGTAGAGCATGTGAGTCAAGGCCTGGTCTGTGTCTCTTTTCTCTTTCTCCTTAGTT
TTCTCATAGCACTAACTAATCTGTTGGGTTCATTATTGGAATTAACCTGGTGCTGGATTG
TATCTAGTGCAGCTGATTTTAACAATACCTACTGTGTTCCTGGCAATAGCGTGTTCCAAT
TAGAAACGACCAATATTAAACTAAGAAAAGATAGGACTTTATTTTCCAGTAGATAGAAAT
CAATAGCTATATCCATGTACTGTAGTCCTTCAGCGTCAATGTTCATTGTCATGTTACTGA
TCATGCATTGTCGAGGTGGTCTGAATGTTCTGACATTAACAGTTTTCCATGAAAACGTTT
TTATTGTGTTTTCAATTTATTTATTAAGATGGATTCTCAGATATTTATATTTTTATTTTA
TTTTTTTCTACCCTGAGGTCTTTCGACATGTGGAAAGTGAATTTGAATGAAAAATTTTAA
GCATTGTTTGCTTATTGTTCCAAGACATTGTCAATAAAAGCATTTAAGTTGAAAAAAAAA
AAAAAAA
CD93 Human DNA
CTTCTCTGCGCCGGAGTGGCTGCAGCTCACCCCTCAGCTCCCCTTGGGGCCCAGCTGGGA
GCCGAGATAGAAGCTCCTGTCGCCGCTGGGCTTCTCGCCTCCCGCAGAGGGCCACACAGA
GACCGGGATGGCCACCTCCATGGGCCTGCTGCTGCTGCTGCTGCTGCTCCTGACCCAGCC
CGGGGCGGGGACGGGAGCTGACACGGAGGCGGTGGTCTGCGTGGGGACCGCCTGCTACAC
GGCCCACTCGGGCAAGCTGAGCGCTGCCGAGGCCCAGAACCACTGCAACCAGAACGGGGG
CAACCTGGCCACTGTGAAGAGCAAGGAGGAGGCCCAGCACGTCCAGCGAGTACTGGCCCA
GCTCCTGAGGCGGGAGGCAGCCCTGACGGCGAGGATGAGCAAGTTCTGGATTGGGCTCCA
GCGAGAGAAGGGCAAGTGCCTGGACCCTAGTCTGCCGCTGAAGGGCTTCAGCTGGGTGGG
CGGGGGGGAGGACACGCCTTACTCTAACTGGCACAAGGAGCTCCGGAACTCGTGCATCTC
CAAGCGCTGTGTGTCTCTGCTGCTGGACCTGTCCCAGCCGCTCCTTCCCAGCCGCCTCCC
CAAGTGGTCTGAGGGCCCCTGTGGGAGCCCAGGCTCCCCCGGAAGTAACATTGAGGGCTT
CGTGTGCAAGTTCAGCTTCAAAGGCATGTGCCGGCCTCTGGCCCTGGGGGGCCCAGGTCA
GGTGACCTACACCACCCCCTTCCAGACCACCAGTTCCTCCTTGGAGGCTGTGCCCTTTGC
CTCTGCGGCCAATGTAGCCTGTGGGGAAGGTGACAAGGACGAGACTCAGAGTCATTATTT
CCTGTGCAAGGAGAAGGCCCCCGATGTGTTCGACTGGGGCAGCTCGGGCCCCCTCTGTGT
CAGCCCCAAGTATGGCTGCAACTTCAACAATGGGGGCTGCCACCAGGACTGCTTTGAAGG
GGGGGATGGCTCCTTCCTCTGCGGCTGCCGACCAGGATTCCGGCTGCTGGATGACCTGGT
GACCTGTGCCTCTCGAAACCCTTGCAGCTCCAGCCCATGTCGTGGGGGGGCCACGTGCGT
CCTGGGACCCCATGGGAAAAACTACACGTGCCGCTGCCCCCAAGGGTACCAGCTGGACTC
GAGTCAGCTGGACTGTGTGGACGTGGATGAATGCCAGGACTCCCCCTGTGCCCAGGAGTG
TGTCAACACCCCTGGGGGCTTCCGCTGCGAATGCTGGGTTGGCTATGAGCCGGGCGGTCC
TGGAGAGGGGGCCTGTCAGGATGTGGATGAGTGTGCTCTGGGTCGCTCGCCTTGCGCCCA
GGGCTGCACCAACACAGATGGCTCATTTCACTGCTCCTGTGAGGAGGGCTACGTCCTGGC
CGGGGAGGACGGGACTCAGTGCCAGGACGTGGATGAGTGTGTGGGCCCGGGGGGCCCCCT
CTGCGACAGCTTGTGCTTCAACACACAAGGGTCCTTCCACTGTGGCTGCCTGCCAGGCTG
GGTGCTGGCCCCAAATGGGGTCTCTTGCACCATGGGGCCTGTGTCTCTGGGACCACCATC
TGGGCCCCCCGATGAGGAGGACAAAGGAGAGAAAGAAGGGAGCACCGTGCCCCGTGCTGC
AACAGCCAGTCCCACAAGGGGCCCCGAGGGCACCCCCAAGGCTACACCCACCACAAGTAG
ACCTTCGCTGTCATCTGACGCCCCCATCACATCTGCCCCACTCAAGATGCTGGCCCCCAG
TGGGTCCCCAGGCGTCTGGAGGGAGCCCAGCATCCATCACGCCACAGCTGCCTCTGGCCC
CCAGGAGCCTGCAGGTGGGGACTCCTCCGTGGCCACACAAAACAACGATGGCACTGACGG
GCAAAAGCTGCTTTTATTCTACATCCTAGGCACCGTGGTGGCCATCCTACTCCTGCTGGC
CCTGGCTCTGGGGCTACTGGTCTATCGCAAGCGGAGAGCGAAGAGGGAGGAGAAGAAGGA
GAAGAAGCCCCAGAATGCGGCAGACAGTTACTCCTGGGTTCCAGAGCGAGCTGAGAGCAG
GGCCATGGAGAACCAGTACAGTCCGACACCTGGGACAGACTGCTGAAAGTGAGGTGGCCC
TAGAGACACTAGAGTCACCAGCCACCATCCTCAGAGCTTTGAACTCCCCATTCCAAAGGG
GCACCCACATTTTTTTGAAAGACTGGACTGGAATCTTAGCAAACAATTGTAAGTCTCCTC
CTTAAAGGCCCCTTGGAACATGCAGGTATTTTCTACGGGTGTTTGATGTTCCTGAAGTGG
AAGCTGTGTGTTGGCGTGCCACGGTGGGGATTTCGTGACTCTATAATGATTGTTACTCCC
CCTCCCTTTTCAAATTCCAATGTGACCAATTCCGGATCAGGGTGTGAGGAGGCCGGGGCT
AAGGGGCTCCCCTGAATATCTTCTCTGCTCACTTCCACCATCTAAGAGGAAAAGGTGAGT
TGCTCATGCTGATTAGGATTGAAATGATTTGTTTCTCTTCCTAGGATGAAAACTAAATCA
ATTAATTATTCAAAAAAAAAAAAAAAAAAAAAAAAAAAAAAAAAAAAA
AAAAAAAAA
CD93 Mouse Protein
MAISTGLFLLLGLLGQPWAGAAADSQAVVCEGTACYTAHWGKLSAAEAQHRCNENGGNLA
TVKSEEEARHVQQALTQLLKTKAPLEAKMGKFWIGLQREKGNCTYHDLPMRGFSWVGGGE
DTAYSNWYKASKSSCIFKRCVSLILDLSLTPHPSHLPKWHESPCGTPEAPGNSIEGFLCK
FNFKGMCRPLALGGPGRVTYTTPFQATTSSLEAVPFASVANVACGDEAKSETHYFLCNEK
TPGIFHWGSSGPLCVSPKFGCSFNNGGCQQDCFEGGDGSFRCGCRPGFRLLDDLVTCASR
NPCSSNPCTGGGMCHSVPLSENYTCRCPSGYQLDSSQVHCVDIDECQDSPCAQDCVNTLG
SFHCECWVGYQPSGPKEEACEDVDECAAANSPCAQGCINTDGSFYCSCKEGYIVSGEDST
QCEDIDECSDARGNPCDSLCFNTDGSFRCGCPPGWELAPNGVFCSRGTVFSELPARPPQK
EDNDDRKESTMPPTEMPSSPSGSKDVSNRAQTTGLFVQSDIPTASVPLEIEIPSEVSDVW
FELGTYLPTTSGHSKPTHEDSVSAHSDTDGQNLLLFYILGTVVAISLLLVLALGILIYHK
RRAKKEEIKEKKPQNAADSYSWVPERAESQAPENQYSPTPGTDC
CD93 Mouse DNA
GAAAGCAGCAGTGCGCCTCTGCTCCCTTCAGAGCACAGCCTGGTGTCAAGGTCCAGGTTC
CACCGGCTGCTGCTGTCACCGCAGGGGAGTCTAGCCCCTCCCAGAAGGAGACACAGAAGA
ATGGCCATCTCAACTGGTTTGTTCCTGCTGCTGGGGCTCCTTGGCCAGCCCTGGGCAGGG
GCTGCTGCTGATTCACAGGCTGTGGTGTGCGAGGGGACTGCCTGCTATACAGCCCATTGG
GGCAAGCTGAGTGCCGCTGAAGCCCAGCATCGCTGCAATGAGAATGGAGGCAATCTTGCC
ACCGTGAAGAGTGAGGAGGAGGCCCGGCATGTTCAGCAAGCCCTGACTCAGCTCCTGAAG
ACCAAGGCACCCTTGGAAGCAAAGATGGGCAAATTCTGGATCGGGCTCCAGCGAGAGAAG
GGCAACTGTACGTACCATGATTTGCCAATGAGGGGCTTCAGCTGGGTGGGTGGTGGAGAG
GACACAGCTTATTCAAACTGGTACAAAGCCAGCAAGAGCTCCTGTATCTTTAAACGCTGT
GTGTCCCTCATACTGGACCTGTCCTTGACACCTCACCCCAGCCATCTGCCCAAGTGGCAT
GAGAGTCCCTGTGGGACCCCCGAAGCTCCAGGTAACAGCATTGAAGGTTTCCTGTGCAAG
TTCAACTTCAAAGGCATGTGTAGGCCACTGGCGCTGGGTGGTCCAGGGCGGGTGACCTAT
ACCACCCCTTTCCAGGCCACTACCTCCTCTCTGGAGGCTGTGCCTTTTGCCTCTGTAGCC
AATGTAGCTTGTGGGGATGAAGCTAAGAGTGAAACCCACTATTTCCTATGCAATGAAAAG
ACTCCAGGAATATTTCACTGGGGCAGCTCAGGCCCACTCTGTGTCAGCCCCAAGTTTGGT
TGCAGTTTCAACAACGGGGGCTGCCAGCAGGATTGCTTCGAAGGTGGCGATGGCTCCTTC
CGCTGCGGCTGCCGGCCTGGATTTCGACTGCTGGATGATCTAGTAACTTGTGCCTCCAGG
AACCCCTGCAGCTCAAACCCATGCACAGGAGGTGGCATGTGCCATTCTGTACCACTCAGT
GAAAACTACACTTGCCGTTGTCCCAGCGGCTACCAGCTGGACTCTAGCCAAGTGCACTGT
GTGGATATAGATGAGTGCCAGGACTCCCCCTGTGCCCAGGATTGTGTCAACACTCTAGGG
AGCTTCCACTGTGAATGTTGGGTTGGTTACCAACCCAGTGGCCCCAAGGAAGAGGCCTGT
GAAGATGTGGATGAGTGTGCAGCTGCCAACTCGCCCTGTGCCCAAGGCTGCATCAACACT
GATGGCTCTTTCTACTGCTCCTGTAAAGAGGGCTATATTGTGTCTGGGGAAGACAGTACC
CAGTGTGAGGATATAGATGAGTGTTCGGACGCAAGGGGCAATCCATGTGATTCCCTGTGC
TTCAACACAGATGGTTCCTTCAGGTGTGGCTGCCCGCCAGGCTGGGAGCTGGCTCCCAAT
GGGGTCTTTTGTAGCAGGGGCACTGTGTTTTCTGAACTACCAGCCAGGCCTCCCCAAAAG
GAAGACAACGATGACAGAAAGGAGAGTACTATGCCTCCTACTGAAATGCCCAGTTCTCCT
AGTGGCTCTAAGGATGTCTCCAACAGAGCACAGACAACAGGTCTCTTCGTCCAATCAGAT
ATTCCCACTGCCTCTGTTCCACTAGAAATAGAAATCCCTAGTGAAGTATCTGATGTCTGG
TTCGAGTTGGGCACATACCTCCCCACGACCTCCGGCCACAGCAAGCCGACACATGAAGAT
TCTGTGTCTGCACACAGTGACACCGATGGGCAGAACCTGCTTCTGTTTTACATCCTGGGG
ACGGTGGTGGCCATCTCACTCTTGCTGGTGCTGGCCCTAGGGATTCTCATTTATCATAAA
CGGAGAGCCAAGAAGGAGGAGATAAAAGAGAAGAAGCCTCAGAATGCAGCCGACAGCTAT
TCCTGGGTTCCAGAGCGAGCAGAGAGCCAAGCCCCGGAGAATCAGTACAGCCCAACACCA
GGGACAGACTGCTGAAGACTATGTGGCCTTAGAGACAGCTGCCACTACCTTCAGAGCTAC
CTTCTTAGATGAGGGGGAAGCCACATCATTCTGAATGACTTGACTGGACTCTCAGCAAAA
AAATTGTGCACCTTCCACTTAAGAACCTGGTGGCTTGGGATAGGCAGGTATTTTCTTGGT
GCCTTTGATATGTCTGGGGGTGAAAGCTGTGTGTTGGTTTGTCATTGTGGGGAGTTTTGT
GGATATTGACAGACCTCACTCAAACACCCTTTTCAAATCCAATAGCAACTGGTTCCTCTG
GTTCCTAATTAGGGGGAAAGGAGTCAGAGGGGTGGGACAGGGTGGGGGGATGGGGCTTCA
AAGTTTTTTCTTATCACTTGATTTATCATCGAAGGAGTTACTGGTGCTAATTACAATGGA
AACAGTTCCTTTCCATCACAGGACAGACACACCTCAATCCTCCATGGGGTCAACAACTAT
ATACCCCCAGTGACCCCTTAGGCAAGGACTTGTTGAGAACTGCATCACATTTTGACCTGT
TCTCAACAGTACCCATCTATTTCAGGTGGGATCTCTGGACCTTTCCTCCTTCCCATCTTG
TCTGCAATGTGGCAAATGGCTTCTTTTTGCATTTTTACTCCGCCCCCACCCCAAGCTGAA
GTTCATTTGCAGATCAGCGATTAAGTCTGAATTGTGTGGTGGTCAGTCTTGTTTCCTTTT
GTCAGGGGTTATTGTAAATGTTAGTAATTTCGCCTCAAGCCCTCAGTAAGAACATAAATA
TTTTAAAATATGTGCGTTTGAAATCTGTTTCATGCATCCTGGAACTGTGGGATGCTCAGG
CAAGAGTGACTTTAGTCTTTCAGTGAATGTTGCCCAGAATGTGGGTAGGGAAGGCTCACA
GGTTACTCTCCTCCTTAGAGCTACAACATAACATTCTGAGGGGAGTCACAGGGTTGCCTT
TAAAAAGTGGGAGCTATGTCATGCTTTGAGCTTTCTGTTAAGCACCTCTCCTAATAAACT
CTGAAAAAAT
FOSB Human DNA
CATTCATAAGACTCAGAGCTACGGCCACGGCAGGGACACGCGGAACCAAGACTTGGAAAC
TTGATTGTTGTGGTTCTTCTTGGGGGTTATGAAATTTCATTAATCTTTTTTTTTTCCGGG
GAGAAAGTTTTTGGAAAGATTCTTCCAGATATTTCTTCATTTTCTTTTGGAGGACCGACT
TACTTTTTTTGGTCTTCTTTATTACTCCCCTCCCCCCGTGGGACCCGCCGGACGCGTGGA
GGAGACCGTAGCTGAAGCTGATTCTGTACAGCGGGACAGCGCTTTCTGCCCCTGGGGGAG
CAACCCCTCCCTCGCCCCTGGGTCCTACGGAGCCTGCACTTTCAAGAGGTACAGCGGCAT
CCTGTGGGGGCCTGGGCACCGCAGGAAGACTGCACAGAAACTTTGCCATTGTTGGAACGG
GACGTTGCTCCTTCCCCGAGCTTCCCCGGACAGCGTACTTTGAGGACTCGCTCAGCTCAC
CGGGGACTCCCACGGCTCACCCCGGACTTGCACCTTACTTCCCCAACCCGGCCATAGCCT
TGGCTTCCCGGCGACCTCAGCGTGGTCACAGGGGCCCCCCTGTGCCCAGGGAAATGTTTC
AGGCTTTCCCCGGAGACTACGACTCCGGCTCCCGGTGCAGCTCCTCACCCTCTGCCGAGT
CTCAATATCTGTCTTCGGTGGACTCCTTCGGCAGTCCACCCACCGCCGCGGCCTCCCAGG
AGTGCGCCGGTCTCGGGGAAATGCCCGGTTCCTTCGTGCCCACGGTCACCGCGATCACAA
CCAGCCAGGACCTCCAGTGGCTTGTGCAACCCACCCTCATCTCTTCCATGGCCCAGTCCC
AGGGGCAGCCACTGGCCTCCCAGCCCCCGGTCGTCGACCCCTACGACATGCCGGGAACCA
GCTACTCCACACCAGGCATGAGTGGCTACAGCAGTGGCGGAGCGAGTGGCAGTGGTGGGC
CTTCCACCAGCGGAACTACCAGTGGGCCTGGGCCTGCCCGCCCAGCCCGAGCCCGGCCTA
GGAGACCCCGAGAGGAGACGCTCACCCCAGAGGAAGAGGAGAAGCGAAGGGTGCGCCGGG
AACGAAATAAACTAGCAGCAGCTAAATGCAGGAACCGGCGGAGGGAGCTGACCGACCGAC
TCCAGGCGGAGACAGATCAGTTGGAGGAAGAAAAAGCAGAGCTGGAGTCGGAGATCGCCG
AGCTCCAAAAGGAGAAGGAACGTCTGGAGTTTGTGCTGGTGGCCCACAAACCGGGCTGCA
AGATCCCCTACGAAGAGGGGCCCGGGCCGGGCCCGCTGGCGGAGGTGAGAGATTTGCCGG
GCTCAGCACCGGCTAAGGAAGATGGCTTCAGCTGGCTGCTGCCGCCCCCGCCACCACCGC
CCCTGCCCTTCCAGACCAGCCAAGACGCACCCCCCAACCTGACGGCTTCTCTCTTTACAC
ACAGTGAAGTTCAAGTCCTCGGCGACCCCTTCCCCGTTGTTAACCCTTCGTACACTTCTT
CGTTTGTCCTCACCTGCCCGGAGGTCTCCGCGTTCGCCGGCGCCCAACGCACCAGCGGCA
GTGACCAGCCTTCCGATCCCCTGAACTCGCCCTCCCTCCTCGCTCGGTGAACTCTTTAGA
CACACAAAACAAACAAACACATGGGGGAGAGAGACTTGGAAGAGGAGGAGGAGGAGGAGA
AGGAGGAGAGAGAGGGGAAGAGACAAAGTGGGTGTGTGGCCTCCCTGGCTCCTCCGTCTG
ACCCTCTGCGGCCACTGCGCCACTGCCATCGGACAGGAGGATTCCTTGTGTTTTGTCCTG
CCTCTTGTTTCTGTGCCCCGGCGAGGCCGGAGAGCTGGTGACTTTGGGGACAGGGGGTGG
GAAGGGGATGGACACCCCCAGCTGACTGTTGGCTCTCTGACGTCAACCCAAGCTCTGGGG
ATGGGTGGGGAGGGGGGCGGGTGACGCCCACCTTCGGGCAGTCCTGTGTGAGGATGAAGG
GACGGGGGTGGGAGGTAGGCTGTGGGGTGGGCTGGAGTCCTCTCCAGAGAGGCTCAACAA
GGAAAAATGCCACTCCCTACCCAATGTCTCCCACACCCACCCTTTTTTTGGGGTGCCCAG
GTTGGTTTCCCCTGCACTCCCGACCTTAGCTTATTGATCCCACATTTCCATGGTGTGAGA
TCCTCTTTACTCTGGGCAGAAGTGAGCCCCCCCTTAAAGGGAATTCGATGCCCCCCTAGA
ATAATCTCATCCCCCCACCCGACTTCTTTTGAAATGTGAACGTCCTTCCTTGACTGTCTA
GCCACTCCCTCCCAGAAAAACTGGCTCTGATTGGAATTTCTGGCCTCCTAAGGCTCCCCA
CCCCGAAATCAGCCCCCAGCCTTGTTTCTGATGACAGTGTTATCCCAAGACCCTGCCCCC
TGCCAGCCGACCCTCCTGGCCTTCCTCGTTGGGCCGCTCTGATTTCAGGCAGCAGGGGCT
GCTGTGATGCCGTCCTGCTGGAGTGATTTATACTGTGAAATGAGTTGGCCAGATTGTGGG
GTGCAGCTGGGTGGGGCAGCACACCTCTGGGGGGATAATGTCCCCACTCCCGAAAGCCTT
TCCTCGGTCTCCCTTCCGTCCATCCCCCTTCTTCCTCCCCTCAACAGTGAGTTAGACTCA
AGGGGGTGACAGAACCGAGAAGGGGGTGACAGTCCTCCATCCACGTGGCCTCTCTCTCTC
TCCTCAGGACCCTCAGCCCTGGCCTTTTTCTTTAAGGTCCCCCGACCAATCCCCAGCCTA
GGACGCCAACTTCTCCCACCCCTTGGCCCCTCACATCCTCTCCAGGAAGGCAGTGAGGGG
CTGTGACATTTTTCCGGAGAAGATTTCAGAGCTGAGGCTTTGGTACCCCCAAACCCCCAA
TATTTTTGGACTGGCAGACTCAAGGGGCTGGAATCTCATGATTCCATGCCCGAGTCCGCC
CATCCCTGACCATGGTTTTGGCTCTCCCACCCCGCCGTTCCCTGCGCTTCATCTCATGAG
GATTTCTTTATGAGGCAAATTTATATTTTTTAATATCGGGGGGTGGACCACGCCGCCCTC
CATCCGTGCTGCATGAAAAACATTCCACGTGCCCCTTGTCGCGCGTCTCCCATCCTGATC
CCAGACCCATTCCTTAGCTATTTATCCCTTTCCTGGTTTCCGAAAGGCAATTATATCTAT
TATGTATAAGTAAATATATTATATATGGATGTGTGTGTGTGCGTGCGCGTGAGTGTGTGA
GCGCTTCTGCAGCCTCGGCCTAGGTCACGTTGGCCCTCAAAGCGAGCCGTTGAATTGGAA
ACTGCTTCTAGAAACTCTGGCTCAGCCTGTCTCGGGCTGACCCTTTTCTGATCGTCTCGG
CCCCTCTGATTGTTCCCGATGGTCTCTCTCCCTCTGTCTTTTCTCCTCCGCCTGTGTCCA
TCTGACCGTTTTCACTTGTCTCCTTTCTGACTGTCCCTGCCAATGCTCCAGCTGTCGTCT
GACTCTGGGTTCGTTGGGGACATGAGATTTTATTTTTTGTGAGTGAGACTGAGGGATCGT
AGATTTTTACAATCTGTATCTTTGACAATTCTGGGTGCGAGTGTGAGAGTGTGAGCAGGG
CTTGCTCCTGCCAACCACAATTCAATGAATCCCCGACCCCCCTACCCCATGCTGTACTTG
TGGTTCTCTTTTTGTATTTTGCATCTGACCCCGGGGGGCTGGGACAGATTGGCAATGGGC
CGTCCCCTCTCCCCTTGGTTCTGCACTGTTGCCAATAAAAAGCTCTTAAAA
ACGC
FOSB Mouse DNA
ATAAATTCTTATTTTGACACTCACCAAAATAGTCACCTGGAAAACCCGCTTTTTGTGACA
AAGTACAGAAGGCTTGGTCACATTTAAATCACTGAGAACTAGAGAGAAATACTATCGCAA
ACTGTAATAGACATTACATCCATAAAAGTTTCCCCAGTCCTTATTGTAATATTGCACAGT
GCAATTGCTACATGGCAAACTAGTGTAGCATAGAAGTCAAAGCAAAAACAAACCAAAGAA
AGGAGCCACAAGAGTAAAACTGTTCAACAGTTAATAGTTCAAACTAAGCCATTGAATCTA
TCATTGGGATCGTTAAAATGAATCTTCCTACACCTTGCAGTGTATGATTTAACTTTTACA
GAACACAAGCCAAGTTTAAAATCAGCAGTAGAGATATTAAAATGAAAAGGTTTGCTAATA
GAGTAACATTAAATACCCTGAAGGAAAAAAAACCTAAATATCAAAATAACTGATTAAAAT
TCACTTGCAAATTAGCACACGAATATGCAACTTGGAAATCATGCAGTGTTTTATTTAAGA
AAACATAAAACAAAACTATTAAAATAGTTTTAGAGGGGGTAAAATCCAGGTCCTCTGCCA
GGATGCTAAAATTAGACTTCAGGGGAATTTTGAAGTCTTCAATTTTGAAACCTATTAAAA
AGCCCATGATTACAGTTAATTAAGAGCAGTGCACGCAACAGTGACACGCCTTTAGAGAGC
ATTACTGTGTATGAACATGTTGGCTGCTACCAGCCACAGTCAATTTAACAAGGCTGCTCA
GTCATGAACTTAATACAGAGAGAGCACGCCTAGGCAGCAAGCACAGCTTGCTGGGCCACT
TTCCTCCCTGTCGTGACACAATCAATCCGTGTACTTGGTGTATCTGAAGCGCACGCTGCA
CCGCGGCACTGCCCGGCGGGTTTCTGGGCGGGGAGCGATCCCCGCGTCGCCCCCCGTGAA
ACCGACAGAGCCTGGACTTTCAGGAGGTACAGCGGCGGTCTGAAGGGGATCTGGGATCTT
GCAGAGGGAACTTGCATCGAAACTTGGGCAGTTCTCCGAACCGGAGACTAAGCTTCCCCG
AGCAGCGCACTTTGGAGACGTGTCCGGTCTACTCCGGACTCGCATCTCATTCCACTCGGC
CATAGCCTTGGCTTCCCGGCGACCTCAGCGTGGTCACAGGGGCCCCCCTGTGCCCAGGGA
AATGTTTCAAGCTTTTCCCGGAGACTACGACTCCGGCTCCCGGTGTAGCTCATCACCCTC
CGCCGAGTCTCAGTACCTGTCTTCGGTGGACTCCTTCGGCAGTCCACCCACCGCCGCCGC
CTCCCAGGAGTGCGCCGGTCTCGGGGAAATGCCCGGCTCCTTCGTGCCAACGGTCACCGC
AATCACAACCAGCCAGGATCTTCAGTGGCTCGTGCAACCCACCCTCATCTCTTCCATGGC
CCAGTCCCAGGGGCAGCCACTGGCCTCCCAGCCTCCAGCTGTTGACCCTTATGACATGCC
AGGAACCAGCTACTCAACCCCAGGCCTGAGTGCCTACAGCACTGGCGGGGCAAGCGGAAG
TGGTGGGCCTTCAACCAGCACAACCACCAGTGGACCTGTGTCTGCCCGTCCAGCCAGAGC
CAGGCCTAGAAGACCCCGAGAAGAGACACTTACCCCAGAAGAAGAAGAAAAGCGAAGGGT
TCGCAGAGAGCGGAACAAGCTGGCTGCAGCTAAGTGCAGGAACCGTCGGAGGGAGCTGAC
AGATCGACTTCAGGCGGAAACTGATCAGCTTGAAGAGGAAAAGGCAGAGCTGGAGTCGGA
GATCGCCGAGCTGCAAAAAGAGAAGGAACGCCTGGAGTTTGTCCTGGTGGCCCACAAACC
GGGCTGCAAGATCCCCTACGAAGAGGGGCCGGGGCCAGGCCCGCTGGCCGAGGTGAGAGA
TTTGCCAGGGTCAACATCCGCTAAGGAAGACGGCTTCGGCTGGCTGCTGCCGCCCCCTCC
ACCACCCCCCCTGCCCTTCCAGAGCAGCCGAGACGCACCCCCCAACCTGACGGCTTCTCT
CTTTACACACAGTGAAGTTCAAGTCCTCGGCGACCCCTTCCCCGTTGTTAGCCCTTCGTA
CACTTCCTCGTTTGTCCTCACCTGCCCGGAGGTCTCCGCGTTCGCCGGCGCCCAACGCAC
CAGCGGCAGCGAGCAGCCGTCCGACCCGCTGAACTCGCCCTCCCTTCTTGCTCTGTAAAC
TCTTTAGACAAACAAAACAAACAAACCCGCAAGGAACAAGGAGGAGGAAGATGAGGAGGA
GAGGGGAGGAAGCAGTCCGGGGGTGTGTGTGTGGACCCTTTGACTCTTCTGTCTGACCAC
CTGCCGCCTCTGCCATCGGACATGACGGAAGGACCTCCTTTGTGTTTTGTGCTCCGTCTC
TGGTTTTCTGTGCCCCGGCGAGACCGGAGAGCTGGTGACTTTGGGGACAGGGGGTGGGGC
GGGGATGGACACCCCTCCTGCATATCTTTGTCCTGTTACTTCAACCCAACTTCTGGGGAT
AGATGGCTGGCTGGGTGGGTAGGGTGGGGTGCAACGCCCACCTTTGGCGTCTTGCGTGAG
GCTGGAGGGGAAAGGGTGCTGAGTGTGGGGTGCAGGGTGGGTTGAGGTCGAGCTGGCATG
CACCTCCAGAGAGACCCAACGAGGAAATGACAGCACCGTCCTGTCCTTCTTTTCCCCCAC
CCACCCATCCACCCTCAAGGGTGCAGGGTGACCAAGATAGCTCTGTTTTGCTCCCTCGGG
CCTTAGCTGATTAACTTAACATTTCCAAGAGGTTACAACCTCCTCCTGGACGAATTGAGC
CCCCGACTGAGGGAAGTCGATGCCCCCTTTGGGAGTCTGCTAACCCCACTTCCCGCTGAT
TCCAAAATGTGAACCCCTATCTGACTGCTCAGTCTTTCCCTCCTGGGAAAACTGGCTCAG
GTTGGATTTTTTTCCTCGTCTGCTACAGAGCCCCCTCCCAACTCAGGCCCGCTCCCACCC
CTGTGCAGTATTATGCTATGTCCCTCTCACCCTCACCCCCACCCCAGGCGCCCTTGGCCG
TCCTCGTTGGGCCTTACTGGTTTTGGGCAGCAGGGGGCGCTGCGACGCCCATCTTGCTGG
AGCGCTTTATACTGTGAATGAGTGGTCGGATTGCTGGGTGCGCCGGATGGGATTGACCCC
CAGCCCTCCAAAACTTTCCCTGGGCCTCCCCTTCTTCCACTTGCTTCCTCCCTCCCCTTG
ACAGGGAGTTAGACTCGAAAGGATGACCACGACGCATCCCGGTGGCCTTCTTGCTCAGGC
CCCAGACTTTTTCTCTTTAAGTCCTTCGCCTTCCCCAGCCTAGGACGCCAACTTCTCCCC
ACCCTGGGAGCCCCGCATCCTCTCACAGAGGTCGAGGCAATTTTCAGAGAAGTTTTCAGG
GCTGAGGCTTTGGCTCCCCTATCCTCGATATTTGAATCCCCAAATATTTTTGGACTAGCA
TACTTAAGAGGGGGCTGAGTTCCCACTATCCCACTCCATCCAATTCCTTCAGTCCCAAAG
ACGAGTTCTGTCCCTTCCCTCCAGCTTTCACCTCGTGAGAATCCCACGAGTCAGATTTCT
ATTTTTTAATATTGGGGAGATGGGCCCTACCGCCCGTCCCCCGTGCTGCATGGAACATTC
CATACCCTGTCCTGGGCCCTAGGTTCCAAACCTAATCCCAAACCCCACCCCCAGCTATTT
ATCCCTTTCCTGGTTCCCAAAAAGCACTTATATCTATTATGTATAAATAAATATATTATA
TATGAGTGTGCGTGTGTGTGCGTGTGCGTGCGTGCGTGCGTGCGTGCGAGCTTCCTTGTT
TTCAAGTGTGCTGTGGAGTTCAAAATCGCTTCTGGGGATTTGAGTCAGACTTTCTGGCTG
TCCCTTTTTGTCACCTTTTTGTTGTTGTCTCGGCTCCTCTGGCTGTTGGAGACAGTCCCG
GCCTCTCCCTTTATCCTTTCTCAAGTCTGTCTCGCTCAGACCACTTCCAACATGTCTCCA
CTCTCAATGACTCTGATCTCCGGTNTGTCTGTTAATTCTGGATTTGTCGGGGACATGCAA
TTTTACTTCTGTAAGTAAGTGTGACTGGGTGGTAGATTTTTTACAATCTATATCGTTGAG
AATTC
FOSB Mouse Protein
MFQAFPGDYDSGSRCSSSPSAESQYLSSVDSFGSPPTAAASQECAGLGEMPGSFVPTVTA
ITTSQDLQWLVQPTLISSMAQSQGQPLASQPPAVDPYDMPGTSYSTPGLSAYSTGGASGS
GGPSTSTTTSGPVSARPARARPRRPREETLTPEEEEKRRVRRERNKLAAAKCRNRRRELT
DRLQAETDQLEEEKAELESEIAELQKEKERLEFVLVAHKPGCKIPYEEGPGPGPLAEVRD
LPGSTSAKEDGFGWLLPPPPPPPLPFQSSRDAPPNLTASLFTHSEVQVLGDPFPVVSPSY
TSSFVLTCPEVSAFAGAQRTSGSEQPSDPLNSPSLLAL
Dusp1 Human DNA
TTTGGGCTGTGTGTGCGACGCGGGTCGGAGGGGCAGTCGGGGGAACCGCGAAGAAGCCGA
GGAGCCCGGAGCCCCGCGTGACGCTCCTCTCTCAGTCCAAAAGCGGCTTTTGGTTCGGCG
CAGAGAGACCCGGGGGTCTAGCTTTTCCTCGAAAAGCGCCGCCCTGCCCTTGGCCCCGAG
AACAGACAAAGAGCACCGCAGGGCCGATCACGCTGGGGGCGCTGAGGCCGGCCATGGTCA
TGGAAGTGGGCACCCTGGACGCTGGAGGCCTGCGGGCGCTGCTGGGGGAGCGAGCGGCGC
AATGCCTGCTGCTGGACTGCCGCTCCTTCTTCGCTTTCAACGCCGGCCACATCGCCGGCT
CTGTCAACGTGCGCTTCAGCACCATCGTGCGGCGCCGGGCCAAGGGCGCCATGGGCCTGG
AGCACATCGTGCCCAACGCCGAGCTCCGCGGCCGCCTGCTGGCCGGCGCCTACCACGCCG
TGGTGTTGCTGGACGAGCGCAGCGCCGCCCTGGACGGCGCCAAGCGCGACGGCACCCTGG
CCCTGGCGGCCGGCGCGCTCTGCCGCGAGGCGCGCGCCGCGCAAGTCTTCTTCCTCAAAG
GAGGATACGAAGCGTTTTCGGCTTCCTGCCCGGAGCTGTGCAGCAAACAGTCGACCCCCA
TGGGGCTCAGCCTTCCCCTGAGTACTAGCGTCCCTGACAGCGCGGAATCTGGGTGCAGTT
CCTGCAGTACCCCACTCTACGATCAGGGTGGCCCGGTGGAAATCCTGCCCTTTCTGTACC
TGGGCAGTGCGTATCACGCTTCCCGCAAGGACATGCTGGATGCCTTGGGCATAACTGCCT
TGATCAACGTCTCAGCCAATTGTCCCAACCATTTTGAGGGTCACTACCAGTACAAGAGCA
TCCCTGTGGAGGACAACCACAAGGCAGACATCAGCTCCTGGTTCAACGAGGCCATTGACT
TCATAGACTCCATCAAGAATGCTGGAGGAAGGGTGTTTGTCCACTGCCAGGCAGGCATTT
CCCGGTCAGCCACCATCTGCCTTGCTTACCTTATGAGGACTAATCGAGTCAAGCTGGACG
AGGCCTTTGAGTTTGTGAAGCAGAGGCGAAGCATCATCTCTCCCAACTTCAGCTTCATGG
GCCAGCTGCTGCAGTTTGAGTCCCAGGTGCTGGCTCCGCACTGTTCGGCAGAGGCTGGGA
GCCCCGCCATGGCTGTGCTCGACCGAGGCACCTCCACCACCACCGTGTTCAACTTCCCCG
TCTCCATCCCTGTCCACTCCACGAACAGTGCGCTGAGCTACCTTCAGAGCCCCATTACGA
CCTCTCCCAGCTGCTGAAAGGCCACGGGAGGTGAGGCTCTTCACATCCCATTGGGACTCC
ATGCTCCTTGAGAGGAGAAATGCAATAACTCTGGGAGGGGCTCGAGAGGGCTGGTCCTTA
TTTATTTAACTTCACCCGAGTTCCTCTGGGTTTCTAAGCAGTTATGGTGATGACTTAGCG
TCAAGACATTTGCTGAACTCAGCACATTCGGGACCAATATATAGTGGGTACATCAAGTCC
ATCTGACAAAATGGGGCAGAAGAGAAAGGACTCAGTGTGTGATCCGGTTTCTTTTTGCTC
GCCCCTGTTTTTTGTAGAATCTCTTCATGCTTGACATACCTACCAGTATTATTCCCGACG
ACACATATACATATGAGAATATACCTTATTTATTTTTGTGTAGGTGTCTGCCTTCACAAA
TGTCATTGTCTACTCCTAGAAGAACCAAATACCTCAATTTTTGTTTTTGAGTACTGTACT
ATCCTGTAAATATATCTTAAGCAGGTTTGTTTTCAGCACTGATGGAAAATACCAGTGTTG
GGTTTTTTTTTAGTTGCCAACAGTTGTATGTTTGCTGATTATTTATGACCTGAAATAATA
TATTTCTTCTTCTAAGAAGACATTTTGTTACATAAGGATGACTTTTTTATACAATGGAAT
AAATTATGGCATTTCTATTG
Dusp1 Mouse DNA
CGGCGGGAGGAAAGCGCGGTGAAGCCAGATTAGGAGCAGCGAGCACTTGGGGACTTAGGG
CCACAGGACACCGCACAAGATCGACCGACTTTTTCTGGAGAACCGCAGAACGGGCACGCT
GGGGTCGCTGGGGCTGGCCATGGTGATGGAGGTGGGCATCCTGGACGCCGGGGGGCTGCG
CGCGCTGCTGCGAGAGGGCGCCGCGCAGTGCCTGTTGTTGGATTGTCGCTCCTTCTTCGC
TTTCAACGCCGGCCACATCGCGGGCTCAGTGAACGTGCGCTTCAGCACCATCGTGCGGCG
CCGCGCCAAGGGCGCCATGGGCCTGGAGCATATCGTGCCCAACGCTGAACTGCGTGGCCG
CCTGCTGGCCGGAGCCTACCACGCCGTGGTGCTGCTGGACGAGCGCAGCGCCTCCCTGGA
CGGCGCCAAGCGCGACGGCACCCTGGCCCTGGCCGCGGGCGCGCTCTGCCGAGAGGCGCG
CTCCACTCAAGTCTTCTTTCTCCAAGGAGGATATGAAGCGTTTTCGGCTTCCTGCCCTGA
GCTGTGCAGCAAACAGTCCACCCCCACGGGGCTCAGCCTCCCCCTGAGTACTAGTGTGCC
TGACAGTGCAGAATCCGGATGCAGCTCCTGTAGTACCCCTCTCTACGATCAGGGGGGCCC
AGTGGAGATCCTGTCCTTCCTGTACCTGGGCAGTGCCTATCACGCTTCTCGGAAGGATAT
GCTTGACGCCTTGGGCATCACCGCCTTGATCAACGTCTCAGCCAATTGTCCTAACCACTT
TGAGGGTCACTACCAGTACAAGAGCATCCCTGTGGAGGACAACCACAAGGCAGACATCAG
CTCCTGGTTCAACGAGGCTATTGACTTCATAGACTCCATCAAGGATGCTGGAGGGAGAGT
GTTTGTTCATTGCCAGGCCGGCATCTCCCGGTCAGCCACCATCTGCCTTGCTTACCTCAT
GAGGACTAACCGGGTAAAGCTGGACGAGGCCTTTGAGTTTGTGAAGCAGAGGCGGAGTAT
CATCTCCCCGAACTTCAGCTTCATGGGCCAGCTGCTGCAGTTTGAGTCCCAAGTGCTAGC
CCCTCACTGCTCTGCTGAAGCTGGGAGCCCTGCCATGGCTGTCCTTGACCGGGGCACCTC
TACTACCACAGTCTTCAACTTCCCTGTTTCCATCCCCGTCCACCCCACGAACAGTGCCCT
GAACTACCTTAAAAGCCCCATCACCACCTCTCCAAGCTGCTGAAGGGCAAGGGGAGGTGT
GGAGTTTCACTTGCCACCGGGTCGCCACTCCTCCTGTGGGAGGAGCAATGCAATAACTCT
GGGAGAGGCTCATGGGAGCTGGTCCTTATTTATTTAACACCCCCCTCACCCCCCAACTCC
TCCTGAGTTCCACTGAGTTCCTAAGCAGTCACAACAATGACTTGACCGCAAGACATTTGC
TGAACTCGGCACATTCGGGACCAATATATTGTGGGTACATCAAGTCCCTCTGACAAAACA
GGGCAGAAGAGAAAGGACTCTGTTTGAGGCAGTTTCTTCGCTTGCCTGTTTTTTTTTTCT
AGAAACTTCATGCTTGACACACCCACCAGTATTAACCATTCCCGATGACATGCGCGTATG
AGAGTTTTTACCTTTATTTATTTTTGTGTAGGTCGGTGGTTTCTGCCTTCACAAATGTCA
TTGTCTACTCATAGAAGAACCAAATACCTCAATTTTGTGTTTGCGTACTGTACTATCTTG
TAAATAGACCCAGAGCAGGTTTGCTTTCGGCACTGACAGACAAAGCCAGTGTAGGTTTGT
AGCTTTCAGTTATCGACAGTTGTATGTTTGTTTATTTATGATCTGAAGTAATATATTTCT
TCTTCTGTGAAGACATTTTGTTACTGGGATGACTTTTTTTATACAACAGAATAAATTATG
ACGTTTCTATTGA
Dusp1 Mouse Protein
MVMEVGILDAGGLRALLREGAAQCLLLDCRSFFAFNAGHIAGSVNVRFSTIVRRRAKGAM
GLEHIVPNAELRGRLLAGAYHAVVLLDERSASLDGAKRDGTLALAAGALCREARSTQVFF
LQGGYEAFSASCPELCSKQSTPTGLSLPLSTSVPDSAESGCSSCSTPLYDQGGPVEILSF
LYLGSAYHASRKDMLDALGITALINVSANCPNHFEGHYQYKSIPVEDNHKADISSWFNEA
IDFIDSIKDAGGRVFVHCQAGISRSATICLAYLMRTNRVKLDEAFEFVKQRRSIISPNFS
FMGQLLQFESQVLAPHCSAEAGSPAMAVLDRGTSTTTVFNFPVSIPVHPTNSALNYLKSP
ITTSPSC
Jun Human DNA
ATGACTGCAAAGATGGAAACGACCTTCTATGACGATGCCCTCAACGCCTCGTTCCTCCCG
TCCGAGAGCGGACCTTATGGCTACAGTAACCCCAAGATCCTGAAACAGAGCATGACCCTG
AACCTGGCCGACCCAGTGGGGAGCCTGAAGCCGCACCTCCGCGCCAAGAACTCGGACCTC
CTCACCTCGCCCGACGTGGGGCTGCTCAAGCTGGCGTCGCCCGAGCTGGAGCGCCTGATA
ATCCAGTCCAGCAACGGGCACATCACCACCACGCCGACCCCCACCCAGTTCCTGTGCCCC
AAGAACGTGACAGATGAGCAGGAGGGCTTCGCCGAGGGCTTCGTGCGCGCCCTGGCCGAA
CTGCACAGCCAGAACACGCTGCCCAGCGTCACGTCGGCGGCGCAGCCGGTCAACGGGGCA
GGCATGGTGGCTCCCGCGGTAGCCTCGGTGTCAGGGGGCAGCGGCAGCGGCGGCTTCAGC
GCCAGCCTGCACAGCGAGCCGCCGGTCTACGCAAACCTCAGCAACTTCAACCCAGGCGCG
CTGAGCAGCGGCGGCGGGGCGCCCTCCTACGGCGCGGCCGGCCTGGCCTTTCCCGCGCAA
CCCCAGCAGCAGCAGCAGCCGCCGCACCACCTGCCCCAGCAGATGCCCGTGCAGCACCCG
CGGCTGCAGGCCCTGAAGGAGGAGCCTCAGACAGTGCCCGAGATGCCCGGCGAGACACCG
CCCCTGTCCCCCATCGACATGGAGTCCCAGGAGCGGATCAAGGCGGAGAGGAAGCGCATG
AGGAACCGCATCGCTGCCTCCAAGTGCCGAAAAAGGAAGCTGGAGAGAATCGCCCGGCTG
GAGGAAAAAGTGAAAACCTTGAAAGCTCAGAACTCGGAGCTGGCGTCCACGGCCAACATG
CTCAGGGAACAGGTGGCACAGCTTAAACAGAAAGTCATGAACCACGTTAACAGTGGGTGC
CAACTCATGCTAACGCAGCAGTTGCAAACATTTTGA
Jun Mouse DNA
GTGACGACTGGTCAGCACCGCCGGAGAGCCGCTGTTGCTGGGACTGGTCTGCGGGCTCCA
AGGAACCGCTGCTCCCCGAGAGCGCTCCGTGAGTGACCGCGACTTTTCAAAGCTCGGCAT
CGCGCGGGAGCCTACCAACGTGAGTGCTAGCGGAGTCTTAACCCTGCGCTCCCTGGAGCA
ACTGGGGAGGAGGGCTCAGGGGGAAGCACTGCCGTCTGGAGCGCACGCTCTAAACAAACT
TTGTTACAGAAGCGGGGACGCGCGGGTATCCCCCCGCTTCCCGGCGCGCTGTTGCGGCCC
CGAAACTTCTGCGCACAGCCCAGGCTAACCCCGCGTGAAGTGACGGACCGTTCTATGACT
GCAAAGATGGAAACGACCTTCTACGACGATGCCCTCAACGCCTCGTTCCTCCAGTCCGAG
AGCGGTGCCTACGGCTACAGTAACCCTAAGATCCTAAAACAGAGCATGACCTTGAACCTG
GCCGACCCGGTGGGCAGTCTGAAGCCGCACCTCCGCGCCAAGAACTCGGACCTTCTCACG
TCGCCCGACGTCGGGCTGCTCAAGCTGGCGTCGCCGGAGCTGGAGCGCCTGATCATCCAG
TCCAGCAATGGGCACATCACCACTACACCGACCCCCACCCAGTTCTTGTGCCCCAAGAAC
GTGACCGACGAGCAGGAGGGCTTCGCCGAGGGCTTCGTGCGCGCCCTGGCTGAACTGCAT
AGCCAGAACACGCTTCCCAGTGTCACCTCCGCGGCACAGCCGGTCAGCGGGGCGGGCATG
GTGGCTCCCGCGGTGGCCTCAGTAGCAGGCGCTGGCGGCGGTGGTGGCTACAGCGCCAGC
CTGCACAGTGAGCCTCCGGTCTACGCCAACCTCAGCAACTTCAACCCGGGTGCGCTGAGC
TGCGGCGGTGGGGCGCCCTCCTATGGCGCGGCCGGGCTGGCCTTTCCCTCGCAGCCGCAG
CAGCAGCAGCAGCCGCCTCAGCCGCCGCACCACTTGCCCCAACAGATCCCGGTGCAGCAC
CCGCGGCTGCAAGCCCTGAAGGAAGAGCCGCAGACCGTGCCGGAGATGCCGGGAGAGACG
CCGCCCCTGTCCCCTATCGACATGGAGTCTCAGGAGCGGATCAAGGCAGAGAGGAAGCGC
ATGAGGAACCGCATTGCCGCCTCCAAGTGCCGGAAAAGGAAGCTGGAGCGGATCGCTCGG
CTAGAGGAAAAAGTGAAAACCTTGAAAGCGCAAAACTCCGAGCTGGCATCCACGGCCAAC
ATGCTCAGGGAACAGGTGGCACAGCTTAAGCAGAAAGTCATGAACCACGTTAACAGTGGG
TGCCAACTCATGCTAACGCAGCAGTTGCAAACGTTTTGAGAACAGACTGTCAGGGCTGAG
GGGCAATGGAAGAAAAAAAATAACAGAGACAAACTTGAGAACTTGACTGGAAGCGACAGA
GAAAAAAAAAGTGTCCGAGTACTGAAGCCAAGGGTACACAAGATGGACTGGGTTGCGACC
TGACGGCGCCCCCAGTGTGCTGGAGTGGGAAGGACGTGGCGCGCCTGGCTTTGGCGTGGA
GCCAGAGAGCAGAGGCCTATTGGCCGGCAGACTTTGCGGACGGGCTGTGCCCGCGCGACC
AGAACGATGGACTTTTCGTTAACATTGACCAAGAACTGCATGGACCTAACATTCGATCTC
ATTCAGTATTAAAGGGGGGTGGGAGGGGTTACAAACTGCAATAGAGACTGTAGATTGCTT
CTGTAGTGCTCCTTAACACAAAGCAGGGAGGGCTGGGAAGGGGGGGGAGGCTTGTAAGTG
CCAGGCTAGACTGCAGATGAACTCCCCTGGCCTGCCTCTCTCAACTGTGTATGTACATAT
ATTTTTTTTTTTAATTTGATGAAAGCTGATTACTGTCAATAAACAGCTTCCGCCTTTGTA
AGTTATTCCATGTTTGTTTGGGTGTCCTGCCCAGTGTTTGTAAATAAGAGATTTGAAGCA
TTCTGAGTTTACCATTTGTAATAAAGTATATAATTTTTTTATGTTTTGTTTCTGAAAATT
TCCAGAAAGGATATTTAAGAAAAATACAATAAACTATTGAAAAGTAGCCCCCAACCTCTT
TGCTGCATTATCCATAGATAATGATAGCTAGATGAAGTGACAGCTGAGTGCCCAATATAC
TAGGGTGAAAGCTGTGTCCCCTGTCTGATTGTAGGAATAGATACCCTGCATGCTATCATT
GGCTCATACTCTCTCCCCCGGCAACACACAAGTCCAGACTGTACACCAGAAGATGGTGTG
GTGTTTCTTAAGGCTGGAAGAAGGGCTGTTGCAAGGGGAGAGGGTCAGCCCGCTGGAAAG
CAGACACTTTGGTTGAAAGCTGTATGAAGTGGCATGTGCTGTGATCATTTATAATCATAG
GAAAGATTTAGTAATTAGCTGTTGATTCTCAAAGCAGGGACCCATGGAAGTTTTTAACAA
AAGGTGTCTCCTTCCAACTTTGAATCTGACAACTCCTAGAAAAAGATGACCTTTGCTTGT
GCATATTTATAATAGCGTTCGTTATCACAATAAATGTATTCAAAT
Jun Mouse Protein
MTAKMETTFYDDALNASFLQSESGAYGYSNPKILKQSMTLNLADPVGSLKPHLRAKNSDL
LTSPDVGLLKLASPELERLIIQSSNGHITTTPTPTQFLCPKNVTDEQEGFAEGFVRALAE
LHSQNTLPSVTSAAQPVSGAGMVAPAVASVAGAGGGGGYSASLHSEPPVYANLSNFNPGA
LSSGGGAPSYGAAGLAFPSQPQQQQQPPQPPHHLPQQIPVQHPRLQALKEEPQTVPEMPG
ETPPLSPIDMESQERIKAERKRMRNRIAASKCRKRKLERIARLEEKVKTLKAQNSELAST
ANMLREQVAQLKQKVMNHVNSGCQLMLTQQLQTF
Dusp6 Human DNA
CCAGCCTCGGAGGGAGGGATTAGAAGCCGCTAGACTTTTTTTCCTCCCCTCTCAGTAGCA
CGGAGTCCGAATTAATTGGATTTCATTCACTGGGGAGGAACAAAAACTATCTGGGCAGCT
TCATTGAGAGAGATTCATTGACACTAAGAGCCAGCGCTGCAGCTGGTGCAGAGAGAACCT
CCGGCTTTGACTTCTGTCTCGTCTGCCCCAAGGCCGCTAGCCTCGGCTTGGGAAGGCGAG
GCGGAATTAAACCCCGCTCCGAGAGCGCACGTTCGCGCGCGGTGCGTCGGCCATTGCCTG
CCCCGAGGGGCGTCTGGTAGGCACCCCGCCCTCTCCCGCAGCTCGACCCCCATGATAGAT
ACGCTCAGACCCGTGCCCTTCGCGTCGGAAATGGCGATCAGCAAGACGGTGGCGTGGCTC
AACGAGCAGCTGGAGCTGGGCAACGAGCGGCTGCTGCTGATGGACTGCCGGCCGCAGGAG
CTATACGAGTCGTCGCACATCGAGTCGGCCATCAACGTGGCCATCCCGGGCATCATGCTG
CGGCGCCTGCAGAAGGGTAACCTGCCGGTGCGCGCGCTCTTCACGCGCGGCGAGGACCGG
GACCGCTTCACCCGGCGCTGTGGCACCGACACAGTGGTGCTCTACGACGAGAGCAGCAGC
GACTGGAACGAGAATACGGGCGGCGAGTCGTTGCTCGGGCTGCTGCTCAAGAAGCTCAAG
GACGAGGGCTGCCGGGCGTTCTACCTGGAAGGTGGCTTCAGTAAGTTCCAAGCCGAGTTC
TCCCTGCATTGCGAGACCAATCTAGACGGCTCGTGTAGCAGCAGCTCGCCGCCGTTGCCA
GTGCTGGGGCTCGGGGGCCTGCGGATCAGCTCTGACTCTTCCTCGGACATCGAGTCTGAC
CTTGACCGAGACCCCAATAGTGCAACAGACTCGGATGGTAGTCCGCTGTCCAACAGCCAG
CCTTCCTTCCCAGTGGAGATCTTGCCCTTCCTCTACTTGGGCTGTGCCAAAGACTCCACC
AACTTGGACGTGTTGGAGGAATTCGGCATCAAGTACATCTTGAACGTCACCCCCAATTTG
CCGAATCTCTTTGAGAACGCAGGAGAGTTTAAATACAAGCAAATCCCCATCTCGGATCAC
TGGAGCCAAAACCTGTCCCAGTTTTTCCCTGAGGCCATTTCTTTCATAGATGAAGCCCGG
GGCAAGAACTGTGGTGTCTTGGTACATTGCTTGGCTGGCATTAGCCGCTCAGTCACTGTG
ACTGTGGCTTACCTTATGCAGAAGCTCAATCTGTCGATGAACGATGCCTATGACATTGTC
AAAATGAAAAAATCCAACATATCCCCTAACTTCAACTTCATGGGTCAGCTGCTGGACTTC
GAGAGGACGCTGGGACTCAGCAGCCCATGTGACAACAGGGTTCCAGCACAGCAGCTGTAT
TTTACCACCCCTTCCAACCAGAATGTATACCAGGTGGACTCTCTGCAATCTACGTGAAAG
ACCCCACACCCCTCCTTGCTGGAATGTGTCTGGCCCTTCAGCAGTTTCTCTTGGCAGCAT
CAGCTGGGCTGCTTTCTTTGTGTGTGGCCCCAGGTGTCAAAATGACACCAGCTGTCTGTA
CTAGACAAGGTTACCAAGTGCGGAATTGGTTAATACTAACAGAGAGATTTGCTCCATTCT
CTTTGGAATAACAGGACATGCTGTATAGATACAGGCAGTAGGTTTGCTCTGTACCCATGT
GTACAGCCTACCCATGCAGGGACTGGGATTCGAGGACTTCCAGGCGCATAGGGTAGAACC
AAATGATAGGGTAGGAGCATGTGTTCTTTAGGGCCTTGTAAGGCTGTTTCCTTTTGCATC
TGGAACTGACTATATAATTGTCTTCAATGAAGACTAATTCAATTTTGCATATAGAGGAGC
CAAAGAGAGATTTCAGCTCTGTATTTGTGGTATCAGTTTGGAAAAAAAAATCTGATACTC
CATTTGATTATTGTAAATATTTGATCTTGAATCACTTGACAGTGTTTGTTTGAATTGTGT
TTGTTTTTTCCTTTGATGGGCTTAAAAGAAATTATCCAAAGGGAGAAAGAGCAGTATGCC
ACTTCTTAA
Dusp6 Mouse DNA
GATCCATTGAGGAGCTGCCTCGCACAGGGGGTGTGCTCTCGCGGAGTCCTAGGGACTGTG
AGCAAACCCAGTCTTGAATAATCCGGCGAGAAACACCGGGTTGGATCCGAGGTGCAGCCT
CAGAGGGAAGGATTAAGAGCCGCTAGACTTTTTTTCTTTTCCCTTTTTCTCCTCTCAGTG
GCACGGAGTCCGAATTAATTGGATTTCATTCACTGGGTAGGAACAAAACTGGGCACCTTC
ATTCAGAGAGAGAGATTCATTGACTCGGAGAGTGATCTGGTGCAGAGGGACCACCGACTT
GACTTCTGTGTCGCTTTCCCTAACCGCTAGCCTCGGCTTGGGAAAGGCGAGGCGGAATCA
AACCCCGCTCCGAGAGCGGGAGCTTCGCGCAGCGTGCTCGGCCTATGCCTGCCTCGAGGG
GCGTCTGCTAGGCACCCCGCCTTCTCCTGCAGCTCGACCCCCATGATAGATACGCTCAGA
CCCGTGCCCTTCGCGTCGGAAATGGCGATCTGCAAGACGGTGTCGTGGCTCAACGAGCAG
CTGGAGCTGGGCAACGAACGGCTTCTGCTGATGGACTGCCGACCACAGGAGCTGTACGAG
TCGTCACACATCGAATCTGCCATTAATGTGGCCATCCCCGGCATCATGCTGCGGCGTCTG
CAGAAGGGCAACCTGCCCGTGCGTGCGCTCTTCACGCGCTGCGAGGACCGGGACCGCTTT
ACCAGGCGCTGCGGCACCGACACCGTGGTGCTGTACGACGAGAATAGCAGCGACTGGAAT
GAGAACACTGGTGGAGAGTCGGTCCTCGGGCTGCTGCTCAAGAAACTCAAAGACGAGGGC
TGCCGGGCGTTCTACCTGGAAGGTGGCTTCAGTAAGTTCCAGGCCGAGTTCGCCCTGCAC
TGCGAGACCAATCTAGACGGCTCGTGCAGCAGCAGTTCCCCGCCTTTGCCAGTGCTGGGG
CTCGGGGGCCTGCGGATCAGCTCGGACTCTTCCTCGGACATTGAGTCTGACCTTGACCGA
GACCCCAATAGTGCAACGGACTCTGATGGCAGCCCGCTGTCCAACAGCCAGCCTTCCTTC
CCGGTGGAGATTTTGCCCTTCCTTTACCTGGGCTGTGCCAAGGACTCGACCAACTTGGAC
GTGTTGGAAGAGTTTGGCATCAAGTACATCTTGAATGTCACCCCCAATTTGCCCAATCTG
TTTGAGAATGCGGGCGAGTTCAAATACAAGCAAATTCCTATCTCGGATCACTGGAGCCAA
AACCTGTCCCAGTTTTTCCCTGAGGCCATTTCTTTCATAGATGAAGCCCGAGGCAAAAAC
TGTGGTGTCCTGGTGCATTGCTTGGCAGGTATCAGCCGCTCTGTCACCGTGACAGTGGCG
TACCTCATGCAGAAGCTCAACCTGTCCATGAACGATGCTTACGACATTGTTAAGATGAAG
AAGTCCAACATCTCCCCCAACTTCAACTTCATGGGCCAGCTGCTTGACTTCGAAAGGACC
CTGGGACTGAGCAGCCCTTGTGACAACCGTGTCCCCACTCCGCAGCTGTACTTCACCACG
CCCTCCAACCAGAACGTCTACCAGGTGGACTCCCTGCAGTCTACGTGAAAGGCACCCACC
TCTCCTAGCCGGGAGTTGTCCCCATTCCTTCAGTTCCTCTTGAGCAGCATCGACCAGGCT
GCTTTCTTTCTGTGTGTGGCCCCGGGTGTCAAAAGTGTCACCAGCTGTCTGTGTTAGACA
AGGTTGCCAAGTGCAAAATTGGTTATTACGGAGGGAGAGATTTGCTCCATTCATTGTTTT
TTTGGAAGGACAGGACATGCTGTCTCTAGATCCAGCAATAGGTTTGCTTCTGTACCCCAG
CCTACCCAAGCAGGGACTGGACATCCATCCAGATAGAGGGTAGCATAGGAATAGGGACAG
GAGCATCTGTTCTTTAAGGCCTTGTATGGCTGTTTCCTGTTGCATCTGGAACTAACTATA
TATATTGTCTTCAGTGAAGACTGATTCAACTTTGGGTATAGTGGAGCCAAAGAGATTTTT
AGCTCTGTATTTGCGGTATCGGTTTAGAAGACAAAAAAAATTAAAACCTGATACTTTTAT
CTGATTATTGTAAATATTTGATCTTCAATCACTTGACAGTGTTTGTTTGGCTTGTATTTG
TTTTTTATCTTTGGGCTTAAAAGAGATCCAAAGAGAGAAAGAGCAGTATGCCACTTCTTA
GAACAAAAGTATAAGGAAAAAAATGTTCTTTTTAATCCAAAGGGTATATTTGCAGCATGC
TTGACCTTGATGTACCAATTCTGACGGCATTTTCGTGGATATTATTATCACTAAGACTTT
GTTATGATGAGGTCTTCAGTCTCTTTCATATATCTTCCTTGTAACTTTTTTTTTCCTCTT
AATGTAGTTTTGACTCTGCCTTACCTTTGTAAATATTTGGCTTACAGTGTCTCAAGGGGT
ATTTTGGAAAGACACCAAAATTGTGGGTTCACTTTTTTTTTTTTTTTAAATAACTTCAGC
TGTGCTAAACAGCATATTACCTCTGTACAAAATTCTTCAGGGAGTGTCACCTCAAATGCA
ATACTTTGGGTTGGTTTCTTTCCTTTTAAAAAAAAAATACGAAACTGGAAGTGTGTGTAT
GTGTGCGAGTATGAGCGCCCATTTGGTGGATGCAACAGGTTGAGAGGAAGGGAGAATTAA
CTTGCTCCATGATGTTCGTGGTGTAAAGTTTTGAGCTGGAATTTATTATAAGAATGTAAA
ACCTTAAATTATTAATAAATAACTATTTTGGCT
Dusp6 Mouse Protein
MIDTLRPVPFASEMAICKTVSWLNEQLELGNERLLLMDCRPQELYESSHIESAINVAIPG
IMLRRLQKGNLPVRALFTRCEDRDRFTRRCGTDTVVLYDENSSDWNENTGGESVLGLLLK
KLKDEGCRAFYLEGGFSKFQAEFALHCETNLDGSCSSSSPPLPVLGLGGLRISSDSSSDI
ESDLDRDPNSATDSDGSPLSNSQPSFPVEILPFLYLGCAKDSTNLDVLEEFGIKYILNVT
PNLPNLFENAGEFKYKQIPISDHWSQNLSQFFPEAISFIDEARGKNCGVLVHCLAGISRS
VTVTVAYLMQKLNLSMNDAYDIVKMKKSNISPNFNFMGQLLDFERTLGLSSPCDNRVPTP
QLYFTTPSNQNVYQVDSLQST
Cdk1 Human DNA
GGGGGGGGGGGGCACTTGGCTTCAAAGCTGGCTCTTGGAAATTGAGCGGAGACGAGCGGC
TTGTTGTAGCTGCCGTGCGGCCGCCGCGGAATAATAAGCCGGGATCTACCATACCATTGA
CTAACTATGGAAGATTATACCAAAATAGAGAAAATTGGAGAAGGTACCTATGGAGTTGTG
TATAAGGGTAGACACAAAACTACAGGTCAAGTGGTAGCCATGAAAAAAATCAGACTAGAA
AGTGAAGAGGAAGGGGTTCCTAGTACTGCAATTCGGGAAATTTCTCTATTAAAGGAACTT
CGTCATCCAAATATAGTCAGTCTTCAGGATGTGCTTATGCAGGATTCCAGGTTATATCTC
ATCTTTGAGTTTCTTTCCATGGATCTGAAGAAATACTTGGATTCTATCCCTCCTGGTCAG
TACATGGATTCTTCACTTGTTAAGAGTTATTTATACCAAATCCTACAGGGGATTGTGTTT
TGTCACTCTAGAAGAGTTCTTCACAGAGACTTAAAACCTCAAAATCTCTTGATTGATGAC
AAAGGAACAATTAAACTGGCTGATTTTGGCCTTGCCAGAGCTTTTGGAATACCTATCAGA
GTATATACACATGAGGTAGTAACACTCTGGTACAGATCTCCAGAAGTATTGCTGGGGTCA
GCTCGTTACTCAACTCCAGTTGACATTTGGAGTATAGGCACCATATTTGCTGAACTAGCA
ACTAAGAAACCACTTTTCCATGGGGATTCAGAAATTGATCAACTCTTCAGGATTTTCAGA
GCTTTGGGCACTCCCAATAATGAAGTGTGGCCAGAAGTGGAATCTTTACAGGACTATAAG
AATACATTTCCCAAATGGAAACCAGGAAGCCTAGCATCCCATGTCAAAAACTTGGATGAA
AATGGCTTGGATTTGCTCTCGAAAATGTTAATCTATGATCCAGCCAAACGAATTTCTGGC
AAAATGGCACTGAATCATCCATATTTTAATGATTTGGACAATCAGATTAAGAAGATGTAG
CTTTCTGACAAAAAGTTTCCATATGTTATG
Cdk1 Mouse DNA
TCCGTCGTAACCTGTTGAGTAACTATGGAAGACTATATCAAAATAGAGAAAATTGGAGAA
GGTACTTACGGTGTGGTGTATAAGGGTAGACACAGAGTCACTGGCCAGATAGTGGCCATG
AAGAAGATCAGACTTGAAAGCGAGGAAGAAGGAGTGCCCAGTACTGCAATTCGGGAAATC
TCTCTATTAAAAGAACTTCGACATCCAAATATAGTCAGCCTGCAGGATGTGCTCATGCAG
GACTCCAGGCTGTATCTCATCTTTGAGTTCCTGTCCATGGACCTCAAGAAGTACCTGGAC
TCCATCCCTCCTGGGCAGTTCATGGATTCTTCACTCGTTAAGAGTTACTTACACCAAATC
CTCCAGGGAATTGTGTTTTGCCACTCCCGGCGAGTTCTTCACAGAGACTTGAAACCTCAA
AATCTATTGATTGATGACAAAGGAACAATCAAACTGGCTGATTTCGGCCTTGCCAGAGCG
TTTGGAATACCGATACGAGTGTACACACACGAGGTAGTGACGCTGTGGTACCGATCTCCA
GAAGTGTTGCTGGGCTCGGCTCGTTACTCCACTCCGGTTGACATCTGGAGTATAGGGACC
ATATTTGCAGAACTGGCCACCAAGAAGCCGCTTTTCCACGGCGACTCAGAGATTGACCAG
CTCTTCAGGATCTTCAGAGCTCTGGGCACTCCTAACAACGAAGTGTGGCCAGAAGTCGAG
TCCCTGCAGGACTACAAGAACACCTTTCCCAAGTGGAAGCCGGGGAGCCTCGCATCCCAC
GTCAAGAACCTGGACGAGAACGGCTTGGATTTGCTCTCAAAAATGCTAGTCTATGATCCT
GCCAAACGAATCTCTGGCAAAATGGCCCTGAAGCACCCGTACTTTGATGACTTGGACAAT
CAGATTAAGAAGATGTAGCCCTCTGGATGGATGTCCCTGTCTGCTGGTCGTAGGGGAAGA
TCG
Cdk1 Mouse Protein
MEDYIKIEKIGEGTYGVVYKGRHRVTGQIVAMKKIRLESEEEGVPSTAIREISLLKELRH
PNIVSLQDVLMQDSRLYLIFEFLSMDLKKYLDSIPPGQFMDSSLVKSYLHQILQGIVFCH
SRRVLHRDLKPQNLLIDDKGTIKLADFGLARAFGIPIRVYTHEVVTLWYRSPEVLLGSAR
YSTPVDIWSIGTIFAELATKKPLFHGDSEIDQLFRIFRALGTPNNEVWPEVESLQDYKNT
FPKWKPGSLASHVKNLDENGLDLLSKMLVYDPAKRISGKMALKHPYFDDLD
NQIKKM
Fignl1 Human DNA
GTCAGTCCCCGCGCTTTTCGGAGGCTGCCAGCGTCCCACACCAGCCGCAGGTGAAAACCG
GCAGAAAGACATTAAGAGATTTTCCTGCAGTCACTGCTGGCAGATGATAGAGCCAGGATT
TGAAAGCAGGCAGCCTGGCTCCAGACCCTGTGCTCTTAACTCCCGTTTTGCATCAAGAAC
AGAATCCTATGAAAGGCTTGTACAGTGCTTGGATAGCAGCATCAAGGAGCATTGTGTACA
TGCAGAAGTGCACAGTACCTGGAGTGAAACTGCTTGTGTTCGATTTCTGATACCATTCAT
AACTGGCTGTGTGATCTCAAAACCTCTAAAATGCAGACCTCCAGCTCTAGATCTGTGCAC
CTGAGTGAATGGCAGAAGAATTACTTCGCAATTACATCTGGCATATGTACCGGACCGAAG
GCAGATGCATACCGTGCACAGATATTACGCATTCAGTATGCATGGGCAAACTCTGAGATT
TCCCAGGTCTGTGCTACCAAACTGTTCAAAAAATATGCAGAGAAATATTCTGCAATTATT
GATTCTGACAATGTTGAATCTGGGTTGAATAATTATGCAGAAAACATTTTAACTTTGGCA
GGATCTCAACAAACAGATAGTGACAAGTGGCAGTCTGGATTGTCAATAAATAATGTTTTC
AAAATGAGTAGTGTACAGAAGATGATGCAAGCTGGCAAAAAATTCAAAGACTCTCTGTTG
GAACCTGCTCTTGCATCAGTGGTAATCCATAAGGAGGCCACTGTCTTTGATCTTCCTAAA
TTTAGTGTTTGTGGTAGTTCTCAAGAGAGTGACTCATTACCTAACTCAGCTCATGATCGA
GACCGGACCCAAGACTTCCCGGAGAGCAATCGTTTGAAACTCCTTCAGAATGCCCAGCCA
CCTATGGTGACTAACACTGCTAGGACTTGTCCTACATTCTCAGCACCTGTAGGTGAGTCA
GCTACTGCAAAATTCCATGTCACACCATTGTTTGGAAATGTCAAAAAGGAAAATCACAGC
TCTGCAAAAGAAAACATAGGACTTAATGTGTTCTTATCTAACCAGTCTTGTTTTCCTGCT
GCCTGTGAAAATCCACAGAGGAAGTCTTTTTATGGTTCTGGCACCATTGATGCACTTTCC
AATCCAATACTGAATAAGGCTTGTAGTAAAACAGAAGATAATGGCCCAAAGGAGGATAGC
AGCCTGCCTACATTTAAAACTGCAAAAGAACAATTATGGGTAGATCAGCAAAAAAAGTAC
CACCAACCTCAGCGTGCATCAGGGTCTTCATATGGTGGTGTAAAAAAGTCTCTAGGAGCT
AGTAGATCCCGAGGGATACTTGGAAAGTTTGTTCCTCCTATACCCAAGCAAGATGGGGGA
GAGCAGAATGGAGGAATGCAATGTAAGCCTTATGGGGCAGGACCTACAGAACCAGCACAT
CCAGTTGATGAGCGTCTGAAGAACTTGGAGCCAAAGATGATTGAACTTATTATGAATGAG
ATTATGGATCATGGACCTCCAGTAAATTGGGAAGATATTGCAGGAGTAGAATTTGCTAAA
GCCACCATAAAGGAAATAGTTGTGTGGCCCATGTTGAGGCCAGACATCTTTACTGGTTTA
AGGGGACCCCCTAAAGGAATTTTGCTCTTTGGTCCTCCTGGGACTGGTAAAACTCTAATT
GGCAAGTGCATTGCTAGTCAGTCTGGGGCAACATTCTTTAGCATCTCTGCTTCATCCTTA
ACTTCTAAATGGGTAGGTGAGGGGGAGAAAATGGTCCGTGCATTGTTTGCTGTTGCAAGG
TGTCAGCAACCAGCTGTGATATTTATTGACGAAATTGATTCCTTGTTATCTCAACGGGGA
GATGGTGAGCATGAATCTTCTAGAAGGATAAAAACAGAATTTTTAGTTCAATTAGATGGA
GCAACAACATCTTCTGAAGATCGTATCCTAGTGGTGGGAGCAACAAATCGGCCACAAGAA
ATTGATGAGGCTGCCCGGAGAAGATTGGTGAAAAGGCTTTATATTCCCCTCCCAGAAGCT
TCAGCCAGGAAACAGATAGTAATTAATCTAATGTCCAAAGAGCAGTGTTGCCTCAGTGAA
GAAGAAATTGAACAGATTGTACAGCAGTCTGATGCGTTTTCAGGAGCAGACATGACACAG
CTTTGCAGGGGGGCTTCTCTTGGTCCTATTCGCAGTTTACAAACTGCTGACATTGCTACC
ATAACACCGGATCAAGTTCGACCCATAGCTTACATTGATTTTGAAAATGCTTTTAGAACT
GTGCGACCTAGTGTTTCTCCAAAAGATTTAGAGCTTTATGAAAACTGGAACAAAACTTTT
GGTTGTGGAAAGTAAGTGGGATACTTGGAATCAAGGCATCTCTGTATTACAGTCTTCTTT
ATTTTTTAGCATAGAAAGTTGGGGATGTGTTAATTGTATTTTTAAGAATATATTCTAAAT
TCTGTACTTCAAATAATAGCACAGATTTTACATCTG
Fignl1 Mouse DNA
CATCGAGAAGTGTTCAGTGCCTGGTAAAGTACATAGACCTTGCTTCACTTGGAACTCGGC
CTTGATTTCTGCCGTTGGTCATAATCAGCAGAGTTCTCTCTAAACCTTTGACATGGAGAC
GTCCAGCTCCATGTCTGTGGAGACGACTAGGTCTGTGCAGGTGGACGAATGGCAGAAGAA
TTACTGTGTGGTTACATCCAGCATATGTACACCAAAGCAGAAGGCCGATGCATACCGTGC
ACTACTACTGCATATTCAGTATGCATATGCCAACTCCGAGATCTCTCAGGTCTTTGCTAC
CAACCTGTTCAAAAGGTATACAGAAAAATACTCTGCAATTATTGATTCTGACAATGTTGT
AACTGGCTTGAATAACTATGCAGAGAGCATTTTTGCTTTGGCAGGATCTCGACAGGCTGA
CAGTAACAAGTGGCAGTCTGGATTGTCAATAGATAATGTTTTCAAAATGAGTTGTGTACA
GGAGATGATGCAGGCTGGCAAGAAATTTGAAGAGTCTCTGTTGGAACCTGCTGATGCATC
AGTAGTCCTGTGTAAAGAGCCCACCGCCTTTGAGGTTCCTCAGCTTAGTGTTTGTGGAGG
TTCTGAAGACGCTGACATATTATCCAGTTCAGGTCATGACACAGATAAGACCCAAGCCAT
TCCAGGGAGCAGTCTGAGATGTTCCCCTTTTCAGAGTGCTCGGCTGCCTAAGGAAACTAA
TACCACTAAGACATGCCTCACCTCCTCAACATCTTTAGGTGAGTCAGCCACTGCAGCATT
TCACATGACACCATTATTTGGAAACACCGAAAAGGACACTCAAAGCTTTCCTAAAACCAG
CACAGGACTAAATATGTTCTTATCTAATCTGTCTTGTGTTCCTTCTGGCTGTGAAAACCC
TCAAGAAAGGAAGGCTTTTAATGACTCTGACATCATTGACATACTTTCCAATCCAACACT
GAACAAGGCTCCTAGTAAAACAGAAGACAGAGGCCGAAGGGAAGATAATAGCCTGCCTAC
CTTTAAAACTGCAAAAGAACAATTATGGGTAGATCAAAAGAAAAAGGGCCATCAATCCCA
GCATACATCTAAATCTTCTAATGGTGTTATGAAAAAGTCTCTGGGAGCTGGGAGGTCGAG
AGGGATATTTGGCAAGTTTGTTCCTCCTGTATCTAATAAGCAAGACGGAAGTGAGCAGCA
TGCCAAGAAGCACAAGTCTAGTAGGGCAGGGTCTGCAGAACCAGCACACCTCACTGATGA
TTGTCTGAAGAACGTGGAGCCAAGGATGGTTGAACTTGTTATGAATGAAATTATGGACCA
TGGGCCTCCAGTACATTGGGACGATATTGCTGGAGTAGAATTTGCCAAAGCCACAATAAA
GGAAATCGTTGTGTGGCCCATGATGAGGCCAGATATCTTTACTGGATTGCGAGGGCCCCC
TAAAGGAATTCTACTCTTTGGCCCTCCAGGGACTGGTAAAACTCTGATTGGCAAGTGCAT
TGCTAGCCAGTCTGGAGCAACATTCTTCAGCATCTCTGCTTCATCGCTGACTTCTAAGTG
GGTAGGTGAGGGAGAAAAAATGGTCCGTGCACTGTTTGCTGTTGCCAGGTGTCAGCAGCC
AGCTGTCATATTTATTGATGAAATTGATTCTTTATTGTCTCAACGAGGAGATGGTGAACA
TGAATCTTCAAGAAGGATAAAAACGGAATTTTTAGTTCAGTTAGATGGAGCAACCACATC
TTCTGAAGACCGGATTCTTGTGGTGGGAGCTACAAATCGGCCCCAAGAGATTGATGAAGC
TGCCCGGAGAAGATTGGTGAAAAGACTTTATATTCCCCTCCCAGAAGCTTCAGCCAGGAA
ACAGATAGTAGGTAATCTAATGTCTAAGGAGCAATGTTGTCTCAGTGATGAAGAAACTGA
TCTGGTAGTGCAGCAGTCTGATGGGTTTTCTGGCGCAGATATGACACAGCTTTGCAGAGA
GGCTTCTCTTGGTCCTATTCGCAGTTTGCACGCTGCTGACATTGCTACCATAAGTCCAGA
TCAAGTTCGACCAATAGCTTATATTGATTTTGAAAATGCTTTTAAAACTGTGCGACCTAC
TGTATCTCCAAAAGACTTGGAGCTTTATGAAAACTGGAATGAAACATTTGGTTGTGGAAA
GTGAATATAGCGATTGAAAGGAGAAGCTGTTATCTAGTAGTCGTCTTTACCTTTAGCCTC
GGAAGCTTGCTGTGCTACTTGTATTGTTTTGGAGTATATCCTGAATTCTGTGCCTCAGAT
TAGAATGATAACAGCTTGACTACTGACTGATATATTAGTATGTTGTATTTG
CC
Fignl1 Mouse Protein
METSSSMSVETTRSVQVDEWQKNYCVVTSSICTPKQKADAYRALLLHIQYAYANSEISQV
FATNLFKRYTEKYSAIIDSDNVVTGLNNYAESIFALAGSRQADSNKWQSGLSIDNVFKMS
CVQEMMQAGKKFEESLLEPADASVVLCKEPTAFEVPQLSVCGGSEDADILSSSGHDTDKT
QAIPGSSLRCSPFQSARLPKETNTTKTCLTSSTSLGESATAAFHMTPLFGNTEKDTQSFP
KTSTGLNMFLSNLSCVPSGCENPQERKAFNDSDIIDILSNPTLNKAPSKTEDRGRREDNS
LPTFKTAKEQLWVDQKKKGHQSQHTSKSSNGVMKKSLGAGRSRGIFGKFVPPVSNKQDGS
EQHAKKHKSSRAGSAEPAHLTDDCLKNVEPRMVELIMNEIMDHGPPVHWDDIAGVEFAKA
TIKEIVVWPMMRPDIFTGLRGPPKGILLFGPPGTGKTLIGKCIASQSGATFFSISASSLT
SKWVGEGEKMVRALFAVARCQQPAVIFIDEIDSLLSQRGDGEHESSRRIKTEFLVQLDGA
TTSSEDRILVVGATNRPQEIDEAARRRLVKRLYIPLPEASARKQIVGNLMSKEQCCLSDE
ETDLVVQQSDGFSGADMTQLCREASLGPIRSLHAADIATISPDQVRPIAYIDFENAFKTV
RPTVSPKDLELYENWNETFGCGK
P1k2 Human DNA
GCGCGCGGCTCCGATGGGAAGCATGACCCGGGTGGCGGGACAAGACTTGCTTCCCGGCCA
CGCGCGCTCGGCCGGCCGTGGGGCGGGGCATAGGCGTGACGTGGTGTCGCGTATCGAGTC
TCCGCCCCCTTCCCGCCTCCCCGTATATAAGACTTCGCCGAGCACTCTCACTCGCACAAG
TGGACCGGGGTGTTGGGTGCTAGTCGGCACCAGAGGCAAGGGTGCGAGGACCACGGCCGG
CTCGGACGTGTGACCGCGCCTAGGGGGTGGCAGCGGGCAGTGCGGGGCGGCAAGGCGACC
ATGGARCTTTTGCGGACTATCACCTACCAGCCAGCCGCCAGCACCAAAATGTGCGAGCAG
GCGCTGGGCAAGGGTTGCGGAGGGGACTCGAAGAAGAAGCGGCCGCCGCAGCCCCCCGAG
GAATCGCAGCCACCTCAGTCCCAGGCGCAAGTGCCCCCGGCGGCCCCTCACCACCATCAC
CACCATTCGCACTCGGGGCCGGAGATCTCGCGGATTATCGTCGACCCCACGACTGGGAAG
CGCTACTGCCGGGGCAAAGTGCTGGGAAAGGGTGGCTTTGCAAAATGTTACGAGATGACA
GATTTGACAAATAACAAAGTCTACGCCGCAAAAATTATTCCTCACAGCAGAGTAGCTAAA
CCTCATCAAAGGGAAAAGATTGACAAAGAAATAGAGCTTCACAGAATTCTTCATCATAAG
CATGTAGTGCAGTTTTACCACTACTTCGAGGACAAAGAAAACATTTACATTCTCTTGGAA
TACTGCAGTAGAAGGTCAATGGCTCATATTTTGAAAGCAAGAAAGGTGTTGACAGAGCCA
GAAGTTCGATACTACCTCAGGCAGATTGTGTCTGGACTGAAATACCTTCATGAACAAGAA
ATCTTGCACAGAGATCTCAAACTAGGGAACTTTTTTATTAATGAAGCCATGGAACTAAAA
GTTGGGGACTTCGGTCTGGCAGCCAGGCTAGAACCCYTGGAACACAGAAGGAGAACGATA
TGTGGTACCCCAAATTATCTCTCTCCTGAAGTCCTCAACAAACAAGGACATGGCTGTGAA
TCAGACATTTGGGCCCTGGGCTGTGTAATGTATACAATGTTACTAGGGAGGCCCCCATTT
GAAACTACAAATCTCAAAGAAACTTATAGGTGCATAAGGGAAGCAAGGTATACAATGCCG
TCCTCATTGCTGGCTCCTGCCAAGCACTTAATTGCTAGTATGTTGTCCAAAAACCCAGAG
GATCGTCCCAGTTTGGATGACATCATTCGACATGACTTTTTTTTGCAGGGCTTCACTCCG
GACAGACTGTCTTCTAGCTGTTGTCATACAGTTCCAGATTTCCACTTATCAAGCCCAGCT
AAGAATTTCTTTAAGAAAGCAGCTGCTGCTCTTTTTGGTGGCAAAAAAGACAAAGCAAGA
TATATTGACACACATAATAGAGTGTCTAAAGAAGATGAAGACATCTACAAGCTTAGGCAT
GATTTGAAAAAGACTTCAATAACTCAGCAACCCAGCAAACACAGGACAGATGAGGAGCTC
CAGCCACCTACCACCACAGTTGCCAGGTCTGGAACACCCGCAGTAGAAAACAAGCAGCAG
ATTGGGGATGCTATTCGGATGATAGTCAGAGGGACTCTTGGCAGCTGTAGCAGCAGCAGT
GAATGCCTTGAAGACAGTACCATGGGAAGTGTTGCAGACACAGTGGCAAGGGTTCTTCGG
GGATGTCTGGAAAACATGCCGGAAGCTGATTGCATTCCCAAAGAGCAGCTGAGCACATCA
TTTCAGTGGGTCACCAAATGGGTTGATTACTCTAACAAATATGGCTTTGGGTACCAGCTC
TCAGACCACACCGTCGGTGTCCTTTTCAACAATGGTGCTCACATGAGCCTCCTTCCAGAC
AAAAAAACAGTTCACTATTACGCAGAGCTTGGCCAATGCTCAGTTTTCCCAGCAACAGAT
GCTCCTGAGCAATTTATTAGTCAAGTGACGGTGCTGAAATACTTTTCTCATTACATGGAG
GAGAACCTCATGGATGGTGGAGATCTGCCTAGTGTTACTGATATTCGAAGACCTCGGCTC
TACCTCCTTCAGTGGCTAAAATCTGATAAGGCCCTAATGATGCTCTTTAATGATGGCACC
TTTCAGGTGAATTTCTACCATGATCATACAAAAATCATCATCTGTAGCCAAAATGAAGAA
TACCTTCTCACCTACATCAATGAGGATAGGATATCTACAACTTTCAGGCTGACAACTCTG
CTGATGTCTGGCTGTTCATCAGAATTAAAAAATCGAATGGAATATGCCCTGAACATGCTC
TTACAAAGATGTAACTGAAAGACTTTTCGAATGGACCCTATGGGACTCCTCTTTTCCACT
GTGAGATCTACAGGGAAGCCAAAAGAATGATCTAGAGTATGTTGAAGAAGATGGACATGT
GGTGGTACGAAAACAATTCCCCTGTGGCCTGCTGGACTGGGTGGAACCCAGAACCAGGCT
AAGGCATACAGTTCTTGACTTTGGACAATCCCAAGAGTGAACCAGAATGCAGTTTTCCTT
GAGATACCTGTTTTAAAAGGTTTTTCAGACAATTTTGCAGAAAGGTGCATTGATTCTTAA
ATTCTCTCTGTTGAGAGCATTTCAGCCAGAGGACTTTGGAACTGTGAATATACTTCCTGA
AGGGGAGGGAGAAGGGAGGAAGCTCCCATGTTGTTTAAAGGCTGTAATTGGAGCAGCTTT
TGGCTGCGTAACTGTGAACTATGGCCATATATAATTTTTTTTCATTAATTTTTGAAGATA
CTTGTGGCTGGAAAAGTGCATTCCTTGTTAATAAACTTTTTATTTATTACAGCCCAAAGA
GCAGTATTTATTATCAAAATGTCTTTTTTTTTATGTTGACCATTTTAAACCGTTGGCAAT
AAAGAGTATGAAAACGCAAAAAAAAAAAAAAA
P1k2 Mouse DNA
CGTAGGGAGAGAGACTGGTGCTCGAGGGACAGGGCTAGCCCGGACGCGTGTCCGCGCCTC
GGAGGTGGCAAGTAGGCAGTGTCGGGTGGCGAGGCAACGATGGAGCTCCTGCGGACTATC
ACCTACCAGCCGGCCGCCGGCACCAAGATGTGCGAGCAGGCTCTGGGCAAAGCTTGCGGC
GGGGACTCAAAGAAGAAGCGACCACAGCAGCCTTCTGAAGATGGGCAGCCCCAAGCCCAG
GTGACCCCGGCGGCCCCGCACCACCATCACCACCATTCCCACTCGGGACCCGAGATCTCG
CGGATTATAGTCGACCCCACGACGGGGAAGCGCTACTGCCGGGGCAAAGTGCTGGGCAAG
GGTGGATTTGCAAAGTGTTACGAAATGACAGATCTGACAAACAACAAAGTCTACGCTGCA
AAAATTATTCCTCACAGCAGAGTAGCTAAACCTCATCAGAGGGAAAAGATCGACAAAGAA
ATCGAGCTTCACAGACTACTGCACCATAAGCATGTCGTGCAGTTTTACCACTACTTTGAA
GACAAAGAAAACATTTACATTCTCTTGGAATACTGCAGTAGAAGGTCCATGGCTCACATC
TTGAAAGCAAGAAAGGTGTTGACAGAGCCAGAAGTCCGATACTACCTCAGGCAGATTGTG
TCAGGACTCAAGTATCTTCACGAACAAGAAATCTTGCACAGGGATCTCAAGCTAGGGAAC
TTTTTTATTAATGAAGCCATGGAGCTGAAGGTGGGAGACTTTGGTTTGGCAGCCAGACTG
GAACCACTGGAACACAGAAGGAGAACAATATGTGGAACCCCAAATTATCTCTCCCCCGAA
GTCCTCAACAAACAAGGACACGGCTGTGAATCAGACATCTGGGCCTTAGGCTGTGTAATG
TATACGATGCTGCTAGGAAGACCTCCATTCGAAACCACAAATCTGAAAGAAACGTACAGG
TGCATAAGGGAAGCAAGGTATACCATGCCGTCCTCATTGCTGGCCCCTGCTAAGCACTTG
ATAGCTAGCATGCTGTCCAAAAACCCAGAGGACCGCCCCAGTTTGGATGACATCATTCGG
CATGACTTCTTCCTGCAGGGTTTCACTCCGGACAGACTCTCTTCCAGCTGTTGCCACACA
GTTCCAGATTTCCACTTGTCAAGCCCAGCCAAGAATTTCTTTAAGAAAGCCGCAGCCGCT
CTTTTTGGTGGCAAGAAGGACAAAGCAAGATATAACGACACACACAATAAGGTGTCTAAG
GAAGATGAAGACATTTACAAGCTTCGGCATGATTTGAAGAAAGTGTCGATAACCCAGCAG
CCTAGCAAACACAGAGCAGACGAGGAGCCCCAGCCGCCTCCCACTACTGTTGCCAGATCT
GGAACGTCCGCAGTGGAAAACAAACAGCAGATTGGGGATGCAATCCGGATGATAGTCAGG
GGGACTCTCGGCAGCTGCAGCAGCAGCAGCGAATGCCTTGAAGACAGCACCATGGGAAGT
GTTGCAGACACAGTGGCAAGAGTCCTTCGAGGATGTCTAGAAAACATGCCGGAAGCTGAC
TGTATCCCCAAAGAGCAGCTGAGCACGTCCTTTCAGTGGGTCACCAAGTGGGTCGACTAC
TCCAACAAATATGGCTTTGGGTACCAGCTCTCGGACCACACTGTTGGCGTCCTTTTCAAC
AACGGGGCTCACATGAGCCTCCTTCCGGACAAAAAGACAGTTCACTATTATGCGGAACTT
GGCCAATGCTCTGTTTTCCCAGCAACAGATGCCCCTGAACAATTTATTAGTCAAGTGACG
GTGCTGAAATACTTTTCTCATTACATGGAGGAGAACCTCATGGATGGTGGTGATCTCCCG
AGTGTTACTGACATTCGAAGACCTCGGCTCTACCTCCTGCAGTGGTTAAAGTCTGATAAA
GCCTTAATGATGCTCTTCAATGACGGCACATTTCAGGTGAATTTCTACCACGATCATACA
AAAATCATCATCTGTAACCAGAGTGAAGAATACCTTCTCACCTACATCAATGAGGACAGG
ATCTCTACAACTTTCAGACTGACGACTCTGCTGATGTCTGGCTGTTCGTTAGAATTGAAA
AATCGAATGGAATATGCCCTGAACATGCTCTTACAGAGATGTAACTGAAAACATTATTAT
TATTATTATTATAATTATTTCGAGCGGACCTCATGGGACTCTTTTCCACTGTGAGATCAA
CAGGGAAGCCAGCGGAAAGATACAGAGCATGTTAGAGAAGTCGGACAGGTGGTGGTACGA
ATACAATTCCTCTGTGGCCTGCTGGACTGCTGGAACCAGACCAGCCTAAGGTGTAGAGTT
GACTTTGGACAATCCTGAGTGTGGAGCCGAGTGCAGTTTTCCCTGAGATACCTGTCGTGA
AAAGGTTTATGGGACAGTTTTTCAGAAAGATGCATTGACTCTGAAGTTCTCTCTGTTGAG
AGCGTCTTCAGTTGGAAGACTTGGAACTGTGAATACACTTCCTGAAGGGGAGGGAGAAGG
GAGGTTGCTCCCTTGCTGTTTAAAGGCTACAATCAGAGCAGCTTTTGGCTGCTTAACTGT
GAACTATGGCCATACATTTTTTTTTTTTTTGGTTATTTTTGAATACACTTGTGGTTGGAA
AAGTGCATTCCTTGTTAATAAACTTTTTATTTATTACAGCCCCAAGAGCAGTATTTATTA
TCAAGATGTTCTCTTTTTTTATGTTGACCATTTCAAACTCTTGGCAATAAAGAGTATGAC
ATAGAAAAAAAA
P1k2 Mouse Protein
MELLRTITYQPAAGTKMCEQALGKACGGDSKKKRPQQPSEDGQPQAQVTPAAPHHHHHHS
HSGPEISRIIVDPTTGKRYCRGKVLGKGGFAKCYEMTDLTNNKVYAAKIIPHSRVAKPHQ
REKIDKEIELHRLLHHKHVVQFYHYFEDKENIYILLEYCSRRSMAHILKARKVLTEPEVR
YYLRQIVSGLKYLHEQEILHRDLKLGNFFINEAMELKVGDFGLAARLEPLEHRRRTICGT
PNYLSPEVLNKQGHGCESDIWALGCVMYTMLLGRPPFETTNLKETYRCIREARYTMPSSL
LAPAKHLIASMLSKNPEDRPSLDDIIRHDFFLQGFTPDRLSSSCCHTVPDFHLSSPAKNF
FKKAAAALFGGKKDKARYNDTHNKVSKEDEDIYKLRHDLKKVSITQQPSKHRADEEPQPP
PTTVARSGTSAVENKQQIGDAIRMIVRGTLGSCSSSSECLEDSTMGSVADTVARVLRGCL
ENMPEADCIPKEQLSTSFQWVTKWVDYSNKYGFGYQLSDHTVGVLFNNGAHMSLLPDKKT
VHYYAELGQCSVFPATDAPEQFISQVTVLKYFSHYMEENLMDGGDLPSVTDIRRPRLYLL
QWLKSDKALMMLFNDGTFQVNFYHDHTKIIICNQSEEYLLTYINEDRISTTFRLTTLLMS
GCSLELKNRMEYALNMLLQRCN
Rsad2 Human DNA
CAGGAAGGGCCATGAAGATTAATAAAGATTTGGACTCAGGGCAAATATTTACTTAGTAGC
AATAACTCAAAGAATTACTGTTGAATAAATAAGCCAATTAAGCAGCCAATCACGTACTAT
GCGGATGCACACAAATGAAACCCTCACTTCAACCTGAAGACATTCGCACATGAGTTACGT
AGAGGGACCTGCAGGAAGCGGTAGAGAAAACATAAGGCTTATGCGTTTAATTTCCACACC
AATTTCAGGATCTTTGTCACTGACAGCAGCACTAAGACTTGTTAACTTTATATAGTTAAG
AAGAACAAGGCTGAGCGCGATGACTCACGCCTGTAAGCCTAGAACTTTGGGAGGCCAAAG
CAGGCAGACTGCTTGAGCCCAGGAGTTCCAGACCAGCCTGGGCAACATGGCAACACCCCA
TCTCTACAAAAAAATACAAGAATCAGCTGGGCGTGGTGATGTGTTCCTGTAATCTCAGCT
ACTCGGGAGGCAGAGGCAGGAGGATTGCTTGAACCCGGGAGGCAGAGGTTGTAGTTAGCC
GAGATCTCGCCACTGCACTCCAGTCTGGACGACAGAGTGAGACTCAGTCTCAAATAAATA
AATAAATACATAAATATAAGGAAAAAAATAAAGCTGCTTTCTCCTCTTCCTCCTCTTTGG
TCTCATCTGGCTCTGCTCCAGGCATCTGCCACAATGTGGGTGCTTACACCTGCTGCTTTT
GCTGGGAAGTTCTTGAGTGTGTTCAGGCAACCTCTGAGCTCTCTGTGGAGGAGCCTGGTC
CCGCTGTTCTGCTGGCTGAGGGCAACCTTCTGGCTGCTAGCTACCAAGAGGAGAAAGCAG
CAGCTGGTCCTGAGAGGGCCAGATGAGACCAAAGAGGAGGAAGAGGACCCTCCTCTGCCC
ACCACCCCAACCAGCGTCAACTATCACTTCACTCGCCAGTGCAACTACAAATGCGGCTTC
TGTTTCCACACAGCCAAAACATCCTTTGTGCTGCCCCTTGAGGAAGCAAAGAGAGGATTG
CTTTTGCTTAAGGAAGCTGGTATGGAGAAGATCAACTTTTCAGGTGGAGAGCCATTTCTT
CAAGACCGGGGAGAATACCTGGGCAAGTTGGTGAGGTTCTGCAAAGTAGAGTTGCGGCTG
CCCAGCGTGAGCATCGTGAGCAATGGAAGCCTGATCCGGGAGAGGTGGTTCCAGAATTAT
GGTGAGTATTTGGACATTCTCGCTATCTCCTGTGACAGCTTTGACGAGGAAGTCAATGTC
CTTATTGGCCGTGGCCAAGGAAAGAAGAACCATGTGGAAAACCTTCAAAAGCTGAGGAGG
TGGTGTAGGGATTATAGAATCCCTTTCAAGATAAATTCTGTCATTAATCGTTTCAACGTG
GAAGAGGACATGACGGAACAGATCAAAGCACTAAACCCTGTCCGCTGGAAAGTGTTCCAG
TGCCTCTTAATTGAAGGTGAGAATTGTGGAGAAGATGCTCTAAGAGAAGCAGAAAGATTT
GTTATTGGTGATGAAGAATTTGAAAGATTCTTGGAGCGCCACAAAGAAGTGTCCTGCTTG
GTGCCTGAATCTAACCAGAAGATGAAAGACTCCTACCTTATTCTGGATGAATATATGCGC
TTTCTGAACTGTAGAAAGGGACGGAAGGACCCTTCCAAGTCCATCCTGGATGTTGGTGTA
GAAGAAGCTATAAAATTCAGTGGATTTGATGAAAAGATGTTTCTGAAGCGAGGAGGAAAA
TACATATGGAGTAAGGCTGATCTGAAGCTGGATTGGTAGAGCGGAAAGTGGAACGAGACT
TCAACACACCAGTGGGAAAACTCCTAGAGTAACTGCCATTGTCTGCAATACTATCCCGTT
GGTATTTCCCAGTGGCTGAAAACCTGATTTTCTGCTGCACGTGGCATCTGATTACCTGTG
GTCACTGAACACACGAATAACTTGGATAGCAAATCCTGAGACAATGGAAAACCATTAACT
TTACTTCATTGGCTTATAACCTTGTTGTTATTGAAACAGCACTTCTGTTTTTGAGTTTGT
TTTAGCTAAAAAGAAGGAATACACACAGGAATAATGACCCCAAAAATGCTTAGATAAGGC
CCCTATACACAGGACCTGACATTTAGCTCAATGATGCGTTTGTAAGAAATAAGCTCTAGT
GATATCTGTGGGGGCAATATTTAATTTGGATTTGATTTTTTAAAACAATGTTTACTGCGA
TTTCTATATTTCCATTTTGAAACTATTTCTTGTTCCAGGTTTGTTCATTTGACAGAGTCA
GTATTTTTTGCCAAATATCCAGATAACCAGTTTTCACATCTGAGACATTACAAAGTATCT
GCCTCAATTATTTCTGCTGGTTATAATGCTTTTTTTTTTTTTTGCTTTTATGCCATTGCA
GTCTTGTACTTTTTACTGTGATGTACAGAAATAGTCAACAGATGTTTCCAAGAACATATG
ATATGATAATCCTACCAATTTTCAAGAAGTCTCTAGAAAGAGATAACACATGGAAAGACG
GCGTGGTGCAGCCCAGCCCACGGTGCCTGTTCCATGAATGCTGGCTACCTATGTGTGTGG
TACCTGTTGTGTCCCTTTCTCTTCAAAGATCCCTGAGCAAAACAAAGATACGCTTTCCAT
TTGATGATGGAGTTGACATGGAGGCAGTGCTTGCATTGCTTTGTTCGCCTATCATCTGGC
CACATGAGGCTGTCAAGCAAAAGAATAGGAGTGTAGTTGAGTAGCTGGTTGGCCCTACAT
TTCTGAGAAGTGACGTTACACTGGGTTGGCATAAGATATCCTAAAATCACGCTGGAACCT
TGGGCAAGGAAGAATGTGAGCAAGAGTAGAGAGAGTGCCTGGATTTCATGTCAGTGAAGC
CATGTCACCATATCATATTTTTGAATGAACTCTGAGTCAGTTGAAATAGGGTACCATCTA
GGTCAGTTTAAGAAGAGTCAGCTCAGAGAAAGCAAGCATAAGGGAAAATGTCACGTAAAC
TAGATCAGGGAACAAAATCCTCTCCTTGTGGAAATATCCCATGCAGTTTGTTGATACAAC
TTAGTATCTTATTGCCTAAAAAAAAATTTCTTATCATTGTTTCAAAAAAGCAAAATCATG
GAAAATTTTTGTTGTCCAGGCAAATAAAAGGTCATTTTAATTTAAAAAAAAAAAAAAAAA
AAAAAAAAAAAAAAAGGCCA
Rsad2 Mouse DNA
CCTATCACCATGGGGATGCTGGTGCCCACTGCTCTAGCTGCTCGGCTGCTGAGCCTGTTC
CAGCAGCAGCTGGGTTCCCTCTGGAGTGGCCTGGCCATCCTGTTCTGCTGGCTGAGAATA
GCATTAGGGTGGCTAGATCCCGGGAAGGAACAGCCACAGGTCCGGGGTGAGCTGGAGGAG
ACCCAGGAGACCCAGGAAGATGGGAACAGCACTCAGCGCACAACCCCCGTGAGTGTCAAC
TACCACTTCACTCGTCAGTGCAACTACAAATGTGGCTTCTGCTTCCACACAGCCAAGACA
TCCTTCGTGCTGCCCCTGGAGGAGGCCAAGCGAGGACTGCTTCTGCTCAAACAGGCTGGT
TTGGAGAAGATCAACTTTTCTGGAGGAGAACCCTTCCTTCAGGACAGGGGTGAATACTTG
GGCAAGCTTGTGAGATTCTGCAAGGAGGAGCTAGCCCTGCCCTCTGTGAGCATAGTGAGC
AATGGCAGCCTTATCCAGGAGAGATGGTTCAAGGACTATGGGGAGTATTTGGACATTCTT
GCTATCTCCTGCGACAGCTTCGATGAGCAGGTTAATGCTCTGATTGGCCGTGGTCAAGGA
AAAAAGAACCACGTGGAAAACCTTCAAAAGCTGAGGAGGTGGTGCAGGGATTACAAGGTG
GCTTTCAAGATCAACTCTGTCATTAATCGCTTCAACGTGGACGAAGACATGAATGAACAC
ATCAAGGCCCTGAGCCCTGTGCGCTGGAAGGTTTTCCAGTGCCTCCTAATTGAGGGTGAG
AACTCAGGAGAAGATGCCCTGAGGGAAGCAGAAAGATTTCTTATAAGCAATGAAGAATTT
GAAACATTCTTGGAGCGTCACAAAGAGGTGTCCTGTTTGGTGCCTGAATCTAACCAGAAG
ATGAAAGACTCCTACCTTATCCTAGATGAATATATGCGCTTTCTGAACTGTACCGGTGGC
CGGAAGGACCCTTCCAAGTCTATTCTGGATGTTGGCGTGGAAGAAGCAATAAAGTTCAGT
GGATTTGATGAGAAGATGTTTCTGAAGCGTGGCGGAAAGTATGTGTGGAGTAAAGCTGAC
CTGAAGCTGGACTGGTGAGGCTGAGATGGGAAGGAAACTCCGACCAGCTACAGGGACATT
CACGCCCAGCTATCCTTCAACAAGCTACATCTTCTGGCTGTCTACAGACTG
TTGTT
Rsad2 Mouse Protein
MGMLVPTALAARLLSLFQQQLGSLWSGLAILFCWLRIALGWLDPGKEQPQVRGEPEDTQE
TQEDGNSTQPTTPVSVNYHFTRQCNYKCGFCFHTAKTSFVLPLEEAKRGLLLLKQAGLEK
INFSGGEPFLQDRGEYLGKLVRFCKEELALPSVSIVSNGSLIRERWFKDYGEYLDILAIS
CDSFDEQVNALIGRGQGKKNHVENLQKLRRWCRDYKVAFKINSVINRFNVDEDMNEHIKA
LSPVRWKVFQCLLIEGENSGEDALREAERFLISNEEFETFLERHKEVSCLVPESNQKMKD
SYLILDEYMRFLNCTGGRKDPSKSILDVGVEEAIKFSGFDEKMFLKRGGKYVWSKADLKL
DW
Sgk1 Human DNA
CACGAGGGAGCGCTAACGTCTTTCTGTCTCCCCGCGGTGGTGATGACGGTGAAAACTGAG
GCTGCTAAGGGCACCCTCACTTACTCCAGGATGAGGGGCATGGTGGCAATTCTCATCGCT
TTCATGAAGCAGAGGAGGATGGGTCTGAACGACTTTATTCAGAAGATTGCCAATAACTCC
TATGCATGCAAACACCCTGAAGTTCAGTCCATCTTGAAGATCTCCCAACCTCAGGAGCCT
GAGCTTATGAATGCCAACCCTTCTCCTCCACCAAGTCCTTCTCAGCAAATCAACCTTGGC
CCGTCGTCCAATCCTCATGCTAAACCATCTGACTTTCACTTCTTGAAAGTGATCGGAAAG
GGCAGTTTTGGAAAGGTTCTTCTAGCAAGACACAAGGCAGAAGAAGTGTTCTATGCAGTC
AAAGTTTTACAGAAGAAAGCAATCCTGAAAAAGAAAGAGGAGAAGCATATTATGTCGGAG
CGGAATGTTCTGTTGAAGAATGTGAAGCACCCTTTCCTGGTGGGCCTTCACTTCTCTTTC
CAGACTGCTGACAAATTGTACTTTGTCCTAGACTACATTAATGGTGGAGAGTTGTTCTAC
CATCTCCAGAGGGAACGCTGCTTCCTGGAACCACGGGCTCGTTTCTATGCTGCTGAAATA
GCCAGTGCCTTGGGCTACCTGCATTCACTGAACATCGTTTATAGAGACTTAAAACCAGAG
AATATTTTGCTAGATTCACAGGGACACATTGTCCTTACTGATTTCGGACTCTGCAAGGAG
AACATTGAACACAACAGCACAACATCCACCTTCTGTGGCACGCCGGAGTATCTCGCACCT
GAGGTGCTTCATAAGCAGCCTTATGACAGGACTGTGGACTGGTGGTGCCTGGGAGCTGTC
TTGTATGAGATGCTGTATGGCCTGCCGCCTTTTTATAGCCGAAACACAGCTGAAATGTAC
GACAACATTCTGAACAAGCCTCTCCAGCTGAAACCAAATATTACAAATTCCGCAAGACAC
CTCCTGGAGGGCCTCCTGCAGAAGGACAGGACAAAGCGGCTCGGGGCCAAGGATGACTTC
ATGGAGATTAAGAGTCATGTCTTCTTCTCCTTAATTAACTGGGATGATCTCATTAATAAG
AAGATTACTCCCCCTTTTAACCCAAATGTGAGTGGGCCCAACGAGCTACGGCACTTTGAC
CCCGAGTTTACCGAAGAGCCTGTCCCCAACTCCATTGGCAAGTCCCCTGACAGCGTCCTC
GTCACAGCCAGCGTCAAGGAAGCTGCCGAGGCTTTCCTAGGCTTTTCCTATGCGCCTCCC
ACGGACTCTTTCCTCTGAACCCTGTTAGGGCTTGGTTTTAAAGGATTTTATGTGTGTTTC
CGAATGTTTTAGTTAGCCTTTTGGTGGAGCCGCCAGCTGACAGGACATCTTACAAGAGAA
TTTGCACATCTCTGGAAGCTTAGCAATCTTATTGCACACTGTTCGCTGGAATTTTTTGAA
GAGCACATTCTCCTCAGTGAGCTCATGAGGTTTTCATTTTTATTCTTCCTTCCAACGTGG
TGCTATCTCTGAAACGAGCGTTAGAGTGCCGCCTTAGACGGAGGCAGGAGTTTCGTTAGA
AAGCGGACCTGTTCTAAAAAAGGTCTCCTGCAGATCTGTCTGGGCTGTGATGACGAATAT
TATGAAATGTGCCTTTTCTGAAGAGATTGTGTTAGCTCCAAAGCTTTTCCTATCGCAGTG
TTTCAGTTCTTTATTTTCCCTTGTGGATATGCTGTGTGAACCGTCGTGTGAGTGTGGTAT
GCCTGATCACAGATGGATTTTGTTATAAGCATCAATGTGACACTTGCAGGACACTACAAC
GTGGGACATTGTTTGTTTCTTCCATATTTGGAAGATAAATTTATGTGTAGACTTTTTTGT
AAGATACGGTTAATAACTAAAATTTATTGAAATGGTCTTGCAATGACTCGTATTCAGATG
CCTAAAGAAAGCATTGCTGCTACAAATATTTCTATTTTTAGAAAGGGTTTTTATGGACCA
ATGCCCCAGTTGTCAGTCAGAGCCGTTGGTGTTTTTCATTGTTTAAAATGTCACCTGTAA
AATGGGCATTATTTATGTTTTTTTTTTTGCATTCCTGATAATTGTATGTATTGTATAAAG
AACGTCTGTACATTGGGTTATAACACTAGTATATTTAAACTTACAGGCTTATTTGTAATG
TAAACCACCATTTTAATGTACTGTAATTAACATGGTTATAATACGTACAATCCTTCCCTC
ATCCCATCACACAACTTTTTTTGTGTGTGATAAACTGATTTTGGTTTGCAATAAAACCTT
GAAAAATAAAAAAAAAAAAAAAAAAAAAAA
Sgk1 Mouse DNA
ACCCACGCGTCCGGCCGGTTTCACTGCTCCCCTCAGTCTCTTTTGGGCTCTTTCCGGGCA
TCGGGACGATGACCGTCAAAGCCGAGGCTGCTCGAAGCACCCTTACCTACTCCAGAATGA
GGGGAATGGTAGCGATTCTCATCGCTTTTATGAAACAGAGAAGGATGGGCCTGAACGATT
TTATTCAGAAGATTGCCAGCAACACCTATGCATGCAAACACGCTGAAGTTCAGTCCATTT
TGAAAATGTCCCATCCTCAGGAGCCGGAGCTTATGAACGCTAACCCCTCTCCTCCGCCAA
GTCCCTCTCAACAAATCAACCTGGGTCCGTCCTCCAACCCTCACGCCAAACCCTCCGACT
TTCACTTCTTGAAAGTGATCGGAAAGGGCAGTTTTGGAAAGGTTCTTCTGGCTAGGCACA
AGGCAGAAGAAGTATTCTATGCAGTCAAAGTTTTACAGAAGAAAGCCATCCTGAAGAAGA
AAGAGGAGAAGCATATTATGTCAGAGCGGAATGTTCTGTTGAAGAATGTGAAGCACCCTT
TCCTGGTGGGCCTTCACTTCTCATTCCAGACCGCTGACAAACTCTACTTTGTCCTGGACT
ACATTAATGGTGGAGAGCTGTTCTACCATCTCCAGAGGGAGCGCTGCTTCCTGGAACCAC
GGGCTCGATTCTACGCAGCTGAAATAGCCAGTGCCTTGGGCTATCTGCACTCCCTAAACA
TCGTTTATAGAGACTTAAAACCTGAGAATATTCTCCTAGACTCCCAGGGGCACATCGTCC
TCACTGACTTTGGGCTCTGCAAAGAGAATATTGAGCATAACGGGACAACATCTACCTTCT
GTGGCACGCCTGAGTATCTGGCTCCTGAGGTCCTCCATAAGCAGCCGTATGACCGGACGG
TGGACTGGTGGTGTCTTGGGGCTGTCCTGTATGAGATGCTCTACGGCCTGCCCCCGTTTT
ATAGCCGGAACACGGCTGAGATGTACGACAATATTCTGAACAAGCCTCTCCAGTTGAAAC
CAAATATTACAAACTCGGCAAGGCACCTCCTGGAAGGCCTCCTGCAGAAGGACCGGACCA
AGAGGCTGGGTGCCAAGGATGACTTTATGGAGATTAAGAGTCATATTTTCTTCTCTTTAA
TTAACTGGGATGATCTCATCAATAAGAAGATTACACCCCCATTTAACCCAAATGTGAGTG
GGCCCAGTGACCTTCGGCACTTTGATCCCGAGTTTACCGAGGAGCCGGTCCCCAGCTCCA
TCGGCAGGTCCCCTGACAGCATCCTTGTCACGGCCAGTGTGAAGGAAGCAGCAGAAGCCT
TCCTCGGCTTCTCCTATGCACCTCCTGTGGATTCCTTCCTCTGAGTGCTCCCGGGATGGT
TCTGAAGGACTTCCTCAGCGTTTCCTAAAGTGTTTTCCTTACCCTTTGGTGGAGGTTGCC
AGCTGACAGAACATTTTAAAAGAATTTGCACACCTGGAAGCTTGGCAGTCTCGCCTGCCC
GGCGTGGCGCGACGCAGCGCGCGCTGCTTGATGGGAGCTTTCCGAAGAGCACACCCTCCT
CTCAATGAGCTTGTGAGGTCTTCTTTTCTTCTCTTCCTTCCAACGTGGTGCTAGCTCCAG
GCGAGCGAGCGTGAGAGTGCCGCCTGAGACAGACACCTTGGTCTCAGTTAGAAGGAAGAT
GCAGGTCTAAGAGGAATCCCCGCAGTCTGTCTGAGCTGTGATCAAGAATATTCTGCAATG
TGCCTTTTCTGAGATCGTGTTAGCTCCAAAGCTTTTTCCTATCGCAGAGTGTTCAGTTTG
TGTTTGTTTGTTTTTGTTTTGTTTTGTTTTTCCCTTGGCGGATTTCCCGTGTGTGCAGTG
GCGTGAGTGTGCTATGCCTGATCACAGACGGTTTTGTTGTGAGCATCAATGTGACACTTG
CAGGACACTACAATGTGGGACATTGTTTGTTTCTTCCACATTTGGAAGATAAATTTATGT
GTAGACTGTTTTGTAAGATATAGTTAATAACTAAAACCTATTGAAACGGTCTTGCAATGA
CGAGCATTCAGATGCTTAAGGAAAGCATTGCTGCTACAAATATTTCTATTTTTAGAAAGG
GTTTTTATGGACCAATGCCCCAGTTGTCAGTCAAAGCCGTTGGTGTTTTCATTGTTTAAA
ATGTCACCTATAAAACGGGCATTATTTATGTTTTTTTTCCCTTTGTTCATATTCTTTTGC
ATTCCTGATTATTGTATGTATCGTGTAAAGGAAGTCTGTACATTGGGTTATAACACTAGA
TATTTAAACTTACAGGCTTATTTGTAAACCATCATTTTAATGTACTGTAATTAACATGGG
TTATAATATGTACAATTCCTCCTCCTTACCACACAACTTTTTTTGTGTGCGATAAACCAA
TTTTGGTTTGCAATAAAATCTTGAAACCT
Sgk1 Mouse Protein
MTVKAEAARSTLTYSRMRGMVAILIAFMKQRRMGLNDFIQKIASNTYACKHAEVQSILKM
SHPQEPELMNANPSPPPSPSQQINLGPSSNPHAKPSDFHFLKVIGKGSFGKVLLARHKAE
EVFYAVKVLQKKAILKKKEEKHIMSERNVLLKNVKHPFLVGLHFSFQTADKLYFVLDYIN
GGELFYHLQRERCFLEPRARFYAAEIASALGYLHSLNIVYRDLKPENILLDSQGHIVLTD
FGLCKENIEHNGTTSTFCGTPEYLAPEVLHKQPYDRTVDWWCLGAVLYEMLYGLPPFYSR
NTAEMYDNILNKPLQLKPNITNSARHLLEGLLQKDRTKRLGAKDDFMEIKSHIFFSLINW
DDLINKKITPPFNPNVSGPSDLRHFDPEFTEEPVPSSIGRSPDSILVTASVKEAAEAFLG
FSYAPPVDSFL
Sdc1 Human DNA
ATGAGACGCGCGGCGCTCTGGCTCTGGCTCTGCGCGCTGGCGCTGAGCCTGCAGCCGGCC
CTGCCGCAAATTGTGGCTACTAATTTGCCCCCTGAAGATCAAGATGGCTCTGGGGATGAC
TCTGACAACTTCTCCGGCTCAGGTGCAGGTGCTTTGCAAGATATCACCTTGTCACAGCAG
ACCCCCTCCACTTGGAAGGACACGCAGCTCCTGACGGCTATTCCCACGTCTCCAGAACCC
ACCGGCCTGGAAGCTACAGCTGCCTCCACCTCCACCCTGCCGGCTGGAGAGGGGCCCAAG
GAGGGAGAGGCTGTAGTCCTGCCAGAAGTGGAGCCTGGCCTCACCGCCCGGGAGCAGGAG
GCCACCCCCCGACCCAGGGAGACCACACAGCTCCCGACCACTCATCAGGCCTCAACGACC
ACAGCCACCACGGCCCAGGAGCCCGCCACCTCCCACCCCCACAGGGACATGCAGCCTGGC
CACCATGAGACCTCAACCCCTGCAGGACCCAGCCAAGCTGACCTTCACACTCCCCACACA
GAGGATGGAGGTCCTTCTGCCACCGAGAGGGCTGCTGAGGATGGAGCCTCCAGTCAGCTC
CCAGCAGCAGAGGGCTCTGGGGAGCAGGACTTCACCTTTGAAACCTCGGGGGAGAATACG
GCTGTAGTGGCCGTGGAGCCTGACCGCCGGAACCAGTCCCCAGTGGATCAGGGGGCCACG
GGGGCCTCACAGGGCCTCCTGGACAGGAAAGAGGTGCTGGGAGGGGTCATTGCCGGAGGC
CTCGTGGGGCTCATCTTTGCTGTGTGCCTGGTGGGTTTCATGCTGTACCGCATGAAGAAG
AAGGACGAAGGCAGCTACTCCTTGGAGGAGCCGAAACAAGCCAACGGCGGTGCCTACCAG
AAACCCACCAAGCAGGAGGAGTTCTACGCC
Sdc1 Mouse DNA
ACTCCGCGGGAGAGGTGCGGGCCAGAGGAGACAGAGCCTAACGCAGAGGAAGGGACCTGG
CAGTCGGGAGCTGACTCCAGCCGGCGAAACCTACAGCCCTCGCTCGAGAGAGCAGCGAGC
TGGGCAGGAGCCTGGGACAGCAAAGCGCAGAGCAATCAGCAGAGCCGGCCCGGAGCTCCG
TGCAACCGGCAACTCGGATCCACGAAGCCCACCGAGCTCCCGCCGCCGGTCTGGGCAGCA
TGAGACGCGCGGCGCTCTGGCTCTGGCTCTGCGCGCTGGCGCTGCGCCTGCAGCCTGCCC
TCCCGCAAATTGTGGCTGTAAATGTTCCTCCTGAAGATCAGGATGGCTCTGGGGATGACT
CTGACAACTTCTCTGGCTCTGGCACAGGTGCTTTGCCAGATACTTTGTCACGGCAGACAC
CTTCCACTTGGAAGGACGTGTGGCTGTTGACAGCCACGCCCACAGCTCCAGAGCCCACCA
GCAGCAACACCGAGACTGCTTTTACCTCTGTCCTGCCAGCCGGAGAGAAGCCCGAGGAGG
GAGAGCCTGTGCTCCATGTAGAAGCAGAGCCTGGCTTCACTGCTCGGGACAAGGAAAAGG
AGGTCACCACCAGGCCCAGGGAGACCGTGCAGCTCCCCATCACCCAACGGGCCTCAACAG
TCAGAGTCACCACAGCCCAGGCAGCTGTCACATCTCATCCGCACGGGGGCATGCAACCTG
GCCTCCATGAGACCTCGGCTCCCACAGCACCTGGTCAACCTGACCATCAGCCTCCACGTG
TGGAGGGTGGCGGCACTTCTGTCATCAAAGAGGTTGTCGAGGATGGAACTGCCAATCAGC
TTCCCGCAGGAGAGGGCTCTGGAGAACAAGACTTCACCTTTGAAACATCTGGGGAGAACA
CAGCTGTGGCTGCCGTAGAGCCCGGCCTGCGGAATCAGCCCCCGGTGGACGAAGGAGCCA
CAGGTGCTTCTCAGAGCCTTTTGGACAGGAAGGAAGTGCTGGGAGGTGTCATTGCCGGAG
GCCTAGTGGGCCTCATCTTTGCTGTGTGCCTGGTGGCTTTCATGCTGTACCGGATGAAGA
AGAAGGACGAAGGCAGCTACTCCTTGGAGGAGCCCAAACAAGCCAATGGCGGTGCCTACC
AGAAACCCACCAAGCAGGAGGAGTTCTACGCCTGATGGGGAAATAGTTCTTTCTCCCCCC
CACAGCCCCTGCCACTCACTAGGCTCCCACTTGCCTCTTCTGTGAAAAACTTCAAGCCCT
GGCCTCCCCACCACTGGGTCATGTCCTCTGCACCCAGGCCCTTCCAGCTGTTCCTGCCCG
AGCGGTCCCAGGGTGTGCTGGGAACTGATTCCCCTCCTTTGACTTCTGCCTAGAAGCTTG
GGTGCAAAGGGTTTCTTGCATCTGATCTTTCTACCACAACCACACCTGTCGTCCACTCTT
CTGACTTGGTTTCTCCAAATGGGAGGAGACCCAGCTCTGGACAGAAAGGGGACCCGACTG
CTTTGGACCTAGATGGCCTATTGCGGCTGGAGGATCCTGAGGACAGGAGAGGGGCTTCGG
CTGACCAGCCATAGCACTTACCCATAGAGACCGCTAGGGTTGGCCGTGCTGTGGTGGGGG
ATGGAGGCCTGAGCTCCTTGGAATCCACTTTTCATTGTGGGGAGGTCTACTTTAGACAAC
TTGGTTTTGCACATATTTTCTCTAATTTCTCTGTTCAGAGCCCCAGCAGACCTTATTACT
GGGGTAAGGCAAGTCTGTTGACTGGTGTCCCTCACCTCGCTTCCCTAATCTACATTCAGG
AGACCGAATCGGGGGTTAATAAGACTTTTTTTGTTTTTTGTTTTTGTTTTTAACCTAGAA
GAACCAAATCTGGACGCCAAAACGTAGGCTTAGTTTGTGTGTTGTCTCTGAGTTTGTGCT
CATGCGTACAACAGGGTATGGACTATCTGTATGGTGCCCCATTTTTGGCGGCCCGTAAGT
AGGCTAGGCTAGTCCAGGATACTGTGGAATAGCCACCTCTTGACCAGTCATGCCTGTGTG
CATGGACTCAGGGCCACGGCCTTGGCCTGGGCCACCGTGACATTGGAAGAGCCTGTGTGA
GAACTTACTCGAAGTTCACAGTCTAGGAGTGGAGGGGAGGAGACTGTAGAGTTTTGGGGG
AGGGGTAGCAAGGGTGCCCAAGCGTCTCCCACCTTTGGTACCATCTCTAGTCATCCTTCC
TCCCGGAAGTTGACAAGACACATCTTGAGTATGGCTGGCACTGGTTCCTCCATCAAGAAC
CAAGTTCACCTTCAGCTCCTGTGGCCCCGCCCCCAGGCTGGAGTCAGAAATGTTTCCCAA
AGAGTGAGTCTTTTGCTTTTGGCAAAACGCTACTTAATCCAATGGGTTCTGTACAGTAGA
TTTTGCAGATGTAATAAACTTTAATATAAAGG
Sdc1 Mouse Protein
MRRAALWLWLCALALRLQPALPQIVAVNVPPEDQDGSGDDSDNFSGSGTGALPDTLSRQT
PSTWKDVWLLTATPTAPEPTSSNTETAFTSVLPAGEKPEEGEPVLHVEAEPGFTARDKEK
EVTTRPRETVQLPITQRASTVRVTTAQAAVTSHPHGGMQPGLHETSAPTAPGQPDHQPPR
VEGGGTSVIKEVVEDGTANQLPAGEGSGEQDFTFETSGENTAVAAVEPGLRNQPPVDEGA
TGASQSLLDRKEVLGGVIAGGLVGLIFAVCLVAFMLYRMKKKDEGSYSLEEPKQANGGAY
QKPTKQEEFYA
Serpine2 Human DNA
ATGAACTGGCATCTCCCCCTCTTCCTCTTGGCCTCTGTGACGCTGCCTTCCATCTGCTCC
CACTTCAATCCTCTGTCTCTCGAGGAACTAGGCTCCAACACGGGGATCCAGGTTTTCAAT
CAGATTGTGAAGTCGAGGCCTCATGACAACATCGTGATCTCTCCCCATGGGATTGCGTCG
GTCCTGGGGATGCTTCAGCTGGGGGCGGACGGCAGGACCAAGAAGCAGCTCGCCATGGTG
ATGAGATACGGCGTAAATGGAGTTGGTAAAATATTAAAGAAGATCAACAAGGCCATCGTC
TCCAAGAAGAATAAAGACATTGTGACAGTGGCTAACGCCGTGTTTGTTAAGAATGCCTCT
GAAATTGAAGTGCCTTTTGTTACAAGGAACAAAGATGTGTTCCAGTGTGAGGTCCGGAAT
GTGAACTTTGAGGATCCAGCCTCTGCCTGTGATTCCATCAATGCATGGGTTAAAAACGAA
ACCAGGGATATGATTGACAATCTGCTGTCCCCAGATCTTATTGATGGTGTGCTCACCAGA
CTGGTCCTCGTCAACGCAGTGTATTTCAAGGGTCTGTGGAAATCACGGTTCCAACCCGAG
AACACAAAGAAACGCACTTTCGTGGCAGCCGACGGGAAATCCTATCAAGTGCCAATGCTG
GCCCAGCTCTCCGTGTTCCGGTGTGGGTCGACAAGTGCCCCCAATGATTTATGGTACAAC
TTCATTGAACTGCCCTACCACGGGGAAAGCATCAGCATGCTGATTGCACTGCCGACTGAG
AGCTCCACTCCGCTGTCTGCCATCATCCCACACATCAGCACCAAGACCATAGACAGCTGG
ATGAGCATCATGGTCCCCAAGAGGGTGCAGGTGATCCTGCCCAAGTTCACAGCTGTAGCA
CAAACAGATTTGAAGGAGCCGCTGAAAGTTCTTGGCATTACTGACATGTTTGATTCATCA
AAGGCAAATTTTGCAAAAATAACAAGGTCAGAAAACCTCCATGTTTCTCATATCTTGCAA
AAAGCAAAAATTGAAGTCAGTGAAGATGGAACCAAAGCTTCAGCAGCAACAACTGCAATT
CTCATTGCAAGATCATCGCCTCCCTGGTTTATAGTAGACAGACCTTTTCTGTTTTTCATC
CGACATAATCCTACAGGTGCTGTGTTATTCATGGGGCAGATAAACAAACC
C
Serpine2 Mouse DNA
AGTGCAGTGGTTGCACGGGAGTGCGGGCTGCACGCGTCACCGTCACCGCCGCCTGTCCCC
CACCGCCGCGCAGCGCCGATCTCCCTCCCGGTTTCGGCCGCCACCTGGGGATCCAAGCGA
GGACGGGCTGTCCTTGTTGGAAGGAACCATGAATTGGCATTTTCCTTTCTTCATCTTGAC
CACAGTGACTTTATACTCTGTGCACTCCCAGTTCAACTCTCTGTCACTGGAGGAACTAGG
CTCCAACACAGGGATCCAGGTCTTCAATCAGATCATCAAGTCACGGCCTCATGAGAACGT
TGTTGTCTCCCCACATGGGATCGCGTCCATCTTGGGCATGCTGCAGCTCGGGGCTGACGG
CAAGACAAAGAAGCAGCTCTCCACGGTGATGCGATATAATGTAAACGGAGTTGGTAAAGT
GCTGAAGAAGATCAACAAGGCTATTGTCTCCAAGAAAAATAAAGACATTGTGACCGTGGC
CAATGCTGTGTTTCTCAGGAATGGCTTTAAAATGGAAGTGCCTTTTGCAGTAAGGAACAA
AGATGTGTTTCAGTGTGAAGTGCAGAATGTGAACTTCCAGGACCCAGCCTCTGCCTCTGA
GTCCATCAATTTTTGGGTCAAAAATGAGACCAGGGGCATGATTGATAATCTGCTTTCCCC
AAATCTGATCGATGGTGCCCTTACCAGGCTGGTCCTCGTTAATGCAGTGTATTTCAAGGG
TTTGTGGAAGTCTCGGTTTCAACCAGAGAGCACAAAGAAACGGACATTCGTGGCAGGTGA
TGGGAAATCCTACCAAGTACCCATGTTGGCTCAGCTCTCTGTGTTCCGCTCAGGGTCTAC
CAGGACCCCGAATGGCTTATGGTACAACTTCATTGAGCTGCCCTACCATGGTGAGAGCAT
CAGCATGCTGATCGCCCTGCCAACAGAGAGCTCCACCCCACTGTCTGCCATCATCCCTCA
CATCACTACCAAGACCATTGATAGCTGGATGAACACCATGGTACCCAAGAGGATGCAGCT
GGTCCTACCCAAGTTCACAGCTGTGGCACAAACAGATCTGAAGGAGCCACTGAAAGCCCT
TGGCATTACTGAGATGTTTGAGCCATCAAAGGCAAATTTTACAAAAATAACAAGGTCAGA
GAGCCTTCATGTCTCTCACATCTTGCAAAAAGCAAAAATTGAAGTCAGTGAAGATGGAAC
CAAAGCTTCAGCAGCAACAACTGCAATCCTAATTGCAAGGTCATCACCTCCCTGGTTTAT
AGTAGACAGGCCTTTCCTGTTTTCCATCCGACACAATCCCACAGGTGCCATCTTGTTCCT
GGGCCAGGTGAACAAGCCCTGAAGGACAGACAAAGGAAAGCCACGCAAAGCCAAGACGAC
TTGGCTCTGAAGAGAGACTCCCTCCCCACATCTTTCATAGTTCTGTTAAATATTTTTATA
TACTGCTTTCTTTTTTGAAACTGGTTCATAGCAGCAGTTAAGTGACGCAAGTGTTTCTGG
TCGGGGCTGTGTCAGAAGAAAGGGCTGGATGCCTGGGATGCTGGATGCCTGGGATGCTGG
ATGCCTGGGATGCTGGATGCCTGGGATGCTGGATGCCTGGGATGCTGGATGCCTGGGATG
CTGTAGTGAAGGATGAGCAGGCCGGTTTCACGATGTCTAGAAGATTTCTTTAAACTACTG
ATCAGTTATCTAGGTTAACAACCCTCTCGAGTATTTGCTGTCTGTCAAGTTCAGCATCTT
TGTTTCATTCCTGTTGATATGTGTGACTTTCCAGGAGAGGATTAATCAGTGTGGCAGGAG
AGGTTAAAAAAAAAAAAGACATTTTATAGTAGTTTTTATGTTTTTATGGAAAACAATATC
ATTTGCCTTTTTAATTCTTTTTCCTCTCACTTCCACCCAAAGGCTTGAGGGTGGCAAGGG
ATGGAGCTAGCAAAAGCCGTAGCCTCTTCGTGTGTTGTTTCTGTTGCTGTTGCTCTTGTT
GTTTTATATACTGCATGTGTTCACTAAAATAAAGTTGGAAAACT
Serpine2 Mouse Protein
MNWHFPFFILTTVTLYSVHSQFNSLSLEELGSNTGIQVFNQIIKSRPHENVVVSPHGIAS
ILGMLQLGADGKTKKQLSTVMRYNVNGVGKVLKKINKAIVSKKNKDIVTVANAVFLRNGF
KMEVPFAVRNKDVFQCEVQNVNFQDPASASESINFWVKNETRGMIDNLLSPNLIDGALTR
LVLVNAVYFKGLWKSRFQPESTKKRTFVAGDGKSYQVPMLAQLSVFRSGSTRTPNGLWYN
FIELPYHGESISMLIALPTESSTPLSAIIPHITTKTIDSWMNTMVPKRMQLVLPKFTAVA
QTDLKEPLKALGITEMFEPSKANFTKITRSESLHVSHILQKAKIEVSEDGTKASAATTAI
LIARSSPPWFIVDRPFLFSIRHNPTGAILFLGQVNKP
Spp1 Human DNA
GACCAGACTCGTCTCAGGCCAGTTGCAGCCTTCTCAGCCAAACGCCGACCAAGGAAAACT
CACTACCATGAGAATTGCAGTGATTTGCTTTTGCCTCCTAGGCATCACCTGTGCCATACC
AGTTAAACAGGCTGATTCTGGAAGTTCTGAGGAAAAGCAGCTTTACAACAAATACCCAGA
TGCTGTGGCCACATGGCTAAACCCTGACCCATCTCAGAAGCAGAATCTCCTAGCCCCACA
GAATGCTGTGTCCTCTGAAGAAACCAATGACTTTAAACAAGAGACCCTTCCAAGTAAGTC
CAACGAAAGCCATGACCACATGGATGATATGGATGATGAAGATGATGATGACCATGTGGA
CAGCCAGGACTCCATTGACTCGAACGACTCTGATGATGTAGATGACACTGATGATTCTCA
CCAGTCTGATGAGTCTCACCATTCTGATGAATCTGATGAACTGGTCACTGATTTTCCCAC
GGACCTGCCAGCAACCGAAGTTTTCACTCCAGTTGTCCCCACAGTAGACACATATGATGG
CCGAGGTGATAGTGTGGTTTATGGACTGAGGTCAAAATCTAAGAAGTTTCGCAGACCTGA
CATCCAGTACCCTGATGCTACAGACGAGGACATCACCTCACACATGGAAAGCGAGGAGTT
GAATGGTGCATACAAGGCCATCCCCGTTGCCCAGGACCTGAACGCGCCTTCTGATTGGGA
CAGCCGTGGGAAGGACAGTTATGAAACGAGTCAGCTGGATGACCAGAGTGCTGAAACCCA
CAGCCACAAGCAGTCCAGATTATATAAGCGGAAAGCCAATGATGAGAGCAATGAGCATTC
CGATGTGATTGATAGTCAGGAACTTTCCAAAGTCAGCCGTGAATTCCACAGCCATGAATT
TCACAGCCATGAAGATATGCTGGTTGTAGACCCCAAAAGTAAGGAAGAAGATAAACACCT
GAAATTTCGTATTTCTCATGAATTAGATAGTGCATCTTCTGAGGTCAATTAAAAGGAGAA
AAAATACAATTTCTCACTTTGCATTTAGTCAAAAGAAAAAATGCTTTATAGCAAAATGAA
AGAGAACATGAAATGCTTCTTTCTCAGTTTATTGGTTGAATGTGTATCTATTTGAGTCTG
GAAATAACTAATGTGTTTGATAATTAGTTTAGTTTGTGGCTTCATGGAAACTCCCTGTAA
ACTAAAAGCTTCAGGGTTATGTCTATGTTCATTCTATAGAAGAAATGCAAACTATCACTG
TATTTTAATATTTGTTATTCTCTCATGAATAGAAATTTATGTAGAAGCAAACAAAATACT
TTTACCCACTTAAAAAGAGAATATAACATTTTATGTCACTATAATCTTTTGTTTTTTAAG
TTAGTGTATATTTTGTTGTGATTATCTTTTTGTGGTGTGAATAA
Spp1 Mouse DNA
CTTGCTTGGGTTTGCAGTCTTCTGCGGCAGGCATTCTCGGAGGAAACCAGCCAAGGACTA
ACTACGACCATGAGATTGGCAGTGATTTGCTTTTGCCTGTTTGGCATTGCCTCCTCCCTC
CCGGTGAAAGTGACTGATTCTGGCAGCTCAGAGGAGAAGCTTTACAGCCTGCACCCAGAT
CCTATAGCCACATGGCTGGTGCCTGACCCATCTCAGAAGCAGAATCTCCTTGCGCCACAG
AATGCTGTGTCCTCTGAAGAAAAGGATGACTTTAAGCAAGAAACTCTTCCAAGCAATTCC
AATGAAAGCCATGACCACATGGACGACGATGATGACGATGATGATGACGATGGAGACCAT
GCAGGGAGCGAGGATTCTGTGGACTCGGATGAATCTGACGAATCTCACCATTCGGATGAG
TCTGATGAGACCGTCACTGCTAGTACACAAGCAGACACTTTCACTCCAATCGTCCCTACA
GTCGATGTCCCCAACGGCCGAGGTGATAGCTTGGCTTATGGACTGAGGTCAAAGTCTAGG
AGTTTCCAGGTTTCTGATGAACAGTATCCTGATGCCACAGATGAGGACCTCACCTCTCAC
ATGAAGAGCGGTGAGTCTAAGGAGTCCCTCGATGTCATCCCTGTTGCCCAGCTTCTGAGC
ATGCCCTCTGATCAGGACAACAACGGAAAGGGCAGCCATGAGTCAAGTCAGCTGGATGAA
CCAAGTCTGGAAACACACAGACTTGAGCATTCCAAAGAGAGCCAGGAGAGTGCCGATCAG
TCGGATGTGATCGATAGTCAAGCAAGTTCCAAAGCCAGCCTGGAACATCAGAGCCACAAG
TTTCACAGCCACAAGGACAAGCTAGTCCTAGACCCTAAGAGTAAGGAAGATGATAGGTAT
CTGAAATTCCGAATTTCTCATGAATTAGAGAGTTCATCTTCTGAGGTCAACTAAAGAAGA
GGCAAAAACACAGTTCCTTACTTTGCATTTAGTAAAAACAAGAAAAAGTGTTAGTGAGGA
TTAAGCAGGAATACTAACTGCTCATTTCTCAGTTCAGTGGATATATGTATGTAGAGAAAG
AGAGGTAATATTTTGGGCTCTTAGCTTAGTCTGTTGTTTCATGCAAACAACCGTTGTAAC
CAAAAGCTTCTGCACTTTGCTTCTGTTCTTCCTGTACAAGAAATGCAAACGGCCACTGCA
TTTTAATGATTGTTATTCTTTTATGAATAAAATGTATGTAGAAACAAGCAAATTTACTGA
AACAAGCAGAATTAAAAGAGAAACTGTAACAGTCTATATCACTATACCCTTTTAGTTTTA
TAATTAGCATATATTTTGTTGTGATTATTTTTTTTGTTGGTGTGAATAAATCTTGTAACG
AATGT
Spp1 Mouse Protein
MRLAVICFCLFGIASSLPVKVTDSGSSEEKLYSLHPDPIATWLVPDPSQKQNLLAPQNAV
SSEEKDDFKQETLPSNSNESHDHMDDDDDDDDDDGDHAESEDSVDSDESDESHHSDESDE
TVTASTQADTFTPIVPTVDVPNGRGDSLAYGLRSKSRSFQVSDEQYPDATDEDLTSHMKS
GESKESLDVIPVAQLLSMPSDQDNNGKGSHESSQLDEPSLETHRLEHSKESQESADQSDV
IDSQASSKASLEHQSHKFHSHKDKLVLDPKSKEDDRYLKFRISHELESSSSEVN
Cdca8 Human DNA
GGTTGACTGTAGAGCCGCTCTCTCTCACTGGCACAGCGAGGTTTTGCTCAGCCCTTGTCT
CGGGACCGCAGGTACGTGTCTGGCGACTTCTTCGGGTGGTCCCCGTCCGCCCTCCTCGTC
CCTACCCAGTTTCTTGCTTCCCTGCCCCATCTCCGCCGCTCCCCGCAGCCTCCGCCGAGC
GCCATGGCTCCTAGGAAGGGCAGTAGTCGGGTGGCCAAGACCAACTCCTTACGGAGGCGG
AAGCTCGCCTCCTTTCTGAAAGACTTCGACCGTGAAGTGGAAATACGAATCAAGCAAATT
GAGTCAGACAGGCAGAACCTCCTCAAGGAGGTGGATAACCTCTACAACATCGAGATCCTG
CGGCTCCCCAAGGCTCTGCGCGAGATGAACTGGCTTGACTACTTCGCCCTTGGAGGAAAC
AAACAGGCCCTGGAAGAGGCGGCAACAGCTGACCTGGATATCACCGAAATAAACAAACTA
ACAGCAGAAGCTATTCAGACACCCCTGAAATCTGCCAAAACACGAAAGGTAATACAGGTA
GATGAAATGATAGTGGAAGAGGGAAGAAGGAGAAGGAAAATTTACGTAAGAATCTTCAAA
CTGCAAGAGTCAAAAGGTGTCCTCCATCCAAGAAGAGAACTCAGTCCATACAAGGCAAAG
GAAAAGGGAAAAGGTCAAGCCGTGCTAACACTGTTACCCCAGCCGTGGGCCGATTGGAGG
TGTCCATGGTCAAACCAACTCCAGGCCTGACACCCAGGTTTGACTCAAGGGTCTTCAAGA
CCCTGGCCTGCGTACTCCAGCAGCAGGAGAGCGGATTTACAACATCTCAGGGAATGGCAG
CCCTCTTGCTGACAGCAAAGAGATCTTCCTCACTGTGCCAGTGGGCGGCGGAGAGAGCCT
GCGATTATTGGCCAGTGACTTGCAGAGGCACAGTATTGCCCAGCTGGATCCAGAGGCCTT
GGGAAACATTAAGAAGCTCTCCAACCGTCTCGCCCAAATCTGCAGCAGCATACGGACCCA
CAAATGAGACACCAAAGTTGACAGGATGGACTTTTAATGGGCACTTCTGGGACCCTGAAG
AGACTTCTTCCCTTCAGGCTTATTGTTTGAGTGTGAAGTTCCAGAGCAAGGAGCCATGTT
CCTCTAAGGGAATTCAGGAATTCAGACGTGCTAGTCCCACACCAGTTAGGTAGAGCTGTC
TGTTCACCCTCCCATCCCAGCTGATCCCAGTCACTGCTTGCTGGGGCCATGCCATGGAAG
CTTCCCATCAGTCTCCCAGCTGAATCCTCCCTGCTCTCTGAGCTGCTGCCTTTTGCCTCC
TGCAACTCAACATCCTCTTCACCCTGCCCTGCCTGCAGTTGAGGGGGCGAAGAAGAACCC
TGTGTTCTCAGGAAGACTGCCTCCACCACCGCTACCCAGAGAACCTCTGCATCTGGCATT
TCTGCTCTCTATGCTTGAGACCGGGAGGTTTAGGCTCAGATAAGTGAGCTCTGGGCCATG
AGAGGGTAGGTCCAGAAGGTGGGGGGAACTGTACAGATCAGCAGAGCAGGACAGTTGGCA
GCAGTGACCTCAGTAGGGAACATGTCCGTCTACCCTCTCGCACTCATGACACCTCCCCCT
ACCAGCCTCTCTCTCTCTCACCTCCTCTGTGGGAGGTGGTCAGTGGGACTTAGGGATCTT
TCACCTGCTGTGCCCAGTAGTTCTGAAGTCTGCTTGTGGAGCAGTGTTTTATGTTTATCC
CTGTTTACTGAAGACCAAATACTGGTTTGGAGACAACTTCCATGTCTTGCTCTTCTACCT
CCCTAGTTAGTGGAAATTTGGATAAGGGAACTGTAGGGCCCAGATTCTGGAGGTTTTATG
TCATTGGCCACAGAATAACTGTCTCTAAGCTATCCATGGTCCAGTGGTCCCTGCCAAGTC
TGTAGACTTCAGAGAGCACTTCTCTCTTATGGGGTTCATGGGAACAGGGGCGGGTGTGAC
TTGCTTGGTGGCCTCATTCCATGTGTGCCTGTGCCTGGGGCATGGACTTTGTTAAGCAGA
GTCAGCAGTGAGGTCCTCATTCTCCAGCCAGCCTCTCTGCCCTGGAGAATCATGTGCTAT
GTTCTAAGAATTTGAGAACTAGAGTCCTCATCCCCAGGCTTGAAGGCACATGGCTTTCTC
ATGTAGGGCTCTCTGTGGTATTTGTTATTATTTTGCAACAAGACCATTTTAGTAAAACAG
TCCTGTTCAAGTTGTATTCTTTTAAGTTCTTTTATTCTCCTTTCCCTGAGATTTTTGTAT
ATATTGTTCTGAGTAATGGTATCTTTGAGCTGATTGTTCTAATCAGAGCTGGTACCTACT
TTCAATAAATTCTGGTTTTGTGTTTTCTTTTGT
Cdca8 Mouse DNA
GGAATTGAATTGGGTGGCGGTTAACCGAGGAGCCGCCCGTCCCTTAGTTGGAGCTGTGAG
GGTTCCTCAGACTGTGTTTTGGGACCTGCAGGTAGGTTTCGGCAGAGTTCTGGAAACCTA
GACTCCAACGACTGAACTTTCTCAGCTCTCCGACCGCTCACACCCTCTCCCCGTCTCAGT
CGCGGAGCCGGCTGCTTGGCCCCTCGCTCGACGCAGCCAGGCGCCATGGCTCCCAAGAAA
CGCAGCAGCCGCGGAACCAGGACCAACACGCTGCGGAGCCGGAAGCTCGCCTCCTTCCTG
AAGGACTTCGACCGCGAGGTGCAAGTTCGAACCAAGCAAATTGAGTCCGACAGACAGACC
CTCCTCAAGGAGGTGGAAAATCTGTACAACATCGAGATCCTTCGGCTCCCCAAGGCGCTG
CAAGGGATGAAGTGGCTTGACTACTTCGCCCTAGGAGGAAACAAGCAGGCCCTGGAAGAG
GCAGCAAAAGCTGATCGAGACATCACAGAAATAAACAATTTAACAGCTGAAGCTATTCAG
ACACCTTTGAAATCTGTTAAAAAGCGAAAGGTAATCGAGGTGGAGGAATCGATAAAGGAA
GAAGAAGAAGAGGAAGAAGAAGGAGGAGGAGAAGGAGGAAGAACAAAAAAGAGCCATAAG
AATCTTCGATCTGCAAAAGTCAAAAGATGCCTTCCATCCAAGAAGAGAACCCAGTCCATA
CAAGGAAGAGGCAGAAGTAAAAGGTTAAGCCATGACTTTGTGACGCCAGCTATGAGCAGG
CTGGAGCCGTCTCTGGTGAAACCAACCCCAGGCATGACACCTAGGTTTGACTCCCGGGTC
TTCAAGACTCCAGGGCTACGCACTCCAGCAGCCAAAGAGCAAGTTTACAACATCTCCATC
AACGGCAGCCCTCTCGCAGACAGCAAAGAGATCTCCCTCAGTGTGCCCATAGGTGGCGGT
GCGAGCTTGCGGTTATTGGCCAGTGACTTGCAAAGGATTGATATTGCTCAGCTGAATCCA
GAGGCCCTGGGAAACATTAGAAAGCTCTCGAGCCGCCTCGCCCAGATCTGCAGCAGCATA
CGGACGGGCCGATGAGAGGACAACAGGACACACAGTGGCAGCAGGGACTGTGGTAGCAGA
GTGCACACATCTGTCCTTCTTCTGTGGGGTCCTTCACTGCCAACACCTGCAACGGTGCTT
TGTCTCTCTGACAGCTATGGTGTCTTGCTGCACACTTCTAGTTAGTGGGAATTTTAGACG
GGGAACACAGGGCTAGTCAGGGCCTTTGTGTGCTTGGTGTGGAGTGACTGAGAACCGTCT
ATGGTTCAAGGTCCCACTGGGGATAAACTGCTTAGAGCACTGTCCTAGAGGGCAAGTGTA
GCCTTCGCCTCCGGGCCCAGGCAGGCTATGCAGTCAGCAGTAGGGTCTGTGCTCCATGCG
GGTCCAGGCGCACGGCTCTCCTATTCTGTTGTCATTTGTGCCCTCTATGGGCAGGTGTGT
TTCAAGTTGGTTTTCTGTTGCTGAGGCTTTCATACACATCAGTTACCATCTCAGCTGATT
TGTCTACTGAAAGCTTGCTGTTTTCAATAAATCTTAGTTTGCCATGGTTTTA
AGTC
Cdca8 Mouse Protein
MAPKKRSSRGTRTNTLRSRKLASFLKDFDREVQVRTKQIESDRQTLLKEVENLYNIEILR
LPKALQGMKWLDYFALGGNKQALEEAAKADRDITEINNLTAEAIQTPLKSVKKRKVIEVE
ESIKEEEEEEEEGGGGGGRTKKSHKNLRSAKVKRCLPSKKRTQSIQGRGRSKRLSHDFVT
PAMSRLEPSLVKPTPGMTPRFDSRVFKTPGLRTPAAKEQVYNISINGSPLADSKEISLSV
PIGGGASLRLLASDLQRIDIAQLNPEALGNIRKLSSRLAQICSSIRTGR
Nrp1 Human DNA
ATGGAGAGGGGGCTGCCGCTCCTCTGCGCCGTGCTCGCCCTCGTCCTCGCCCCGGCCGGC
GCTTTTCGCAACGATGAATGTGGCGATACTATAAAAATTGAAAGCCCCGGGTACCTTACA
TCTCCTGGTTATCCTCATTCTTATCACCCAAGTGAAAAATGCGAATGGCTGATTCAGGCT
CCGGACCCATACCAGAGAATTATGATCAACTTCAACCCTCACTTCGATTTGGAGGACAGA
GACTGCAAGTATGACTACGTGGAAGTCTTCGATGGAGAAAATGAAAATGGACATTTTAGG
GGAAAGTTCTGTGGAAAGATAGCCCCTCCTCCTGTTGTGTCTTCAGGGCCATTTCTTTTT
ATCAAATTTGTCTCTGACTACGAAACACATGGTGCAGGATTTTCCATACGTTATGAAATT
TTCAAGAGAGGTCCTGAATGTTCCCAGAACTACACAACACCTAGTGGAGTGATAAAGTCC
CCCGGATTCCCTGAAAAATATCCCAACAGCCTTGAATGCACTTATATTGTCTTTGCGCCA
AAGATGTCAGAGATTATCCTGGAATTTGAAAGCTTTGACCTGGAGCCTGACTCAAATCCT
CCAGGGGGGATGTTCTGTCGCTACGACCGGCTAGAAATCTGGGATGGATTCCCTGATGTT
GGCCCTCACATTGGGCGTTACTGTGGACAGAAAACACCAGGTCGAATCCGATCCTCATCG
GGCATTCTCTCCATGGTTTTTTACACCGACAGCGCGATAGCAAAAGAAGGTTTCTCAGCA
AACTACAGTGTCTTGCAGAGCAGTGTCTCAGAAGATTTCAAATGTATGGAAGCTCTGGGC
ATGGAATCAGGAGAAATTCATTCTGACCAGATCACAGCTTCTTCCCAGTATAGCACCAAC
TGGTCTGCAGAGCGCTCCCGCCTGAACTACCCTGAGAATGGGTGGACTCCCGGAGAGGAT
TCCTACCGAGAGTGGATACAGGTAGACTTGGGCCTTCTGCGCTTTGTCACGGCTGTCGGG
ACACAGGGCGCCATTTCAAAAGAAACCAAGAAGAAATATTATGTCAAGACTTACAAGATC
GACGTTAGCTCCAACGGGGAAGACTGGATCACCATAAAAGAAGGAAACAAACCTGTTCTC
TTTCAGGGAAACACCAACCCCACAGATGTTGTGGTTGCAGTATTCCCCAAACCACTGATA
ACTCGATTTGTCCGAATCAAGCCTGCAACTTGGGAAACTGGCATATCTATGAGATTTGAA
GTATACGGTTGCAAGATAACAGATTATCCTTGCTCTGGAATGTTGGGTATGGTGTCTGGA
CTTATTTCTGACTCCCAGATCACATCATCCAACCAAGGAGACAGAAACTGGATGCCTGAA
AACATCCGCCTGGTAACCAGTCGCTCTGGCTGGGCACTTCCACCCGCACCTCATTCCTAC
ATCAATGAGTGGCTCCAAATAGACCTGGGGGAGGAGAAGATCGTGAGGGGCATCATCATT
CAGGGTGGGAAGCACCGAGAGAACAAGGTGTTCATGAGGAAGTTCAAGATCGGGTACAGC
AACAACGGCTCGGACTGGAAGATGATCATGGATGACAGCAAACGCAAGGCGAAGTCTTTT
GAGGGCAACAACAACTATGATACACCTGAGCTGCGGACTTTTCCAGCTCTCTCCACGCGA
TTCATCAGGATCTACCCCGAGAGAGCCACTCATGGCGGACTGGGGCTCAGAATGGAGCTG
CTGGGCTGTGAAGTGGAAGCCCCTACAGCTGGACCGACCACTCCCAACGGGAACTTGGTG
GATGAATGTGATGACGACCAGGCCAACTGCCACAGTGGAACAGGTGATGACTTCCAGCTC
ACAGGTGGCACCACTGTGCTGGCCACAGAAAAGCCCACGGTCATAGACAGCACCATACAA
TCAGAGTTTCCAACATATGGTTTTAACTGTGAATTTGGCTGGGGCTCTCACAAGACCTTC
TGCCACTGGGAACATGACAATCACGTGCAGCTCAAGTGGAGTGTGTTGACCAGCAAGACG
GGACCCATTCAGGATCACACAGGAGATGGCAACTTCATCTATTCCCAAGCTGACGAAAAT
CAGAAGGGCAAAGTGGCTCGCCTGGTGAGCCCTGTGGTTTATTCCCAGAACTCTGCCCAC
TGCATGACCTTCTGGTATCACATGTCTGGGTCCCACGTCGGCACACTCAGGGTCAAACTG
CGCTACCAGAAGCCAGAGGAGTACGATCAGCTGGTCTGGATGGCCATTGGACACCAAGGT
GACCACTGGAAGGAAGGGCGTGTCTTGCTCCACAAGTCTCTGAAACTTTATCAGGTGATT
TTCGAGGGCGAAATCGGAAAAGGAAACCTTGGTGGGATTGCTGTGGATGACATTAGTATT
AATAACCACATTTCACAAGAAGATTGTGCAAAACCAGCAGACCTGGATAAAAAGAACCCA
GAAATTAAAATTGATGAAACAGGGAGCACGCCAGGATACGAAGGTGAAGGAGAAGGTGAC
AAGAACATCTCCAGGAAGCCAGGCAATGTGTTGAAGACCTTAGAACCCATCCTCATCACC
ATCATAGCCATGAGCGCCCTGGGGGTCCTCCTGGGGGCTGTCTGTGGGGTCGTGCTGTAC
TGTGCCTGTTGGCATAATGGGATGTCAGAAAGAAACTTGTCTGCCCTGGAGAACTATAAC
TTTGAACTTGTGGATGGTGTGAAGTTGAAAAAAGACAAACTGAATACACAGAGTACTTAT
TCGGAGGCATGA
Nrp1 Mouse DNA
TTTTTTTTTTTTTTTTTTTTTTTTTTTTTTTTTTTCCTCCTTCTTCTTCTTCCTGAGACA
TGGCCCGGGCAGTGGCTCCTGGAAGAGGAACAAGTGTGGGAAAAGGGAGAGGAAATCGGA
GCTAAATGACAGGATGCAGGCGACTTGAGACACAAAAAGAGAAGCGCTTCTCGCGAATTC
AGGCATTGCCTCGCCGCTAGCCTTCCCCGCCAAGACCCGCTGAGGATTTTATGGTTCTTA
GGCGGACTTAAGAGCGTTTCGGATTGTTAAGATTATCGTTTGCTGGTTTTTCGTCCGCGC
AATCGTGTTCTCCTGCGGCTGCCTGGGGACTGGCTTGGCGAAGGAGGATGGAGAGGGGGC
TGCCGTTGCTGTGCGCCACGCTCGCCCTTGCCCTCGCCCTGGCGGGCGCTTTCCGCAGCG
ACAAATGTGGCGGGACCATAAAAATCGAAAACCCAGGGTACCTCACATCTCCCGGTTACC
CTCATTCTTACCATCCAAGTGAGAAGTGTGAATGGCTAATCCAAGCTCCGGAACCCTACC
AGAGAATCATAATCAACTTCAACCCACATTTCGATTTGGAGGACAGAGACTGCAAGTATG
ACTACGTGGAAGTAATTGATGGGGAGAATGAAGGCGGCCGCCTGTGGGGGAAGTTCTGTG
GGAAGATTGCACCTTCTCCTGTGGTGTCTTCAGGGCCCTTTCTCTTCATCAAATTTGTCT
CTGACTATGAGACACATGGGGCAGGGTTTTCCATCCGCTATGAAATCTTCAAGAGAGGGC
CCGAATGTTCTCAGAACTATACAGCACCTACTGGAGTGATAAAGTCCCCTGGGTTCCCTG
AAAAATACCCCAACTGCTTGGAGTGCACCTACATCATCTTTGCACCAAAGATGTCTGAGA
TAATCCTGGAGTTTGAAAGTTTTGACCTGGAGCAAGACTCGAATCCTCCCGGAGGAATGT
TCTGTCGCTATGACCGGCTGGAGATCTGGGATGGATTCCCTGAAGTTGGCCCTCACATTG
GGCGTTATTGTGGGCAGAAAACTCCTGGCCGGATCCGCTCCTCTTCAGGCGTTCTATCCA
TGGTCTTTTACACTGACAGCGCAATAGCAAAAGAAGGTTTCTCAGCCAACTACAGTGTGC
TACAGAGCAGCATCTCTGAAGATTTTAAGTGTATGGAGGCTCTGGGCATGGAATCTGGAG
AGATCCATTCTGATCAGATCACTGCATCTTCACAGTATGGTACCAACTGGTCTGTAGAGC
GCTCCCGCCTGAACTACCCTGAAAATGGGTGGACTCCAGGAGAAGACTCCTACAAGGAGT
GGATCCAGGTGGACTTGGGCCTCCTGCGATTCGTTACTGCTGTAGGGACACAGGGTGCCA
TTTCCAAGGAAACCAAGAAGAAATATTATGTCAAGACTTACAGAGTAGACATCAGCTCCA
ACGGAGAGGACTGGATCTCCCTGAAAGAGGGAAATAAAGCCATTATCTTTCAGGGAAACA
CCAACCCCACAGATGTTGTCTTAGGAGTTTTCTCCAAACCACTGATAACTCGATTTGTCC
GAATCAAACCTGTATCCTGGGAAACTGGTATATCTATGAGATTTGAAGTTTATGGCTGCA
AGATAACAGATTATCCTTGCTCTGGAATGTTGGGCATGGTGTCTGGACTTATTTCAGACT
CCCAGATTACAGCATCCAATCAAGCCGACAGGAATTGGATGCCAGAAAACATCCGTCTGG
TGACCAGTCGTACCGGCTGGGCACTGCCACCCTCACCCCACCCATACACCAATGAATGGC
TCCAAGTGGACCTGGGAGATGAGAAGATAGTAAGAGGTGTCATCATTCAGGGTGGGAAGC
ACCGAGAAAACAAGGTGTTCATGAGGAAGTTCAAGATCGCCTATAGTAACAATGGCTCTG
ACTGGAAAACTATCATGGATGACAGCAAGCGCAAGGCTAAGTCGTTCGAAGGCAACAACA
ACTATGACACACCTGAGCTTCGGACGTTTTCACCTCTCTCCACAAGGTTCATCAGGATCT
ACCCTGAGAGAGCCACACACAGTGGGCTTGGGCTGAGGATGGAGCTACTGGGCTGTGAAG
TGGAAGCACCTACAGCTGGACCAACCACACCCAATGGGAACCCAGTGCATGAGTGTGACG
ACGACCAGGCCAACTGCCACAGTGGCACAGGTGATGACTTCCAGCTCACAGGAGGCACCA
CTGTCCTGGCCACAGAGAAGCCAACCATTATAGACAGCACCATCCAATCAGAGTTCCCGA
CATACGGTTTTAACTGCGAGTTTGGCTGGGGCTCTCACAAGACATTCTGCCACTGGGAGC
ATGACAGCCATGCACAGCTCAGGTGGAGTGTGCTGACCAGCAAGACAGGGCCGATTCAGG
ACCATACAGGAGATGGCAACTTCATCTATTCCCAAGCTGATGAAAATCAGAAAGGCAAAG
TAGCCCGCCTGGTGAGCCCTGTGGTCTATTCCCAGAGCTCTGCCCACTGTATGACCTTCT
GGTATCACATGTCCGGCTCTCATGTGGGTACACTGAGGGTCAAACTACGCTACCAGAAGC
CAGAGGAATATGATCAACTGGTCTGGATGGTGGTTGGGCACCAAGGAGACCACTGGAAAG
AAGGACGTGTCTTGCTGCACAAATCTCTGAAACTATATCAGGTTATTTTTGAAGGTGAAA
TCGGAAAAGGAAACCTTGGTGGAATTGCTGTGGATGATATCAGTATTAACAACCATATTT
CTCAGGAAGACTGTGCAAAACCAACAGACCTAGATAAAAAGAACACAGAAATTAAAATTG
ATGAAACAGGGAGCACTCCAGGATATGAAGGAGAAGGGGAAGGTGACAAGAACATCTCCA
GGAAGCCAGGCAATGTGCTTAAGACCCTGGATCCCATCCTGATCACCATCATAGCCATGA
GTGCCCTGGGAGTACTCCTGGGTGCAGTCTGTGGAGTTGTGCTGTACTGTGCCTGTTGGC
ACAATGGGATGTCAGAAAGGAACCTATCTGCCCTGGAGAACTATAACTTTGAACTTGTGG
ATGGTGTAAAGTTGAAAAAAGATAAACTGAACCCACAGAGTAATTACTCAGAGGCGTGAA
GGCACGGAGCTGGAGGGAACAAGGGAGGAGCACGGCAGGAGAACAGGTGGAGGCATGGGG
ACTCTGTTACTCTGCTTTCACTGTAAGCTGGGAAGGGCGGGGACTCTGTTACTCCGCTTT
CACTGTAAGCTCGGAAGGGCATCCACGATGCCATGCCAGGCTTTTCTCAGGAGCTTCAAT
GAGCGTCACCTACAGACACAAGCAGGTGACTGCGGTAACAACAGGAATCATGTACAAGCC
TGCTTTCTTCTCTTGGTTTCATTTGGGTAATCAGAAGCCATTTGAGACCAAGTGTGACTG
ACTTCATGGTTCATCCTACTAGCCCCCTTTTTTCCTCTCTTTCTCCTTACCCTGTGGTGG
ATTCTTCTCGGAAACTGCAAAATCCAAGATGCTGGCACTAGGCGTTATTCAGTGGGCCCT
TTTGATGGACATGTGACCTGTAGCCCAGTGCCCAGAGCATATTATCATAACCACATTTCA
GGGGACGCCAACGTCCATCCACCTTTGCATCGCTACCTGCAGCGAGCACA
GG
Nrp1 Mouse Protein
MERGLPLLCATLALALALAGAFRSDKCGGTIKIENPGYLTSPGYPHSYHPSEKCEWLIQA
PEPYQRIMINFNPHFDLEDRDCKYDYVEVIDGENEGGRLWGKFCGKIAPSPVVSSGPFLF
IKFVSDYETHGAGFSIRYEIFKRGPECSQNYTAPTGVIKSPGFPEKYPNSLECTYIIFAP
KMSEIILEFESFDLEQDSNPPGGMFCRYDRLEIWDGFPEVGPHIGRYCGQKTPGRIRSSS
GVLSMVFYTDSAIAKEGFSANYSVLQSSISEDFKCMEALGMESGEIHSDQITASSQYGTN
WSVERSRLNYPENGWTPGEDSYKEWIQVDLGLLRFVTAVGTQGAISKETKKKYYVKTYRV
DISSNGEDWISLKEGNKAIIFQGNTNPTDVVLGVFSKPLITRFVRIKPVSWETGISMRFE
VYGCKITDYPCSGMLGMVSGLISDSQITASNQADRNWMPENIRLVTSRTGWALPPSPHPY
TNEWLQVDLGDEKIVRGVIIQGGKHRENKVFMRKFKIAYSNNGSDWKTIMDDSKRKAKSF
EGNNNYDTPELRTFSPLSTRFIRIYPERATHSGLGLRMELLGCEVEAPTAGPTTPNGNPV
DECDDDQANCHSGTGDDFQLTGGTTVLATEKPTIIDSTIQSEEPTYGENCEFGWGSHKTF
CHWEHDSHAQLRWSVLTSKTGPIQDHTGDGNFIYSQADENQKGKVARLVSPVVYSQSSAH
CMTFWYHMSGSHVGTLRVKLRYQKPEEYDQLVWMVVGHQGDHWKEGRVLLHKSLKLYQVI
FEGEIGKGNLGGIAVDDISINNHISQEDCAKPTDLDKKNTEIKIDETGSTPGYEGEGEGD
KNISRKPGNVLKTLDPILITIIAMSALGVLLGAVCGVVLYCACWHNGMSERNLSALENYN
FELVDGVKLKKDKLNPQSNYSEA
Mcam Human DNA
GGGAAGCATGGGGCTTCCCAGGCTGGTCTGCGCCTTCTTGCTCGCCGCCTGCTGCTGCTG
TCCTCGCGTCGCGGGTGTGCCCGGAGAGGCTGAGCAGCCTGCGCCTGAGCTGGTGGAGGT
GGAAGTGGGCAGCACAGCCCTTCTGAAGTGCGGCCTCTCCCAGTCCCAAGGCAACCTCAG
CCATGTCGACTGGTTTTCTGTCCACAAGGAGAAGCGGACGCTCATCTTCCGTGTGCGCCA
GGGCCAGGGCCAGAGCGAACCTGGGGAGTACGAGCAGCGGCTCAGCCTCCAGGACAGAGG
GGCTACTCTGGCCCTGACTCAAGTCACCCCCCAAGACGAGCGCATCTTCTTGTGCCAGGG
CAAGCGCCCTCGGTCCCAGGAGTACCGCATCCAGCTCCGCGTCTACAAAGCTCCGGAGGA
GCCAAACATCCAGGTCAACCCCCTGGGCATCCCTGTGAACAGTAAGGAGCCTGAGGAGGT
CGCTACCTGTGTAGGGAGGAACGGGTACCCCATTCCTCAAGTCATCTGGTACAAGAATGG
CCGGCCTCTGAAGGAGGAGAAGAACCGGGTCCACATTCAGTCGTCCCAGACTGTGGAGTC
GAGTGGTTTGTACACCTTGCAGAGTATTCTGAAGGCACAGCTGGTTAAAGAAGACAAAGA
TGCCCAGTTTTACTGTGAGCTCAACTACCGGCTGCCCAGTGGGAACCACATGAAGGAGTC
CAGGGAAGTCACCGTCCCTGTTTTCTACCCGACAGAAAAAGTGTGGCTGGAAGTGGAGCC
CGTGGGAATGCTGAAGGAAGGGGACCGCGTGGAAATCAGGTGTTTGGCTGATGGCAACCC
TCCACCACACTTCAGCATCAGCAAGCAGAACCCCAGCACCAGGGAGGCAGAGGAAGAGAC
AACCAACGACAACGGGGTCCTGGTGCTGGAGCCTGCCCGGAAGGAACACAGTGGGCGCTA
TGAATGTCAGGCCTGGAACTTGGACACCATGATATCGCTGCTGAGTGAACCACAGGAACT
ACTGGTGAACTATGTGTCTGACGTCCGAGTGAGTCCCGCAGCCCCTGAGAGACAGGAAGG
CAGCAGCCTCACCCTGACCTGTGAGGCAGAGAGTAGCCAGGACCTCGAGTTCCAGTGGCT
GAGAGAAGAGACAGACCAGGTGCTGGAAAGGGGGCCTGTGCTTCAGTTGCATGACCTGAA
ACGGGAGGCAGGAGGCGGCTATCGCTGCGTGGCGTCTGTGCCCAGCATACCCGGCCTGAA
CCGCACACAGCTGGTCAAGCTGGCCATTTTTGGCCCCCCTTGGATGGCATTCAAGGAGAG
GAAGGTGTGGGTGAAAGAGAATATGGTGTTGAATCTGTCTTGTGAAGCGTCAGGGCACCC
CCGGCCCACCATCTCCTGGAACGTCAACGGCACGGCAAGTGAACAAGACCAAGATCCACA
GCGAGTCCTGAGCACCCTGAATGTCCTCGTGACCCCGGAGCTGTTGGAGACAGGTGTTGA
ATGCACGGCCTCCAACGACCTGGGCAAAAACACCAGCATCCTCTTCCTGGAGCTGGTCAA
TTTAACCACCCTCACACCAGACTCCAACACAACCACTGGCCTCAGCACTTCCACTGCCAG
TCCTCATACCAGAGCCAACAGCACCTCCACAGAGAGAAAGCTGCCGGAGCCGGAGAGCCG
GGGCGTGGTCATCGTGGCTGTGATTGTGTGCATCCTGGTCCTGGCGGTGCTGGGCGCTGT
CCTCTATTTCCTCTATAAGAAGGGCAAGCTGCCGTGCAGGCGCTCAGGGAAGCAGGAGAT
CACGCTGCCCCCGTCTCGTAAGACCGAACTTGTAGTTGAAGTTAAGTCAGATAAGCTCCC
AGAAGAGATGGGCCTCCTGCAGGGCAGCAGCGGTGACAAGAGGGCTCCGGGAGACCAGGG
AGAGAAATACATCGATCTGAGGCATTAGCCCCGAATCACTTCAGCTCCCTTCCCTGCCTG
GACCATTCCCAGCTCCCTGCTCACTCTTCTCTCAGCCAAAGCTCAAAGGGACTAGAGAGA
AGCCTCCTGCTCCCCTCGCCTGCACACCCCCTTTCAGAGGGCCACTGGGTTAGGACCTGA
GGACCTCACTTGGCCCTGCAAGGCCCGCTTTTCAGGGACCAGTCCACCACCATCTCCTCC
ACGTTGAGTGAAGCTCATCCCAAGCAAGGAGCCCCAGTCTCCCGAGCGGGTAGGAGAGTT
TCTTGCAGAACGTGTTTTTTCTTTACACACATTATGCTGTAAATACGCTCGTCCTGCCAG
CAGCTGAGCTGGGTAGCCTCTCTGAGCTGGTTTCCTGCCCCAAAGGCTGGCATTCCACCA
TCCAGGTGCACCACTGAAGTGAGGACACACCGGAGCCAGGCGCCTGCTCATGTTGAAGTG
CGCTGTTCACACCCGCTCCGGAGAGCACCCCAGCAGCATCCAGAAGCAGCTGCAGTGCAA
GCTTGCATGCCTGCGTGTTGCTGCACCACCCTCCTGTCTGCCTCTTCAAAGTCTCCTGTG
ACATTTTTTCTTTGGTCAGAGGCCAGGAACTGTGTCATTCCTTAAAGATACGTGCCGGGG
CCAGGTGTGGCTCACGCCTGTAATCCCAGCACTTTGGGAGGCCGAGGCGGCGGATCACAA
AGTCAGACGAGACCATCCTGGCTAACACGGTGAAACCCTGTCTCTACTAAAAATACAAAA
AAAAATTAGCTAGGCGTAGTGGTTGGCACCTATAGTCCCAGCTACTCGGAAGGCTGAAGC
AGGAGAATGGTATGAATCCAGGAGGTGGAGCTTGCAGTGAGCCGAGACCGTGCCACTGCA
CTCCAGCCTGGGCAACACAGCGAGACTCCGTCTCGAGCCGGCCGGTTGCGCGGGCCCTCG
GACCCTCAGAGAGGCGAGGGTTCGAGGGCACGAGTTCGAGGCCAACCTGGTCCACATGGG
TTG
Mcam Mouse DNA
CGCCCTCCGTCGGGGAAGCATGGGGCTGCCCAAACTGGTGTGCGTCTTCTTGTTCGCTGC
CTGCTGCTGCTGTCGCCGTGCCGCGGGTGTGCCAGGAGAGGAAAAGCAGCCAGTACCCAC
GCCCGACCTGGTGGAGGCAGAAGTGGGCAGCACAGCCCTTCTCAAGTGTGGCCCCTCACG
GGCCTCAGGCAACTTCAGCCAAGTGGACTGGTTTTTGATTCACAAGGAGAGGCAGATACT
GATTTTCCGTGTGCACCAAGGCAAGGGCCAGCGGGAACCTGGTGAATATGAGCACCGCCT
TAGCCTCCAAGACTCGGTGGCTACTCTGGCCCTGAGTCACGTCACTCCCCATGATGAGCG
AATGTTCCTGTGTAAGAGCAAGCGACCACGGCTCCAGGATCACTACGTTGAGCTTCAGGT
CTTCAAAGCCCCAGAGGAACCAACTATTCAAGCCAATGTCGTGGGCATCCATGTGGACAG
GCAAGAGCTCAGGGAGGTTGCTACCTGTGTGGGGAGAAACGGCTACCCCATTCCTCAAGT
CCTATGGTACAAGAACAGTCTGCCCTTGCAAGAGGAGGAGAACCGAGTTCATATCCAGTC
ATCACAGATTGTCGAGTCCAGTGGCTTGTACACCTTGAAGAGTGTTCTGAGTGCACGCCT
AGTTAAGGAAGACAAAGATGCCCAGTTTTACTGTGAACTCAGCTACCGGCTACCCAGTGG
GAACCACATGAAGGAATCTAAGGAGGTCACTGTCCCTGTTTTCTACCCTGCAGAAAAAGT
GTGGGTGGAGGTAGAGCCTGTGGGGCTGCTGAAGGAAGGGGATCATGTGACAATCAGGTG
TCTGACAGATGGCAACCCTCAACCCCACTTCACTATCAACAAGAAGGACCCCAGCACTGG
GGAGATGGAAGAGGAGAGCACCGATGAAAATGGGCTCCTGTCCTTGGAGCCTGCCGAAAA
GCACCATAGCGGGCTCTACCAGTGTCAGAGTCTGGACCTGGAAACTACCATCACACTGTC
AAGTGACCCCCTGGAGCTGCTGGTGAACTATGTGTCTGATGTTCAAGTGAATCCAACTGC
CCCTGAAGTCCAGGAAGGTGAGAGCCTCACGCTGACCTGCGAGGCAGAAAGTAACCAGGA
CCTTGAGTTTGAGTGGCTGAGAGACAAGACAGGCCAGCTGCTGGGAAAGGGTCCCGTCCT
CCAGCTAAACAACGTGAGACGGGAAGCAGGGGGACGGTATCTCTGCATGGCATCTGTCCC
CAGAGTTCCTGGCTTGAATCGTACCCAGCTGGTCAGCGTGGGCATTTTTGGGTCCCCATG
GATGGCATTAAAGGAGAGGAAGGTGTGGGTGCAAGAGAATGCAGTGCTGAATCTGTCTTG
TGAGGCTTCAGGACATCCTCAGCCCACCATCTCCTGGAATGTCAATGGTTCGGCAACTGA
ATGGAACCCAGATCCACAGACAGTAGTGAGCACCTTGAATGTCCTTGTGACGCCAGAGCT
TCTGGAGACAGGTGCAGAGTGTACAGCCTCCAACTCCCTGGGCTCAAACACCACCACCAT
TGTTCTGAAGCTGGTCACTTTAACCACCCTCATACCTGACTCCAGCCAAACCACTGGCCT
CAGCACCCTCACAGTCAGTCCTCACACCAGAGCCAACAGCACCTCCACAGAGAAAAAGCT
GCCACAGCCAGAGAGCAAAGGTGTGGTCATCGTGGCTGTGATAGTGTGTACCTTGGTGCT
TGCTGTGCTGGGTGCTGCTCTCTATTTCCTCTACAAGAAGGGCAAGCTGCCATGTGGACG
CTCGGGAAAACAGGAGATCACGCTGCCCCCGACTCGTAAGAGTGAATTTGTAGTTGAAGT
TAAGTCAGATAAGCTCCCAGAAGAGATGGCTCTCCTTCAGGGCAGCAACGGTGACAAGAG
GGCTCCAGGAGACCAGGGAGAGAAATACATCGATCTGAGGCATTAGATGGCTCCCATTGC
ACTGCTCGCAGCTCCCTGCTCAGACTTCACCCCAAGCTGAAGCCTCCAGAGGGACAGCAG
GGACGAGCCACACTCAACCCCCCCCCTGCACATCAGGTCTGAGAGCTAGGAGCTGGGACA
GGAGTCGTCTGCAGGAGCTCAGTTGGCCACAGAGGCCTGGTTTTAGAGACCAAGCCCTCC
TCTGTGTCCAGTAAATAATGCTTATCCCAAGGGGCCCGTCTCCCAGGGCATTTCCCCCTC
CCGTGCACAGCCATTGGTGGCAAATCCTTCTGCCATCAGCTGTGTGGGCTTGCCTCTTTG
AGCTCATCTCCCCTCACAGGCTGTCTTCATGATGCAGGACCTGGGCACATGGTCACATTA
TTCCGTTCACATTGGTCCTTGTGAGAACCTCACAGTCTGGAGGCGGCTGCTTTTGTACCT
TCCTGCCTGCTACTAATTCAGGGTCTCATTTGGAACATTTTTCCTTTGGGTAGTGGTCAG
GAACTGGTGTAAGTCCTCCAGACACATCCCTGTGTAAGGAAGCCAGGGCACTGTTTCTCT
GAGTTTTGTTGTTTTGTTTTCTTTGAAGGCTACTGAGCCCAAGCTTCCCGCATTCCCTTA
GTAACAAGAGACAGGACAGAGAGAAGGTCTACTGTTCATGGGGATTAGGCTTATAGGAAT
GTTAGTACCAAATTTCTACATGTGAGCTTTGGGGGCCAGGTCCTAGAGAGCCCAAGTGGG
AGAATGGTATTTAGGAGATGAAAAACCTGGCCTAGCAAGAGCTTTTGAGGTGTGTGTGTG
TGTGTGTGTATACATATATGTGTGTATATATATATATATATATATAGGTTTTGTCTGTAA
ATTTGCAAATTTTTCCTTTTATATGTGTGTTAGAAAAATAAAGTGTTATTGTCCCAAAAA
AAAAAAAAAA
Mcam Mouse Protein
MGLPKLVCVFLFAACCCCRRAAGVPGEEKQPVPTPDLVEAEVGSTALLKCGPSRASGNFS
QVDWFLIHKERQILIFRVHQGKGQREPGEYEHRLSLQDSVATLALSHVTPHDERMFLCKS
KRPRLQDHYVELQVFKAPEEPTIQANVVGIHVDRQELREVATCVGRNGYPIPQVLWYKNS
LPLQEEENRVHIQSSQIVESSGLYTLKSVLSARLVKEDKDAQFYCELSYRLPSGNHMKES
KEVTVPVFYPAEKVWVEVEPVGLLKEGDHVTIRCLTDGNPQPHFTINKKDPSTGEMEEES
TDENGLLSLEPAEKHHSGLYQCQSLDLETTITLSSDPLELLVNYVSDVQVNPTAPEVQEG
ESLTLTCEAESNQDLEFEWLRDKTGQLLGKGPVLQLNNVRREAGGRYLCMASVPRVPGLN
RTQLVSVGIFGSPWMALKERKVWVQENAVLNLSCEASGHPQPTISWNVNGSATEWNPDPQ
TVVSTLNVLVTPELLETGAECTASNSLGSNTTTIVLKLVTLTTLIPDSSQTTGLSTLTVS
PHTRANSTSTEKKLPQPESKGVVIVAVIVCTLVLAVLGAALYFFYKKGKLPCGRSGKQEI
TLPPTRKSEFVVEVKSDKLPEEMALLQGSNGDKRAPGDQGEKYIDLRH
Pbk Human DNA
GTAAGAAAGCCAGGAGGGTTCGAATTGCAACGGCAGCTGCCGGGCGTATGTGTTGGTGCT
AGAGGCAGCTGCAGGGTCTCGCTGGGGGCCGCTCGGGACCAATTTTGAAGAGGTACTTGG
CCACGACTTATTTTCACCTCCGACCTTTCCTTCCAGGCGGTGAGACTCTGGACTGAGAGT
GGCTTTCACAATGGAAGGGATCAGTAATTTCAAGACACCAAGCAAATTATCAGAAAAAAA
GAAATCTGTATTATGTTCAACTCCAACTATAAATATCCCGGCCTCTCCGTTTATGCAGAA
GCTTGGCTTTGGTACTGGGGTAAATGTGTACCTAATGAAAAGATCTCCAAGAGGTTTGTC
TCATTCTCCTTGGGCTGTAAAAAAGATTAATCCTATATGTAATGATCATTATCGAAGTGT
GTATCAAAAGAGACTAATGGATGAAGCTAAGATTTTGAAAAGCCTTCATCATCCAAACAT
TGTTGGTTATCGTGCTTTTACTGAAGCCAATGATGGCAGTCTGTGTCTTGCTATGGAATA
TGGAGGTGAAAAGTCTCTAAATGACTTAATAGAAGAACGATATAAAGCCAGCCAAGATCC
TTTTCCAGCAGCCATAATTTTAAAAGTTGCTTTGAATATGGCAAGAGGGTTAAAGTATCT
GCACCAAGAAAAGAAACTGCTTCATGGAGACATAAAGTCTTCAAATGTTGTAATTAAAGG
CGATTTTGAAACAATTAAAATCTGTGATGTAGGAGTCTCTCTACCACTGGATGAAAATAT
GACTGTGACTGACCCTGAGGCTTGTTACATTGGCACAGAGCCATGGAAACCCAAAGAAGC
TGTGGAGGAGAATGGTGTTATTACTGACAAGGCAGACATATTTGCCTTTGGCCTTACTTT
GTGGGAAATGATGACTTTATCGATTCCACACATTAATCTTTCAAATGATGATGATGATGA
AGATAAAACTTTTGATGAAAGTGATTTTGATGATGAAGCATACTATGCAGCCTTGGGAAC
TAGGCCACCTATTAATATGGAAGAACTGGATGAATCATACCAGAAAGTAATTGAACTCTT
CTCTGTATGCACTAATGAAGACCCTAAAGATCGTCCTTCTGCTGCACACATTGTTGAAGC
TCTGGAAACAGATGTCTAGTGATCATCTCAGCTGAAGTGTGGCTTGCGTAAATAACTGTT
TATTCCAAAATATTTACATAGTTACTATCAGTAGTTATTAGACTCTAAAATTGGCATATT
TGAGGACCATAGTTTCTTGTTAACATATGGATAACTATTTCTAATATGAAATATGCTTAT
ATTGGCTATAAGCACTTGGAATTGTACTGGGTTTTCTGTAAAGTTTTAGAAACTAGCTAC
ATAAGTACTTTGATACTGCTCATGCTGACTTAAAACACTAGCAGTAAAACGCTGTAAACT
GTAACATTAAATTGAATGACCATTACTTTTATTAATGATCTTTCTTAAATATTCTATATT
TTAATGGATCTACTGACATTAGCACTTTGTACAGTACAAAATAAAGTCTACATTTGTTTA
AAACAAAAAAAAAAAAAAAAAA
Pbk Mouse DNA
GAGGGGAGCTGTTCCTGCATTTTCTGGAGCGAGTCTTCTGACTGCTTTTAGTTAGAACTC
CAGTGCCCCTCGGCGGGCCGCGGCCTTTGAAAATGCGCGCGCCCTAAACGCTGCGGCGGT
TACGCTGTTGGCGGGAGGGAGCTGAGCCTGCACTTTCCGGACTAGGTGTCCAGACAGCTT
TGAGCCAGCCCGTCACTTTCACCTTTTTACCCGAGCGTGCGAGCGTGGACCTAACGTGAT
TGCTACAATGGAAGGAATTAATAATTTCAAGACGCCAAACAAATCTGAAAAAAGGAAATC
TGTATTATGTTCCACTCCATGTGTAAATATCCCTGCCTCTCCATTTATGCAGAAGCTTGG
CTTTGGGACTGGGGTCAGCGTTTACCTAATGAAAAGATCTCCAAGAGGGTTGTCTCATTC
TCCTTGGGCCGTGAAAAAGATAAGTCTTTTATGCGATGATCATTATCGAACTGTGTATCA
GAAGAGACTAACTGATGAAGCTAAGATTTTAAAAAACCTTAATCACCCAAACATTATAGG
ATATCGTGCTTTTACTGAAGCCAGTGATGGTAGTCTGTGCCTTGCTATGGAGTATGGAGG
TGAAAAGTCTCTGAATGACTTAATAGAAGAGCGGAACAAAGACAGTGGAAGTCCTTTTCC
AGCAGCTGTAATTCTCAGAGTTGCTTTGCACATGGCCAGAGGGCTAAAGTACCTGCACCA
AGAAAAGAAGCTGCTTCATGGAGACATAAAGTCTTCAAATGTTGTAATTAAAGGTGATTT
TGAAACAATTAAAATCTGTGATGTAGGAGTCTCTCTGCCATTGGATGAAAATATGACTGT
GACTGATCCTGAGGCCTGTTATATTGGTACTGAGCCATGGAAACCCAAGGAAGCGTTGGA
AGAAAATGGCATCATTACTGACAAGGCAGATGTGTTTGCTTTTGGCCTTACTCTGTGGGA
AATGATGACTTTATGTATTCCACACGTCAATCTTCCAGATGATGATGTTGATGAAGATGC
AACCTTTGATGAGAGTGACTTCGATGATGAAGCATATTATGCAGCTCTGGGGACAAGGCC
ATCCATCAACATGGAAGAGCTGGATGACTCCTACCAGAAGGCCATTGAACTCTTCTGTGT
GTGCACTAATGAGGATCCTAAAGATCGCCCGTCTGCTGCACACATCGTTGAAGCTTTGGA
ACTAGATGGCCAATGTTGTGGTCTAAGCTCAAAGCATTAACTTGTATGGGAACTGTTAAC
TAGATATATGTAGTTAATATAACTTATGGTAGCTAGATTCTAGAAGTAGCTTTAACACTA
GTGACCCCTGTCTAAGATGACTTAAGAATCAAGGGACCATTGCTTTGTTACAGATCTTTT
TAGATATTCTTGCTTCTTTAGTGGGTTACTAAAAATTTCACTACGTACATGTGGTACAGA
TATCTGTCTGCTCATAGTGTCAGTCCTTCAGCTGGCCTGTCAGCCCATGCGCCCTGGGAC
TTGAGAAGAGTTCATAAACGTAGCTCCTAGGGTGTCTTGCCTCTCTACACTTAGCTTCTA
ATTTATTACTTTGTTTCTACTGATTGTGTCTTAAGTCTTTTAAAATAAATGTAAGAATAA
ACAATAAAAGACAGTTTTAGTACCAGGCAAAAAAAAAAAAAAAAAA
Pbk Mouse Protein
MEGINNFKTPNKSEKRKSVLCSTPCVNIPASPFMQKLGFGTGVSVYLMKRSPRGLSHSPW
AVKKISLLCDDHYRTVYQKRLTDEAKILKNLNHPNIIGYRAFTEASDGSLCLAMEYGGEK
SLNDLIEERNKDSGSPFPAAVILRVALHMARGLKYLHQEKKLLHGDIKSSNVVIKGDFET
IKICDVGVSLPLDENMTVTDPEACYIGTEPWKPKEALEENGIITDKADVFAFGLTLWEMM
TLCIPHVNLPDDDVDEDATFDESDFDDEAYYAALGTRPSINMEELDDSYQKAIELFCVCT
NEDPKDRPSAAHIVEALELDGQCCGLSSKH
Akr1c1 Human DNA
CCAGAAATGGATTCGAAATATCAGTGTGTGAAGCTGAATGATGGTCACTTCATGCCTGTC
CTGGGATTTGGCACCTATGCGCCTGCAGAGGTTCCTAAAAGTAAAGCTTTAGAGGCCACC
AAATTGGCAATTGAAGCTGGCTTCCGCCATATTGATTCTGCTCATTTATACAATAATGAG
GAGCAGGTTGGACTGGCCATCCGAAGCAAGATTGCAGATGGCAGTGTGAAGAGAGAAGAC
ATATTCTACACTTCAAAGCTTTGGTGCAATTCCCATCGACCAGAGTTGGTCCGACCAGCC
TTGGAAAGGTCACTGAAAAATCTTCAATTGGATTATGTTGACCTCTACCTTATTCATTTT
CCAGTGTCTGTAAAGCCAGGTGAGGAAGTGATCCCAAAAGATGAAAATGGAAAAATACTA
TTTGACACAGTGGATCTCTGTGCCACGTGGGAGGCCGTGGAGAAGTGTAAAGATGCAGGA
TTGGCCAAGTCCATCGGGGTGTCCAACTTCAACCGCAGGCAGCTGGAGATGATCCTCAAC
AAGCCAGGGCTCAAGTACAAGCCTGTCTGCAACCAGGTGGAATGTCATCCTTACTTCAAC
CAGAGAAAACTGCTGGATTTCTGCAAGTCAAAAGACATTGTTCTGGTTGCCTATAGTGCT
CTGGGATCCCACCGAGAAGAACCATGGGTGGACCCGAACTCCCCGGTGCTCTTGGAGGAC
CCAGTCCTTTGTGCCTTGGCAAAAAAGCACAAGCGAACCCCAGCCCTGATTGCCCTGCGC
TACCAGCTACAGCGTGGGGTTGTGGTCCTGGCCAAGAGCTACAATGAGCAGCGCATCAGA
CAGAACGTGCAGGTGTTTGAATTCCAGTTGACTTCAGAGGAGATGAAAGCCATAGATGGC
CTAAACAGAAATGTGCGATATTTGACCCTTGATATTTTTGCTGGCCCCCCTAATTATCCA
TTTTCTGATGAATATTAACATGGAGGGCATTGCATGAGGTCTGCCAGAAGGCCCTGCGTG
TGGATGGTGACACAGAGGATGGCTCTATGCTGGTGACTGGACACATCGCCTCTGGTTAAA
TCTCTCCTGCTTGGTGATTTCAGCAAGCTACAGCAAAGCCCATTGGCCAGAAAGGAAAGA
CAATAATTTTGTTTTTTCATTTTGAAAAAATTAAATGCTCTCTCCTAAAGATTCTTCACC
TAAAAAA
Akr1c1 Human Protein
MDSKYQCVKLNDGHFMPVLGFGTYAPAEVPKSKALEATKLAIEAGFRHIDSAHLYNNEEQ
VGLAIRSKIADGSVKREDIFYTSKLWCNSHRPELVRPALERSLKNLQLDYVDLYLIHFPV
SVKPGEEVIPKDENGKILFDTVDLCATWEAVEKCKDAGLAKSIGVSNFNRRQLEMILNKP
GLKYKPVCNQVECHPYFNQRKLLDFCKSKDIVLVAYSALGSHREEPWVDPNSPVLLEDPV
LCALAKKHKRTPALIALRYQLQRGVVVLAKSYNEQRIRQNVQVFEFQLTSEEMKAIDGLN
RNVRYLTLDIFAGPPNYPFSDEY
Akr1c1 Mouse DNA
TTGTCCTGACTCTGTTCTGCAGCCCTGATTGATTAGTAGCAGCTTGGTTACAATACATTT
TTGTCATCTGCATTGACCTGGTCTTTAAGTTATATTGGATTTATGTTGGATTTAAGTGGA
CCCACAACACTTTGAGGAAGAAGAAGACACTCTTCTTACTTTGGAGTACCCAGTGATATC
AGGAAAGTCAGAGGCAGAGCCTGCAGATGAATCCCAAGCGCTACATGGAACTAAGTGATG
GCCACCACATTCCTGTGCTTGGCTTTGGAACCTTTGTCCCAGGAGAGGTTTCCAAGAGTA
TGGTTGCAAAAGCCACCAAAATAGCTATAGATGCTGGATTCCGCCATATTGACTCAGCTT
ATTTCTACCAAAATGAGGAGGAAGTAGGGCTGGCCATCCGAAGCAAGGTTGCTGATGGCA
CTGTGAGGAGAGAAGATATATTCTACACTTCAAAGCTTCCCTGCACATGTCATAGACCAG
AGCTGGTCCAGCCTTGCTTGGAACAATCCCTGAGAAAGCTTCAGCTGGATTATGTTGATC
TGTACCTTATTCACTGCCCAGTGTCCATGAAGCCAGGCAATGATCTTATTCCAACAGATG
AAAATGGGAAATTATTATTTGACACAGTGGATCTCTGTGACACATGGGAGGCCATGGAGA
AGTGTAAGGATTCAGGGTTAGCCAAGTCCATTGGTGTGTCCAACTTTAACCGGAGGCAGC
TGGAGATGATCCTGAACAAGCCAGGGCTCAGGTACAAGCCTGTGTGCAACCAGGTAGAGT
GTCACCCTTATCTGAACCAGAGCAAGCTCCTGGACTACTGCAAGTCAAAAGACATCGTTC
TGGTTGCCTATGGTGCTCTTGGCAGCCAACGGTGTAAGAACTGGATAGAGGAGAATGCCC
CATATCTCTTGGAAGACCCAACTCTGTGTGCCATGGCGGAAAAGCACAAGCAAACTCCGG
CCCTAATTTCCCTCCGGTATCTGCTGCAGCGTGGGATTGTCATTGTCACCAAGAGTTTCA
ATGAGAAGCGGATCAAGGAGAACCTGAAGGTCTTTGAGTTCCACTTGCCAGCAGAGGACA
TGGCAGTTATAGATAGGCTGAACAGAAACTACCGATATGCTACTGCTCGTATTATTTCTG
CTCACCCCAATTATCCATTTTTGGATGAATATTAACGCGGAAGCCTTTGTTGTGACATCG
CTCAGAGGGAGCAATGTGGGAGATGCTGTGGATGTTGATCAGCATCACCTCTGGTCGACG
TCGACATCACCGTCAACCCACACTGAACTGGATGGAGAGGGGTGGCCATGGTGTTTTGTG
ATACTTTGAAGACAATAAAGTTTTGGTCTATGAGGT
Akr1c1 Mouse Protein
MNPKRYMELSDGHHIPVLGFGTFVPGEVSKSMVAKATKIAIDAGFRHIDSAYFYQNEEEV
GLAIRSKVADGTVRREDIFYTSKLPCTCHRPELVQPCLEQSLRKLQLDYVDLYLIHCPVS
MKPGNDLIPTDENGKLLFDTVDLCDTWEAMEKCKDSGLAKSIGVSNFNRRQLEMILNKPG
LRYKPVCNQVECHPYLNQSKLLDYCKSKDIVLVAYGALGSQRCKNWIEENAPYLLEDPTL
CAMAEKHKQTPALISLRYLLQRGIVIVTKSFNEKRIKENLKVFEFHLPAEDMAVIDRLNR
NYRYATARIISAHPNYPFLDEY
Cyp1 1a1 Human DNA
GGGCGCTGAAGTGGAGCAGGTACAGTCACAGCTGTGGGGACAGCATGCTGGCCAAGGGTC
TTCCCCCACGCTCAGTCCTGGTCAAAGGCTACCAGACCTTTCTGAGTGCCCCCAGGGAGG
GGCTGGGGCGTCTCAGGGTGCCCACTGGCGAGGGAGCTGGCATCTCCACCCGCAGTCCTC
GCCCCTTCAATGAGATCCCCTCTCCTGGTGACAATGGCTGGCTAAACCTGTACCATTTCT
GGAGGGAGACGGGCACACACAAAGTCCACCTTCACCATGTCCAGAATTTCCAGAAGTATG
GCCCGATTTACAGGGAGAAGCTCGGCAACGTGGAGTCGGTTTATGTCATCGACCCTGAAG
ATGTGGCCCTTCTCTTTAAGTCCGAGGGCCCCAACCCAGAACGATTCCTCATCCCGCCCT
GGGTCGCCTATCACCAGTATTACCAGAGACCCATAGGAGTCCTGTTGAAGAAGTCGGCAG
CCTGGAAGAAAGACCGGGTGGCCCTGAACCAGGAGGTGATGGCTCCAGAGGCCACCAAGA
ACTTTTTGCCCCTGTTGGATGCAGTGTCTCGGGACTTCGTCAGTGTCCTGCACAGGCGCA
TCAAGAAGGCGGGCTCCGGAAATTACTCGGGGGACATCAGTGATGACCTGTTCCGCTTTG
CCTTTGAGTCCATCACTAACGTCATTTTTGGGGAGCGCCAGGGGATGCTGGAGGAAGTAG
TGAACCCCGAGGCCCAGCGATTCATTGATGCCATCTACCAGATGTTCCACACCAGCGTCC
CCATGCTCAACCTTCCCCCAGACCTGTTCCGTCTGTTCAGGACCAAGACCTGGAAGGACC
ATGTGGCTGCATGGGACGTGATTTTCAGTAAAGCTGACATATACACCCAGAACTTCTACT
GGGAATTGAGACAGAAAGGAAGTGTTCACCACGATTACCGTGGCATGCTCTACAGACTCC
TGGGAGACAGCAAGATGTCCTTCGAGGACATCAAGGCCAACGTCACAGAGATGCTGGCAG
GAGGGGTGGACACGACGTCCATGACCCTGCAGTGGCACTTGTATGAGATGGCACGCAACC
TGAAGGTGCAGGATATGCTGCGGGCAGAGGTCTTGGCTGCGCGGCACCAGGCCCAGGGAG
ACATGGCCACGATGCTACAGCTGGTCCCCCTCCTCAAAGCCAGCATCAAGGAGACACTAA
GACTTCACCCCATCTCCGTGACCCTGCAGAGATATCTTGTAAATGACTTGGTTCTTCGAG
ATTACATGATTCCTGCCAAGACACTGGTGCAAGTGGCCATCTATGCTCTGGGCCGAGAGC
CCACCTTCTTCTTCGACCCGGAAAATTTTGACCCAACCCGATGGCTGAGCAAAGACAAGA
ACATCACCTACTTCCGGAACTTGGGCTTTGGCTGGGGTGTGCGGCAGTGTCTGGGACGGC
GGATCGCTGAGCTAGAGATGACCATCTTCCTCATCAATATGCTGGAGAACTTCAGAGTTG
AAATCCAACACCTCAGCGATGTGGGCACCACATTCAACCTCATTCTGATGCCTGAAAAGC
CCATCTCCTTCACCTTCTGGCCCTTTAACCAGGAAGCAACCCAGCAGTGATCAGAGAGGA
TGGCCTGCAGCCACATGGGAGGAAGGCCCAGGGGTGGGGCCCATGGGGTCTCTGCATCTT
CAGTCGTCTGTCCCAAGTCCTGCTCCTTTCTGCCCAGCCTGCTCAGCAGGTTGAATGGGT
TCTCAGTGGTCACCTTCCTCAGCTCAGCTGGGCCACTCCTCTTCACCCACCCCATGGAGA
CAATAAACAGCTGAACCATCG
Cyp1 1a1 Mouse DNA
AAGTGGCAGTCGTGGGGACAGTATGCTGGCTAAAGGACTTTCCCTGCGCTCAGTGCTGGT
CAAAGGCTGCCAACCTTTCCTGAGCCCTACGTGGCAGGGTCCAGTGCTGAGTACTGGAAA
GGGAGCTGGTACCTCTACTAGCAGTCCTAGGTCCTTCAATGAGATCCCTTCCCCTGGCGA
CAATGGTTGGCTAAACCTGTACCACTTCTGGAGGGAGAGTGGCACACAGAAAATCCATTA
CCATCAGATGCAGAGTTTCCAAAAGTATGGCCCCATTTACAGGGAGAAGCTGGGCACTTT
GGAGTCAGTTTACATCGTGGACCCCAAGGATGCGTCGATACTCTTCTCATGCGAGGGTCC
CAACCCGGAGCGGTTCCTTGTGCCCCCCTGGGTGGCCTATCACCAGTATTATCAGAGGCC
CATTGGGGTCCTGTTTAAGAGTTCAGATGCCTGGAAGAAAGACCGAATCGTCCTAAACCA
AGAGGTGATGGCGCCTGGAGCCATCAAGAACTTCGTGCCCCTGCTGGAAGGTGTAGCTCA
GGACTTCATCAAAGTCTTACACAGACGCATCAAGCAGCAAAATTCTGGAAATTTCTCAGG
GGTCATCAGTGATGACCTATTCCGCTTTTCCTTTGAGTCCATCAGCAGTGTTATATTTGG
GGAGCGCATGGGGATGCTGGAGGAGATCGTGGATCCCGAGGCCCAGCGGTTCATCAATGC
TGTCTACCAGATGTTCCACACCAGTGTCCCCATGCTCAACCTGCCTCCAGACTTCTTTCG
ACTCCTCAGAACTAAGACCTGGAAGGACCATGCAGCTGCCTGGGATGTGATTTTCAATAA
AGCTGATGAGTACACCCAGAACTTCTACTGGGACTTAAGGCAGAAGCGAGACTTCAGCCA
GTACCCTGGTGTCCTTTATAGCCTCCTGGGGGGCAACAAGCTGCCCTTCAAGAACATCCA
GGCCAACATTACCGAGATGCTGGCAGGAGGGGTGGACACGACCTCCATGACCCTGCAGTG
GAACCTTTATGAGATGGCACACAACTTGAAGGTACAGGAGATGCTGCGGGCTGAAGTCCT
GGCTGCCCGGCGCCAGGCCCAGGGAGACATGGCCAAGATGGTACAGTTGGTTCCACTCCT
CAAAGCCAGCATCAAGGAGACACTGAGACTCCACCCCATCTCCGTGACCTTGCAGAGGTA
CACTGTGAATGACCTGGTGCTTCGTAATTACAAGATTCCAGCCAAGACTTTGGTACAGGT
GGCTAGCTTTGCCATGGGTCGAGATCCGGGCTTCTTTCCCAATCCAAACAAGTTTGACCC
AACTCGTTGGCTGGAAAAAAGCCAAAATACCACCCACTTCCGGTACTTGGGCTTTGGCTG
GGGTGTTCGGCAGTGTCTGGGCCGGCGGATTGCGGAGCTGGAGATGACCATCCTCCTTAT
CAATCTGCTGGAGAACTTCAGAATTGAAGTTCAAAATCTCCGTGATGTGGGGACCAAGTT
CAGCCTCATCCTGATGCCTGAGAACCCCATCCTCTTCAACTTCCAGCCTCTCAAGCAGGA
CCTGGGCCCAGCCGTGACCAGAAAAGACAACACTGTGAACTGAAGGCTGGAGTCACATGG
GGAGGTGGCCCATGGGGCATTTGAGGGTGGTATCTCTGTATCTTCAGAAACAGCACTCTG
TGATTACCTGCCCAGGTTAGCTGGGCTCTCCTCTCCTTCATCCTCTTTCCCTCTTTCCCT
ACCCAGGGAGTTAATAAACACTTGAACACTGAGG
Cyp1 1a1 Mouse Protein
MLAKGLSLRSVLVKGCQPFLSPTWQGPVLSTGKGAGTSTSSPRSFNEIPSPGDNGWLNLY
HFWRESGTQKIHYHQMQSFQKYGPIYREKLGTLESVYIVDPKDASILFSCEGPNPERFLV
PPWVAYHQYYQRPIGVLFKSSDAWKKDRIVLNQEVMAPGAIKNFVPLLEGVAQDFIKVLH
RRIKQQNSGNFSGVISDDLFRFSFESISSVIFGERMGMLEEIVDPEAQRFINAVYQMFHT
SVPMLNLPPDFFRLLRTKTWKDHAAAWDVIFNKADEYTQNFYWDLRQKRDFSQYPGVLYS
LLGGNKLPFKNIQANITEMLAGGVDTTSMTLQWNLYEMAHNLKVQEMLRAEVLAARRQAQ
GDMAKMVQLVPLLKASIKETLRLHPISVTLQRYTVNDLVLRNYKIPAKTLVQVASFAMGR
DPGFFPNPNKFDPTRWLEKSQNTTHFRYLGFGWGVRQCLGRRIAELEMTILLINLLENFR
IEVQNLRDVGTKFSLILMPENPILFNFQPLKQDLGPAVTRKDNTVN
```

1. An isolated, non-native highly engraftable hematopoietic stem cell (heHSC), wherein the heHSC is Sca-1+, c-kit+ and Lin− (SKL).

2.-7. (canceled)

8. The isolated heHSC of claim 1, wherein the heHSC is prepared by contacting hematopoietic stem cells and/or progenitor cells with at least one CXCR2 agonist and at least one CXCR4 antagonist, VLA-4 antagonist, a t antagonist, α₉β₁ integrin/VLA-4 antagonist or combination thereof.

9.-14. (canceled)

15. The isolated heHSC of claim 8, wherein the at least one CXCR2 agonist is GROβ or an analog or derivative thereof, and wherein the at least one CXCR4 antagonist is plerixafor or an analog or derivative thereof.

16.-20. (canceled)

21. The isolated heHSC of claim 1, wherein the heHSC is substantially pure.

22.-26. (canceled)

27. An isolated population of cells comprising a plurality of heHSC's of claim 1, wherein the isolated population has a unique cell surface marker expression profile as compared to a naturally occurring population of HSC.

28.-36. (canceled)

37. A method of treating a stem cell or progenitor cell disorder comprising administering a cell population comprising the isolated heHSC of claim 1 to a subject in need thereof, wherein the administered heHSC population engrafts in the subject's bone marrow compartment, thereby treating the stem cell or progenitor cell disorder.

38.-42. (canceled)

43. The method of claim 37, wherein the stem cell or progenitor cell disorder is a malignant hematologic disease or a non-malignant disease.

44-73. (canceled)

74. The isolated heHSC of claim 1; wherein the heHSC is prepared by mobilizing hematopoietic stem cells and/or progenitor cells from a bone marrow compartment of a subject to a peripheral compartment of the subject by administering at least one CXCR2 agonist and at least one CXCR4 antagonist, VLA-4 antagonist, α₉β₁ antagonist, α₉β₁ integrin/VLA-4 antagonist or combination thereof to the subject, and isolating the mobilized hematopoietic stem cells and/or progenitor cells from the peripheral compartment of the subject.

75.-82. (canceled)

83. The isolated heHSC of claim 74, wherein the at least one CXCR2 agonist is GROβ or an analog or derivative thereof, and wherein the CXCR4 antagonist is plerixafor or an analog or derivative thereof.

84.-92. (canceled)

93. The isolated heHSC of claim 74, wherein the heHSC differentially express one or more of the genes selected from the group consisting of Fos, CD93, Fosb, Dusp1, Jun, Dusp6, Cdk1, Fignl1, Plk2, Rsad2, Sgk1, Sdc1, Serpine2, Spp1, Cdca8, Nrp1, Mcam, Pbk, Akr1cl and Cyp11a1, relative to one or more genes expressed in hematopoietic stem cells (HSCs) mobilized using G-CSF.

94.-101. (canceled)

102. A method of identifying an heHSC cell population comprising
a. mobilizing hematopoietic stem cells and/or progenitor cells from a bone marrow compartment of a subject to a peripheral compartment of the subject by administering at least one CXCR2 agonist and at least one CXCR4 antagonist, VLA-4 antagonist, α₉β₁ antagonist, α₉β₁ integrin/VLA-4 antagonist or combination thereof to the subject, and isolating the mobilized hematopoietic stem cells and/or progenitor cells from the peripheral compartment of the subject;
b. mobilizing hematopoietic stem cells and/or progenitor cells from a bone marrow compartment of a subject to a peripheral compartment of the subject by a mobilization regimen not comprising a CXCR2 agonist, and isolating the mobilized hematopoietic stem cells and/or progenitor cells from the peripheral compartment of the subject;
c. comparing one or more immunophenotypical and/or functional properties of the isolated cell population of step (a) to the isolated cell population of step (b); and
d. identifying a subpopulation of the mobilized cell population of step (a) with one or more immunophenotypical and/or functional properties different than the isolated cell population of step (b).

103. The method of claim 102, wherein step (a) comprises administering at least one CXCR2 agonist and at least one CXCR4 antagonist.

104. The method of claim 102, wherein the mobilization regimen not comprising a CXCR2 agonist consists of G-CSF.

105.-173. (canceled)

174. A method of identifying an heHSC cell population comprising determining a transcriptomic signature of a population of hematopoietic stem cells (HSCs) and comparing the transcriptomic signature with a transcriptomic signature from a G-CSF mobilized population of HSCs, wherein the population of HSCs is identified as an heHSC population when the transcriptomic signature comprises a differential signature of one or more genes selected from the group consisting of Fos, CD93, Fosb, Dusp1, Jun, Dusp6, Cdk1, Fignl1, Plk2, Rsad2, Sgk1, Sdc1, Serpine2, Spp1, Cdca8, Nrp1, Mcam, Pbk, Akr1cl and Cyp11a1, relative to one or more of the genes expressed by hematopoietic stem cells mobilized using G-CSF.

175. The method of claim 174, wherein the transcriptomic signature is determined using FACs.

176. The method of claim 174, wherein the heHSC population is administered to a human subject having a stem cell or progenitor cell disorder.

177. The method of claim 176, wherein the stem cell or progenitor cell disorder is a malignant hematologic disease.

178. The method claim 177, wherein the malignant hematologic disease is selected from the group consisting of acute lymphoid leukemia, acute myeloid leukemia, chronic lymphoid leukemia, chronic myeloid leukemia, diffuse large B-cell non-Hodgkin's lymphoma, mantle cell lymphoma, lymphoblastic lymphoma, Burkitt's lymphoma, follicular B-cell non-Hodgkin's lymphoma, lymphocyte predominant nodular Hodgkin's lymphoma, multiple myeloma, and juvenile myelomonocytic leukemia.

179. The method of claim 174, further comprising transforming the population of heHSCs with an expression vector comprising a polynucleotide.

180. The method of claim 179, wherein the transformed heHSC population is administered to a human subject in need thereof.
